(19) United States
(12) Patent Application Publication
(10) Pub. No.: US 2021/0388087 A1
(43) Pub. Date: Dec. 16, 2021

(54) ANTIGEN-BINDING MOLECULES CAPABLE OF BINDING CD3 AND CD137 BUT NOT SIMULTANEOUSLY

(71) Applicant: Chugai Seiyaku Kabushiki Kaisha, Tokyo (JP)

(72) Inventors: Shu Wen Samantha Ho, Singapore (SG); Shu Feng, Singapore (SG)

(73) Assignee: Chugai Seiyaku Kabushiki Kaisha, Tokyo (JP)

(21) Appl. No.: 17/272,972

(22) PCT Filed: Sep. 27, 2019
(86) PCT No.: PCT/JP2019/038138

§ 371 Date: Mar. 3, 2021

(30) Foreign Application Priority Data
Sep. 28, 2018 (JP) 2018-185120
Jun. 4, 2019 (JP) 2019-104308

(51) Int. Cl. C07K 16/28; C07K 16/30; G01N 33/68

(52) U.S. Cl. CPC C07K 16/2809; C07K 16/2878; C07K 16/303; G01N 33/6857; A61K 2039/505; C07K 2317/75; C07K 2317/31; C07K 2317/55; C07K 2317/92

(57) ABSTRACT

The present invention relates to antigen-binding molecules binding to CD3 and CD137 (4-1BB); compositions comprising the antigen-binding molecule; and methods of using the same. The present invention provides antigen-binding molecules comprising: an antibody variable region that is capable of binding to CD3 and CD137 (4-1BB), but does not bind to CD3 and CD137 at the same time; and a variable region binding to a third antigen different from CD3 and CD137. Such antigen binding molecules exhibit enhanced T-cell dependent cytotoxity activity induced by these antigen-binding molecules through binding to the three different antigens.

### TECHNICAL FIELD

The present invention relates to antigen-binding molecules binding to CD3 and CD137 (4-1BB) and methods of using the same.

### BACKGROUND ART

Antibodies have received attention as drugs because of having high stability in plasma and producing few adverse reactions (Nat. Biotechnol. (2005) 23, 1073-1078 (NPL 1) and Eur J Pharm Biopharm. (2005) 59 (3), 389-396 (NPL 2)). The antibodies not only have an antigen-binding effect and an agonist or antagonist effect, but induce cytotoxic activity mediated by effector cells (also referred to as effector functions), such as ADCC (antibody dependent cytotoxicity), ADCP (antibody dependent cell phagocytosis), or CDC (complement dependent cytotoxicity). Particularly, antibodies of IgG1 subclass exhibit the effector functions for cancer cells. Therefore, a large number of antibody drugs have been developed in the field of oncology.

For exerting the ADCC, ADCP, or CDC of the antibodies, their Fc regions must bind to antibody receptors (Fc gamma R) present on effector cells (such as NK cells or macrophages) and various complement components. In humans, Fc gamma RIa, Fc gamma RIIa, Fc gamma RIIb, Fc gamma RIIIa, and Fc gamma RIIIb isoforms have been reported as the protein family of Fc gamma R, and their respective allotypes have also been reported (Immunol. Lett. (2002) 82, 57-65 (NPL 3)). Of these isoforms, Fc gamma RIa, Fc gamma RIIa, and Fc gamma RIIIa have, in their intracellular domains, a domain called ITAM (immunoreceptor tyrosine-based activation motif), which transduces activation signals. By contrast, only Fc gamma RIIb has, in its intracellular domain, a domain called ITIM (immunoreceptor tyrosine-based inhibitory motif), which transduces inhibition signals. These isoforms of Fc gamma R are all known to transduce signals through cross-linking by immune complexes or the like (Nat. Rev. Immunol. (2008) 8, 34-47 (NPL 4)). In fact, when the antibodies exert effector functions against cancer cells, Fc gamma R molecules on effector cell membranes are clustered by the Fc regions of a plurality of antibodies bound onto cancer cell membranes and thereby transduce activation signals through the effector cells. As a result, a cell-killing effect is exerted. In this respect, the cross-linking of Fc gamma R is restricted to effector cells located near the cancer cells, showing that the activation of immunity is localized to the cancer cells (Ann. Rev. Immunol. (1988). 6. 251-81 (NPL 5)).

Naturally occurring immunoglobulins bind to antigens through their variable regions and bind to receptors such as Fc gamma R, FcRn, Fc alpha R, and Fc epsilon R or complements through their constant regions. Each molecule of FcRn (binding molecule that interacts with an IgG Fc region) binds to each heavy chain of an antibody in a one-to-one connection. Hence, two molecules of FcRn reportedly bind to one IgG-type antibody molecule. Unlike FcRn, etc., Fc gamma R interacts with an antibody hinge region and CH2 domains, and only one molecule of Fc gamma R binds to one IgG-type antibody molecule (J. Bio. Chem., (20001) 276, 16469-16477). For the binding between Fc gamma R and the Fc region of an antibody, some amino acid residues in the hinge region and the CH2 domains of the antibody and sugar chains added to Asn 297 (EU numbering) of the CH2 domains have been found to be important (Chem. Immunol. (1997), 65, 88-110 (NPL 6), Eur. J. Immunol. (1993) 23, 1098-1104 (NPL 7), and Immunol. (1995) 86, 319-324 (NPL 8)). Fc region variants having various Fc gamma R-binding properties have previously been studied by focusing on this binding site, to yield Fc region variants having higher binding activity against activating Fc gamma R (WO2000/042072 (PTL 1) and WO2006/019447 (PTL 2)). For example, Lazar et al. have successfully increased the binding activity of human IgG1 against human Fc gamma RIIIa (V158) to approximately 370 times by substituting Ser 239, Ala 330, and Ile 332 (EU numbering) of the human IgG1 by Asn, Leu, and Glu, respectively (Proc. Natl. Acad. Sci. U.S.A. (2006) 103, 4005-4010 (NPL 9) and WO2006/019447 (PTL 2)). This altered form has approximately 9 times the binding activity of a wild type in terms of the ratio of Fc gamma RIIIa to Fc gamma IIb (A/I ratio). Alternatively, Shinkawa et al. have successfully increased binding activity against Fc gamma RIIIa to approximately 100 times by deleting fucose of the sugar chains added to Asn 297 (EU numbering) (J. Biol. Chem. (2003) 278, 3466-3473 (NPL 10)). These methods can drastically improve the ADCC activity of human IgG1 compared with naturally occurring human IgG1.

A naturally occurring IgG-type antibody typically recognizes and binds to one epitope through its variable region (Fab) and can therefore bind to only one antigen. Meanwhile, many types of proteins are known to participate in cancer or inflammation, and these proteins may crosstalk with each other. For example, some inflammatory cytokines (TNF, IL1, and IL6) are known to participate in immunological disease (Nat. Biotech., (2011) 28, 502-10 (NPL 11)). Also, the activation of other receptors is known as one mechanism underlying the acquisition of drug resistance by cancer (Endocr Relat Cancer (2006) 13, 45-51 (NPL 12)). In such a case, the usual antibody, which recognizes one epitope, cannot inhibit a plurality of proteins.

Antibodies that bind to two or more types of antigens by one molecule (these antibodies are referred to as bispecific antibodies) have been studied as molecules inhibiting a plurality of targets. Binding activity against two different antigens (first antigen and second antigen) can be conferred by the modification of naturally occurring IgG-type antibodies (mAbs. (2012) March 1, 4 (2)). Therefore, such an antibody has not only the effect of neutralizing these two or more types of antigens by one molecule but the effect of enhancing antitumor activity through the cross-linking of cells having cytotoxic activity to cancer cells. A molecule with an antigen-binding site added to the N or C terminus of an antibody (DVD-Ig, TCB and scFv-IgG), a molecule having different sequences of two Fab regions of an antibody (common L-chain bispecific antibody and hybrid hybridoma), a molecule in which one Fab region recognizes two antigens (two-in-one IgG and DutaMab), and a molecule having a CH3 domain loop as another antigen-binding site (Fcab) have previously been reported as molecular forms of the bispecific antibody (Nat. Rev. (2010), 10, 301-316 (NPL 13) and Peds (2010), 23 (4), 289-297 (NPL 14)). Since any of these bispecific antibodies interact at their Fc regions with Fc gamma R, antibody effector functions are preserved therein.

Provided that all the antigens recognized by the bispecific antibody are antigens specifically expressed in cancer, the bispecific antibody binding to any of the antigens exhibits cytotoxic activity against cancer cells and can therefore be expected to have a more efficient anticancer effect than that of the conventional antibody drug that recognizes one antigen. However, in the case where any one of the antigens recognized by the bispecific antibody is expressed in a normal tissue or is a cell expressed on immunocytes, damage on the normal tissue or release of cytokines occurs due to cross-linking with Fc gamma R (J. Immunol. (1999) August 1, 163 (3), 1246-52 (NPL 15)). As a result, strong adverse reactions are induced.

For example, catumaxomab is known as a bispecific antibody that recognizes a protein expressed on T cells and a protein expressed on cancer cells (cancer antigen). Catumaxomab binds, at two Fabs, the cancer antigen (EpCAM) and a CD3 epsilon chain expressed on T cells, respectively. Catumaxomab induces T cell-mediated cytotoxic activity through binding to the cancer antigen and the CD3 epsilon at the same time and induces NK cell- or antigen-presenting cell (e.g., macrophage)-mediated cytotoxic activity through binding to the cancer antigen and Fc gamma R at the same time. By use of these two cytotoxic activities, catumaxomab exhibits a high therapeutic effect on malignant ascites by intraperitoneal administration and has thus been approved in Europe (Cancer Treat Rev. (2010) October 36 (6), 458-67 (NPL 16)). In addition, the administration of catumaxomab reportedly yields cancer cell-reactive antibodies in some cases, demonstrating that acquired immunity is induced (Future Oncol. (2012) January 8 (1), 73-85 (NPL 17)). From this result, such antibodies having both of T cell-mediated cytotoxic activity and the effect brought about by cells such as NK cells or macrophages via Fc gamma R (these antibodies are particularly referred to as trifunctional antibodies) have received attention because a strong antitumor effect and induction of acquired immunity can be expected.

The trifunctional antibodies, however, bind to CD3 epsilon and Fc gamma R at the same time even in the absence of a cancer antigen and therefore cross-link CD3 epsilon-expressing T cells to Fc gamma R-expressing cells even in a cancer cell-free environment to produce various cytokines in large amounts. Such cancer antigen-independent induction of production of various cytokines restricts the current administration of the trifunctional antibodies to an intraperitoneal route (Cancer Treat Rev. 2010 October 36 (6), 458-67 (NPL 16)). The trifunctional antibodies are very difficult to administer systemically due to serious cytokine storm-like adverse reactions (Cancer Immunol Immunother. 2007 September; 56 (9): 1397-406 (NPL 18)).

The bispecific antibody of the conventional technique is capable of binding to both antigens, i.e., a first antigen cancer antigen (EpCAM) and a second antigen CD3 epsilon, at the same time with binding to Fc gamma R, and therefore, cannot circumvent, in view of its molecular structure, such adverse reactions caused by the binding to Fc gamma R and the second antigen CD3 epsilon at the same time.

In recent years, a modified antibody that causes cytotoxic activity mediated by T cells while circumventing adverse reactions has been provided by use of an Fc region having reduced binding activity against Fc gamma R (WO2012/073985).

Even such an antibody, however, fails to act on two immunoreceptors, i.e., CD3 epsilon and Fc gamma R, while binding to the cancer antigen, in view of its molecular structure.

An antibody that exerts both of cytotoxic activity mediated by T cells and cytotoxic activity mediated by cells other than the T cells in a cancer antigen-specific manner while circumventing adverse reactions has not yet been known.

T cells play important roles in tumor immunity, and are known to be activated by two signals: 1) binding of a T cell receptor (TCR) to an antigenic peptide presented by major histocompatibility complex (MHC) class I molecules and activation of TCR; and 2) binding of a costimulator on the surface of T cells to the ligands on antigen-presenting cells and activation of the costimulator. Furthermore, activation of molecules belonging to the tumor necrosis factor (TNF) superfamily and the TNF receptor superfamily, such as CD137(4-1BB) on the surface of T cells, has been described as important for T cell activation (Vinay, 2011, Cellular & Molecular Immunology, 8, 281-284 (NPL 19)).

CD137 agonist antibodies have already been demonstrated to show anti-tumor effects, and this has been shown experimentally to be mainly due to activation of CD8-positive T cells and NK cells (Houot, 2009, Blood, 114, 3431-8 (NPL 20)). It is also understood that T cells engineered to have chimeric antigen receptor molecules (CAR-T cells) which consist of a tumor antigen-binding domain as an extracellular domain and the CD3 and CD137 signal transducing domains as intracellular domains can enhance the persistence of the efficacy (Porter, N ENGL J MED, 2011, 365; 725-733 (NPL 21)). However, side effects of such CD137 agonist antibodies due to their non-specific hepatotoxicity have been a problem clinically and non-clinically, and development of pharmaceutical agents has not advanced (Dubrot, Cancer Immunol. Immunother., 2010, 28, 512-22 (NPL 22)). The main cause of the side effects has been suggested to involve binding of the antibody to the Fc gamma receptor via the antibody constant region (Schabowsky, Vaccine, 2009, 28, 512-22 (NPL 23)). Furthermore, it has been reported that for agonist antibodies targeting receptors that belong to the TNF receptor superfamily to exert an agonist activity in vivo, antibody crosslinking by Fc gamma receptor-expressing cells (Fc gamma RII-expressing cells) is necessary (Li, Proc Natl Acad Sci USA. 2013, 110(48), 19501-6 (NPL 24)). WO2015/156268 (PTL 3) describes that a bispecific antibody which has a binding domain with CD137 agonistic activity and a binding domain to a tumor specific antigen can exert CD137 agonistic activity and activate immune cells only in the presence of cells expressing the tumor specific antigen, by which hepatotoxic adverse events of CD137 agonist antibody can be avoided while retaining the anti-tumor activity of the antibody. WO2015/156268 further describes that the anti-tumor activity can be further enhanced and these adverse events can be avoided by using this bispecific antibody in combination with another bispecific antibody which has a binding domain with CD3 agonistic activity and a binding domain to a tumor specific antigen. A tri-specific antibody which has three binding domains to CD137, CD3 and a tumor specific antigen (EGFR) has also been reported (WO2014/116846 (PTL 4)). However, an antibody that exerts both cytotoxic activity mediated by T cells and activation activity of T cells and other immune cells via CD137 in a cancer antigen-specific manner while circumventing adverse reactions has not yet been known.

### CITATION LIST

### Patent Literature

[PTL 1] WO2000/042072
[PTL 2] WO2006/019447
[PTL 3] WO2015/156268
[PTL 4] WO2014/116846

### Non Patent Literature

[NPL 1] Nat. Biotechnol. (2005) 23, 1073-1078
[NPL 2] Eur J Pharm Biopharm. (2005) 59 (3), 389-396
[NPL 3] Immunol. Lett. (2002) 82, 57-65
[NPL 4] Nat. Rev. Immunol. (2008) 8, 34-47
[NPL 5] Ann. Rev. Immunol. (1988). 6. 251-81
[NPL 6] Chem. Immunol. (1997), 65, 88-110
[NPL 7] Eur. J. Immunol. (1993) 23, 1098-1104
[NPL 8] Immunol. (1995) 86, 319-324
[NPL 9] Proc. Natl. Acad. Sci. U.S.A. (2006) 103, 4005-4010
[NPL 10] J. Biol. Chem. (2003) 278, 3466-3473
[NPL 11] Nat. Biotech., (2011) 28, 502-10
[NPL 12] Endocr Relat Cancer (2006) 13, 45-51
[NPL 13] Nat. Rev. (2010), 10, 301-316
[NPL 14] Peds (2010), 23 (4), 289-297
[NPL 15] J. Immunol. (1999) August 1, 163 (3), 1246-52
[NPL 16] Cancer Treat Rev. (2010) October 36 (6), 458-67
[NPL 17] Future Oncol. (2012) Jan. 8 (1), 73-85
[NPL 18] Cancer Immunol Immunother. 2007 September; 56 (9): 1397-406
[NPL 19] Vinay, 2011, Cellular & Molecular Immunology, 8, 281-284
[NPL 20] Houot, 2009, Blood, 114, 3431-8
[NPL 21] Porter, N ENGL J MED, 2011, 365; 725-733
[NPL 22] Dubrot, Cancer Immunol. Immunother., 2010, 28, 512-22
[NPL 23] Schabowsky, Vaccine, 2009, 28, 512-22
[NPL 24] Li, Proc Natl Acad Sci USA. 2013, 110(48), 19501-6

### SUMMARY OF INVENTION

### Technical Problem

Tri-specific antibodies comprising a tumor-specific antigen (EGFR)-binding domain, a CD137-binding domain, and a CD3-binding domain were already reported (WO2014116846). However, since antibodies with such a molecular format can bind to three different antigens at the same time, the present inventors speculated that those tri-specific antibodies could result in cross-linking between CD3 epsilon-expressing T cells and CD137-expressing cells (e.g. T cells, B cells, NK cells, DCs etc.) by binding to CD3 and CD137 at the same time.

Furthermore, it was already reported that bispecific antibodies against CD8 and CD3 epsilon induced mutual cytotoxicity among CD8 positive T cells because the antibodies cross-linked them (Wong, Clin. Immunol. Immunopathol. 1991, 58(2), 236-250). Therefore, the present inventors speculated that bispecific antibodies against a molecule expressed on T cells and CD3 epsilon would also induce mutual cytotoxicity among T cells because they would cross-link cells expressing the molecule and CD3 epsilon.

### Solution to Problem

The present invention provides antigen-binding domains binding to CD3 and CD137 and methods of using the same. The invention also provides methods to obtain antigen binding molecules which induce T-cell dependent cytotoxity more efficiently.

The present inventors have successfully prepared an antigen-binding molecule comprising: an antibody variable region that is capable of binding to CD3 and CD137 (4-1BB), but does not bind to CD3 and CD137 at the same time; and a variable region binding to a third antigen different from CD3 and CD137, preferably a molecule specifically expressed in a cancer tissue, more preferably Glypican-3 (GPC3). By improving the binding activity to CD3 and/or CD137, the inventors have successfully prepared antigen binding molecules that exhibit enhanced T-cell dependent cytotoxity activity induced by these antigen-binding molecules through binding to the three different antigens. Such antigen-binding molecules could be used in immunotherapy while capable of circumventing the cross-linking between different cells resulting from the binding of a conventional multispecific antigen-binding molecule to antigens expressed on the different cells, which is considered to be responsible for adverse reactions when the multispecific antigen-binding molecule is used as a drug.

More specifically, the present invention provides the following:

[1] An antigen-binding molecule comprising:

an antibody variable region that is capable of binding to CD3 and CD137, but does not bind to CD3 and CD137 at the same time; wherein the antigen-binding molecule binds to CD137 with an equilibrium dissociation constant (KD) of less than 5×10⁻⁶ M, less than 5×10⁻⁷ M, less than 5×10⁸M or less than 3×10⁸ M; preferably as measured by SPR at the following condition:

37 degrees C., pH 7.4, 20 mM ACES, 150 mM NaCl, 0.05% Tween 20, 0.005% NaN3; the antigen-binding molecule is immobilized on a CM4 sensor chip, the antigen serves as analyte.

[1A] The antigen-binding molecule of [1], wherein the antigen-binding molecule binds to CD137 with an equilibrium dissociation constant (KD) between 5×10⁻⁶ M and 3×10⁻⁸M; preferably as measured by SPR at the following condition: 37 degrees C., pH 7.4, 20 mM ACES, 150 mM NaCl, 0.05% Tween 20, 0.005% NaN3; the antigen-binding molecule is immobilized on a CM4 sensor chip, the antigen serves as analyte. [1B] The antigen-binding molecule of [1] to [1A], wherein the antigen-binding molecule binds to CD3 with an equilibrium dissociation constant (KD) between 2×10⁻⁶ M and 1×10⁻⁸M, preferably as measured by SPR at the following condition: 25 degrees C., pH 7.4, 20 mM ACES, 150 mM NaCl, 0.05% Tween 20, 0.005% NaN3; the antigen-binding molecule is immobilized on a CM4 sensor chip, the antigen serves as analyte.

[2] The antigen-binding molecule of [1] to [1B], wherein the antigen-binding molecule binds to

(a) at least one, two, three or more amino acid residues of the extracellular domain of CD3 epsilon (CD3 epsilon) comprising the amino acid sequence of SEQ ID NO: 159; and/or
(b) at least one, two, three or more amino acid residues of the N-terminal region of CD137 comprising the amino acid sequence of LQDPCSNCPAGTFCDNNRNQICSPCPPNSFSSAGGQRTCDICRQCKGVFRTRKECSSTSNAEC (SEQ ID NO: 152), preferably LQDPCSN, NNRNQI and/or GQRTCDI of human CD137.

[3] The antigen-binding molecule of [1] to [2], wherein the antibody variable region is an antibody variable region having alteration of 1 to 25 amino acids, wherein the amino acid to be altered is an amino acid in a loop, an amino acid in a FR3 region, or an amino acid selected from Kabat numbering positions 31 to 35, 50 to 65, 71 to 74, and 95 to 102 in an antibody H chain variable domain, and Kabat numbering positions 24 to 34, 50 to 56, and 89 to 97 in an L chain variable domain.

[3A] The antigen-binding molecule of any of [1] to [3], wherein the heavy chain variable domain (VH) and/or the light chain variable domain (VL) comprise(s) one or more amino acid substitution selected from Table 1.3 (a) to Table 1.3 (d), wherein the one or more amino acid substitution shows at least 0.2, 0.3, 0.5, 0.8, 1, 1.5 or 2-fold binding affinity increase to CD3 and/or CD137 as set forth in Table 1.3 (a) to Table 1.3 (d). In some embodiments, it is preferable that the antibody variable region comprises:

(a) a heavy chain variable domain amino acid sequence comprising, at each of the following positions (all by Kabat numbering), one or more of the following amino acid residues indicated for that position:
A, D, E, I, G, K, L, M, N, R, T, W or Y at the amino acid position 26;
D, F, G, I, M or L, at the amino acid position 27;
D, E, F, G, H, I, K, L, M, N, P, Q, R, S, T, V, W or Y at the amino acid position 28;
F or W at the amino acid position 29;
A, D, E, F, G, H, I, K, L, M, N, P, Q, R, S, T, V, W or Y at the amino acid position 30;
F, I, N, R, S, T or V at the amino acid position 31;
A, H, I, K, L, N, Q, R, S, T or V at the amino acid position 32;
W at the amino acid position 33;
F, I, L, M or V at the amino acid position 34;
F, H, S, T, V or Y at the amino acid position 35;
E, F, H, I, K, L, M, N, Q, S, T, W or Y at the amino acid position 50;
I, K or V at the amino acid position 51;
K, M, R, or T at the amino acid position 52;
A, E, F, G, H, I, K, L, M, N, P, Q, R, S, V, W or Y at the amino acid position 52b;
A, D, E, F, G, H, I, K, L, M, N, P, Q, R, S, T, V, W or Y at the amino acid position 52c;
A, E, F, H, K, L, M, N, Q, R, S, T, V, W or Y at the amino acid position 53;
A, D, E, F, G, H, I, K, L, M, N, Q, R, S, T, V, W or Y at the amino acid position 54;
E, F, G, H, L, M, N, Q, W or Y at the amino acid position 55;
A, D, E, F, G, H, I, K, L, M, N, Q, R, S, T, V, W or Y at the amino acid position 56;
A, D, E, G, H, I, K, L, M, N, P, Q, R, S, T or V at the amino acid position 57;
A, F, H, K, N, P, R or Y at the amino acid position 58;
A, D, E, F, G, H, I, K, L, M, N, P, Q, R, S, T, V, W or Y at the amino acid position 59;
A, D, E, F, G, H, I, K, L, M, N, P, Q, R, S, T, V, W or Y at the amino acid position 60;
A, D, E, F, G, H, I, K, L, M, N, P, Q, R, S, T, V, W or Y at the amino acid position 61;
A, D, E, F, G, H, I, K, L, M, N, P, Q, R, S, T, V, W or Y at the amino acid position 62;
A, D, E, F, G, H, I, K, L, M, N, P, Q, R, S, T, V, W or Y at the amino acid position 63;
A, D, E, F, G, H, I, K, L, M, N, P, Q, R, S, T, V, W or Y at the amino acid position 64;
A, D, E, F, G, H, I, K, L, M, N, P, Q, R, S, T, V, W or Y at the amino acid position 65;
H or R at the amino acid position 93;
F, G, H, L, M, S, T, V or Y at the amino acid position 94;
I or V at the amino acid position 95;
F, H, I, K, L, M, T, V, W or Y at the amino acid position 96;
F, Y or W at the amino acid position 97;
A, F, G, H, I, K, L, M, N, Q, R, S, T, V, W or Y at the amino acid position 98;
A, F, G, H, I, K, L, M, N, P, Q, R, S, T, V, W or Y at the amino acid position 99;
A, D, E, F, G, H, I, K, L, M, N, P, Q, R, S, T, V, W or Y at the amino acid position 100;
A, D, E, F, G, H, I, K, L, M, N, P, Q, R, S, T, V, W or Y at the amino acid position 100a;
A, D, E, F, G, H, I, K, L, M, N, P, Q, R, S, T, V, W or Y at the amino acid position 100b;
A, D, E, F, G, H, I, K, L, M, N, P, Q, R, S, T, V, W or Y at the amino acid position 100c;
A, D, E, F, G, H, I, K, L, M, N, P, Q, R, S, T, V, W or Y at the amino acid position 100d;
A, D, E, F, G, H, I, K, L, M, P, Q, R, S, T, V, W or Y at the amino acid position 100e;
A, E, F, G, H, I, K, L, M, N, P, Q, R, S, T, V, W or Y at the amino acid position 100f;
A, E, F, G, H, I, K, L, M, N, P, Q, R, S, T, V, W or Y at the amino acid position 100g;
A, D, E, G, H, I, L, M, N, P, S, T or V at the amino acid position 100h;
A, D, E, F, G, H, I, K, L, M, N, P, Q, R, S, T, V, W or Y at the amino acid position 100i;
A, D, F, I, L, M, N, Q, S, T or V at the amino acid position 101;
A, D, E, F, G, H, I K, L, M, N, Q, R, S, T, V, W or Y at the amino acid position 102; and/or
(b) a light chain variable domain amino acid sequence comprising, at each of the following positions (all by Kabat numbering), one or more of the following amino acid residues indicated for that position:
A, D, F, G, H, I, K, L, M, N, Q, R, S, T, V, W or Y at the amino acid position 24;
A, G, N, P, S, T or V at the amino acid position 25;
A, D, E, F, G, I, K, L, M, N, Q, R, S, T or V at the amino acid position 26;
A, D, E, F, G, H, I, K, L, M, N, Q, R, S, T, V, W or Y at the amino acid position 27;
A, D, E, F, G, H, I, K, L, M, N, P, Q, R, S, T, V, W or Y at the amino acid position 27a;
A, I, L, M, P, T or V at the amino acid position 27b;
A, E, F, H, I, K, L, M, N, P, Q, R, T, W or Y at the amino acid position 27c;
A, E, G, H, I, K, L, M, N, P, Q, R, S, T, V, W or Y at the amino acid position 27d;
A, D, E, F, G, H, I, K, L, M, N, P, Q, R, S, T, V, W or Y at the amino acid position 27e; G, N, S or T at the amino acid position 28;
A, F, G, H, K, L, M, N, Q, R, S, T, W or Y at the amino acid position 29;
A, F, G, H, I, K, L, M, N, Q, R, V, W or Y at the amino acid position 30;
I, L, Q, S, T or V at the amino acid position 31;
F, W or Y at the amino acid position 32;
A, F, H, L, M, Q or V at the amino acid position 33;
A, H or S at the amino acid position 34;
I, K, L, M or R at the amino acid position 50;
A, E, I, K, L, M, Q, R, S, T or V at the amino acid position 51;
A, D, E, F, G, H, I, K, L, M, N, Q, R, S, T, V, W or Y at the amino acid position 52;
A, E, F, G, H, K, L, M, N, P, Q, R, S, V, W or Y at the amino acid position 53;
A, D, E, F, G, H, I, K, L, M, N, P, Q, R, S, T, V, W or Y at the amino acid position 54;
A, D, E, F, G, H, I, K, L, M, N, P, Q, R, S, T, V or Y at the amino acid position 55;
A, D, E, F, G, H, I, K, L, M, N, P, Q, R, S, T, V, W or Y at the amino acid position 56;
A, G, K, S or Y at the amino acid position 89;
Q at the amino acid position 90;
G at the amino acid position 91;
A, D, H, K, N, Q, R, S or T at the amino acid position 92;
A, D, E, F, G, H, I, K, L, M, N, Q, R, S, T, V, W or Y at the amino acid position 93;
A, D, H, I, M, N, P, Q, R, S, T or V at the amino acid position 94;
P at the amino acid position 95;
F or Y at the amino acid position 96; and
A, D, E, G, H, I, K, L, M, N, Q, R, S, T or V at the amino acid position 97.

[4] The antigen-binding molecule of any of [1]-[3A], wherein the antibody variable region comprises any one of the following:

(a1) a heavy chain complementarity determining region 1 (HCDR1) comprising an amino acid sequence that is at least 70%, 80% or 90% identical to SEQ ID NO: 16, a heavy chain complementarity determining region 2 (HCDR2) comprising an amino acid sequence that is at least 70%, 80% or 90% identical to SEQ ID NO: 30, a heavy chain complementarity determining region 3 (HCDR3) comprising an amino acid sequence that is at least 70%, 80% or 90% identical to SEQ ID NO: 44, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;
(a2) a heavy chain complementarity determining region 1 (HCDR1) comprising an amino acid sequence that is at least 70%, 80% or 90% identical to SEQ ID NO: 17, a heavy chain complementarity determining region 2 (HCDR2) comprising an amino acid sequence that is at least 70%, 80% or 90% identical to SEQ ID NO: 31, a heavy chain complementarity determining region 3 (HCDR3) comprising an amino acid sequence that is at least 70%, 80% or 90% identical to SEQ ID NO: 45, a light chain complementarity determining region 1 (LCDR1) comprising an amino acid sequence that is at least 70%, 80% or 90% identical to SEQ ID NO: 64, a light chain complementarity determining region 2 (LCDR2) comprising an amino acid sequence that is at least 70%, 80% or 90% identical to SEQ ID NO: 69, and a light chain complementarity determining region 3 (LCDR3) comprising an amino acid sequence that is at least 70%, 80% or 90% identical to SEQ ID NO: 74;
(a3) a heavy chain complementarity determining region 1 (HCDR1) comprising an amino acid sequence that is at least 70%, 80% or 90% identical to SEQ ID NO: 18, a heavy chain complementarity determining region 2 (HCDR2) comprising an amino acid sequence that is at least 70%, 80% or 90% identical to SEQ ID NO: 32, a heavy chain complementarity determining region 3 (HCDR3) comprising an amino acid sequence that is at least 70%, 80% or 90% identical to SEQ ID NO: 46, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;
(a4) a heavy chain complementarity determining region 1 (HCDR1) comprising an amino acid sequence that is at least 70%, 80% or 90% identical to SEQ ID NO: 19, a heavy chain complementarity determining region 2 (HCDR2) comprising an amino acid sequence that is at least 70%, 80% or 90% identical to SEQ ID NO: 33, a heavy chain complementarity determining region 3 (HCDR3) comprising an amino acid sequence that is at least 70%, 80% or 90% identical to SEQ ID NO: 47, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;
(a5) a heavy chain complementarity determining region 1 (HCDR1) comprising an amino acid sequence that is at least 70%, 80% or 90% identical to SEQ ID NO: 19, a heavy chain complementarity determining region 2 (HCDR2) comprising an amino acid sequence that is at least 70%, 80% or 90% identical to SEQ ID NO:33, a heavy chain complementarity determining region 3 (HCDR3) comprising an amino acid sequence that is at least 70%, 80% or 90% identical to SEQ ID NO: 47, a light chain complementarity determining region 1 (LCDR1) comprising an amino acid sequence that is at least 70%, 80% or 90% identical to SEQ ID NO: 65, a light chain complementarity determining region 2 (LCDR2) comprising an amino acid sequence that is at least 70%, 80% or 90% identical to SEQ ID NO: 70, and a light chain complementarity determining region 3 (LCDR3) comprising an amino acid sequence that is at least 70%, 80% or 90% identical to SEQ ID NO: 75;
(a6) a heavy chain complementarity determining region 1 (HCDR1) comprising an amino acid sequence that is at least 70%, 80% or 90% identical to SEQ ID NO: 20, a heavy chain complementarity determining region 2 (HCDR2) comprising an amino acid sequence that is at least 70%, 80% or 90% identical to SEQ ID NO: 34, a heavy chain complementarity determining region 3 (HCDR3) comprising an amino acid sequence that is at least 70%, 80% or 90% identical to SEQ ID NO: 48, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;
(a7) a heavy chain complementarity determining region 1 (HCDR1) comprising an amino acid sequence that is at least 70%, 80% or 90% identical to SEQ ID NO: 22, a heavy chain complementarity determining region 2 (HCDR2) comprising an amino acid sequence that is at least 70%, 80% or 90% identical to SEQ ID NO: 36, a heavy chain complementarity determining region 3 (HCDR3) comprising an amino acid sequence that is at least 70%, 80% or 90% identical to SEQ ID NO: 50, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;
(a8) a heavy chain complementarity determining region 1 (HCDR1) comprising an amino acid sequence that is at least 70%, 80% or 90% identical to SEQ ID NO: 23, a heavy chain complementarity determining region 2 (HCDR2) comprising an amino acid sequence that is at least 70%, 80% or 90% identical to SEQ ID NO: 37, a heavy chain complementarity determining region 3 (HCDR3) comprising an amino acid sequence that is at least 70%, 80% or 90% identical to SEQ ID NO: 51, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;
(a9) a heavy chain complementarity determining region 1 (HCDR1) comprising an amino acid sequence that is at least 70%, 80% or 90% identical to SEQ ID NO: 23, a heavy chain complementarity determining region 2 (HCDR2) comprising an amino acid sequence that is at least 70%, 80% or 90% identical to SEQ ID NO: 37, a heavy chain complementarity determining region 3 (HCDR3) comprising an amino acid sequence that is at least 70%, 80% or 90% identical to SEQ ID NO: 51, a light chain complementarity determining region 1 (LCDR1) comprising an amino acid sequence that is at least 70%, 80% or 90% identical to SEQ ID NO: 66, a light chain complementarity determining region 2 (LCDR2) comprising an amino acid sequence that is at least 70%, 80% or 90% identical to SEQ ID NO: 71, and a light chain complementarity determining region 3 (LCDR3) comprising an amino acid sequence that is at least 70%, 80% or 90% identical to SEQ ID NO: 76;
(a10) a heavy chain complementarity determining region 1 (HCDR1) comprising an amino acid sequence that is at least 70%, 80% or 90% identical to SEQ ID NO: 24, a heavy chain complementarity determining region 2 (HCDR2) comprising an amino acid sequence that is at least 70%, 80% or 90% identical to SEQ ID NO: 38, a heavy chain complementarity determining region 3 (HCDR3) comprising an amino acid sequence that is at least 70%, 80% or 90% identical to SEQ ID NO: 52, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;
(a11) a heavy chain complementarity determining region 1 (HCDR1) comprising an amino acid sequence that is at least 70%, 80% or 90% identical to SEQ ID NO: 25, a heavy chain complementarity determining region 2 (HCDR2) comprising an amino acid sequence that is at least 70%, 80% or 90% identical to SEQ ID NO: 39, a heavy chain complementarity determining region 3 (HCDR3) comprising an amino acid sequence that is at least 70%, 80% or 90% identical to SEQ ID NO: 53, a light chain complementarity determining region 1 (LCDR1) comprising an amino acid sequence that is at least 70%, 80% or 90% identical to SEQ ID NO: 66, a light chain complementarity determining region 2 (LCDR2) comprising an amino acid sequence that is at least 70%, 80% or 90% identical to SEQ ID NO: 71, and a light chain complementarity determining region 3 (LCDR3) comprising an amino acid sequence that is at least 70%, 80% or 90% identical to SEQ ID NO: 76;
(a12) a heavy chain complementarity determining region 1 (HCDR1) comprising an amino acid sequence that is at least 70%, 80% or 90% identical to SEQ ID NO: 26, a heavy chain complementarity determining region 2 (HCDR2) comprising an amino acid sequence that is at least 70%, 80% or 90% identical to SEQ ID NO: 40, a heavy chain complementarity determining region 3 (HCDR3) comprising an amino acid sequence that is at least 70%, 80% or 90% identical to SEQ ID NO: 54, a light chain complementarity determining region 1 (LCDR1) comprising an amino acid sequence that is at least 70%, 80% or 90% identical to SEQ ID NO: 66, a light chain complementarity determining region 2 (LCDR2) comprising an amino acid sequence that is at least 70%, 80% or 90% identical to SEQ ID NO: 71, and a light chain complementarity determining region 3 (LCDR3) comprising an amino acid sequence that is at least 70%, 80% or 90% identical to SEQ ID NO: 76;
(a13) a heavy chain complementarity determining region 1 (HCDR1) comprising an amino acid sequence that is at least 70%, 80% or 90% identical to SEQ ID NO: 26, a heavy chain complementarity determining region 2 (HCDR2) comprising an amino acid sequence that is at least 70%, 80% or 90% identical to SEQ ID NO: 40, a heavy chain complementarity determining region 3 (HCDR3) comprising an amino acid sequence that is at least 70%, 80% or 90% identical to SEQ ID NO: 54, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;
(a14) a heavy chain complementarity determining region 1 (HCDR1) comprising an amino acid sequence that is at least 70%, 80% or 90% identical to SEQ ID NO:27, a heavy chain complementarity determining region 2 (HCDR2) comprising an amino acid sequence that is at least 70%, 80% or 90% identical to SEQ ID NO: 41, a heavy chain complementarity determining region 3 (HCDR3) comprising an amino acid sequence that is at least 70%, 80% or 90% identical to SEQ ID NO: 55, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;
(a15) a heavy chain complementarity determining region 1 (HCDR1) comprising an amino acid sequence that is at least 70%, 80% or 90% identical to SEQ ID NO: 28, a heavy chain complementarity determining region 2 (HCDR2) comprising an amino acid sequence that is at least 70%, 80% or 90% identical to SEQ ID NO: 42, a heavy chain complementarity determining region 3 (HCDR3) comprising an amino acid sequence that is at least 70%, 80% or 90% identical to SEQ ID NO: 56, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;
(b1) a HCDR1 comprising an amino acid sequence of SEQ ID NO: 16, a HCDR2 comprising an amino acid sequence of SEQ ID NO: 30, a HCDR3 comprising an amino acid sequence of SEQ ID NO: 44, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b2) a HCDR1 comprising an amino acid sequence of SEQ ID NO: 17, a HCDR2 comprising an amino acid sequence of SEQ ID NO: 31, a HCDR3 comprising an amino acid sequence of SEQ ID NO: 45, a LCDR1 comprising an amino acid sequence of SEQ ID NO: 64, a LCDR2 comprising an amino acid sequence of SEQ ID NO: 69, and a LCDR3 comprising an amino acid sequence of SEQ ID NO: 74;
(b3) a HCDR1 comprising an amino acid sequence of SEQ ID NO: 18, a HCDR2 comprising an amino acid sequence of SEQ ID NO: 32, a HCDR3 comprising an amino acid sequence of SEQ ID NO: 46, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b4) a HCDR1 comprising an amino acid sequence of SEQ ID NO: 19, a HCDR2 comprising an amino acid sequence of SEQ ID NO: 33, a HCDR3 comprising an amino acid sequence of SEQ ID NO: 47, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b5) a HCDR1 comprising an amino acid sequence of SEQ ID NO: 19, a HCDR2 comprising an amino acid sequence of SEQ ID NO: 33, a HCDR3 comprising an amino acid sequence of SEQ ID NO: 47, a LCDR1 comprising an amino acid sequence of SEQ ID NO: 65, a LCDR2 comprising an amino acid sequence of SEQ ID NO: 70, and a LCDR3 comprising an amino acid sequence of SEQ ID NO: 75;
(b6) a HCDR1 comprising an amino acid sequence of SEQ ID NO: 20, a HCDR2 comprising an amino acid sequence of SEQ ID NO: 34, a HCDR3 comprising an amino acid sequence of SEQ ID NO: 48, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b7) a HCDR1 comprising an amino acid sequence of SEQ ID NO: 22, a HCDR2 comprising an amino acid sequence of SEQ ID NO: 36, a HCDR3 comprising an amino acid sequence of SEQ ID NO: 50, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b8) a HCDR1 comprising an amino acid sequence of SEQ ID NO: 23, a HCDR2 comprising an amino acid sequence of SEQ ID NO: 37, a HCDR3 comprising an amino acid sequence of SEQ ID NO: 51, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b9) a HCDR1 comprising an amino acid sequence of SEQ ID NO: 23, a HCDR2 comprising an amino acid sequence of SEQ ID NO: 37, a HCDR3 comprising an amino acid sequence of SEQ ID NO: 51, a LCDR1 comprising an amino acid sequence of SEQ ID NO: 66, a LCDR2 comprising an amino acid sequence of SEQ ID NO: 71, and a LCDR3 comprising an amino acid sequence of SEQ ID NO: 76;
(b10) a HCDR1 comprising an amino acid sequence of SEQ ID NO: 24, a HCDR2 comprising an amino acid sequence of SEQ ID NO: 38, a HCDR3 comprising an amino acid sequence of SEQ ID NO: 52, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b11) a HCDR1 comprising an amino acid sequence of SEQ ID NO: 25, a HCDR2 comprising an amino acid sequence of SEQ ID NO: 39, a HCDR3 comprising an amino acid sequence of SEQ ID NO: 53, a LCDR1 comprising an amino acid sequence of SEQ ID NO: 66, a LCDR2 comprising an amino acid sequence of SEQ ID NO: 71, and a LCDR3 comprising an amino acid sequence of SEQ ID NO: 76;
(b12) a HCDR1 comprising an amino acid sequence of SEQ ID NO: 26, a HCDR2 comprising an amino acid sequence of SEQ ID NO: 40, a HCDR3 comprising an amino acid sequence of SEQ ID NO: 54, a LCDR1 comprising an amino acid sequence of SEQ ID NO: 66, a LCDR2 comprising an amino acid sequence of SEQ ID NO: 71, and a LCDR3 comprising an amino acid sequence of SEQ ID NO: 76;
(b13) a HCDR1 comprising an amino acid sequence of SEQ ID NO: 26, a HCDR2 comprising an amino acid sequence of SEQ ID NO: 40, a HCDR3 comprising an amino acid sequence of SEQ ID NO: 54, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b14) a HCDR1 comprising an amino acid sequence of SEQ ID NO: 27, a HCDR2 comprising an amino acid sequence of SEQ ID NO: 41, a HCDR3 comprising an amino acid sequence of SEQ ID NO: 55, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b15) a HCDR1 comprising an amino acid sequence of SEQ ID NO: 28, a HCDR2 comprising an amino acid sequence of SEQ ID NO: 42, a HCDR3 comprising an amino acid sequence of SEQ ID NO: 56, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(c1) a heavy chain variable domain (VH) comprising an amino acid sequence that is at least 70%, 80% or 90% identical to SEQ ID NO: 2, and a light chain variable domain (VL) comprising an amino acid sequence that is at least 70%, 80% or 90% identical to SEQ ID NO: 58;
(c2) a heavy chain variable domain (VH) comprising an amino acid sequence that is at least 70%, 80% or 90% identical to SEQ ID NO: 3, and a light chain variable domain (VL) comprising an amino acid sequence that is at least 70%, 80% or 90% identical to SEQ ID NO: 59;
(c3) a heavy chain variable domain (VH) comprising an amino acid sequence that is at least 70%, 80% or 90% identical to SEQ ID NO: 4, and a light chain variable domain (VL) comprising an amino acid sequence that is at least 70%, 80% or 90% identical to SEQ ID NO: 58;
(c4) a heavy chain variable domain (VH) comprising an amino acid sequence that is at least 70%, 80% or 90% identical to SEQ ID NO: 5, and a light chain variable domain (VL) comprising an amino acid sequence that is at least 70%, 80% or 90% identical to SEQ ID NO: 58;
(c5) a heavy chain variable domain (VH) comprising an amino acid sequence that is at least 70%, 80% or 90% identical to SEQ ID NO: 5, and a light chain variable domain (VL) comprising an amino acid sequence that is at least 70%, 80% or 90% identical to SEQ ID NO: 60;
(c6) a heavy chain variable domain (VH) comprising an amino acid sequence that is at least 70%, 80% or 90% identical to SEQ ID NO: 6, and a light chain variable domain (VL) comprising an amino acid sequence that is at least 70%, 80% or 90% identical to SEQ ID NO: 58;
(c7) a heavy chain variable domain (VH) comprising an amino acid sequence that is at least 70%, 80% or 90% identical to SEQ ID NO: 8, and a light chain variable domain (VL) comprising an amino acid sequence that is at least 70%, 80% or 90% identical to SEQ ID NO: 58;
(c8) a heavy chain variable domain (VH) comprising an amino acid sequence that is at least 70%, 80% or 90% identical to SEQ ID NO: 9, and a light chain variable domain (VL) comprising an amino acid sequence that is at least 70%, 80% or 90% identical to SEQ ID NO: 58;
(c9) a heavy chain variable domain (VH) comprising an amino acid sequence that is at least 70%, 80% or 90% identical to SEQ ID NO: 9, and a light chain variable domain (VL) comprising an amino acid sequence that is at least 70%, 80% or 90% identical to SEQ ID NO: 61;
(c10) a heavy chain variable domain (VH) comprising an amino acid sequence that is at least 70%, 80% or 90% identical to SEQ ID NO: 10, and a light chain variable domain (VL) comprising an amino acid sequence that is at least 70%, 80% or 90% identical to SEQ ID NO: 58;
(c11) a heavy chain variable domain (VH) comprising an amino acid sequence that is at least 70%, 80% or 90% identical to SEQ ID NO: 11, and a light chain variable domain (VL) comprising an amino acid sequence that is at least 70%, 80% or 90% identical to SEQ ID NO: 61;
(c12) a heavy chain variable domain (VH) comprising an amino acid sequence that is at least 70%, 80% or 90% identical to SEQ ID NO: 12, and a light chain variable domain (VL) comprising an amino acid sequence that is at least 70%, 80% or 90% identical to SEQ ID NO: 61;
(c13) a heavy chain variable domain (VH) comprising an amino acid sequence that is at least 70%, 80% or 90% identical to SEQ ID NO: 12, and a light chain variable domain (VL) comprising an amino acid sequence that is at least 70%, 80% or 90% identical to SEQ ID NO: 58;
(c14) a heavy chain variable domain (VH) comprising an amino acid sequence that is at least 70%, 80% or 90% identical to SEQ ID NO: 13, and a light chain variable domain (VL) comprising an amino acid sequence that is at least 70%, 80% or 90% identical to SEQ ID NO: 58;
(c15) a heavy chain variable domain (VH) comprising an amino acid sequence that is at least 70%, 80% or 90% identical to SEQ ID NO: 14, and a light chain variable domain (VL) comprising an amino acid sequence that is at least 70%, 80% or 90% identical to SEQ ID NO: 58;
(d1) a heavy chain variable domain (VH) of SEQ ID NO: 2, and a light chain variable domain (VL) of SEQ ID NO: 58;
(d2) a heavy chain variable domain (VH) of SEQ ID NO: 3, and a light chain variable domain (VL) of SEQ ID NO: 59;
(d3) a heavy chain variable domain (VH) of SEQ ID NO: 4, and a light chain variable domain (VL) of SEQ ID NO: 58;
(d4) a heavy chain variable domain (VH) of SEQ ID NO: 5, and a light chain variable domain (VL) of SEQ ID NO: 58;
(d5) a heavy chain variable domain (VH) of SEQ ID NO: 5, and a light chain variable domain (VL) of SEQ ID NO: 60;
(d6) a heavy chain variable domain (VH) of SEQ ID NO: 6, and a light chain variable domain (VL) of SEQ ID NO: 58;
(d7) a heavy chain variable domain (VH) of SEQ ID NO: 8, and a light chain variable domain (VL) of SEQ ID NO: 58;
(d8) a heavy chain variable domain (VH) of SEQ ID NO: 9, and a light chain variable domain (VL) of SEQ ID NO: 58;
(d9) a heavy chain variable domain (VH) of SEQ ID NO: 9, and a light chain variable domain (VL) of SEQ ID NO: 61;
(d10) a heavy chain variable domain (VH) of SEQ ID NO: 10, and a light chain variable domain (VL) of SEQ ID NO: 58;
(d11) a heavy chain variable domain (VH) of SEQ ID NO: 11, and a light chain variable domain (VL) of SEQ ID NO: 61;
(d12) a heavy chain variable domain (VH) of SEQ ID NO: 12, and a light chain variable domain (VL) of SEQ ID NO: 61;
(d13) a heavy chain variable domain (VH) of SEQ ID NO: 12, and a light chain variable domain (VL) of SEQ ID NO: 58;
(d14) a heavy chain variable domain (VH) of SEQ ID NO: 13, and a light chain variable domain (VL) of SEQ ID NO: 58;
(d15) a heavy chain variable domain (VH) of SEQ ID NO: 14, and a light chain variable domain (VL) of SEQ ID NO: 58;
(e) an antibody variable region that competes for binding to CD3 with any one of the antibody variable regions of (a1) to (d15);
(f) an antibody variable region that competes for binding to CD137 with any one of the antibody variable regions of (a1) to (d15);
(g) an antibody variable region that binds to the same epitope on CD3 with any one of the antibody variable regions of (a1) to (d15);
(h) an antibody variable region that binds to the same epitope on CD137 with any one of the antibody variable regions of (a1) to (d15).

[4A] The antigen-binding molecule of [4][c1]-[c15], wherein the heavy chain variable domain (VH) and/or the light chain variable domain (VL) comprise(s) one or more amino acid substitution selected from Table 1.3 (a) to Table 1.3 (d), wherein the one or more amino acid substitution shows at least 0.2, 0.3, 0.5, 0.8, 1, 1.5 or 2-fold binding affinity increase to CD3 and/or CD137 as set forth in Table 1.3 (a) to Table 1.3 (d).

[4B] The antigen-binding molecule of [4A], wherein the antibody variable region comprises:

(a) a heavy chain variable domain amino acid sequence comprising, at each of the following positions (all by Kabat numbering), one or more of the following amino acid residues indicated for that position:
A, D, E, I, G, K, L, M, N, R, T, W or Y at the amino acid position 26;
D, F, G, I, M or L, at the amino acid position 27;
D, E, F, G, H, I, K, L, M, N, P, Q, R, S, T, V, W or Y at the amino acid position 28;
F or W at the amino acid position 29;
A, D, E, F, G, H, I, K, L, M, N, P, Q, R, S, T, V, W or Y at the amino acid position 30;
F, I, N, R, S, T or V at the amino acid position 31;
A, H, I, K, L, N, Q, R, S, T or V at the amino acid position 32;
W at the amino acid position 33;
F, I, L, M or V at the amino acid position 34;

F, H, S, T, V or Y at the amino acid position 35;

E, F, H, I, K, L, M, N, Q, S, T, W or Y at the amino acid position 50;
I, K or V at the amino acid position 51;
K, M, R, or T at the amino acid position 52;
A, E, F, G, H, I, K, L, M, N, P, Q, R, S, V, W or Y at the amino acid position 52b;
A, D, E, F, G, H, I, K, L, M, N, P, Q, R, S, T, V, W or Y at the amino acid position 52c;
A, E, F, H, K, L, M, N, Q, R, S, T, V, W or Y at the amino acid position 53;
A, D, E, F, G, H, I, K, L, M, N, Q, R, S, T, V, W or Y at the amino acid position 54;
E, F, G, H, L, M, N, Q, W or Y at the amino acid position 55;
A, D, E, F, G, H, I, K, L, M, N, Q, R, S, T, V, W or Y at the amino acid position 56;
A, D, E, G, H, I, K, L, M, N, P, Q, R, S, T or V at the amino acid position 57;
A, F, H, K, N, P, R or Y at the amino acid position 58;
A, D, E, F, G, H, I, K, L, M, N, P, Q, R, S, T, V, W or Y at the amino acid position 59;
A, D, E, F, G, H, I, K, L, M, N, P, Q, R, S, T, V, W or Y at the amino acid position 60;
A, D, E, F, G, H, I, K, L, M, N, P, Q, R, S, T, V, W or Y at the amino acid position 61;
A, D, E, F, G, H, I, K, L, M, N, P, Q, R, S, T, V, W or Y at the amino acid position 62;
A, D, E, F, G, H, I, K, L, M, N, P, Q, R, S, T, V, W or Y at the amino acid position 63;
A, D, E, F, G, H, I, K, L, M, N, P, Q, R, S, T, V, W or Y at the amino acid position 64;
A, D, E, F, G, H, I, K, L, M, N, P, Q, R, S, T, V, W or Y at the amino acid position 65;
H or R at the amino acid position 93;
F, G, H, L, M, S, T, V or Y at the amino acid position 94;
I or V at the amino acid position 95;
F, H, I, K, L, M, T, V, W or Y at the amino acid position 96;
F, Y or W at the amino acid position 97;
A, F, G, H, I, K, L, M, N, Q, R, S, T, V, W or Y at the amino acid position 98;
A, F, G, H, I, K, L, M, N, P, Q, R, S, T, V, W or Y at the amino acid position 99;
A, D, E, F, G, H, I, K, L, M, N, P, Q, R, S, T, V, W or Y at the amino acid position 100;
A, D, E, F, G, H, I, K, L, M, N, P, Q, R, S, T, V, W or Y at the amino acid position 100a;
A, D, E, F, G, H, I, K, L, M, N, P, Q, R, S, T, V, W or Y at the amino acid position 100b;
A, D, E, F, G, H, I, K, L, M, N, P, Q, R, S, T, V, W or Y at the amino acid position 100c;
A, D, E, F, G, H, I, K, L, M, N, P, Q, R, S, T, V, W or Y at the amino acid position 100d;
A, D, E, F, G, H, I, K, L, M, P, Q, R, S, T, V, W or Y at the amino acid position 100e;
A, E, F, G, H, I, K, L, M, N, P, Q, R, S, T, V, W or Y at the amino acid position 100f;
A, E, F, G, H, I, K, L, M, N, P, Q, R, S, T, V, W or Y at the amino acid position 100g;
A, D, E, G, H, I, L, M, N, P, S, T or V at the amino acid position 100h;
A, D, E, F, G, H, I, K, L, M, N, P, Q, R, S, T, V, W or Y at the amino acid position 100i;
A, D, F, I, L, M, N, Q, S, T or V at the amino acid position 101;
A, D, E, F, G, H, I K, L, M, N, Q, R, S, T, V, W or Y at the amino acid position 102; and/or
(b) a light chain variable domain amino acid sequence comprising, at each of the following positions (all by Kabat numbering), one or more of the following amino acid residues indicated for that position:
A, D, F, G, H, I, K, L, M, N, Q, R, S, T, V, W or Y at the amino acid position 24;
A, G, N, P, S, T or V at the amino acid position 25;
A, D, E, F, G, I, K, L, M, N, Q, R, S, T or V at the amino acid position 26;
A, D, E, F, G, H, I, K, L, M, N, Q, R, S, T, V, W or Y at the amino acid position 27;
A, D, E, F, G, H, I, K, L, M, N, P, Q, R, S, T, V, W or Y at the amino acid position 27a;
A, I, L, M, P, T or V at the amino acid position 27b;
A, E, F, H, I, K, L, M, N, P, Q, R, T, W or Y at the amino acid position 27c;
A, E, G, H, I, K, L, M, N, P, Q, R, S, T, V, W or Y at the amino acid position 27d;
A, D, E, F, G, H, I, K, L, M, N, P, Q, R, S, T, V, W or Y at the amino acid position 27e;
G, N, S or T at the amino acid position 28;
A, F, G, H, K, L, M, N, Q, R, S, T, W or Y at the amino acid position 29;
A, F, G, H, I, K, L, M, N, Q, R, V, W or Y at the amino acid position 30;
I, L, Q, S, T or V at the amino acid position 31;
F, W or Y at the amino acid position 32;
A, F, H, L, M, Q or V at the amino acid position 33;
A, H or S at the amino acid position 34;
I, K, L, M or R at the amino acid position 50;
A, E, I, K, L, M, Q, R, S, T or V at the amino acid position 51;
A, D, E, F, G, H, I, K, L, M, N, Q, R, S, T, V, W or Y at the amino acid position 52;
A, E, F, G, H, K, L, M, N, P, Q, R, S, V, W or Y at the amino acid position 53;
A, D, E, F, G, H, I, K, L, M, N, P, Q, R, S, T, V, W or Y at the amino acid position 54;
A, D, E, F, G, H, I, K, L, M, N, P, Q, R, S, T, V or Y at the amino acid position 55;
A, D, E, F, G, H, I, K, L, M, N, P, Q, R, S, T, V, W or Y at the amino acid position 56;
A, G, K, S or Y at the amino acid position 89;
Q at the amino acid position 90;
G at the amino acid position 91;
A, D, H, K, N, Q, R, S or T at the amino acid position 92;
A, D, E, F, G, H, I, K, L, M, N, Q, R, S, T, V, W or Y at the amino acid position 93;
A, D, H, I, M, N, P, Q, R, S, T or V at the amino acid position 94;
P at the amino acid position 95;
F or Y at the amino acid position 96; and
A, D, E, G, H, I, K, L, M, N, Q, R, S, T or V at the amino acid position 97.

[5] The antigen-binding molecule of any one of [1] to [4B], wherein the antigen-binding molecule has at least one characteristic selected from the group consisting of (1) to (3) below:

(1) the antigen-binding molecule does not bind to CD3 and CD137 each expressed on a different cell, at the same time.
(2) the antigen-binding molecule has an agonistic activity against CD137; and
(3) the antigen-binding molecule has equivalent or 10-fold, 20-fold, 50-fold, 100-fold lower KD value for binding to human CD137, as compared to a reference antibody comprising a VH sequence of SEQ ID NO: 1 and a VL sequence of SEQ ID NO: 57, wherein the KD value is preferably measured by SPR at the following condition: 37 degrees C., pH 7.4, 20 mM ACES, 150 mM NaCl, 0.05% Tween 20, 0.005% NaN3; the antigen-binding molecule is immobilized on a CM4 sensor chip, the antigen serves as analyte.

[6] The antigen-binding molecule of any one of [1] to [5], which further comprises an antibody variable region that is capable of binding to a third antigen different from CD3 and CD137.

[7] The antigen-binding molecule of [6], wherein the third antigen is a molecule specifically expressed in a cancer tissue.

[7A] The antigen-binding molecule of any one of [6] to [7], wherein the third antigen is Glypican-3 (GPC3).

[7B] The antigen-binding molecule of [7A], wherein the antibody variable region that is capable of binding to Glypican-3 (GPC3) comprises a VH sequence having the amino acid sequence of SEQ ID NO: 206 and a VL sequence having the amino acid sequence of SEQ ID NO: 207.

[7C] The antigen-binding molecule of any one of [6] to [7B], wherein the antigen-binding molecule has at least one characteristic selected from the group consisting of (1) to (5) below:

(1) the antigen-binding molecule induces CD3 activation of a T cell against a cell expressing the molecule of the third antigen, but does not induce CD3 activation of a T cell against a cell expressing CD137;
(2) the antigen-binding molecule induces cytotoxicity of a T cell against a cell expressing the molecule of the third antigen, but does not induce cytotoxicity of a T cell against a cell expressing CD137;
(3) the antigen-binding molecule does not induce a cytokine release from PBMC in the absence of a cell expressing the molecule of the third antigen;
(4) the antigen-binding molecule induces equivalent to or 2-fold, 5-fold, 10-fold, 20-fold or 100-fold greater CD137 activation and/or cytotoxicity of a T cell against a cell expressing the molecule of the third antigen, as compared to a reference antibody comprising a VH sequence of SEQ ID NO: 1 and a VL sequence of SEQ ID NO: 57; and/or
(5) the antigen-binding molecule induces 2-fold, 5-fold, 10-fold, 20-fold or 100-fold greater cytotoxicity of a T cell against a cell expressing the molecule of the third antigen while does not induce a cytokine (IL-6) release from PBMC, as compared to a reference bispecific antibody targeting the third antigen and CD3.

[8] The antigen-binding molecule of any one of [1] to [7C], further comprising an antibody Fc region.

[9] The antigen-binding molecule of [8], wherein the Fc region is an Fc region having reduced binding activity against Fc gamma R as compared with the Fc region of a naturally occurring human IgG1 antibody.

[10] A pharmaceutical composition comprising the antigen-binding molecule according to any of [1] to [9] and a pharmaceutically acceptable carrier.

[10A] The pharmaceutical composition of [10] or the antigen-binding molecule of [1] to [9], for use in the treatment of cancer.

[10B] Use of the pharmaceutical composition of [10] or the antigen-binding molecule of [1] to [9], for the manufacture of a medicament for use in the treatment of cancer.

[10C] A method for preventing, treating or inhibiting cancer comprising: administering to a mammalian subject suffering from cancer the pharmaceutical composition of [10] or the antigen-binding molecule of [5] to [9].

[10D] A method for inducing cytotoxicity, preferably T-cell dependent cytotoxicity in a subject, comprising: administering to a mammalian subject suffering from cancer the pharmaceutical composition of [10] or the antigen-binding molecule of [5] to [9].

[10E] A method for reducing or killing cancer cell a subject, comprising: administering to a mammalian subject suffering from cancer the pharmaceutical composition of [10] or the antigen-binding molecule of [5] to [9].

[10F] A method for extending lifespan or survival rate of a cancer patient, comprising: administering to a mammalian subject suffering from cancer the pharmaceutical composition of [10] or the antigen-binding [5] to [9].

[10G] The pharmaceutical composition or antigen-binding molecule for use, the use, or the method according to any of [10A] to [10F], wherein the cancer is characterized by expression or upregulated expression of the third antigen, preferably Glypican-3 (GPC3).

[11] An isolated polynucleotide comprising a nucleotide sequence that encodes the antigen-binding molecule of any of [1] to [9].

[12] An expression vector comprising the polynucleotide according to [11].

[13] A host cell transformed or transfected with the polynucleotide according to [11] or the expression vector according to [12].

[14] A method of producing a multispecific antigen-binding molecule or a multispecific antibody comprising culturing the host cell of [13].

[15] A multispecific antigen-binding molecule or a multispecific antibody produced by the method of [14].

[16] A method of obtaining or screening for an antibody variable region that is capable of binding to CD3 and CD137, but does not bind to CD3 and CD137 at the same time, comprising:

(a) providing a library comprising a plurality of antibody variable region,
(b) contacting the library provided in step (a) with either CD3 or CD137 as a first antigen and collecting antibody variable regions bound to the first antigen,
(c) contacting the antibody variable regions collected in step (b) with a second antigen out of CD3 and CD137 and collecting antibody variable regions bound to the second antigen, and
(d) selecting an antibody variable region which:
(1) binds to CD137 with an equilibrium dissociation constant (KD) of less than about 5×10⁻⁶ M or between 5×10⁻⁶ M and 3×10⁻⁸M, preferably as measured by SPR at the following condition: 37 degrees C., pH 7.4, 20 mM ACES, 150 mM NaCl, 0.05% Tween 20, 0.005% NaN3; the antigen-binding molecule is immobilized on a CM4 sensor chip, the antigen serves as analyte; and/or
(2) binds to CD3 with an equilibrium dissociation constant (KD) of between 2×10⁻⁶ M and 1×10⁻⁸M, preferably as measured by SPR at the following condition: 25 degrees C., pH 7.4, 20 mM ACES, 150 mM NaCl, 0.05% Tween 20, 0.005% NaN3; the antigen-binding molecule is immobilized on a CM4 sensor chip, the antigen serves as analyte.

[16A] The method of [16], wherein from step (c) to (d), further comprises introducing one or more amino acid alteration to the antibody variable regions collected in step (c).

[17] The method of any of [16] or [16A], wherein the antibody variable region in step (a) or from step (c) to (d), is an antibody variable region having alteration of 1 to 25 amino acids, wherein the amino acid to be altered is an amino acid in a loop, an amino acid in a FR3 region, or an amino acid selected from Kabat numbering positions 31 to 35, 50 to 65, 71 to 74, and 95 to 102 in an antibody H chain variable domain, and Kabat numbering positions 24 to 34, 50 to 56, and 89 to 97 in an L chain variable domain.

[18] The method of [17], wherein the heavy chain variable domain (VH) and/or the light chain variable domain (VL) comprise(s) one or more amino acid substitution selected from Table 1.3 (a) to Table 1.3 (d), wherein the one or more amino acid substitution shows at least 0.2, 0.3, 0.5, 0.8, 1, 1.5 or 2-fold binding affinity increase to CD3 and/or CD137 as set forth in Table 1.3 (a) to Table 1.3 (d). In some embodiments, it is preferable that the antibody variable region comprises:

(a) a heavy chain variable domain amino acid sequence comprising, at each of the following positions (all by Kabat numbering), one or more of the following amino acid residues indicated for that position:
A, D, E, I, G, K, L, M, N, R, T, W or Y at the amino acid position 26;
D, F, G, I, M or L, at the amino acid position 27;
D, E, F, G, H, I, K, L, M, N, P, Q, R, S, T, V, W or Y at the amino acid position 28;
F or W at the amino acid position 29;
A, D, E, F, G, H, I, K, L, M, N, P, Q, R, S, T, V, W or Y at the amino acid position 30;
F, I, N, R, S, T or V at the amino acid position 31;
A, H, I, K, L, N, Q, R, S, T or V at the amino acid position 32;
W at the amino acid position 33;
F, I, L, M or V at the amino acid position 34;
F, H, S, T, V or Y at the amino acid position 35;
E, F, H, I, K, L, M, N, Q, S, T, W or Y at the amino acid position 50;
I, K or V at the amino acid position 51;
K, M, R, or T at the amino acid position 52;
A, E, F, G, H, I, K, L, M, N, P, Q, R, S, V, W or Y at the amino acid position 52b;
A, D, E, F, G, H, I, K, L, M, N, P, Q, R, S, T, V, W or Y at the amino acid position 52c;
A, E, F, H, K, L, M, N, Q, R, S, T, V, W or Y at the amino acid position 53;
A, D, E, F, G, H, I, K, L, M, N, Q, R, S, T, V, W or Y at the amino acid position 54;
E, F, G, H, L, M, N, Q, W or Y at the amino acid position 55;
A, D, E, F, G, H, I, K, L, M, N, Q, R, S, T, V, W or Y at the amino acid position 56;
A, D, E, G, H, I, K, L, M, N, P, Q, R, S, T or V at the amino acid position 57;
A, F, H, K, N, P, R or Y at the amino acid position 58;
A, D, E, F, G, H, I, K, L, M, N, P, Q, R, S, T, V, W or Y at the amino acid position 59;
A, D, E, F, G, H, I, K, L, M, N, P, Q, R, S, T, V, W or Y at the amino acid position 60;
A, D, E, F, G, H, I, K, L, M, N, P, Q, R, S, T, V, W or Y at the amino acid position 61;
A, D, E, F, G, H, I, K, L, M, N, P, Q, R, S, T, V, W or Y at the amino acid position 62;
A, D, E, F, G, H, I, K, L, M, N, P, Q, R, S, T, V, W or Y at the amino acid position 63;
A, D, E, F, G, H, I, K, L, M, N, P, Q, R, S, T, V, W or Y at the amino acid position 64;
A, D, E, F, G, H, I, K, L, M, N, P, Q, R, S, T, V, W or Y at the amino acid position 65;
H or R at the amino acid position 93;
F, G, H, L, M, S, T, V or Y at the amino acid position 94;
I or V at the amino acid position 95;
F, H, I, K, L, M, T, V, W or Y at the amino acid position 96;
F, Y or W at the amino acid position 97;
A, F, G, H, I, K, L, M, N, Q, R, S, T, V, W or Y at the amino acid position 98;
A, F, G, H, I, K, L, M, N, P, Q, R, S, T, V, W or Y at the amino acid position 99;
A, D, E, F, G, H, I, K, L, M, N, P, Q, R, S, T, V, W or Y at the amino acid position 100;
A, D, E, F, G, H, I, K, L, M, N, P, Q, R, S, T, V, W or Y at the amino acid position 100a;
A, D, E, F, G, H, I, K, L, M, N, P, Q, R, S, T, V, W or Y at the amino acid position 100b;
A, D, E, F, G, H, I, K, L, M, N, P, Q, R, S, T, V, W or Y at the amino acid position 100c;
A, D, E, F, G, H, I, K, L, M, N, P, Q, R, S, T, V, W or Y at the amino acid position 100d;
A, D, E, F, G, H, I, K, L, M, P, Q, R, S, T, V, W or Y at the amino acid position 100e;
A, E, F, G, H, I, K, L, M, N, P, Q, R, S, T, V, W or Y at the amino acid position 100f;
A, E, F, G, H, I, K, L, M, N, P, Q, R, S, T, V, W or Y at the amino acid position 100g;
A, D, E, G, H, I, L, M, N, P, S, T or V at the amino acid position 100h;
A, D, E, F, G, H, I, K, L, M, N, P, Q, R, S, T, V, W or Y at the amino acid position 100i;
A, D, F, I, L, M, N, Q, S, T or V at the amino acid position 101;
A, D, E, F, G, H, I K, L, M, N, Q, R, S, T, V, W or Y at the amino acid position 102; and/or
(b) a light chain variable domain amino acid sequence comprising, at each of the following positions (all by Kabat numbering), one or more of the following amino acid residues indicated for that position:
A, D, F, G, H, I, K, L, M, N, Q, R, S, T, V, W or Y at the amino acid position 24;
A, G, N, P, S, T or V at the amino acid position 25;
A, D, E, F, G, I, K, L, M, N, Q, R, S, T or V at the amino acid position 26;
A, D, E, F, G, H, I, K, L, M, N, Q, R, S, T, V, W or Y at the amino acid position 27;
A, D, E, F, G, H, I, K, L, M, N, P, Q, R, S, T, V, W or Y at the amino acid position 27a;
A, I, L, M, P, T or V at the amino acid position 27b;
A, E, F, H, I, K, L, M, N, P, Q, R, T, W or Y at the amino acid position 27c;
A, E, G, H, I, K, L, M, N, P, Q, R, S, T, V, W or Y at the amino acid position 27d;
A, D, E, F, G, H, I, K, L, M, N, P, Q, R, S, T, V, W or Y at the amino acid position 27e;
G, N, S or T at the amino acid position 28;
A, F, G, H, K, L, M, N, Q, R, S, T, W or Y at the amino acid position 29;
A, F, G, H, I, K, L, M, N, Q, R, V, W or Y at the amino acid position 30;
I, L, Q, S, T or V at the amino acid position 31;
F, W or Y at the amino acid position 32;
A, F, H, L, M, Q or V at the amino acid position 33;
A, H or S at the amino acid position 34;
I, K, L, M or R at the amino acid position 50;
A, E, I, K, L, M, Q, R, S, T or V at the amino acid position 51;
A, D, E, F, G, H, I, K, L, M, N, Q, R, S, T, V, W or Y at the amino acid position 52;
A, E, F, G, H, K, L, M, N, P, Q, R, S, V, W or Y at the amino acid position 53;
A, D, E, F, G, H, I, K, L, M, N, P, Q, R, S, T, V, W or Y at the amino acid position 54;
A, D, E, F, G, H, I, K, L, M, N, P, Q, R, S, T, V or Y at the amino acid position 55;
A, D, E, F, G, H, I, K, L, M, N, P, Q, R, S, T, V, W or Y at the amino acid position 56;
A, G, K, S or Y at the amino acid position 89;
Q at the amino acid position 90;
G at the amino acid position 91;
A, D, H, K, N, Q, R, S or T at the amino acid position 92;
A, D, E, F, G, H, I, K, L, M, N, Q, R, S, T, V, W or Y at the amino acid position 93;
A, D, H, I, M, N, P, Q, R, S, T or V at the amino acid position 94;
P at the amino acid position 95;
F or Y at the amino acid position 96; and
A, D, E, G, H, I, K, L, M, N, Q, R, S, T or V at the amino acid position 97.
In another aspect, the present invention relates to n antigen-binding molecule, such as an antibody, which binds to at least one, two, three or more amino acid residues of the N-terminal region of CD137 comprising the amino acid sequence of LQDPCSNCPAGTFCDNNRNQICSPCPPNSFSSAGGQRTCDICRQCKGVFRTRKECSSTSNAE C (SEQ ID NO: 152), preferably LQDPCSN, NNRNQI and/or GQRTCDI of human CD137.

In some embodiments, the antigen binding molecule of the present invention can activate T cells by its agonistic activity on CD3, and it can induce cytotoxicity of T cells against target cells, and strengthen T-cell activation, survival, and differentiation into memory T cells by its co-stimulatory agonistic activity on CD137 and CD3. Meanwhile, the antigen binding molecule of the present invention can avoid the adverse events caused by cross-linking of CD137 and CD3 because it does not bind to CD3 and CD137 at the same time.

In some embodiments, the antigen binding molecule of the present invention can also activate immune cells expressing CD137 and strengthen the immune response to target cells by the agonistic activity on CD137.

### BRIEF DESCRIPTION OF DRAWINGS

FIG. 1.1 Measurement of CD3 agonistic activity of affinity matured GPC3/Dual-Ig variants trispecific antibodies. Mean Luminescence units+/−standard deviation (s.d.) detected by SK-pca60 cell line co-cultured with NFAT-luc2 Jurkat reporter cells by selected antibodies divided into plate 1 (upper panel) and plate 2 (lower panel) E:T ratio 5 for 24 hours. Antibodies were added at 0.02, 0.2 and 2 nM.

FIG. 1.2 Measurement of CD137 agonistic activity of affinity matured GPC3/Dual-Ig variants trispecific antibodies. Mean Luminescence units+/−standard deviation (s.d.) detected by SK-pca60 cell line co-cultured with Jurkat NF kappa B reporter cells overexpressing CD137 by selected antibodies divided into plate 1 (upper panel) and plate 2 (lower panel) E:T ratio 5 for 5 hours. Antibodies were added at 0.5, 2.5 and 5 nM.

FIG. 1.3a Cytotoxicity on SK-pca60 cell line expressing GPC3 by co-culture with PBMCs in the presence of selected GPC3/Dual-Ig trispecific molecules (plate 1). Mean Cell Growth Inhibition (%) values+/−s.d. obtained at approximately 120 h were plotted.

FIG. 1.3b Cytotoxicity on SK-pca60 cell line expressing GPC3 by co-culture with PBMCs in the presence of selected GPC3/Dual-Ig trispecific molecules (plate 2). Mean Cell Growth Inhibition (%) values+/−s.d. obtained at approximately 120 h were plotted.

FIG. 1.3c Cytokine (IFN gamma) release measured in the co-culture of SK-pca60 cell line expressing GPC3 with PBMCs in the presence of selected GPC3/Dual-Ig trispecific molecules. Supernatant of the co-culture was analysed at 48h timepoint. The graph shows mean concentration+/−s.d. of IFN gamma. The antibodies were divided into plate 1 (upper panel) and plate 2 (lower panel) for evaluation.

FIG. 1.3d Cytokine (IL-2) release measured in the co-culture of SK-pca60 cell line expressing GPC3 with PBMCs in the presence of selected GPC3/Dual-Ig trispecific molecules. Supernatant of the co-culture was analysed at 48h timepoint. The graph shows mean concentration+/−s.d. of IL-2. The antibodies were divided into plate 1 (upper panel) and plate 2 (lower panel) for evaluation.

FIG. 1.3e Cytokine (IL-6) release measured in the co-culture of SK-pca60 cell line expressing GPC3 with PBMCs in the presence of selected GPC3/Dual-Ig trispecific molecules. Supernatant of the co-culture was analysed at 48h timepoint. The graph shows mean concentration+/−s.d. of IL-6. The antibodies were divided into plate 1 (upper panel) and plate 2 (lower panel) for evaluation.

FIG. 2.1 Design and construction of trispecific antibodies (mAb AB)

FIG. 2.2 Naming rule of prepared trispecific antibodies

FIG. 2.3a Antigen independent Jurkat activation on GPC3 negative cells. Parental CHO cells were co-cultured with NFAT-luc2 Jurkat reporter cells, E:T 5 for 24h. Graph depicting Mean Luminescence units+/−standard deviation (s.d.) of different antibody formats incubated at 0.5, 5 and 50 nM.

FIG. 2.3b Antigen independent Jurkat activation on GPC3 negative cells. CHO cells overexpressing CD137 were co-cultured with NFAT-luc2 Jurkat reporter cells, E:T 5 for 24h. Graph depicting Mean Luminescence units+/−standard deviation (s.d.) of different antibody formats incubated at 0.5, 5 and 50 nM.

FIG. 2.4a Antigen independent cytokine (IFN gamma) release in PBMC solution. Supernatant of affinity matured GPC3/Dual-Ig variants or GPC3/CD137×CD3 tri-specific antibodies that were added at 3.2, 16 and 80 nM to PBMC solution was analysed at 48h timepoint. Graph shows mean concentration+/−s.d. of IFN gamma. Antibodies were divided into plate 1 (upper panel) and plate 2 (lower panel) for evaluation.

FIG. 2.4b Antigen independent cytokine (TNF alpha) release in PBMC solution. Supernatant of affinity matured GPC3/Dual-Ig variants or GPC3/CD137×CD3 tri-specific antibodies that were added at 3.2, 16 and 80 nM to PBMC solution was analysed at 48h timepoint. Graph shows mean concentration+/−s.d. of TNF alpha. Antibodies were divided into plate 1 (upper panel) and plate 2 (lower panel) for evaluation.

FIG. 2.4c Antigen independent cytokine (IL-6) release in PBMC solution. Supernatant of affinity matured GPC3/Dual-Ig variants or GPC3/CD137×CD3 tri-specific antibodies that were added at 3.2, 16 and 80 nM to PBMC solution was analysed at 48h timepoint. Graph shows mean concentration+/−s.d. of IL-6. Antibodies were divided into plate 1 (upper panel) and plate 2 (lower panel) for evaluation.

FIG. 3.1a In vivo efficacy of antibodies against LLC1/hGPC3 xenograft in humanised CD3/CD137 mice model. Y-axis means the tumor volume (mm³) and X-axis means the days after tumor implantation.

FIG. 3.1b In vivo efficacy of antibodies against LLC1/hGPC3 xenograft in humanised CD3/CD137 mice model. Y-axis means the tumor volume (mm³) and X-axis means the days after tumor implantation.

FIG. 3.1c Plasma IL-6 concentration. Mice were bled at 2h after antibody injection and plasma IL-6 concentration was measured using Bio-Plea Pro Mouse Cytokine Th1 Panel.

FIG. 3.2 In vivo efficacy of antibodies against sk-pca-13a xenograft in huNOG mice model. Y-axis means the tumor volume (mm³) and X-axis means the days after tumor implantation.

FIG. 3.3a Epitope of the H0868L0581 Fab contact region on the CD137. Epitope mapping in the CD137 amino acid sequence (black: closer than 3.0 angstrom, stripes: closer than 4.5 angstrom from H0868L0581).

FIG. 3.3b Epitope of the H0868L0581 Fab contact region on the CD137. Epitope mapping in the crystal structure (dark gray spheres: closer than 3.0 angstrom, light gray sticks: closer than 4.5 angstrom from H0868L0581).

FIG. 4 A drawing showing a design of C3 NP1-27, CD3 epsilon peptide antigen which is biotin-labeled through disulfide-bond linker.

FIG. 5 A graph showing the result of phage ELISA of clones obtained with phage display to CD3 and CD137.Y axis means the specificity to CD137-Fc and X axis means the specificity to CD3 of each clone.

FIG. 6 A graph showing the result of phage ELISA of clones obtained with phage display to CD3 and CD137.Y axis means the specificity to CD137-Fc in beads ELISA and X axis means the specificity to CD3 in plate ELISA as same as FIG. 5 of each clone.

FIG. 7 A drawing showing a comparison data of human CD137 amino acids sequence with cynomolgus monkey CD137 amino acids sequence.

FIG. 8 A graph showing the result of ELISA of IgGs obtained with phage display to CD3 and CD137.Y axis means the specificity to cyno CD137-Fc and X axis means the specificity to human CD137 of each clone.

FIG. 9 A graph showing the result of ELISA of IgGs obtained with phage display to CD3 and CD137.Y axis means the specificity to CD3e.

FIG. 10 A graph showing the result of competitive ELISA of IgGs obtained with phage display to CD3 and CD137. Y axis means the response of ELISA to biotin-human CD137-Fc or biotin-human Fc. Excess amount of human CD3 or human Fc were used as competitor.

FIG. 11A A set of graphs showing the result of phage ELISA of phage display panning output pools to CD3 and CD137.Y axis means the specificity to human CD137. X axis means the panning output pools, Primary is a pool before phage display panning, and R1 to R6 means panning output pool after phage display panning Round1 to Round6, respectively.

FIG. 11B A set of graphs showing the result of phage ELISA of phage display panning output pools to CD3 and CD137.Y axis means the specificity to cyno CD137. X axis means the panning output pools, Primary is a pool before phage display panning, and R1 to R6 means panning output pool after phage display panning Round1 to Round6, respectively.

FIG. 11C A set of graphs showing the result of phage ELISA of phage display panning output pools to CD3 and CD137.Y axis means the specificity to CD3. X axis means the panning output pools, Primary is a pool before phage display panning, and R1 to R6 means panning output pool after phage display panning Round1 to Round6, respectively.

FIG. 12.1 A set of graphs showing the result of ELISA of IgGs obtained with phage display to CD3 and CD137.Y axis means the specificity to human CD137-Fc and X axis means the specificity to cyno CD137 or CD3 of each clone.

FIG. 12.21A set of graphs showing the result of ELISA of IgGs obtained with phage display to CD3 and CD137. Y axis means the specificity to human CD137-Fc and X axis means the specificity to cyno CD137 or CD3 of each clone.

FIG. 12.31A set of graphs showing the result of ELISA of IgGs obtained with phage display to CD3 and CD137. Y axis means the specificity to human CD137-Fc and X axis means the specificity to cyno CD137 or CD3 of each clone.

FIG. 131A set of graphs showing the result of ELISA of IgGs obtained with phage display to CD3 and CD137.Y axis means the specificity to human CD137-Fc and X axis means the specificity to cyno CD137 or CD3 of each clone.

FIG. 141A graph showing the result of competitive ELISA of IgGs obtained with phage display to CD3 and CD137.Y axis means the response of ELISA to biotin-human CD137-Fc or biotin-human Fc. Excess amount of human CD3 were used as competitor.

FIG. 151A graph showing the result of ELISA of IgGs obtained with phage display to CD3 and CD137 to identify the epitope domain of each clones. Y axis means the response of ELISA to each domain of human CD137.

FIG. 161A set of graphs showing the result of ELISA of IgGs obtained with phage display affinity maturation to CD3 and CD137. Y axis means the specificity to human CD137-Fc and X axis means the specificity to cyno CD137 or CD3 of each clone.

FIG. 17.1 A set of graphs showing the result of competitive ELISA of IgGs obtained with phage display to CD3 and CD137. Y axis means the response of ELISA to biotin-human CD137-Fc or biotin-human Fc. An excess amount of human CD3 was used as a competitor.

FIG. 17.21A set of graphs showing the result of competitive ELISA of IgGs obtained with phage display to CD3 and CD137.Y axis means the response of ELISA to biotin-human CD137-Fc or biotin-human Fc. An excess amount of human CD3 was used as a competitor.

FIG. 17.31A set of graphs showing the result of competitive ELISA of IgGs obtained with phage display to CD3 and CD137.Y axis means the response of ELISA to biotin-human CD137-Fc or biotin-human Fc. An excess amount of human CD3 was used as a competitor.

FIG. 17.41A set of graphs showing the result of competitive ELISA of IgGs obtained with phage display to CD3 and CD137.Y axis means the response of ELISA to biotin-human CD137-Fc or biotin-human Fc. An excess amount of human CD3 was used as a competitor.

FIG. 17.51A set of graphs showing the result of competitive ELISA of IgGs obtained with phage display to CD3 and CD137.Y axis means the response of ELISA to biotin-human CD137-Fc or biotin-human Fc. An excess amount of human CD3 was used as a competitor.

FIG. 18A A drawing schematically showing the mechanism of IL-6 secretion from the activated B cell via anti-human GPC3/Dual-Fab antibodies.

FIG. 18B A graph showing the results of assessing the CD137-mediated agonist activity of various anti-human GPC3/Dual-Fab antibodies by the level of production of IL-6 which is secreted from the activated B cells. Ctrl indicates the negative control human IgG1 antibody.

FIG. 19A A drawing schematically showing the mechanism of Luciferase expression in the activated Jurkat T cell via anti-human GPC3/Dual-Fab antibodies.

FIG. 19B A set of graphs showing the results of assessing the CD3 mediated agonist activity of various anti-human GPC3/Dual-Fab antibodies by the level of production of Luciferase which is expressed in the activated Jurkat T cells. Ctrl indicates the negative control human IgG1 antibody.

FIG. 20 A set of graphs showing the results of assessing the cytokine (IL-2, IFN-gamma and TNF-alpha) release from human PBMC derived T cells in the presence of each immobilized antibodies. Y axis means the concentration of secreted each cytokines and X-axis means the concentration of immobilized antibodies. Control antiCD137 antibody (B), control anti-CD3 antibody (CE115), negative control antibody (Ctrl) and one of the dual antibody (H183L072) were used for assay.

FIG. 21 A set of graphs showing the results of assessing the T-cell dependent cellular cytotoxicity (TDCC) against GPC3 positive target cells (SK-pca60 and SK-pca13a) with each bi-specific antibodies. Y axis means the ratio of Cell Growth Inhibition (CGI) and X-axis means the concentration of each bi-specific antibodies. Anti-GPC3/Dual Bi-specific antibody (GC33/H183L072), Negative control/Dual Bi-specific antibody (Ctrl/H183L072), Anti-GPC3/Anti-CD137 Bi-specific antibody (GC33/B) and Negative control/Anti-CD137 Bi-specific antibody (Ctrl/B) were used for this assay. 5-fold amount of effector(E) cells were added on tumor(T) cells (ET5).

FIG. 221A graph showing results of cell-ELISA of CE115 for CD3e.

FIG. 231A diagram showing the molecular form of EGFR_ERY22_CE115.

FIG. 241A graph showing results of TDCC (SK-pca13a) of EGFR_ERY22_CE115.

FIG. 25 An exemplary sensorgram of an antibody having a ratio of the amounts bound of less than 0.8.

FIG. 26 is a set of graphs showing the results of Biacore analysis of simultaneous binding of GPC3/CD137×CD3 trispecific antibody and antiGPC3/dual-Fab antibody. Y-axis means the binding response to each antigen. At first human CD3 (hCD3) was used as analyte, and then also hCD3 (shown as broken line) or mixture of human CD137 (hCD137) and hCD3 (shown as solid line) were used as analyte.

FIG. 27 is a set of sensorgrams showing the results of FACS analysis to CD137 positive CHO cells or Jurkat cells of each antibodies. FIGS. 27(a) and (c) are the results of binding to human CD137 positive CHO cells, and FIGS. 27(b) and (d) are the results to parental CHO cells. In FIGS. 27(a) and (b), solid line shows the result of anti-GPC3/dual antibody (GC33/H183L072, i.e. GPC33/H183L072) and filled shows the result of control antibody (Ctrl). In FIGS. 27(c) and (d), solid line, filled with dark gray and filled with light grey shows the results of GPC3/CD137×Ctrl trispecific antibody, GPC3/CD137×CD3 trispecific antibody and Ctrl/Ctrl×CD3 trispecific antibody, respectively. FIGS. 27(e) and (f) are the results of binding to Jurkat CD3 positive cells. In FIG. 27(e), solid line and filled shows the result of antiGPC3/dual antibody (GC33/H183L072, i.e. GPC33/H183L072) and control antibody (Ctrl), respectively. In FIG. 27(f), solid line, filled with dark gray and filled with light grey shows the results of GPC3/Ctrl×CD3 trispecific antibody, GPC3/CD137×CD3 trispecific antibody and Ctrl/CD137×Ctrl trispecific antibody, respectively.

FIG. 28 presents graphs showing the results of assessing the CD3 mediated agonist activity of various a antibodies to GPC3 positive target cell SK-pca60 by the level of production of Luciferase which is expressed in the activated Jurkat T cells. Six kinds of tri-specific antibodies, anti-GPC3/Dual-Fab antibody (GPC3/H183L072) and control/Dual-Fab antibody (Ctrl/H183L072) were used for this assay. X-axis means the concentration used of each antibodies.

FIG. 29 presents graphs showing the results of assessing the CD3 mediated agonist activity of various a antibodies to human CD137 positive CHO cells and parental CHO cells by the level of production of Luciferase which is expressed in the activated Jurkat T cells. Six kinds of tri-specific antibodies, anti-GPC3/Dual-Fab antibody (GPC3/H183L072) and control/Dual-Fab antibody (Ctrl/H183L072) were used for this assay. X-axis means the concentration used of each antibodies.

FIG. 30 is a set of graphs showing the results of assessing the cytokine (IL-2, IFN-gamma and TNF-alpha) release from human PBMCs in the presence of each soluble antibodies. Y axis means the concentration of secreted each cytokines and X-axis means the concentration of antibodies used. Ctrl/CD137×CD3 trispecific antibody and control/Dual-Fab antibody (Ctrl/H183L072) were used for this assay.

### DESCRIPTION OF EMBODIMENTS

In the present invention, the “antibody variable region” usually means a region comprising a domain constituted by four framework regions (FRs) and three complementarity-determining regions (CDRs) flanked thereby, and also includes a partial sequence thereof as long as the partial sequence has the activity of binding to a portion or the whole of an antigen. Particularly, a region comprising an antibody light chain variable domain (VL) and an antibody heavy chain variable domain (VH) is preferred. The antibody variable region of the present invention may have an arbitrary sequence and may be a variable region derived from any antibody such as a mouse antibody, a rat antibody, a rabbit antibody, a goat antibody, a camel antibody, and a humanized antibody obtained by the humanization of any of these nonhuman antibodies, and a human antibody. The “humanized antibody”, also called reshaped human antibody, is obtained by grafting complementarity determining regions (CDRs) of a non-human mammal-derived antibody, for example, a mouse antibody to human antibody CDRs. Methods for identifying CDRs are known in the art (Kabat et al., Sequence of Proteins of Immunological Interest (1987), National Institute of Health, Bethesda, Md.; and Chothia et al., Nature (1989) 342: 877). General gene recombination approaches therefor are also known in the art (see European Patent Application Publication No. EP 125023 and WO 96/02576).

The “antibody variable region” of the present invention that does “not bind to CD3 and CD137 (4-1BB) at the same time” means that the antibody variable region of the present invention cannot bind to CD137 in a state bound with CD3 whereas the variable region cannot bind to CD3 in a state bound with CD137. In this context, the phrase “not bind to CD3 and CD137 at the same time” also includes not cross-linking a cell expressing CD3 to a cell expressing CD137, or not binding to CD3 and CD137 each expressed on a different cell, at the same time. This phrase further includes the case where the variable region is capable of binding to both CD3 and CD137 at the same time when CD3 and CD137 are not expressed on cell membranes, as with soluble proteins, or both reside on the same cell, but cannot bind to CD3 and CD137 each expressed on a different cell, at the same time. Such an antibody variable region is not particularly limited as long as the antibody variable region has these functions. Examples thereof can include variable regions derived from an IgG-type antibody variable region by the alteration of a portion of its amino acids so as to bind to the desired antigen. The amino acid to be altered is selected from, for example, amino acids whose alteration does not cancel the binding to the antigen, in an antibody variable region binding to CD3 or CD137.

In this context, the phrase “expressed on different cells” merely means that the antigens are expressed on separate cells. The combination of such cells may be, for example, the same types of cells such as a T cell and another T cell, or may be different types of cells such as a T cell and an NK cell.

In the present invention, one amino acid alteration may be used alone, or a plurality of amino acid alterations may be used in combination.

In the case of using a plurality of amino acid alterations in combination, the number of the alterations to be combined is not particularly limited and can be appropriately set within a range that can attain the object of the invention. The number of the alterations to be combined is, for example, 2 or more and 30 or less, preferably 2 or more and 25 or less, 2 or more and 22 or less, 2 or more and 20 or less, 2 or more and 15 or less, 2 or more and 10 or less, 2 or more and 5 or less, or 2 or more and 3 or less.

The plurality of amino acid alterations to be combined may be added to only the antibody heavy chain variable domain or light chain variable domain or may be appropriately distributed to both of the heavy chain variable domain and the light chain variable domain.

One or more amino acid residues in the variable region are acceptable as the amino acid residue to be altered as long as the antigen-binding activity is maintained. In the case of altering an amino acid in the variable region, the resulting variable region preferably maintains the binding activity of the corresponding unaltered antibody and preferably has, for example, 50% or higher, more preferably 80% or higher, further preferably 100% or higher, of the binding activity before the alteration, though the variable region according to the present invention is not limited thereto. The binding activity may be increased by the amino acid alteration and may be, for example, 2 times, 5 times, or 10 times the binding activity before the alteration.

Examples of the region preferred for the amino acid alteration include solvent-exposed regions and loops in the variable region. Among others, CDR1, CDR2, CDR3, FR3, and loops are preferred. Specifically, Kabat numbering positions 31 to 35, 50 to 65, 71 to 74, and 95 to 102 in the H chain variable domain and Kabat numbering positions 24 to 34, 50 to 56, and 89 to 97 in the L chain variable domain are preferred. Kabat numbering positions 31, 52a to 61, 71 to 74, and 97 to 101 in the H chain variable domain and Kabat numbering positions 24 to 34, 51 to 56, and 89 to 96 in the L chain variable domain are more preferred. Also, an amino acid that increases antigen-binding activity may be further introduced at the time of the amino acid alteration.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).
Unless otherwise indicated, HVR residues and other residues in the variable domain (e.g., FR residues) are numbered herein according to Kabat et al., supra.

In the present invention, the “loop” means a region containing residues that are not involved in the maintenance of an immunoglobulin beta barrel structure.

In the present invention, the amino acid alteration means substitution, deletion, addition, insertion, or modification, or a combination thereof. In the present invention, the amino acid alteration can be used interchangeably with amino acid mutation and used in the same sense therewith.

The substitution of an amino acid residue is carried out by replacement with another amino acid residue for the purpose of altering, for example, any of the following (a) to (c): (a) the polypeptide backbone structure of a region having a sheet structure or helix structure; (b) the electric charge or hydrophobicity of a target site; and (c) the size of a side chain.

Amino acid residues are classified into the following groups on the basis of general side chain properties: (1) hydrophobic residues: norleucine, Met, Ala, Val, Leu, and Ile; (2) neutral hydrophilic residues: Cys, Ser, Thr, Asn, and Gln; (3) acidic residues: Asp and Glu; (4) basic residues: His, Lys, and Arg; (5) residues that influence chain orientation: Gly and Pro; and (6) aromatic residues: Trp, Tyr, and Phe.

The substitution of amino acid residues within each of these groups is called conservative substitution, while the substitution of an amino acid residue in one of these groups by an amino acid residue in another group is called non-conservative substitution.

The substitution according to the present invention may be the conservative substitution or may be the non-conservative substitution. Alternatively, the conservative substitution and the non-conservative substitution may be combined.

The alteration of an amino acid residue also includes: the selection of a variable region that is capable of binding to CD3 and CD137, but cannot bind to these antigens at the same time, from those obtained by the random alteration of amino acids whose alteration does not cancel the binding to the antigen, in the antibody variable region binding to CD3 or CD137; and alteration to insert a peptide previously known to have binding activity against the desired antigen, to the region mentioned above.

In the antibody variable region of the present invention, the alteration mentioned above may be combined with alteration known in the art. For example, the modification of N-terminal glutamine of the variable region to pyroglutamic acid by pyroglutamylation is a modification well known to those skilled in the art. Thus, the antibody of the present invention having glutamine at the N terminus of its heavy chain may contain a variable region with this N-terminal glutamine modified to pyroglutamic acid.

Such an antibody variable region may further have amino acid alteration to improve, for example, antigen binding, pharmacokinetics, stability, or antigenicity. The antibody variable region of the present invention may be altered so as to have pH dependent binding activity against an antigen and be thereby capable of repetitively binding to the antigen (WO2009/125825).

Also, amino acid alteration to change antigen-binding activity according to the concentration of a target tissue-specific compound may be added to, for example, such an antibody variable region binding to a third antigen (WO2013/180200).

The variable region may be further altered for the purpose of, for example, enhancing binding activity, improving specificity, reducing pI, conferring pH-dependent antigen-binding properties, improving the thermal stability of binding, improving solubility, improving stability against chemical modification, improving heterogeneity derived from a sugar chain, avoiding a T cell epitope identified by use of in silico prediction or in vitro T cell-based assay for reduction in immunogenicity, or introducing a T cell epitope for activating regulatory T cells (mAbs 3: 243-247, 2011).

Whether the antibody variable region of the present invention is “capable of binding to CD3 and CD137” can be determined by a method known in the art.

This can be determined by, for example, an electrochemiluminescence method (ECL method) (BMC Research Notes 2011, 4: 281).

Specifically, for example, a low-molecular antibody composed of a region capable of binding to CD3 and CD137, for example, a Fab region, of a biotin-labeled antigen-binding molecule to be tested, or a monovalent antibody (antibody lacking one of the two Fab regions carried by a usual antibody) thereof is mixed with CD3 or CD137 labeled with sulfo-tag (Ru complex), and the mixture is added onto a streptavidin-immobilized plate. In this operation, the biotin-labeled antigen-binding molecule to be tested binds to streptavidin on the plate. Light is developed from the sulfo-tag, and the luminescence signal can be detected using Sector Imager 600 or 2400 (MSD K.K.) or the like to thereby confirm the binding of the aforementioned region of the antigen-binding molecule to be tested to CD3 or CD137.

Alternatively, this assay may be conducted by ELISA, FACS (fluorescence activated cell sorting), ALPHAScreen (amplified luminescent proximity homogeneous assay screen), the BIACORE method based on a surface plasmon resonance (SPR) phenomenon, etc. (Proc. Natl. Acad. Sci. USA (2006) 103 (11), 4005-4010).

Specifically, the assay can be conducted using, for example, an interaction analyzer Biacore (GE Healthcare Japan Corp.) based on a surface plasmon resonance (SPR) phenomenon. The Biacore analyzer includes any model such as Biacore T100, T200, X100, A100, 4000, 3000, 2000, 1000, or C. Any sensor chip for Biacore, such as a CM7, CM5, CM4, CM3, C1, SA, NTA, L1, HPA, or Au chip, can be used as a sensor chip. Proteins for capturing the antigen-binding molecule of the present invention, such as protein A, protein G, protein L, anti-human IgG antibodies, anti-human IgG-Fab, anti-human L chain antibodies, anti-human Fc antibodies, antigenic proteins, or antigenic peptides, are immobilized onto the sensor chip by a coupling method such as amine coupling, disulfide coupling, or aldehyde coupling. CD3 or CD137 is injected thereon as an analyte, and the interaction is measured to obtain a sensorgram. In this operation, the concentration of CD3 or CD137 can be selected within the range of a few micro M to a few pM according to the interaction strength (e.g., KD) of the assay sample.

Alternatively, CD3 or CD137 may be immobilized instead of the antigen-binding molecule onto the sensor chip, with which the antibody sample to be evaluated is in turn allowed to interact. Whether the antibody variable region of the antigen-binding molecule of the present invention has binding activity against CD3 or CD137 can be confirmed on the basis of a dissociation constant (KD) value calculated from the sensorgram of the interaction or on the basis of the degree of increase in the sensorgram after the action of the antigen-binding molecule sample over the level before the action.

In some embodiments, binding activity or affinity of the antibody variable region of the present invention to the antigen of interest (i.e. CD3 or CD137) are assessed at 37 degrees C. (for CD137) or 25 degrees C. (for CD3) using e.g., Biacore T200 instrument (GE Healthcare) or Biacore 8K instrument (GE Healthcare). Anti-human Fc (e.g., GE Healthcare) is immobilized onto all flow cells of a CM4 sensor chip using amine coupling kit (e.g, GE Healthcare). The antigen binding molecules or antibody variable regions are captured onto the anti-Fc sensor surfaces, then the antigen (CD3 or CD137) is injected over the flow cell. The capture levels of the antigen binding molecules or antibody variable regions may be aimed at 200 resonance unit (RU). Recombinant human CD3 or CD137 may be injected at 400 to 25 nM prepared by two-fold serial dilution, followed by dissociation. All antigen binding molecules or antibody variable regions and analytes are prepared in ACES pH 7.4 containing 20 mM ACES, 150 mM NaCl, 0.05% Tween 20, 0.005% NaN3. Sensor surface is regenerated each cycle with 3 M MgCl2. Binding affinity are determined by processing and fitting the data to 1:1 binding model using e.g., Biacore T200 Evaluation software, version 2.0 (GE Healthcare) or Biacore 8K Evaluation software (GE Healthcare). The KD values are calculated for assessing the specific binding activity or affinity of the antigen binding domains of the present invention.

The ALPHAScreen is carried out by the ALPHA technology using two types of beads (donor and acceptor) on the basis of the following principle: luminescence signals are detected only when these two beads are located in proximity through the biological interaction between a molecule bound with the donor bead and a molecule bound with the acceptor bead. A laser-excited photosensitizer in the donor bead converts ambient oxygen to singlet oxygen having an excited state. The singlet oxygen diffuses around the donor bead and reaches the acceptor bead located in proximity thereto to thereby cause chemiluminescent reaction in the bead, which finally emits light. In the absence of the interaction between the molecule bound with the donor bead and the molecule bound with the acceptor bead, singlet oxygen produced by the donor bead does not reach the acceptor bead. Thus, no chemiluminescent reaction occurs.

One (ligand) of the substances between which the interaction is to be observed is immobilized onto a thin gold film of a sensor chip. The sensor chip is irradiated with light from the back such that total reflection occurs at the interface between the thin gold film and glass. As a result, a site having a drop in reflection intensity (SPR signal) is formed in a portion of reflected light. The other (analyte) of the substances between which the interaction is to be observed is injected on the surface of the sensor chip. Upon binding of the analyte to the ligand, the mass of the immobilized ligand molecule is increased to change the refractive index of the solvent on the sensor chip surface. This change in the refractive index shifts the position of the SPR signal (on the contrary, the dissociation of the bound molecules gets the signal back to the original position). The Biacore system plots on the ordinate the amount of the shift, i.e., change in mass on the sensor chip surface, and displays time-dependent change in mass as assay data (sensorgram). The amount of the analyte bound to the ligand captured on the sensor chip surface (amount of change in response on the sensorgram between before and after the interaction of the analyte) can be determined from the sensorgram. However, since the amount bound also depends on the amount of the ligand, the comparison must be performed under conditions where substantially the same amounts of the ligand are used. Kinetics, i.e., an association rate constant (ka) and a dissociation rate constant (kd), can be determined from the curve of the sensorgram, while affinity (KD) can be determined from the ratio between these constants. Inhibition assay is also preferably used in the BIACORE method. Examples of the inhibition assay are described in Proc. Natl. Acad. Sci. USA (2006) 103 (11), 4005-4010.

Whether the antigen-binding molecule of the present invention does “not bind to CD3 and CD137 at the same time” can be confirmed by: confirming the antigen-binding molecule to have binding activity against both CD3 and CD137; then allowing either CD3 or CD137 to bind in advance to the antigen-binding molecule comprising the variable region having this binding activity; and then determining the presence or absence of its binding activity against the other one by the method mentioned above. Alternatively, this can also be confirmed by determining whether the binding of the antigen-binding molecule to either CD3 or CD137 immobilized on an ELISA plate or a sensor chip is inhibited by the addition of the other one into the solution. In some embodiments, the binding of the antigen-binding molecule of the present invention to either CD3 or CD137 is inhibited by binding of the antigen-binding molecule to the other by at least 50%, preferably 60% or more, more preferably 70% or more, more preferably 80% or more, further preferably 90% or more, or even more preferably 95% or more.

In one aspect, while one antigen (e.g. CD3) is immobilized, the inhibition of the binding of the antigen-binding molecule to CD3 can be determined in the presence of the other antigen (e.g. CD137) by methods known in prior art (i.e. ELISA, BIACORE, and so on). In another aspect, while CD137 is immobilized, the inhibition of the binding of the antigen-binding molecule to CD137 also can be determined in the presence of CD3. When either one of two aspects mentioned above is conducted, the antigen-binding molecule of the present invention is determined not to bind to CD3 and CD137 at the same time if the binding is inhibited by at least 50%, preferably 60% or more, preferably 70% or more, further preferably 80% or more, further preferably 90% or more, or even more preferably 95% or more.

In some embodiments, the concentration of the antigen injected as an analyte is at least 1-fold, 2-fold, 5-fold, 10-fold, 30-fold, 50-fold, or 100-fold higher than the concentration of the other antigen to be immobilized.

In preferable manner, the concentration of the antigen injected as an analyte is 100-fold higher than the concentration of the other antigen to be immobilized and the binding is inhibited by at least 80%.

In one embodiment, the ratio of the KD value for the CD3 (analyte)-binding activity of the antigen-binding molecule to the CD137 (immobilized)-binding activity of the antigen-binding molecule (KD (CD3)/KD (CD137)) is calculated and the CD3 (analyte) concentration which is 10-fold, 50-fold, 100-fold, or 200-fold of the ratio of the KD value (KD(CD3)/KD(CD137) higher than the CD137 (immobilized) concentration can be used for the competition measurement above. (e.g. 1-fold, 5-fold, 10-fold, or 20-fold higher concentration can be selected when the ratio of the KD value is 0.1. Furthermore, 100-fold, 500-fold, 1000-fold, or 2000-fold higher concentration can be selected when the ratio of the KD value is 10.)

In one aspect, while one antigen (e.g. CD3) is immobilized, the attenuation of the binding signal of the antigen-binding molecule to CD3 can be determined in the presence of the other antigen (e.g. CD137) by methods known in prior art (i.e. ELISA, ECL and so on). In another aspect, while CD137 is immobilized, the attenuation of the binding signal of the antigen-binding molecule to CD137 also can be determined in the presence of CD3. When either one of two aspects mentioned above is conducted, the antigen-binding molecule of the present invention is determined not to bind to CD3 and CD137 at the same time if the binding signal is attenuated by at least 50%, preferably 60% or more, preferably 70% or more, further preferably 80% or more, further preferably 90% or more, or even more preferably 95% or more.

In some embodiments, the concentration of the antigen injected as an analyte is at least 1-fold, 2-fold, 5-fold, 10-fold, 30-fold, 50-fold, or 100-fold higher than the concentration of the other antigen to be immobilized.

In preferable manner, the concentration of the antigen injected as an analyte is 100-fold higher than the concentration of the other antigen to be immobilized and the binding is inhibited by at least 80%.

In one embodiment, the ratio of the KD value for the CD3 (analyte)-binding activity of the antigen-binding molecule to the CD137 (immobilized)-binding activity of the antigen-binding molecule (KD (CD3)/KD (CD137)) is calculated and the CD3 (analyte) concentration which is 10-fold, 50-fold, 100-fold, or 200-fold of the ratio of the KD value (KD(CD3)/KD(CD137) higher than the CD137 (immobilized) concentration can be used for the measurement above. (e.g. 1-fold, 5-fold, 10-fold, or 20-fold higher concentration can be selected when the ratio of the KD value is 0.1. Furthermore, 100-fold, 500-fold, 1000-fold, or 2000-fold higher concentration can be selected when the ratio of the KD value is 10.)

Specifically, in the case of using, for example, the ECL method, a biotin-labeled antigen-binding molecule to be tested, CD3 labeled with sulfo-tag (Ru complex), and an unlabeled CD137 are prepared. When the antigen-binding molecule to be tested is capable of binding to CD3 and CD137, but does not bind to CD3 and CD137 at the same time, the luminescence signal of the sulfo-tag is detected in the absence of the unlabeled CD137 by adding the mixture of the antigen-binding molecule to be tested and labeled CD3 onto a streptavidin-immobilized plate, followed by light development. By contrast, the luminescence signal is decreased in the presence of unlabeled CD137. This decrease in luminescence signal can be quantified to determine relative binding activity. This analysis may be similarly conducted using the labeled CD137 and the unlabeled CD3.

In the case of the ALPHAScreen, the antigen-binding molecule to be tested interacts with CD3 in the absence of the competing CD137 to generate signals of 520 to 620 nm. The untagged CD137 competes with CD3 for the interaction with the antigen-binding molecule to be tested. Decrease in fluorescence caused as a result of the competition can be quantified to thereby determine relative binding activity. The polypeptide biotinylation using sulfo-NHS-biotin or the like is known in the art. CD3 can be tagged with GST by an appropriately adopted method which involves, for example: fusing a polynucleotide encoding CD3 in flame with a polynucleotide encoding GST; and allowing the resulting fusion gene to be expressed by cells or the like harboring vectors capable of expression thereof, followed by purification using a glutathione column. The obtained signals are preferably analyzed using, for example, software GRAPHPAD PRISM (GraphPad Software, Inc., San Diego) adapted to a one-site competition model based on nonlinear regression analysis. This analysis may be similarly conducted using the tagged CD137 and the untagged CD3.

Alternatively, a method using fluorescence resonance energy transfer (FRET) may be used. FRET is a phenomenon in which excitation energy is transferred directly between two fluorescent molecules located in proximity to each other by electron resonance. When FRET occurs, the excitation energy of a donor (fluorescent molecule having an excited state) is transferred to an acceptor (another fluorescent molecule located near the donor) so that the fluorescence emitted from the donor disappears (to be precise, the lifetime of the fluorescence is shortened) and instead, the fluorescence is emitted from the acceptor. By use of this phenomenon, whether or not bind to CD3 and CD137 at the same time can be analyzed. For example, when CD3 carrying a fluorescence donor and CD137 carrying a fluorescence acceptor bind to the antigen-binding molecule to be tested at the same time, the fluorescence of the donor disappears while the fluorescence is emitted from the acceptor. Therefore, change in fluorescence wavelength is observed. Such an antibody is confirmed to bind to CD3 and CD137 at the same time. On the other hand, if the mixing of CD3, CD137, and the antigen-binding molecule to be tested does not change the fluorescence wavelength of the fluorescence donor bound with CD3, this antigen-binding molecule to be tested can be regarded as antigen binding domain that is capable of binding to CD3 and CD137, but does not bind to CD3 and CD137 at the same time.

For example, a biotin-labeled antigen-binding molecule to be tested is allowed to bind to streptavidin on the donor bead, while CD3 tagged with glutathione S transferase (GST) is allowed to bind to the acceptor bead. The antigen-binding molecule to be tested interacts with CD3 in the absence of the competing second antigen to generate signals of 520 to 620 nm. The untagged second antigen competes with CD3 for the interaction with the antigen-binding molecule to be tested. Decrease in fluorescence caused as a result of the competition can be quantified to thereby determine relative binding activity. The polypeptide biotinylation using sulfo-NHS-biotin or the like is known in the art. CD3 can be tagged with GST by an appropriately adopted method which involves, for example: fusing a polynucleotide encoding CD3 in flame with a polynucleotide encoding GST; and allowing the resulting fusion gene to be expressed by cells or the like harboring vectors capable of expression thereof, followed by purification using a glutathione column. The obtained signals are preferably analyzed using, for example, software GRAPHPAD PRISM (GraphPad Software, Inc., San Diego) adapted to a one-site competition model based on nonlinear regression analysis.

The tagging is not limited to the GST tagging and may be carried out with any tag such as, but not limited to, a histidine tag, MBP, CBP, a Flag tag, an HA tag, a V5 tag, or a c-myc tag. The binding of the antigen-binding molecule to be tested to the donor bead is not limited to the binding using biotin-streptavidin reaction. Particularly, when the antigen-binding molecule to be tested comprises Fc, a possible method involves allowing the antigen-binding molecule to be tested to bind via an Fc-recognizing protein such as protein A or protein G on the donor bead.

Also, the case where the variable region is capable of binding to CD3 and CD137 at the same time when CD3 and CD137 are not expressed on cell membranes, as with soluble proteins, or both reside on the same cell, but cannot bind to CD3 and CD137 each expressed on a different cell, at the same time can also be assayed by a method known in the art.

Specifically, the antigen-binding molecule to be tested has been confirmed to be positive in ECL-ELISA for detecting binding to CD3 and CD137 at the same time is also mixed with a cell expressing CD3 and a cell expressing CD137. The antigen-binding molecule to be tested can be shown to be incapable of binding to CD3 and CD137 expressed on different cells, at the same time unless the antigen-binding molecule and these cells bind to each other at the same time. This assay can be conducted by, for example, cell-based ECL-ELISA. The cell expressing CD3 is immobilized onto a plate in advance. After binding of the antigen-binding molecule to be tested thereto, the cell expressing CD137 is added to the plate. A different antigen expressed only on the cell expressing CD137 is detected using a sulfo-tag-labeled antibody against this antigen. A signal is observed when the antigen-binding molecule binds to the two antigens respectively expressed on the two cells, at the same time. No signal is observed when the antigen-binding molecule does not bind to these antigens at the same time.

Alternatively, this assay may be conducted by the ALPHAScreen method. The antigen-binding molecule to be tested is mixed with a cell expressing CD3 bound with the donor bead and a cell expressing CD137 bound with the acceptor bead. A signal is observed when the antigen-binding molecule binds to the two antigens expressed on the two cells respectively, at the same time. No signal is observed when the antigen-binding molecule does not bind to these antigens at the same time.
Alternatively, this assay may also be conducted by an Octet interaction analysis method. First, a cell expressing CD3 tagged with a peptide tag is allowed to bind to a biosensor that recognizes the peptide tag. A cell expressing CD137 and the antigen-binding molecule to be tested are placed in wells and analyzed for interaction. A large wavelength shift caused by the binding of the antigen-binding molecule to be tested and the cell expressing CD137 to the biosensor is observed when the antigen-binding molecule binds to the two antigens expressed on the two cells respectively, at the same time. A small wavelength shift caused by the binding of only the antigen-binding molecule to be tested to the biosensor is observed when the antigen-binding molecule does not bind to these antigens at the same time.

Instead of these methods based on the binding activity, assay based on biological activity may be conducted. For example, a cell expressing CD3 and a cell expressing CD137 are mixed with the antigen-binding molecule to be tested, and cultured. The two antigens expressed on the two cells respectively are mutually activated via the antigen-binding molecule to be tested when the antigen-binding molecule binds to these two antigens at the same time. Therefore, change in activation signal, such as increase in the respective downstream phosphorylation levels of the antigens, can be detected. Alternatively, cytokine production is induced as a result of the activation. Therefore, the amount of cytokines produced can be measured to thereby confirm whether or not to bind to the two cells at the same time. Alternatively, cytotoxicity against a cell expressing CD137 is induced as a result of the activation. Alternatively, the expression of a reporter gene is induced by a promoter which is activated at the downstream of the signal transduction pathway of CD137 or CD3 as a result of the activation. Therefore, the cytotoxicity or the amount of reporter proteins produced can be measured to thereby confirm whether or not to bind to the two cells at the same time.

In an embodiment, the cellular cytotoxicity is T cell-dependent cellular cytotoxicity (TDCC). In another embodiment, the cytotoxicity is a cellular cytotoxicity towards cells expressing CD3 or CD137 on their surfaces. The (cellular) cytotoxicity or TDCC of an antibody (or antigen-binding molecule) of the present invention can be evaluated by any suitable method known in the art. For example, TDCC can be measured by real-time cell growth inhibition assay as described in Example 2.3.2. In this assay, target cells are incubated with T cells (e.g. PBMCs) or expanded T cells in the presence of a test antibody on a 96-well plate, and the growth of the target cells is monitored by methods known in the art, for example, by using a suitable analyzing instrument (e.g. xCELLigence Real-Time Cell Analyzer). The rate of cell growth inhibition (CGI: %) is determined from the cell index value according to the formulation given as CGI (%)=100−(CI_{Ab}×100/CI_{NoAb}). “CI_{Ab}” represents the cell index value of wells with the antibody on a specific experimental time and “CI_{NoAb}” represents the average cell index value of wells without the antibody. If the CGI rate of the antibody is high, i.e., has a significantly positive value, it can be said that the antibody has TDCC activity.

In a preferred aspect, T cell activation can be assayed by methods known in the art, such as a method using an engineered T cell line that expresses a reporter gene (e.g. luciferase) in response to its activation (e.g. Jurkat/NFAT-RE Reporter Cell Line (T Cell Activation Bioassay, Promega)). In this method, target cells (e.g. a cell expressing CD3 and a cell expressing CD137) are cultured with T cells in the presence of a test antibody, and then the level or activity of the expression product of the reporter gene is measured by appropriate methods as an index of T cell activation. When the reporter gene is a luciferase gene, luminescence arising from reaction between luciferase and its substrate may be measured as an index of T cell activation. If T cell activation measured as described above is higher, the test antibody is determined to have higher T cell activation activity. In one aspect, when recombinant T cells that express a reporter gene in response to CD3 signaling are co-cultured with cells expressing CD137 in the presence of an antigen-binding molecule, the antigen-binding molecule is determined not to induce activation of T cells against cells expressing CD137 if the expression of the reporter gene or the activity of the reporter gene product is at most about 50%, 30%, 20%, 10%, 5% or 1%, where 100% activation is the level of activation achieved by an antigen-binding molecule which binds to CD3 and CD137 at the same time. In one aspect, when recombinant T cells that express a reporter gene in response to CD3 signaling are co-cultured with cells expressing CD137 in the presence of an antigen-binding molecule, the antigen-binding molecule is determined not to induce activation of T cells against cells expressing CD137 if the expression of the reporter gene or the activity of the reporter gene product is at most about 50%, 30%, 20%, 10%, 5% or 1%, where 100% activation is the level of activation achieved by the same antigen-binding molecule against cells expressing the molecule of a third antigen.

In one embodiments, whether an antigen-binding molecule does not induce release of cytokines can be determined by, for example, incubating PBMCs with the antigen-binding molecule, and measuring cytokines such as IL-2, IFN gamma, and TNF alpha released from the PBMCs into the culture supernatant using methods known in the art. If no significant levels of cytokines are detected or no significant induction of cytokines expression occurred in the culture supernatant of PBMCs that have been incubated with an antigen-binding molecule, the antigen-binding molecule is determined not to induce a cytokine release from PBMCs n. In one aspect, “no significant levels of cytokines” also refers to the level of cytokines concentration that is about at most 50%, 30%, 20%, 10%, 5% or 1%, where 100% is the cytokine concentration achieved by an antigen-binding molecule which binds to CD3 and CD137 at the same time. In one aspect, “no significant levels of cytokines” also refers to the level of cytokines concentration that is about at most 50%, 30%, 20%, 10%, 5% or 1%, where 100% is the cytokine concentration achieved in the presence of cells expressing the molecule of a third antigen. In one aspect, “no significant induction of cytokines expression” also refers to the level of cytokines concentration increase that is at most 5-fold, 2-fold or 1-fold of the concentration of each cytokines before adding the antigen-binding molecules.

In the present invention, the “Fc region” refers to a region comprising a fragment consisting of a hinge or a portion thereof and CH2 and CH3 domains in an antibody molecule. The Fc region of IgG class means, but is not limited to, a region from, for example, cysteine 226 (EU numbering (also referred to as EU index herein)) to the C terminus or proline 230 (EU numbering) to the C terminus. The Fc region can be preferably obtained by the partial digestion of, for example, an IgG1, IgG2, IgG3, or IgG4 monoclonal antibody with a proteolytic enzyme such as pepsin followed by the re-elution of a fraction adsorbed on a protein A column or a protein G column. Such a proteolytic enzyme is not particularly limited as long as the enzyme is capable of digesting a whole antibody to restrictively form Fab or F(ab′)₂ under appropriately set reaction conditions (e.g., pH) of the enzyme. Examples thereof can include pepsin and papain.

In some embodiments, the “antigen-binding molecule” is not particularly limited as long as the molecule comprises the “antibody variable region” of the present invention. The antigen-binding molecule may further comprise a peptide or a protein having a length of approximately 5 or more amino acids. The peptide or the protein is not limited to a peptide or a protein derived from an organism, and may be, for example, a polypeptide consisting of an artificially designed sequence. Also, a natural polypeptide, a synthetic polypeptide, a recombinant polypeptide, or the like may be used.

In some embodiments, the “antigen-binding molecule” of the present invention is not particularly limited to a molecule comprising the “antibody variable region”. In certain embodiments, antigen-binding molecules that are other than antibodies comprising a variable region and can bind to two different antigens, for example, Affibody and so on, may be obtained by methods generally known to those skilled in the art (PLoS One. 2011; 6(10):e25791; PLoS One. 2012; 7(8):e42288; J Mol Biol. 2011 Aug. 5; 411(1):201-19; Proc Natl Acad Sci USA. 2011 Aug. 23; 108(34):14067-72).

Preferred examples of the antigen-binding molecule of the present invention can include an antigen-binding molecule comprising an antibody Fc region.

An Fc region derived from, for example, naturally occurring IgG can be used as the “Fc region” of the present invention. In this context, the naturally occurring IgG means a polypeptide that contains an amino acid sequence identical to that of IgG found in nature and belongs to a class of an antibody substantially encoded by an immunoglobulin gamma gene. The naturally occurring human IgG means, for example, naturally occurring human IgG1, naturally occurring human IgG2, naturally occurring human IgG3, or naturally occurring human IgG4. The naturally occurring IgG also includes variants or the like spontaneously derived therefrom. A plurality of allotype sequences based on gene polymorphism are described as the constant regions of human IgG1, human IgG2, human IgG3, and human IgG4 antibodies in Sequences of proteins of immunological interest, NIH Publication No. 91-3242, any of which can be used in the present invention. Particularly, the sequence of human IgG1 may have DEL or EEM as an amino acid sequence of EU numbering positions 356 to 358.

The antibody Fc region is found as, for example, an Fc region of IgA1, IgA2, IgD, IgE, IgG1, IgG2, IgG3, IgG4, or IgM type. For example, an Fc region derived from a naturally occurring human IgG antibody can be used as the antibody Fc region of the present invention. For example, an Fc region derived from a constant region of naturally occurring IgG, specifically, a constant region (SEQ ID NO: 208) originated from naturally occurring human IgG1, a constant region (SEQ ID NO: 209) originated from naturally occurring human IgG2, a constant region (SEQ ID NO: 210) originated from naturally occurring human IgG3, or a constant region (SEQ ID NO: 211) originated from naturally occurring human IgG4 can be used as the Fc region of the present invention. The constant region of naturally occurring IgG also includes variants or the like spontaneously derived therefrom.

The Fc region of the present invention is particularly preferably an Fc region having reduced binding activity against an Fc gamma receptor. In this context, the Fc gamma receptor (also referred to as Fc gamma R herein) refers to a receptor capable of binding to the Fc region of IgG1, IgG2, IgG3, or IgG4 and means any member of the protein family substantially encoded by Fc gamma receptor genes. In humans, this family includes, but is not limited to: Fc gamma RI (CD64) including isoforms Fc gamma RIa, Fc gamma RIb, and Fc gamma RIc; Fc gamma RII (CD32) including isoforms Fc gamma RIIa (including allotypes H131 (H type) and R131 (R type)), Fc gamma RIIb (including Fc gamma RIIb-1 and Fc gamma RIIb-2), and Fc gamma RIIc; and Fc gamma RIII (CD16) including isoforms Fc gamma RIIIa (including allotypes V158 and F158) and Fc gamma RIIIb (including allotypes Fc gamma RIIIb-NA1 and Fc gamma RIIIb-NA2); and any yet-to-be-discovered human Fc gamma R or Fc gamma R isoform or allotype. The Fc gamma R includes those derived from humans, mice, rats, rabbits, and monkeys. The Fc gamma R is not limited to these molecules and may be derived from any organism. The mouse Fc gamma Rs include, but are not limited to, Fc gamma RI (CD64), Fc gamma RII (CD32), Fc gamma RIII (CD16), and Fc gamma RIII-2 (CD16-2), and any yet-to-be-discovered mouse Fc gamma R or Fc gamma R isoform or allotype. Preferred examples of such Fc gamma receptors include human Fc gamma RI (CD64), Fc gamma RIIa (CD32), Fc gamma RIIb (CD32), Fc gamma RIIIa (CD16), and/or Fc gamma RIIIb (CD16).

The Fc gamma R is found in the forms of an activating receptor having ITAM (immunoreceptor tyrosine-based activation motif) and an inhibitory receptor having ITIM (immunoreceptor tyrosine-based inhibitory motif). The Fc gamma R is classified into activating Fc gamma R (Fc gamma RI, Fc gamma RIIa R, Fc gamma RIIa H, Fc gamma RIIIa, and Fc gamma RIIIb) and inhibitory Fc gamma R (Fc gamma RIIb). The polynucleotide sequence and the amino acid sequence of Fc gamma RI are described in NM_000566.3 and NP_000557.1, respectively; the polynucleotide sequence and the amino acid sequence of Fc gamma RIIa are described in BCO20823.1 and AAH20823.1, respectively; the polynucleotide sequence and the amino acid sequence of Fc gamma RIIb are described in BC146678.1 and AAI46679.1, respectively; the polynucleotide sequence and the amino acid sequence of Fc gamma RIIIa are described in BC033678.1 and AAH33678.1, respectively; and the polynucleotide sequence and the amino acid sequence of Fc gamma RIIIb are described in BC128562.1 and AAI28563.1, respectively (RefSeq registration numbers). Fc gamma RIIa has two types of gene polymorphisms that substitute the 131st amino acid of Fc gamma RIIa by histidine (H type) or arginine (R type) (J. Exp. Med, 172, 19-25, 1990). Fc gamma RIIb has two types of gene polymorphisms that substitute the 232nd amino acid of Fc gamma RIIb by isoleucine (I type) or threonine (T type) (Arthritis. Rheum. 46: 1242-1254 (2002)). Fc gamma RIIIa has two types of gene polymorphisms that substitute the 158th amino acid of Fc gamma RIIIa by valine (V type) or phenylalanine (F type) (J. Clin. Invest. 100 (5): 1059-1070 (1997)). Fc gamma RIIIb has two types of gene polymorphisms (NA1 type and NA2 type) (J. Clin. Invest. 85: 1287-1295 (1990)).

The reduced binding activity against an Fc gamma receptor can be confirmed by a well-known method such as FACS, ELISA format, ALPHAScreen (amplified luminescent proximity homogeneous assay screen), or the BIACORE method based on a surface plasmon resonance (SPR) phenomenon (Proc. Natl. Acad. Sci. USA (2006) 103 (11), 4005-4010).

The ALPHAScreen method is carried out by the ALPHA technology using two types of beads (donor and acceptor) on the basis of the following principle: luminescence signals are detected only when these two beads are located in proximity through the biological interaction between a molecule bound with the donor bead and a molecule bound with the acceptor bead. A laser-excited photosensitizer in the donor bead converts ambient oxygen to singlet oxygen having an excited state. The singlet oxygen diffuses around the donor bead and reaches the acceptor bead located in proximity thereto to thereby cause chemiluminescent reaction in the bead, which finally emits light. In the absence of the interaction between the molecule bound with the donor bead and the molecule bound with the acceptor bead, singlet oxygen produced by the donor bead does not reach the acceptor bead. Thus, no chemiluminescent reaction occurs.

For example, a biotin-labeled antigen-binding molecule is allowed to bind to the donor bead, while a glutathione S transferase (GST)-tagged Fc gamma receptor is allowed to bind to the acceptor bead. In the absence of a competing antigen-binding molecule having a mutated Fc region, an antigen-binding molecule having a wild-type Fc region interacts with the Fc gamma receptor to generate signals of 520 to 620 nm. The untagged antigen-binding molecule having a mutated Fc region competes with the antigen-binding molecule having a wild-type Fc region for the interaction with the Fc gamma receptor. Decrease in fluorescence caused as a result of the competition can be quantified to thereby determine relative binding affinity. The antigen-binding molecule (e.g., antibody) biotinylation using sulfo-NHS-biotin or the like is known in the art. The Fc gamma receptor can be tagged with GST by an appropriately adopted method which involves, for example: fusing a polynucleotide encoding the Fc gamma receptor in flame with a polynucleotide encoding GST; and allowing the resulting fusion gene to be expressed by cells or the like harboring vectors capable of expression thereof, followed by purification using a glutathione column. The obtained signals are preferably analyzed using, for example, software GRAPHPAD PRISM (GraphPad Software, Inc., San Diego) adapted to a one-site competition model based on nonlinear regression analysis.

One (ligand) of the substances between which the interaction is to be observed is immobilized onto a thin gold film of a sensor chip. The sensor chip is irradiated with light from the back such that total reflection occurs at the interface between the thin gold film and glass. As a result, a site having a drop in reflection intensity (SPR signal) is formed in a portion of reflected light. The other (analyte) of the substances between which the interaction is to be observed is injected on the surface of the sensor chip. Upon binding of the analyte to the ligand, the mass of the immobilized ligand molecule is increased to change the refractive index of the solvent on the sensor chip surface. This change in the refractive index shifts the position of the SPR signal (on the contrary, the dissociation of the bound molecules gets the signal back to the original position). The Biacore system plots on the ordinate the amount of the shift, i.e., change in mass on the sensor chip surface, and displays time-dependent change in mass as assay data (sensorgram). Kinetics, i.e., an association rate constant (ka) and a dissociation rate constant (kd), can be determined from the curve of the sensorgram, while affinity (KD) can be determined from the ratio between these constants. Inhibition assay is also preferably used in the BIACORE method. Examples of the inhibition assay are described in Proc. Natl. Acad. Sci. USA (2006) 103 (11), 4005-4010.

In the present specification, the reduced binding activity against an Fc gamma receptor means that the antigen-binding molecule to be tested exhibits binding activity of, for example, 50% or lower, preferably 45% or lower, 40% or lower, 35% or lower, 30% or lower, 20% or lower, or 15% or lower, particularly preferably 10% or lower, 9% or lower, 8% or lower, 7% or lower, 6% or lower, 5% or lower, 4% or lower, 3% or lower, 2% or lower, or 1% or lower, compared with the binding activity of a control antigen-binding molecule comprising an Fc region on the basis of the analysis method described above.

An antigen-binding molecule having an IgG1, IgG2, IgG3, or IgG4 monoclonal antibody Fc region can be appropriately used as the control antigen-binding molecule. The structure of the Fc region is described in SEQ ID NO: 212 (RefSeq registration No. AAC82527.1 with A added to the N terminus), SEQ ID NO: 213 (RefSeq registration No. AAB59393.1 with A added to the N terminus), SEQ ID NO: 214 (RefSeq registration No. CAA27268.1 with A added to the N terminus), or SEQ ID NO: 215 (RefSeq registration No. AAB59394.1 with A added to the N terminus). In the case of using an antigen-binding molecule having a variant of the Fc region of an antibody of a certain isotype as a test substance, an antigen-binding molecule having the Fc region of the antibody of this certain isotype is used as a control to test the effect of the mutation in the variant on the binding activity against an Fc gamma receptor. The antigen-binding molecule having the Fc region variant thus confirmed to have reduced binding activity against an Fc gamma receptor is appropriately prepared.

For example, a 231A-238S deletion (WO 2009/011941), C226S, C229S, P238S, (C220S) (J. Rheumatol (2007) 34, 11), C226S, C229S (Hum. Antibod. Hybridomas (1990) 1 (1), 47-54), C226S, C229S, E233P, L234V, or L235A (Blood (2007) 109, 1185-1192) (these amino acids are defined according to the EU numbering) variant is known in the art as such a variant.

Preferred examples thereof include antigen-binding molecules having an Fc region derived from the Fc region of an antibody of a certain isotype by the substitution of any of the following constituent amino acids: amino acids at positions 220, 226, 229, 231, 232, 233, 234, 235, 236, 237, 238, 239, 240, 264, 265, 266, 267, 269, 270, 295, 296, 297, 298, 299, 300, 325, 327, 328, 329, 330, 331, and 332 defined according to the EU numbering. The isotype of the antibody from which the Fc region is originated is not particularly limited, and an Fc region originated from an IgG1, IgG2, IgG3, or IgG4 monoclonal antibody can be appropriately used. An Fc region originated from a naturally occurring human IgG1 antibody is preferably used.
For example, an antigen-binding molecule having an Fc region derived from an IgG1 antibody Fc region by any of the following substitution groups of the constituent amino acids (the number represents the position of an amino acid residue defined according to the EU numbering; the one-letter amino acid code positioned before the number represents an amino acid residue before the substitution; and the one-letter amino acid code positioned after the number represents an amino acid residue before the substitution):

### (a) L234F, L235E, and P331S,

### (b) C226S, C229S, and P238S,

### (c) C226S and C229S, and

### (d) C226S, C229S, E233P, L234V, and L235A

or by the deletion of an amino acid sequence from positions 231 to 238 defined according to the EU numbering can also be appropriately used.

An antigen-binding molecule having an Fc region derived from an IgG2 antibody Fc region by any of the following substitution groups of the constituent amino acids (the number represents the position of an amino acid residue defined according to the EU numbering; the one-letter amino acid code positioned before the number represents an amino acid residue before the substitution; and the one-letter amino acid code positioned after the number represents an amino acid residue before the substitution):

(e) H268Q, V309L, A330S, and P331S,

(f) V234A,

(g) G237A,

(h) V234A and G237A,

(i) A235E and G237A, and

(j) V234A, A235E, and G237A

defined according to the EU numbering can also be appropriately used.

An antigen-binding molecule having an Fc region derived from an IgG3 antibody Fc region by any of the following substitution groups of the constituent amino acids (the number represents the position of an amino acid residue defined according to the EU numbering; the one-letter amino acid code positioned before the number represents an amino acid residue before the substitution; and the one-letter amino acid code positioned after the number represents an amino acid residue before the substitution):

### (k) F241A,

### (l) D265A, and

### (m) V264A

defined according to the EU numbering can also be appropriately used.

An antigen-binding molecule having an Fc region derived from an IgG4 antibody Fc region by any of the following substitution groups of the constituent amino acids (the number represents the position of an amino acid residue defined according to the EU numbering; the one-letter amino acid code positioned before the number represents an amino acid residue before the substitution; and the one-letter amino acid code positioned after the number represents an amino acid residue before the substitution):

(n) L235A, G237A, and E318A,

(o) L235E, and

(p) F234A and L235A

defined according to the EU numbering can also be appropriately used.

Other preferred examples thereof include antigen-binding molecules having an Fc region derived from the Fc region of a naturally occurring human IgG1 antibody by the substitution of any of the following constituent amino acids: amino acids at positions 233, 234, 235, 236, 237, 327, 330, and 331 defined according to the EU numbering, by an amino acid at the corresponding EU numbering position in the Fc region of the counterpart IgG2 or IgG4.

Other preferred examples thereof include antigen-binding molecules having an Fc region derived from the Fc region of a naturally occurring human IgG1 antibody by the substitution of any one or more of the following constituent amino acids: amino acids at positions 234, 235, and 297 defined according to the EU numbering, by a different amino acid. The type of the amino acid present after the substitution is not particularly limited. An antigen-binding molecule having an Fc region with any one or more of amino acids at positions 234, 235, and 297 substituted by alanine is particularly preferred.

Other preferred examples thereof include antigen-binding molecules having an Fc region derived from an IgG1 antibody Fc region by the substitution of the constituent amino acid at position 265 defined according to the EU numbering, by a different amino acid. The type of the amino acid present after the substitution is not particularly limited. An antigen-binding molecule having an Fc region with an amino acid at position 265 substituted by alanine is particularly preferred.

One preferred form of the “antigen-binding molecule” of the present invention can be, for example, a multispecific antibody comprising the antibody variable region of the present invention.

A technique of suppressing the unintended association between H chains by introducing electric charge repulsion to the interface between the second constant domains (CH2) or the third constant domains (CH3) of the antibody H chains (WO2006/106905) can be applied to association for the multispecific antibody.

In the technique of suppressing the unintended association between H chains by introducing electric charge repulsion to the CH2 or CH3 interface, examples of amino acid residues contacting with each other at the interface between the H chain constant domains can include a residue at EU numbering position 356, a residue at EU numbering position 439, a residue at EU numbering position 357, a residue at EU numbering position 370, a residue at EU numbering position 399, and a residue at EU numbering position 409 in one CH3 domain, and their partner residues in another CH3 domain.

More specifically, for example, an antibody comprising two H chain CH3 domains can be prepared as an antibody in which one to three pairs of amino acid residues selected from the following amino acid residue pairs (1) to (3) in the first H chain CH3 domain carry the same electric charge: (1) amino acid residues at EU numbering positions 356 and 439 contained in the H chain CH3 domain; (2) amino acid residues at EU numbering positions 357 and 370 contained in the H chain CH3 domain; and (3) amino acid residues at EU numbering positions 399 and 409 contained in the H chain CH3 domain.

The antibody can be further prepared as an antibody in which one to three pairs of amino acid residues are selected from the amino acid residue pairs (1) to (3) in the second H chain CH3 domain different from the first H chain CH3 domain so as to correspond to the amino acid residue pairs (1) to (3) carrying the same electric charge in the first H chain CH3 domain and to carry opposite electric charge from their corresponding amino acid residues in the first H chain CH3 domain.

Each amino acid residue described in the pairs (1) to (3) is located close to its partner in the associated H chains. Those skilled in the art can find positions corresponding to the amino acid residues described in each of the pairs (1) to (3) as to the desired H chain CH3 domains or H chain constant domains by homology modeling or the like using commercially available software and can appropriately alter amino acid residues at the positions.

In the antibody described above, each of the “amino acid residues carrying electric charge” is preferably selected from, for example, amino acid residues included in any of the following groups (a) and (b):

(a) glutamic acid (E) and aspartic acid (D); and
(b) lysine (K), arginine (R), and histidine (H).

In the antibody described above, the phrase “carrying the same electric charge” means that, for example, all of two or more amino acid residues are amino acid residues included in any one of the groups (a) and (b). The phrase “carrying opposite electric charge” means that, for example, at least one amino acid residue among two or more amino acid residues may be an amino acid residue included in any one of the groups (a) and (b), while the remaining amino acid residue(s) is amino acid residue(s) included in the other group.

In a preferred embodiment, the antibody may have the first H chain CH3 domain and the second H chain CH3 domain cross-linked through a disulfide bond.

The amino acid residue to be altered according to the present invention is not limited to the amino acid residues in the antibody variable region or the antibody constant region mentioned above. Those skilled in the art can find amino acid residues constituting the interface as to a polypeptide variant or a heteromultimer by homology modeling or the like using commercially available software and can alter amino acid residues at the positions so as to regulate the association.

The association for the multispecific antibody of the present invention can also be carried out by an alternative technique known in the art. An amino acid side chain present in the variable domain of one antibody H chain is substituted by a larger side chain (knob), and its partner amino acid side chain present in the variable domain of the other H chain is substituted by a smaller side chain (hole). The knob can be placed into the hole to efficiently associate the polypeptides of the Fc domains differing in amino acid sequence (WO1996/027011; Ridgway J B et al., Protein Engineering (1996) 9, 617-621; and Merchant A M et al. Nature Biotechnology (1998) 16, 677-681).

In addition to this technique, a further alternative technique known in the art may be used for forming the multispecific antibody of the present invention. A portion of CH3 of one antibody H chain is converted to its counterpart IgA-derived sequence, and its complementary portion in CH3 of the other H chain is converted to its counterpart IgA-derived sequence. Use of the resulting strand-exchange engineered domain CH3 can cause efficient association between the polypeptides differing in sequence through complementary CH3 association (Protein Engineering Design & Selection, 23; 195-202, 2010). By use of this technique known in the art, the multispecific antibody of interest can also be efficiently formed.

Alternatively, the multispecific antibody may be formed by, for example, an antibody preparation technique using antibody CH1-CL association and VH-VL association as described in WO2011/028952, a technique of preparing a bispecific antibody using separately prepared monoclonal antibodies (Fab arm exchange) as described in WO2008/119353 and WO2011/131746, a technique of controlling the association between antibody heavy chain CH3 domains as described in WO2012/058768 and WO2013/063702, a technique of preparing a bispecific antibody constituted by two types of light chains and one type of heavy chain as described in WO2012/023053, or a technique of preparing a bispecific antibody using two bacterial cell lines each expressing an antibody half-molecule consisting of one H chain and one L chain as described in Christoph et al. (Nature Biotechnology Vol. 31, p. 753-758 (2013)). In addition to these association techniques, CrossMab technology, a known hetero light chain association technique of associating a light chain forming a variable region binding to a first epitope and a light chain forming a variable region binding to a second epitope to a heavy chain forming the variable region binding to the first epitope and a heavy chain forming the variable region binding to the second epitope, respectively (Scaefer et al., Proc. Natl. Acad. Sci. U.S.A. (2011) 108, 11187-11192), can also be used for preparing a multispecific or multiparatopic antigen-binding molecule provided by the present invention. Examples of the technique of preparing a bispecific antibody using separately prepared monoclonal antibodies can include a method which involves promoting antibody heterodimerization by placing monoclonal antibodies with a particular amino acid substituted in a heavy chain CH3 domain under reductive conditions to obtain the desired bispecific antibody. Examples of the amino acid substitution site preferred for this method can include a residue at EU numbering position 392 and a residue at EU numbering position 397 in the CH3 domain. Furthermore, the bispecific antibody can also be prepared by use of an antibody in which one to three pairs of amino acid residues selected from the following amino acid residue pairs (1) to (3) in the first H chain CH3 domain carry the same electric charge: (1) amino acid residues at EU numbering positions 356 and 439 contained in the H chain CH3 domain; (2) amino acid residues at EU numbering positions 357 and 370 contained in the H chain CH3 domain; and (3) amino acid residues at EU numbering positions 399 and 409 contained in the H chain CH3 domain. The bispecific antibody can also be prepared by use of the antibody in which one to three pairs of amino acid residues are selected from the amino acid residue pairs (1) to (3) in the second H chain CH3 domain different from the first H chain CH3 domain so as to correspond to the amino acid residue pairs (1) to (3) carrying the same electric charge in the first H chain CH3 domain and to carry opposite electric charge from their corresponding amino acid residues in the first H chain CH3 domain.

Even if the multispecific antibody of interest cannot be formed efficiently, the multispecific antibody of the present invention may be obtained by the separation and purification of the multispecific antibody of interest from among produced antibodies. For example, the previously reported method involves introducing amino acid substitution to the variable domains of two types of H chains to impart thereto difference in isoelectric point so that two types of homodimers and the heterodimerized antibody of interest can be separately purified by ion-exchanged chromatography (WO2007114325). A method using protein A to purify a heterodimerized antibody consisting of a mouse IgG2a H chain capable of binding to protein A and a rat IgG2b H chain incapable of binding to protein A has previously been reported as a method for purifying the heterodimer (WO98050431 and WO95033844). Alternatively, amino acid residues at EU numbering positions 435 and 436 that constitute the protein A-binding site of IgG may be substituted by amino acids, such as Tyr and His, which offer the different strength of protein A binding, and the resulting H chain is used to change the interaction of each H chain with protein A. As a result, only the heterodimerized antibody can be efficiently purified by use of a protein A column.

A plurality of, for example, two or more of these techniques may be used in combination. Also, these techniques can be appropriately applied separately to the two H chains to be associated. On the basis of, but separately from the form thus altered, the antigen-binding molecule of the present invention may be prepared as an antigen-binding molecule having an amino acid sequence identical thereto.

The alteration of an amino acid sequence can be performed by various methods known in the art. Examples of these methods that may be performed can include, but are not limited to, methods such as site-directed mutagenesis (Hashimoto-Gotoh, T, Mizuno, T, Ogasahara, Y, and Nakagawa, M. (1995) An oligodeoxyribonucleotidedirected dual amber method for site-directed mutagenesis. Gene 152, 271-275; Zoller, M J, and Smith, M. (1983) Oligonucleotide-directed mutagenesis of DNA fragments cloned into M13 vectors. Methods Enzymol. 100, 468-500; Kramer, W, Drutsa, V, Jansen, H W, Kramer, B, Pflugfelder, M, and Fritz, H J (1984) The gapped duplex DNA approach to oligonucleotide-directed mutation construction. Nucleic Acids Res. 12, 9441-9456; Kramer W, and Fritz H J (1987) Oligonucleotide-directed construction of mutations via gapped duplex DNA Methods. Enzymol. 154, 350-367; and Kunkel, T A (1985) Rapid and efficient site-specific mutagenesis without phenotypic selection. Proc Natl Acad Sci USA. 82, 488-492), PCR mutagenesis, and cassette mutagenesis.

The “antigen-binding molecule” of the present invention may be an antibody fragment that comprises both of a heavy chain and a light chain constituting the “antibody variable region” of the present invention in a single polypeptide chain, but lacks a constant region. Such an antibody fragment may be, for example, diabody (Db), a single-chain antibody, or sc(Fab′)2.

Db is a dimer constituted by two polypeptide chains (e.g., Holliger P et al., Proc. Natl. Acad. Sci. USA 90: 6444-6448 (1993); EP404,097; and WO93/11161). These polypeptide chains are linked through a linker as short as, for example, approximately 5 residues, such that an L chain variable domain (VL) and an H chain variable domain (VH) on the same polypeptide chain cannot be paired with each other.

Because of this short linker, VL and VH encoded on the same polypeptide chain cannot form single-chain Fv and instead, are dimerized with VH and VL, respectively, on another polypeptide chain, to form two antigen-binding sites.

Examples of the single-chain antibody include sc(Fv)2. The sc(Fv)2 is a single-chain antibody having one chain constituted by four variable domains, i.e., two VLs and two VHs, linked via linkers such as peptide linkers (J Immunol. Methods (1999) 231 (1-2), 177-189). These two VHs and VLs may be derived from different monoclonal antibodies. Preferred examples thereof include bispecific sc(Fv)2, which recognizes two types of epitopes present in the same antigen, as disclosed in Journal of Immunology (1994) 152 (11), 5368-5374. The sc(Fv)2 can be prepared by a method generally known to those skilled in the art. For example, the sc(Fv)2 can be prepared by connecting two scFvs via a linker such as a peptide linker.

Examples of the configuration of the antigen-binding domains constituting the sc(Fv)2 described herein include an antibody in which two VHs and two VLs are aligned as VH, VL, VH, and VL (i.e., [VH]-linker-[VL]-linker-[VH]-linker-[VL]) in this order starting at the N-terminus of the single-chain polypeptide. The order of two VHs and two VLs is not particularly limited to the configuration described above and may be any order of arrangement. Examples thereof can also include the following arrangements:

[VL]-linker-[VH]-linker-[VH]-linker-[VL],

[VH]-linker-[VL]-linker-[VL]-linker-[VH],

[VH]-linker-[VH]-linker-[VL]-linker-[VL],

[VL]-linker-[VL]-linker-[VH]-linker-[VH], and

[VL]-linker-[VH]-linker-[VL]-linker-[VH].

The molecular form of the sc(Fv)2 is also described in detail in WO2006/132352. On the basis of the description therein, those skilled in the art can appropriately prepare the desired sc(Fv)2 in order to prepare the antigen-binding molecule disclosed in the present specification.

The antigen-binding molecule of the present invention may be conjugated with a carrier polymer such as PEG or an organic compound such as an anticancer agent. Also, a sugar chain can be preferably added to the antigen-binding molecule of the present invention by the insertion of a glycosylation sequence for the purpose of producing the desired effects.

For example, an arbitrary peptide linker that can be introduced by genetic engineering, or a synthetic compound linker (e.g., a linker disclosed in Protein Engineering, 9 (3), 299-305, 1996) can be used as the linker to link the antibody variable domains. In the present invention, a peptide linker is preferred. The length of the peptide linker is not particularly limited and can be appropriately selected by those skilled in the art according to the purpose. The length is preferably 5 or more amino acids (the upper limit is not particularly limited and is usually 30 or less amino acids, preferably 20 or less amino acids), particularly preferably 15 amino acids. When the sc(Fv)2 contains three peptide linkers, all of these peptide linkers used may have the same lengths or may have different lengths.

Examples of the peptide linker can include

Ser,
Gly-Ser,
Gly-Gly-Ser,
Ser-Gly-Gly,
(SEQ ID NO: 216)
Gly-Gly-Gly-Ser,
(SEQ ID NO: 217)
Ser-Gly-Gly-Gly,
(SEQ ID NO: 218)
Gly-Gly-Gly-Gly-Ser,
(SEQ ID NO: 219)
Ser-Gly-Gly-Gly-Gly,
(SEQ ID NO: 220)
Gly-Gly-Gly-Gly-Gly-Ser,
(SEQ ID NO: 221)
Ser-Gly-Gly-Gly-Gly-Gly,
(SEQ ID NO: 222)
Gly-Gly-Gly-Gly-Gly-Gly-Ser,
(SEQ ID NO: 223)
Ser-Gly-Gly-Gly-Gly-Gly-Gly,
(SEQ ID NO: 218)
(Gly-Gly-Gly-Gly-Ser)n,
and
(SEQ ID NO: 219)
(Ser-Gly-Gly-Gly-Gly)n,

wherein n is an integer of 1 or larger.

However, the length or sequence of the peptide linker can be appropriately selected by those skilled in the art according to the purpose.

The synthetic compound linker (chemical cross-linking agent) is a cross-linking agent usually used in the cross-linking of peptides, for example, N-hydroxysuccinimide (NHS), disuccinimidyl suberate (DSS), bis(sulfosuccinimidyl) suberate (BS3), dithiobis(succinimidyl propionate) (DSP), dithiobis(sulfosuccinimidyl propionate) (DTSSP), ethylene glycol bis(succinimidyl succinate) (EGS), ethylene glycol bis(sulfosuccinimidyl succinate) (sulfo-EGS), disuccinimidyl tartrate (DST), disulfosuccinimidyl tartrate (sulfo-DST), bis[2-(succinimidoxycarbonyloxy)ethyl]sulfone (BSOCOES), or bis[2-(sulfosuccinimidoxycarbonyloxy)ethyl]sulfone (sulfo-BSOCOES).

These cross-linking agents are commercially available.

Three linkers are usually necessary for linking four antibody variable domains. All of these linkers used may be the same linkers or may be different linkers.

The F(ab′)2 comprises two light chains and two heavy chains containing a constant region (CH1 domains and a portion of CH2 domains) so as to form the interchain disulfide bond between these two heavy chains. The F(ab′)2 constituting a polypeptide associate disclosed in the present specification can be preferably obtained by the partial digestion of, for example, a whole monoclonal antibody having the desired antigen-binding domains with a proteolytic enzyme such as pepsin followed by the removal of an Fc fragment adsorbed on a protein A column. Such a proteolytic enzyme is not particularly limited as long as the enzyme is capable of digesting a whole antibody to restrictively form F(ab′)₂ under appropriately set reaction conditions (e.g., pH) of the enzyme. Examples thereof can include pepsin and ficin.

The antigen-binding molecule of the present invention can further contain additional alteration in addition to the amino acid alteration mentioned above. The additional alteration can be selected from, for example, amino acid substitution, deletion, and modification, and a combination thereof.

For example, the antigen-binding molecule of the present invention can be further altered arbitrarily, substantially without changing the intended functions of the molecule. Such a mutation can be performed, for example, by the conservative substitution of amino acid residues. Alternatively, even alteration to change the intended functions of the antigen-binding molecule of the present invention may be carried out as long as the functions changed by such alteration fall within the object of the present invention.

The alteration of an amino acid sequence according to the present invention also includes posttranslational modification. Specifically, the posttranslational modification can refer to the addition or deletion of a sugar chain. The antigen-binding molecule of the present invention, for example, having an IgG1-type constant region, can have a sugar chain-modified amino acid residue at EU numbering position 297. The sugar chain structure for use in the modification is not limited. In general, antibodies expressed by eukaryotic cells involve sugar chain modification in their constant regions. Thus, antibodies expressed by the following cells are usually modified with some sugar chain:

mammalian antibody-producing cells; and

eukaryotic cells transformed with expression vectors comprising antibody-encoding DNAs.

In this context, the eukaryotic cells include yeast and animal cells. For example, CHO cells or HEK293H cells are typical animal cells for transformation with expression vectors comprising antibody-encoding DNAs. On the other hand, the antibody of the present invention also includes antibodies lacking sugar chain modification at the position. The antibodies having sugar chain-unmodified constant regions can be obtained by the expression of genes encoding these antibodies in prokaryotic cells such as E. coli.

The additional alteration according to the present invention may be more specifically, for example, the addition of sialic acid to a sugar chain in an Fc region (mAbs. 2010 September-October; 2 (5): 519-27).

When the antigen-binding molecule of the present invention has an Fc region, for example, amino acid substitution to improve binding activity against FcRn (J Immunol. 2006 Jan. 1; 176 (1): 346-56; J Biol Chem. 2006 Aug. 18; 281 (33): 23514-24; Int Immunol. 2006 December; 18 (12): 1759-69; Nat Biotechnol. 2010 February; 28 (2): 157-9; WO2006/019447; WO2006/053301; and WO2009/086320) or amino acid substitution to improve antibody heterogeneity or stability ((WO2009/041613)) may be added thereto.

In the present invention, the term “antibody” is used in the broadest sense and also includes any antibody such as monoclonal antibodies (including whole monoclonal antibodies), polyclonal antibodies, antibody variants, antibody fragments, multispecific antibodies (e.g., bispecific antibodies), chimeric antibodies, and humanized antibodies as long as the antibody exhibits the desired biological activity.

The antibody of the present invention is not limited by the type of its antigen, its origin, etc., and may be any antibody. Examples of the origin of the antibody can include, but are not particularly limited to, human antibodies, mouse antibodies, rat antibodies, and rabbit antibodies.

The antibody can be prepared by a method well known to those skilled in the art. For example, the monoclonal antibodies may be produced by a hybridoma method (Kohler and Milstein, Nature 256: 495 (1975)) or a recombination method (U.S. Pat. No. 4,816,567). Alternatively, the monoclonal antibodies may be isolated from phage-displayed antibody libraries (Clackson et al., Nature 352: 624-628 (1991); and Marks et al., J. Mol. Biol. 222: 581-597 (1991)). Also, the monoclonal antibodies may be isolated from single B cell clones (N. Biotechnol. 28 (5): 253-457 (2011)).

The humanized antibodies are also called reshaped human antibodies. Specifically, for example, a humanized antibody consisting of a non-human animal (e.g., mouse) antibody CDR-grafted human antibody is known in the art. General gene recombination approaches are also known for obtaining the humanized antibodies. Specifically, for example, overlap extension PCR is known in the art as a method for grafting mouse antibody CDRs to human FRs.

DNAs encoding antibody variable domains each comprising three CDRs and four FRs linked and DNAs encoding human antibody constant domains can be inserted into expression vectors such that the variable domain DNAs are fused in frame with the constant domain DNAs to prepare vectors for humanized antibody expression. These vectors having the inserts are transferred to hosts to establish recombinant cells. Then, the recombinant cells are cultured for the expression of the DNAs encoding the humanized antibodies to produce the humanized antibodies into the cultures of the cultured cells (see European Patent Publication No. EP 239400 and International Publication No. WO1996/002576).

If necessary, FR amino acid residue(s) may be substituted such that the CDRs of the reshaped human antibody form an appropriate antigen-binding site. For example, the amino acid sequence of FR can be mutated by the application of the PCR method used in the mouse CDR grafting to the human FRs.

The desired human antibody can be obtained by DNA immunization using transgenic animals having all repertoires of human antibody genes (see International Publication Nos. WO1993/012227, WO1992/003918, WO1994/002602, WO1994/025585, WO1996/034096, and WO1996/033735) as immunized animals.

In addition, a technique of obtaining human antibodies by panning using human antibody libraries is also known. For example, a human antibody V region is expressed as a single-chain antibody (scFv) on the surface of phages by a phage display method. A phage expressing antigen-binding scFv can be selected. The gene of the selected phage can be analyzed to determine a DNA sequence encoding the V region of the antigen-binding human antibody. After the determination of the DNA sequence of the antigen-binding scFv, the V region sequence can be fused in frame with the sequence of the desired human antibody C region and then inserted to appropriate expression vectors to prepare expression vectors. The expression vectors are transferred to the preferred expression cells listed above for the expression of the genes encoding the human antibodies to obtain the human antibodies. These methods are already known in the art (see International Publication Nos. WO1992/001047, WO1992/020791, WO1993/006213, WO1993/011236, WO1993/019172, WO1995/001438, and WO1995/015388).

In addition to the phage display technique, for example, a technique using a cell-free translation system, a technique of displaying an antigen-binding molecule on the surface of a cell or a virus, and a technique using an emulsion are known as techniques for obtaining a human antibody by panning using a human antibody library. For example, a ribosome display method which involves forming a complex of mRNA and a translated protein via a ribosome by the removal of a stop codon, etc., a cDNA or mRNA display method which involves covalently binding a translated protein to a gene sequence using a compound such as puromycin, or a CIS display method which involves forming a complex of a gene and a translated protein using a nucleic acid-binding protein, can be used as the technique using a cell-free translation system. The phage display method as well as an E. coli display method, a gram-positive bacterium display method, a yeast display method, a mammalian cell display method, a virus display method, or the like can be used as the technique of displaying an antigen-binding molecule on the surface of a cell or a virus. For example, an in vitro virus display method using a gene and a translation-related molecule enclosed in an emulsion can be used as the technique using an emulsion. These methods have already been known in the art (Nat Biotechnol. 2000 December; 18 (12): 1287-92; Nucleic Acids Res. 2006; 34 (19): e127; Proc Natl Acad Sci USA. 2004 Mar. 2; 101 (9): 2806-10; Proc Natl Acad Sci USA. 2004 Jun. 22; 101 (25): 9193-8; Protein Eng Des Sel. 2008 April; 21 (4): 247-55; Proc Natl Acad Sci USA. 2000 September 26; 97 (20): 10701-5; MAbs. 2010 September-October; 2 (5): 508-18; and Methods Mol Biol. 2012; 911: 183-98).

The variable regions binding to a third antigen of the present invention can be variable regions that recognize an arbitrary antigen. The variable regions binding to a third antigen of the present invention can be variable regions that recognize a molecule specifically expressed in a cancer tissue.

In the present specification, the “third antigen” is not particularly limited and may be any antigen. Examples of the antigen include 17-IA, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAMS, ADAMS, ADAMTS, ADAMTS4, ADAMTS5, Addressins, adiponectin, ADP ribosyl cyclase-1, aFGF, AGE, ALCAM, ALK, ALK-1, ALK-7, allergen, alpha1-antichemotrypsin, alpha1-antitrypsin, alpha-synuclein, alphaV/beta-1 antagonist, aminin, amylin, amyloid beta, amyloid immunoglobulin heavy chain variable region. amyloid immunoglobulin light chain variable region, Androgen, ANG, angiotensinogen, Angiopoietin ligand-2, anti-Id, antithrombinIII, Anthrax, APAF-1, APE, APJ, apo A1, apo serum amyloid A, Apo-SAA, APP, APRIL, AR, ARC, ART, Artemin, ASPARTIC, Atrial natriuretic factor, Atrial natriuretic peptide, atrial natriuretic peptides A, atrial natriuretic peptides B, atrial natriuretic peptides C, av/b3 integrin, Axl, B7-1, B7-2, B7-H, BACE, BACE-1, Bacillus anthracis protective antigen, Bad, BAFF, BAFF-R, Bag-1, BAK, Bax, BCA-1, BCAM, BcI, BCMA, BDNF, b-ECGF, beta-2-microglobulin, betalactamase, bFGF, BID, Bik, BIM, BLC, BL-CAM, BLK, B-lymphocyte Stimulator (BIyS), BMP, BMP-2 (BMP-2a), BMP-3 (Osteogenin), BMP-4 (BMP-2b), BMP-5, BMP-6 (Vgr-1), BMP-7 (OP-1), BMP-8 (BMP-8a), BMPR, BMPR-IA (ALK-3), BMPR-IB (ALK-6), BMPR-II (BRK-3), BMPs, BOK, Bombesin, Bone-derived neurotrophic factor, bovine growth hormone, BPDE, BPDE-DNA, BRK-2, BTC, B-lymphocyte cell adhesion molecule, C10, C1-inhibitor, C1q, C3, C3a, C4, C5, C5a(complement 5a), CA125, CAD-8, Cadherin-3, Calcitonin, cAMP, Carbonic anhydrase-IX, carcinoembryonic antigen (CEA), carcinoma-associated antigen, Cardiotrophin-1, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1/I-309, CCL11/Eotaxin, CCL12/MCP-5, CCL13/MCP-4, CCL14/HCC-1, CCL15/HCC-2, CCL16/HCC-4, CCL17/TARC, CCL18/PARC, CCL19/ELC, CCL2/MCP-1, CCL20/MIP-3-alpha, CCL21/SLC, CCL22/MDC, CCL23/MPIF-1, CCL24/Eotaxin-2, CCL25/TECK, CCL26/Eotaxin-3, CCL27/CTACK, CCL28/MEC, CCL3/M1P-1-alpha, CCL3 L1/LD-78-beta, CCL4/MIP-1-beta, CCL5/RANTES, CCL6/C10, CCL7/MCP-3, CCL8/MCP-2, CCL9/10/MTP-1-gamma, CCR, CCR1, CCR10, CCR2, CCR3, CCR4, CCR5, CCR6, CCR7, CCR8, CCR9, CD1, CD10, CD105, CD11a, CD11b, CD11c, CD123, CD13, CD137, CD138, CD14, CD140a, CD146, CD147, CD148, CD15, CD152, CD16, CD164, CD18, CD19, CD2, CD20, CD21, CD22, CD23, CD25, CD26, CD27L, CD28, CD29, CD3, CD30, CD30L, CD32, CD33 (p67 proteins), CD34, CD37, CD38, CD3E, CD4, CD40, CD40L, CD44, CD45, CD46, CD49a, CD49b, CD5, CD51, CD52, CD54, CD55, CD56, CD6, CD61, CD64, CD66e, CD7, CD70, CD74, CD8, CD80 (B7-1), CD89, CD95, CD105, CD158a, CEA, CEACAMS, CFTR, cGMP, CGRP receptor, CINC, CKb8-1, Claudin18, CLC, Clostridium botulinum toxin, Clostridium difficile toxin, Clostridium perfringens toxin, c-Met, CMV, CMV UL, CNTF, CNTN-1, complement factor 3 (C3), complement factor D, corticosteroid-binding globulin, Colony stimulating factor1 receptor, COX, C-Ret, CRG-2, CRTH2, CT-1, CTACK, CTGF, CTLA-4, CX3 CL1/Fractalkine, CX3 CR1, CXCL, CXCL1/Gro-alpha, CXCL10, CXCL11/I-TAC, CXCL12/SDF-1-alphafbeta, CXCL13/BCA-1, CXCL14/BRAK, CXCL15/Lungkine. CXCL16, CXCL16, CXCL2/Gro-beta CXCL3/Gro-gamma, CXCL3, CXCL4/PF4, CXCL5/ENA-78, CXCL6/GCP-2, CXCL7/NAP-2, CXCL8/IL-8, CXCL9/Mig, CXCL1O/IP-10, CXCR, CXCR1, CXCR2, CXCR3, CXCR4, CXCR5, CXCR6, cystatin C, cytokeratin tumor-associated antigen, DAN, DCC, DcR3, DC-SIGN, Decay accelerating factor, Delta-like protein ligand 4, des(1-3)-IGF-1 (brain IGF-1), Dhh, DHICA oxidase, Dickkopf-1, digoxin, Dipeptidyl peptidase IV, DK1, DNAM-1, Dnase, Dpp, DPPIV/CD26, Dtk, ECAD, EDA, EDAAl, EDA-A2, EDAR, EGF, EGFR (ErbB-1), EGF like domain containing protein 7, Elastase, elastin, EMA, EMMPRIN, ENA, ENA-78, Endosialin, endothelin receptor, endotoxin, Enkephalinase, eNOS, Eot, Eotaxin, Eotaxin-2, eotaxini, EpCAM, Ephrin B2/EphB4, Epha2 tyrosine kinase receptor, epidermal growth factor receptor (EGFR), ErbB2 receptor, ErbB3 tyrosine kinase receptor, ERCC, EREG, erythropoietin (EPO), Erythropoietin receptor, E-selectin, ET-1, Exodus-2, F protein of RSV, F10, F11, F12, F13, F5, F9, Factor Ia, Factor IX, Factor Xa, Factor VII, factor VIII, Factor VIIIc, Fas, FcalphaR, FcepsilonRI, FcgammaIIb, FcgammaRI, FcgammaRIIa, FcgammaRIIIa, FcgammaRIIIb, FcRn, FEN-1, Ferritin, FGF, FGF-19, FGF-2, FGF-2 receptor, FGF-3, FGF-8, FGF-acidic, FGF-basic, Fibrin, fibroblast activation protein (FAP), fibroblast growth factor, fibroblast growth factor-10, fibronectin, FL, FLIP, Flt-3, FLT3 ligand, Folate receptor, follicle stimulating hormone (FSH), Fractalkine (CX3C), free heavy chain, free light chain, FZD1, FZD10, FZD2, FZD3, FZD4, FZD5, FZD6, FZD7, FZD8, FZD9, G250, Gas 6, GCP-2, GCSF, G-CSF, G-CSF receptor, GD2, GD3, GDF, GDF-1, GDF-15 (MIC-1), GDF-3 (Vgr-2), GDF-5 (BMP-14/CDMP-1), GDF-6 (BMP-13/CDMP-2), GDF-7 (BMP-12/CDMP-3), GDF-8 (Myostatin), GDF-9, GDNF, Gelsolin, GFAP, GF-CSF, GFR-alpha1, GFR-alpha2, GFR-alpha3, GF-beta 1, gH envelope glycoprotein, GITR, Glucagon, Glucagon receptor, Glucagon-like peptide 1 receptor, Glut 4, Glutamate carboxypeptidase II, glycoprotein hormone receptors, glycoprotein IIb/IIIa (GP IIb/IIIa), Glypican-3, GM-CSF, GM-CSF receptor, gp130, gp140, gp72, granulocyte-CSF (G-CSF), GRO/MGSA, Growth hormone releasing factor, GRO-beta, GRO-gamma, H. pylori, Hapten (NP-cap or NIP-cap), HB-EGF, HCC, HCC 1, HCMV gB envelope glycoprotein, HCMV UL, Hemopoietic growth factor (HGF), Hep B gp120, heparanase, heparin cofactor II, hepatic growth factor, Bacillus anthracis protective antigen, Hepatitis C virus E2 glycoprotein, Hepatitis E, Hepcidin, Herl, Her2/neu (ErbB-2), Her3 (ErbB-3), Her4 (ErbB-4), herpes simplex virus (HSV) gB glycoprotein, HGF, HGFA, High molecular weight melanoma-associated antigen (HMW-MAA), HIV envelope proteins such as GP120, HIV MIB gp 120 V3 loop, HLA, HLA-DR, HM1.24, HMFG PEM, HMGB-1, HRG, Hrk, HSP47, Hsp90, HSV gD glycoprotein, human cardiac myosin, human cytomegalovirus (HCMV), human growth hormone (hGH), human serum albumin, human tissue-type plasminogen activator (t-PA), Huntingtin, HVEM, IAP, ICAM, ICAM-1, ICAM-3, ICE, ICOS, IFN-alpha, IFN-beta, IFN-gamma, IgA, IgA receptor, IgE, IGF, IGF binding proteins, IGF-1, IGF-1 R, IGF-2, IGFBP, IGFR, IL, IL-1, IL-10, IL-10 receptors, IL-11, IL-11 receptors, IL-12, IL-12 receptors, IL-13, IL-13 receptors, IL15, IL-15 receptors, IL-16, IL-16 receptors, IL-17, IL-17 receptors, IL-18 (IGIF), IL18 receptors, IL-1alpha, IL-1beta, IL-1 receptors, IL-2, IL-2 receptors, IL-20, IL-20 receptors, IL-21, IL-21 receptors, IL-23, IL-23 receptors, IL-2 receptors, IL-3, IL-3 receptors, IL-31, IL-31 receptors, IL-3 receptors, IL-4, IL-4 receptors IL-5, IL-5 receptors, IL-6, IL-6 receptors, IL-7, IL-7 receptors, IL-8, IL-8 receptors, IL-9, IL-9 receptors, immunoglobulin immune complex, immunoglobulins, INF-alpha, INF-alpha receptors, INF-beta, INF-beta receptors, INF-gamma, INF-gamma receptors, IFN type-I, IFN type-I receptor, influenza, inhibin, Inhibin alpha, Inhibin beta, iNOS, insulin, Insulin A-chain, Insulin B-chain, Insulin-like growth factor 1, insulin-like growth factor 2, insulin-like growth factor binding proteins, integrin, integrin alpha2, integrin alpha3, integrin alpha4, integrin alpha4/beta1, integrin alpha-V/beta-3, integrin alpha-V/beta-6, integrin alpha4/beta7, integrin alpha5/beta1, integrin alpha5/beta3, integrin alpha5/beta6, integrin alpha sigma (alphaV), integrin alpha theta, integrin beta1, integrin beta2, integrin beta3 (GPIIb-IIIa), IP-10, I-TAC, JE, kalliklein, Kallikrein 11, Kallikrein 12, Kallikrein 14, Kallikrein 15, Kallikrein 2, Kallikrein 5, Kallikrein 6, Kallikrein L1, Kallikrein L2, Kallikrein L3, Kallikrein L4, kallistatin, KC, KDR, Keratinocyte Growth Factor (KGF), Keratinocyte Growth Factor-2 (KGF-2), KGF, killer immunoglobulin-like receptor, kit ligand (KL), Kit tyrosine kinase, laminin 5, LAMP, LAPP (Amylin, islet-amyloid polypeptide), LAP (TGF-1), latency associated peptide, Latent TGF-1, Latent TGF-1 bpl, LBP, LDGF, LDL, LDL receptor, LECT2, Lefty, Leptin, leutinizing hormone (LH), Lewis-Y antigen, Lewis-Y related antigen, LFA-1, LFA-3, LFA-3 receptors, Lfo, LIF, LIGHT, lipoproteins, LIX, LKN, Lptn, L-Selectin, LT-a, LT-b, LTB4, LTBP-1, Lung surfactant, Luteinizing hormone, Lymphotactin, Lymphotoxin Beta Receptor, Lysosphingolipid receptor, Mac-1, macrophage-CSF (M-CSF), MAdCAM, MAG, MAP2, MARC, maspin, MCAM, MCK-2, MCP, MCP-1, MCP-2, MCP-3, MCP-4, MCP-I (MCAF), M-CSF, MDC, MDC (67a.a.), MDC (69a.a.), megsin, Mer, MET tyrosine kinase receptor family, METALLOPROTEASES, Membrane glycoprotein OX2, Mesothelin, MGDF receptor, MGMT, MHC (HLA-DR), microbial protein, MIF, MIG, MIP, MIP-1 alpha, MIP-1 beta, MIP-3 alpha, MIP-3 beta, MIP-4, MK, MMAC1, MMP, MMP-1, MMP-10, MMP-11, MMP12, MMP-13, MMP-14, MMP-15, MMP-2, MMP-24, MMP-3, MMP-7, MMP-8, MMP-9, monocyte attractant protein, monocyte colony inhibitory factor, mouse gonadotropin-associated peptide, MPIF, Mpo, MSK, MSP, MUC-16, MUC18, mucin (Mud), Muellerian-inhibiting substance, Mug, MuSK, Myelin associated glycoprotein, myeloid progenitor inhibitor factor-1 (MPIF-I), NAIP, Nanobody, NAP, NAP-2, NCA 90, NCAD, N-Cadherin, NCAM, Neprilysin, Neural cell adhesion molecule, neroserpin, Neuronal growth factor (NGF), Neurotrophin-3, Neurotrophin-4, Neurotrophin-6, Neuropilin 1, Neurturin, NGF-beta, NGFR, NKG20, N-methionyl human growth hormone, nNOS, NO, Nogo-A, Nogo receptor, non-structural protein type 3 (NS3) from the hepatitis C virus, NOS, Npn, NRG-3, NT, NT-3, NT-4, NTN, OB, OGG1, Oncostatin M, OP-2, OPG, OPN, OSM, OSM receptors, osteoinductive factors, osteopontin, OX40L, OX40R, oxidized LDL, p150, p95, PADPr, parathyroid hormone, PARC, PARP, PBR, PBSF, PCAD, P-Cadherin, PCNA, PCSK9, PDGF, PDGF receptor, PDGF-AA, PDGF-AB, PDGF-BB, PDGF-D, PDK-1, PECAM, PEDF, PEM, PF-4, PGE, PGF, PGI2, PGD2, PIGF, PIN, PLA2, Placenta g rowth factor, placental alkaline phosphatase (PLAP), placental lactogen, plasminogen activator inhibitor-1, platelet-growth factor, plgR, PLP, poly glycol chains of different size(e.g. PEG-20, PEG-30, PEG40), PP14, prekallikrein, prion protein, procalcitonin, Programmed cell death protein 1, proinsulin, prolactin, Proprotein convertase PC9, prorelaxin, prostate specific membrane antigen (PSMA), Protein A, Protein C, Protein D, Protein S, Protein Z, PS, PSA, PSCA, PsmAr, PTEN, PTHrp, Ptk, PTN, P-selectin glycoprotein ligand-1, R51, RAGE, RANK, RANKL, RANTES, relaxin, Relaxin A-chain, Relaxin B-chain, renin, respiratory syncytial virus (RSV) F, Ret, reticulon 4, Rheumatoid factors, RLI P76, RPA2, RPK-1, RSK, RSV Fgp, 5100, RON-8, SCF/KL, SCGF, Sclerostin, SDF-1, SDF1 alpha, SDF1 beta, SERINE, Serum Amyloid P, Serum albumin, sFRP-3, Shh, Shiga like toxin II, SIGIRR, SK-1, SLAM, SLPI, SMAC, SMDF, SMOH, SOD, SPARC, sphingosine 1-phosphate receptor 1, Staphylococcal lipoteichoic acid, Stat, STEAP, STEAP-II, stem cell factor (SCF), streptokinase, superoxide dismutase, syndecan-1, TACE, TACI, TAG-72 (tumor-associated glycoprotein-72), TARC, TB, TCA-3, T-cell receptor alpha/beta, TdT, TECK, TEM1, TEM5, TEM7, TEM8, Tenascin, TERT, testicular PLAP-like alkaline phosphatase, TfR, TGF, TGF-alpha, TGF-beta, TGF-beta Pan Specific, TGF-beta RII, TGF-beta Rub, TGF-beta RIII, TGF-beta R1 (ALK-5), TGF-beta1, TGF-beta2, TGF-beta3, TGFbeta4, TGF-beta5, TGF-I, Thrombin, thrombopoietin (TPO), Thymic stromal lymphoprotein receptor, Thymus Ck-1, thyroid stimulating hormone (TSH), thyroxine, thyroxine-binding globulin, Tie, TIMP, TIQ, Tissue Factor, tissue factor protease inhibitor, tissue factor protein, TMEFF2, Tmpo, TMPRSS2, TNF receptor I, TNF receptor II, TNF-alpha, TNF-beta, TNF-beta2, TNFc, TNF-RI, TNF-RII, TNFRSF10A (TRAIL R1 Apo-2/DR4), TNFRSF10B (TRAIL R2 DR5/KILLER/TRICK-2A/TRICK-B), TNFRSF10C (TRAIL R3 DcRl/LIT/TRID), TNFRSF10D (TRAIL R4 DcR2/TRUNDD), TNFRSF11A (RANK ODF R/TRANCE R), TNFRSF11B (OPG OCIF/TR1), TNFRSF12 (TWEAK R FN14), TNFRSF12A, TNFRSF13B (TACI), TNFRSF13C (BAFF R), TNFRSF14 (HVEM ATAR/HveA/LIGHT R/TR2), TNFRSF16 (NGFR p75NTR), TNFRSF17 (BCMA), TNFRSF18 (GITR ATTR), TNFRSF19 (TROY TAJ/TRADE), TNFRSF19L (RELT), TNFRSF1A (TNF R1 CD120a/p55-60), TNFRSF1B (TNF RII CD120b/p75-80), TNFRSF21 (DR6), TNFRSF22 (DcTRAIL R2 TNFRH2), TNFRSF25 (DR3 Apo3/LARD/TR-3/TRAMP/WSL-1), TNFRSF26 (TNFRH3), TNFRSF3 (LTbR TNF RIII/TNFC R), TNFRSF4 (OX40 ACT35/TXGP1 R), TNFRSF5 (CD40 p50), TNFRSF6 (Fas Apo-1/APT1/CD95), TNFRSF6B (DcR3 M68/TR6), TNFRSF7 (CD27), TNFRSF8 (CD30), TNFRSF9 (4-1 BB CD137/ILA), TNFRST23 (DcTRAIL R1 TNFRH1), TNFSF10 (TRAIL Apo-2 Ligand/TL2), TNFSF11 (TRANCE/RANK Ligand ODF/OPG Ligand), TNFSF12 (TWEAK Apo-3 Ligand/DR3 Ligand), TNFSF13 (APRIL TALL2), TNFSF13B (BAFF BLYS/TALL1/THANK/TNFSF20), TNFSF14 (LIGHT HVEM Ligand/LTg), TNFSF15 (TL1A/VEGI), TNFSF18 (GITR Ligand AITR Ligand/TL6), TNFSF1A (TNF-α Conectin/DIF/TNFSF2), TNFSF1B (TNF-b LTa/TNFSF1), TNFSF3 (LTb TNFC/p33), TNFSF4 (OX40 Ligand gp34/TXGP1), TNFSF5 (CD40 Ligand CD154/gp39/HIGM1/IMD3/TRAP), TNFSF6 (Fas Ligand Apo-1 Ligand/APT1 Ligand), TNFSF7 (CD27 Ligand CD70), TNFSF8 (CD30 Ligand CD153), TNFSF9 (4-1 BB Ligand CD137 Ligand), TNF-alpha, TNF-beta, TNIL-I, toxic metabolite, TP-1, t-PA, Tpo, TRAIL, TRAIL R, TRAIL-R1, TRAIL-R2, TRANCE, transferrin receptor, transforming growth factors (TGF) such as TGF-alpha and TGF-beta, Transmembrane glycoprotein NMB, Transthyretin, TRF, Trk, TROP-2, Trophoblast glycoprotein, TSG, TSLP, Tumor Necrosis Factor (TNF), tumor-associated antigen CA 125, tumor-associated antigen expressing Lewis Y related carbohydrate, TWEAK, TXB2, Ung, uPAR, uPAR-1, Urokinase, VAP-1, vascular endothelial growth factor (VEGF), vaspin, VCAM, VCAM-1, VECAD, VE-Cadherin, VE-Cadherin-2, VEFGR-1 (flt-1), VEFGR-2, VEGF receptor (VEGFR), VEGFR-3 (flt-4), VEGI, VIM, Viral antigens, VitB12 receptor, Vitronectin receptor, VLA, VLA-1, VLA-4, VNR integrin, von Willebrand Factor (vWF), WIF-1, WNT1, WNT10A, WNT10B, WNT11, WNT16, WNT2, WNT2B/13, WNT3, WNT3A, WNT4, WNTSA, WNTSB, WNT6, WNT7A, WNT7B, WNT8A, WNT8B, WNT9A, WNT9B, XCL1, XCL2/SCM-1-beta, XCL1/Lymphotactin, XCR1, XEDAR, XIAP, and XPD.

Specific examples of the molecule specifically expressed on a T cell include CD3 and T cell receptors. Particularly, CD3 is preferred. In the case of, for example, human CD3, a site in the CD3 to which the antigen-binding molecule of the present invention binds may be any epitope present in a gamma chain, delta chain, or epsilon chain sequence constituting the human CD3. Particularly, an epitope present in the extracellular region of an epsilon chain in a human CD3 complex is preferred. The polynucleotide sequences of the gamma chain, delta chain, and epsilon chain structures constituting CD3 are shown in SEQ ID NOs: 224 (NM_000073.2), 226 (NM_000732.4), and 228 (NM_000733.3), and the polypeptide sequences thereof are shown in SEQ ID NOs: 225 (NP_000064.1), 227 (NP_000723.1), and 229 (NP_000724.1) (RefSeq registration numbers are shown within the parentheses).

One of the two variable regions of the antibody included in the antigen-binding molecule of the present invention binds to a “third antigen” that is different from the “CD3” and the “CD137” mentioned above. In some embodiments, the third antigen is derived from humans, mice, rats, monkeys, rabbits, or dogs. In some embodiments, the third antigen is a molecule specifically expressed on the cell or the organ derived from humans, mice, rats, monkeys, rabbits, or dogs. The third antigen is preferably, a molecule not systemically expressed on the cell or the organ. The third antigen is preferably, for example, a tumor cell-specific antigen and also includes an antigen expressed in association with the malignant alteration of cells as well as an abnormal sugar chain that appears on cell surface or a protein molecule during the malignant transformation of cells. Specific examples thereof include ALK receptor (pleiotrophin receptor), pleiotrophin, KS 1/4 pancreatic cancer antigen, ovary cancer antigen (CA125), prostatic acid phosphate, prostate-specific antigen (PSA), melanoma-associated antigen p97, melanoma antigen gp75, high-molecular-weight melanoma antigen (HMW-MAA), prostate-specific membrane antigen, carcinoembryonic antigen (CEA), polymorphic epithelial mucin antigen, human milk fat globule antigen, colorectal tumor-associated antigen (e.g., CEA, TAG-72, CO17-1A, GICA 19-9, CTA1, and LEA), Burkitt's lymphoma antigen 38.13, CD19, human B lymphoma antigen CD20, CD33, melanoma-specific antigen (e.g., ganglioside GD2, ganglioside GD3, ganglioside GM2, and ganglioside GM3), tumor-specific transplantation antigen (TSTA), T antigen, virus-induced tumor antigen (e.g., envelope antigens of DNA tumor virus and RNA tumor virus), colon CEA, oncofetal antigen alpha-fetoprotein (e.g., oncofetal trophoblastic glycoprotein 5T4 and oncofetal bladder tumor antigen), differentiation antigen (e.g., human lung cancer antigens L6 and L20), fibrosarcoma antigen, human T cell leukemia-associated antigen Gp37, newborn glycoprotein, sphingolipid, breast cancer antigen (e.g., EGFR (epithelial growth factor receptor)), NYBR-16, NY-BR-16 and HER2 antigen (p185HER2), polymorphic epithelial mucin (PEM), malignant human lymphocyte antigen APO-1, differentiation antigen such as I antigen found in fetal erythrocytes, primary endoderm I antigen found in adult erythrocytes, I (Ma) found in embryos before transplantation or gastric cancer, M18 found in mammary gland epithelium, M39, SSEA-1 found in bone marrow cells, VEP8, VEP9, Myl, VIM-D5, D156-22 found in colorectal cancer, TRA-1-85 (blood group H), SCP-1 found in testis and ovary cancers, C14 found in colon cancer, F3 found in lung cancer, AH6 found in gastric cancer, Y hapten, Ley found in embryonic cancer cells, TL5 (blood group A), EGF receptor found in A431 cells, E1 series (blood group B) found in pancreatic cancer, FC10.2 found in embryonic cancer cells, gastric cancer antigen, CO-514 (blood group Lea) found in adenocarcinoma, NS-10 found in adenocarcinoma, CO-43 (blood group Leb), G49 found in A431 cell EGF receptor, MH2 (blood group ALeb/Ley) found in colon cancer, 19.9 found in colon cancer, gastric cancer mucin, T5A7 found in bone marrow cells, R24 found in melanoma, 4.2, GD3, D1.1, OFA-1, GM2, OFA-2, GD2, and M1:22:25:8 found in embryonic cancer cells, SSEA-3 and SSEA-4 found in 4-cell to 8-cell embryos, cutaneous T cell lymphoma-associated antigen, MART-1 antigen, sialyl Tn (STn) antigen, colon cancer antigen NYCO-45, lung cancer antigen NY-LU-12 variant A, adenocarcinoma antigen ART1, paraneoplastic associated brain-testis-cancer antigen (onconeuronal antigen MA2 and paraneoplastic neuronal antigen), neuro-oncological ventral antigen 2 (NOVA2), blood cell cancer antigen gene 520, tumor-associated antigen CO-029, tumor-associated antigen MAGE-C1 (cancer/testis antigen CT7), MAGE-B1 (MAGE-XP antigen), MAGE-B2 (DAM6), MAGE-2, MAGE-4a, MAGE-4b MAGE-X2, cancer-testis antigen (NY-EOS-1), YKL-40, and any fragment of these polypeptides, and modified structures thereof (aforementioned modified phosphate groups, sugar chains, etc.), EpCAM, EREG, CA19-9, CA15-3, sialyl SSEA-1 (SLX), HER2, PSMA, CEA, and CLEC12A.

The term “CD137” herein, also called 4-1BB, is a member of the tumor necrosis factor (TNF) receptor family. Examples of factors belonging to the TNF superfamily or the TNF receptor superfamily include CD137, CD137L, CD40, CD40L, OX40, OX40L, CD27, CD70, HVEM, LIGHT, RANK, RANKL, CD30, CD153, GITR, and GITRL.

In one aspect, an antigen-binding molecule of the present invention has at least one characteristic selected from the group consisting of (1) to (4) below:

(1) the variable region binds to an extracellular domain of CD3 epsilon (epsilon) comprising the amino acid sequence of SEQ ID NO: 159,

(2) the antigen-binding molecule has an agonistic activity against CD137,

(3) the antigen-binding molecule induces CD3 activation of a T cell against a cell expressing the molecule of the third antigen, but does not induce activation of a T cell against a cell expressing CD137, and

(4) the antigen-binding molecule does not induce release of a cytokine from PBMC in the absence of a cell expressing the molecule of the third antigen.

In one aspect, an antigen-binding molecule of the present invention has at least one characteristic selected from the group consisting of (1) to (4) below:

(1) the variable region binds to an extracellular domain of CD3 epsilon (epsilon) comprising the amino acid sequence of SEQ ID NO: 159,

(2) the antigen-binding molecule has an agonistic activity against CD137,

(3) the antigen-binding molecule induces cytotoxicity of a T cell against a cell expressing the molecule of the third antigen, but does not induce activation of a T cell against a cell expressing CD137, and

(4) the antigen-binding molecule does not induce release of a cytokine from PBMC in the absence of a cell expressing the molecule of the third antigen.

In some embodiments, an antigen-binding molecule of the present invention has at least one characteristic selected from the group consisting of (1) to (2) below:

(1) the antigen-binding molecule does not compete for binding to CD137 with CD137 ligand, and

(2) the antigen-binding molecule induces cytotoxicity of a T cell against a cell expressing the molecule of the third antigen, but does not induce cytotoxicity of a T cell against a cell expressing CD137.

In one aspect, the “CD137 agonist antibody” or “antigen-binding molecule having an agonistic activity against CD137” of the present invention refers to an antibody or an antigen-binding molecule that activates cells expressing CD137 by at least about 5%, specifically at least about 10%, or more specifically at least about 15% when added to the cells, tissues, or living bodies that express CD137, where 0% activation is the background level (e.g. IL6 secretion and so on) of the non-activation cells expressing CD137. In various specific examples, the CD137 agonist antibody for use as a pharmaceutical composition of the present invention can activate the activity of the cells by at least about 20%, 30%, 40%, 50%, 60%, 70%, 80%, 90%, 100%, 125%, 150%, 175%, 200%, 250%, 300%, 350%, 400%, 450%, 500%, 750%, or 1000%.

In one aspect, the “CD137 agonist antibody” or “antigen-binding molecule having an agonistic activity against CD137” of the present invention also refers to an antibody or an antigen-binding molecule that activates cells expressing CD137 by at least about 5%, specifically at least about 10%, or more specifically at least about 15% when added to the cells, tissues, or living bodies that express CD137, where 100% activation is the level of activation achieved by an equimolar amount of a binding partner under physiological conditions. In various specific examples, the CD137 agonist antibody for use as a pharmaceutical composition of the present invention can activate the activity of the cells by at least about 5%, 10%, 20%, 30%, 40%, 50%, 60%, 70%, 80%, 90%, 100%, 125%, 150%, 175%, 200%, 250%, 300%, 350%, 400%, 450%, 500%, 750%, or 1000%. In some embodiments, “a binding partner” used herein is a molecule which is known to bind to CD137 and induce the activation of cells expressing CD137. In further embodiments, examples of the binding partner include Urelumab (CAS Registry No. 934823-49-1) and its variants described in WO2005/035584A1, Utomilumab (CAS Registry No. 1417318-27-4) and its variants described in WO2012/032433A1, and various known CD137 agonist antibodies. In certain embodiments, examples of the binding partner include CD137 ligands. In further embodiments, the activation of cells expressing CD137 by an anti-CD137 agonist antibody may be determined using an ELISA to characterize IL6 secretion (See, e.g., Reference Example 5-2, herein). The anti-CD137 antibody used as the binding partner and the antibody concentration for the measurements can be referred to Reference Example 5-2, where 100% activation is the level of activation achieved by the antibody. In further embodiments, an antibody comprising the heavy chain amino acid sequence of SEQ ID NO: 142 and the light chain amino acid sequence of SEQ ID NO: 144 can be used at 30 micro g/mL for the measurements as the binding partner (See, e.g., Reference Example 5-2, herein). In some embodiments, the activation of cells expressing CD137 by an anti-CD137 agonist antibody may be determined, for example, using recombinant T cells that express a reporter gene (e.g. luciferase) in response to CD137 signaling, and detecting the expression of the reporter gene or the activity of the reporter gene product as an index of the activation of the T cells. When recombinant T cells that express a reporter gene in response to CD137 signaling are co-cultured with an antigen-binding molecule, the antigen-binding molecule is determined to induce activation of T cells against cells expressing CD137 if the expression of the reporter gene or the activity of the reporter gene product is above 10%, 20%, 30%, 40% 50%, 90%, 100% or more that of negative control (See, e.g., Example 2.2, herein).

As a non-limiting embodiment, the present invention provides a “CD137 agonist antibody” comprising an Fc region, wherein the Fc region has an enhanced binding activity towards an inhibitory Fc gamma receptor.

As a non-limiting embodiment, the CD137 agonistic activity can be confirmed using B cells, which are known to express CD137 on their surface. As a non-limiting embodiment, HDLM-2 B cell line can be used as B cells. The CD137 agonistic activity can be evaluated by the amount of human Interleukin-6 (IL-6) produced because the expression of IL-6 is induced as a result of the activation of CD137. In this evaluation, it is possible to determine how much % of CD137 agonistic activity the evaluated molecule has by evaluating the increased amount of IL-6 expression by using the amount of IL-6 from non-activating B cells as 0% background level.

In some embodiments, the antigen-binding molecule of the present invention induces CD3 activation of T cells against cells expressing the molecule of a third antigen, but does not induce CD3 activation of T cells against cells expressing CD137. Whether an antigen-binding molecule induces CD3 activation of T cells against cells expressing a third antigen can be determined by, for example, co-culturing T cells with cells expressing the third antigen in the presence of the antigen-binding molecule, and assaying CD3 activation of the T cells. T cell activation can be assayed by, for example, using recombinant T cells that express a reporter gene (e.g. luciferase) in response to CD3 signaling, and detecting the expression of the reporter gene or the activity of the reporter gene product as an index of the activation of the T cells. When recombinant T cells that express a reporter gene in response to CD3 signaling are co-cultured with cells expressing a third antigen in the presence of an antigen-binding molecule, detection of the expression of the reporter gene or the activity of the reporter gene product in a manner dependent on the dose of the antigen-binding molecule indicates that the antigen-binding molecule induces activation of T cells against cells expressing the third antigen. Similarly, whether an antigen-binding molecule does not induce CD3 activation of T cells against cells expressing CD137 can be determined by, for example, co-culturing T cells with cells expressing CD137 in the presence of the antigen-binding molecule, and assaying CD3 activation of the T cells as described above. When recombinant T cells that express a reporter gene in response to CD3 signaling are co-cultured with cells expressing CD137 in the presence of an antigen-binding molecule, the antigen-binding molecule is determined not to induce activation of T cells against cells expressing CD137 if the expression of the reporter gene or the activity of the reporter gene product is absent or below a detection limit or below that of negative control. In one aspect, when recombinant T cells that express a reporter gene in response to CD3 signaling are co-cultured with cells expressing CD137 in the presence of an antigen-binding molecule, the antigen-binding molecule is determined not to induce activation of T cells against cells expressing CD137 if the expression of the reporter gene or the activity of the reporter gene product is at most about 50%, 30%, 20%, 10%, 5% or 1%, where 100% activation is the level of activation achieved by an antigen-binding molecule which binds to CD3 and CD137 at the same time. In one aspect, when recombinant T cells that express a reporter gene in response to CD3 signaling are co-cultured with cells expressing CD137 in the presence of an antigen-binding molecule, the antigen-binding molecule is determined not to induce activation of T cells against cells expressing CD137 if the expression of the reporter gene or the activity of the reporter gene product is at most about 50%, 30%, 20%, 10%, 5% or 1%, where 100% activation is the level of activation achieved by the same antigen-binding molecule against cells expressing the molecule of a third antigen.

In some embodiments, the antigen-binding molecule of the present invention does not induce a cytokine release from PBMCs in the absence of cells expressing the molecule of a third antigen. Whether an antigen-binding molecule does not induce release of cytokines in the absence of cells expressing a third antigen can be determined by, for example, incubating PBMCs with the antigen-binding molecule in the absence of cells expressing a third antigen, and measuring cytokines such as IL-2, IFN gamma, and TNF alpha released from the PBMCs into the culture supernatant using methods known in the art. If no significant levels of cytokines are detected or no significant induction of cytokines expression occurred in the culture supernatant of PBMCs that have been incubated with an antigen-binding molecule in the absence of cells expressing a third antigen, the antigen-binding molecule is determined not to induce a cytokine release from PBMCs in the absence of cells expressing a third antigen. In one aspect, “no significant levels of cytokines” also refers to the level of cytokines concentration that is about at most 50%, 30%, 20%, 10%, 5% or 1%, where 100% is the cytokine concentration achieved by an antigen-binding molecule which binds to CD3 and CD137 at the same time. In one aspect, “no significant levels of cytokines” also refers to the level of cytokines concentration that is about at most 50%, 30%, 20%, 10%, 5% or 1%, where 100% is the cytokine concentration achieved in the presence of cells expressing the molecule of a third antigen. In one aspect, “no significant induction of cytokines expression” also refers to the level of cytokines concentration increase that is at most 5-fold, 2-fold or 1-fold of the concentration of each cytokines before adding the antigen-binding molecules.

In some embodiments, an antigen-binding molecule of the present invention competes for binding to CD137, or binds to the same epitope on CD137, with an antibody selected from the group consisting of:

(a1) a heavy chain complementarity determining region 1 (HCDR1) comprising an amino acid sequence that is at least 70%, 80% or 90% identical to SEQ ID NO: 16, a heavy chain complementarity determining region 2 (HCDR2) comprising an amino acid sequence that is at least 70%, 80% or 90% identical to SEQ ID NO: 30, a heavy chain complementarity determining region 3 (HCDR3) comprising an amino acid sequence that is at least 70%, 80% or 90% identical to SEQ ID NO: 44, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;

(a2) a heavy chain complementarity determining region 1 (HCDR1) comprising an amino acid sequence that is at least 70%, 80% or 90% identical to SEQ ID NO: 17, a heavy chain complementarity determining region 2 (HCDR2) comprising an amino acid sequence that is at least 70%, 80% or 90% identical to SEQ ID NO: 31, a heavy chain complementarity determining region 3 (HCDR3) comprising an amino acid sequence that is at least 70%, 80% or 90% identical to SEQ ID NO: 45, a light chain complementarity determining region 1 (LCDR1) comprising an amino acid sequence that is at least 70%, 80% or 90% identical to SEQ ID NO: 64, a light chain complementarity determining region 2 (LCDR2) comprising an amino acid sequence that is at least 70%, 80% or 90% identical to SEQ ID NO: 69, and a light chain complementarity determining region 3 (LCDR3) comprising an amino acid sequence that is at least 70%, 80% or 90% identical to SEQ ID NO: 74;

(a3) a heavy chain complementarity determining region 1 (HCDR1) comprising an amino acid sequence that is at least 70%, 80% or 90% identical to SEQ ID NO: 18, a heavy chain complementarity determining region 2 (HCDR2) comprising an amino acid sequence that is at least 70%, 80% or 90% identical to SEQ ID NO: 32, a heavy chain complementarity determining region 3 (HCDR3) comprising an amino acid sequence that is at least 70%, 80% or 90% identical to SEQ ID NO: 46, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;

(a4) a heavy chain complementarity determining region 1 (HCDR1) comprising an amino acid sequence that is at least 70%, 80% or 90% identical to SEQ ID NO: 19, a heavy chain complementarity determining region 2 (HCDR2) comprising an amino acid sequence that is at least 70%, 80% or 90% identical to SEQ ID NO: 33, a heavy chain complementarity determining region 3 (HCDR3) comprising an amino acid sequence that is at least 70%, 80% or 90% identical to SEQ ID NO: 47, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;

(a5) a heavy chain complementarity determining region 1 (HCDR1) comprising an amino acid sequence that is at least 70%, 80% or 90% identical to SEQ ID NO: 19, a heavy chain complementarity determining region 2 (HCDR2) comprising an amino acid sequence that is at least 70%, 80% or 90% identical to SEQ ID NO:33, a heavy chain complementarity determining region 3 (HCDR3) comprising an amino acid sequence that is at least 70%, 80% or 90% identical to SEQ ID NO: 47, a light chain complementarity determining region 1 (LCDR1) comprising an amino acid sequence that is at least 70%, 80% or 90% identical to SEQ ID NO: 65, a light chain complementarity determining region 2 (LCDR2) comprising an amino acid sequence that is at least 70%, 80% or 90% identical to SEQ ID NO: 70, and a light chain complementarity determining region 3 (LCDR3) comprising an amino acid sequence that is at least 70%, 80% or 90% identical to SEQ ID NO: 75;

(a6) a heavy chain complementarity determining region 1 (HCDR1) comprising an amino acid sequence that is at least 70%, 80% or 90% identical to SEQ ID NO: 20, a heavy chain complementarity determining region 2 (HCDR2) comprising an amino acid sequence that is at least 70%, 80% or 90% identical to SEQ ID NO: 34, a heavy chain complementarity determining region 3 (HCDR3) comprising an amino acid sequence that is at least 70%, 80% or 90% identical to SEQ ID NO: 48, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;

(a7) a heavy chain complementarity determining region 1 (HCDR1) comprising an amino acid sequence that is at least 70%, 80% or 90% identical to SEQ ID NO: 22, a heavy chain complementarity determining region 2 (HCDR2) comprising an amino acid sequence that is at least 70%, 80% or 90% identical to SEQ ID NO: 36, a heavy chain complementarity determining region 3 (HCDR3) comprising an amino acid sequence that is at least 70%, 80% or 90% identical to SEQ ID NO: 50, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;

(a8) a heavy chain complementarity determining region 1 (HCDR1) comprising an amino acid sequence that is at least 70%, 80% or 90% identical to SEQ ID NO: 23, a heavy chain complementarity determining region 2 (HCDR2) comprising an amino acid sequence that is at least 70%, 80% or 90% identical to SEQ ID NO: 37, a heavy chain complementarity determining region 3 (HCDR3) comprising an amino acid sequence that is at least 70%, 80% or 90% identical to SEQ ID NO: 51, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;

(a9) a heavy chain complementarity determining region 1 (HCDR1) comprising an amino acid sequence that is at least 70%, 80% or 90% identical to SEQ ID NO: 23, a heavy chain complementarity determining region 2 (HCDR2) comprising an amino acid sequence that is at least 70%, 80% or 90% identical to SEQ ID NO: 37, a heavy chain complementarity determining region 3 (HCDR3) comprising an amino acid sequence that is at least 70%, 80% or 90% identical to SEQ ID NO: 51, a light chain complementarity determining region 1 (LCDR1) comprising an amino acid sequence that is at least 70%, 80% or 90% identical to SEQ ID NO: 66, a light chain complementarity determining region 2 (LCDR2) comprising an amino acid sequence that is at least 70%, 80% or 90% identical to SEQ ID NO: 71, and a light chain complementarity determining region 3 (LCDR3) comprising an amino acid sequence that is at least 70%, 80% or 90% identical to SEQ ID NO: 76;

(a10) a heavy chain complementarity determining region 1 (HCDR1) comprising an amino acid sequence that is at least 70%, 80% or 90% identical to SEQ ID NO: 24, a heavy chain complementarity determining region 2 (HCDR2) comprising an amino acid sequence that is at least 70%, 80% or 90% identical to SEQ ID NO: 38, a heavy chain complementarity determining region 3 (HCDR3) comprising an amino acid sequence that is at least 70%, 80% or 90% identical to SEQ ID NO: 52, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;

(a11) a heavy chain complementarity determining region 1 (HCDR1) comprising an amino acid sequence that is at least 70%, 80% or 90% identical to SEQ ID NO: 25, a heavy chain complementarity determining region 2 (HCDR2) comprising an amino acid sequence that is at least 70%, 80% or 90% identical to SEQ ID NO: 39, a heavy chain complementarity determining region 3 (HCDR3) comprising an amino acid sequence that is at least 70%, 80% or 90% identical to SEQ ID NO: 53, a light chain complementarity determining region 1 (LCDR1) comprising an amino acid sequence that is at least 70%, 80% or 90% identical to SEQ ID NO: 66, a light chain complementarity determining region 2 (LCDR2) comprising an amino acid sequence that is at least 70%, 80% or 90% identical to SEQ ID NO: 71, and a light chain complementarity determining region 3 (LCDR3) comprising an amino acid sequence that is at least 70%, 80% or 90% identical to SEQ ID NO: 76;

(a12) a heavy chain complementarity determining region 1 (HCDR1) comprising an amino acid sequence that is at least 70%, 80% or 90% identical to SEQ ID NO: 26, a heavy chain complementarity determining region 2 (HCDR2) comprising an amino acid sequence that is at least 70%, 80% or 90% identical to SEQ ID NO: 40, a heavy chain complementarity determining region 3 (HCDR3) comprising an amino acid sequence that is at least 70%, 80% or 90% identical to SEQ ID NO: 54, a light chain complementarity determining region 1 (LCDR1) comprising an amino acid sequence that is at least 70%, 80% or 90% identical to SEQ ID NO: 66, a light chain complementarity determining region 2 (LCDR2) comprising an amino acid sequence that is at least 70%, 80% or 90% identical to SEQ ID NO: 71, and a light chain complementarity determining region 3 (LCDR3) comprising an amino acid sequence that is at least 70%, 80% or 90% identical to SEQ ID NO: 76;

(a13) a heavy chain complementarity determining region 1 (HCDR1) comprising an amino acid sequence that is at least 70%, 80% or 90% identical to SEQ ID NO: 26, a heavy chain complementarity determining region 2 (HCDR2) comprising an amino acid sequence that is at least 70%, 80% or 90% identical to SEQ ID NO: 40, a heavy chain complementarity determining region 3 (HCDR3) comprising an amino acid sequence that is at least 70%, 80% or 90% identical to SEQ ID NO: 54, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;

(a14) a heavy chain complementarity determining region 1 (HCDR1) comprising an amino acid sequence that is at least 70%, 80% or 90% identical to SEQ ID NO:27, a heavy chain complementarity determining region 2 (HCDR2) comprising an amino acid sequence that is at least 70%, 80% or 90% identical to SEQ ID NO: 41, a heavy chain complementarity determining region 3 (HCDR3) comprising an amino acid sequence that is at least 70%, 80% or 90% identical to SEQ ID NO: 55, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;

(a15) a heavy chain complementarity determining region 1 (HCDR1) comprising an amino acid sequence that is at least 70%, 80% or 90% identical to SEQ ID NO: 28, a heavy chain complementarity determining region 2 (HCDR2) comprising an amino acid sequence that is at least 70%, 80% or 90% identical to SEQ ID NO: 42, a heavy chain complementarity determining region 3 (HCDR3) comprising an amino acid sequence that is at least 70%, 80% or 90% identical to SEQ ID NO: 56, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;

(b1) a HCDR1 comprising an amino acid sequence of SEQ ID NO: 16, a HCDR2 comprising an amino acid sequence of SEQ ID NO: 30, a HCDR3 comprising an amino acid sequence of SEQ ID NO: 44, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b2) a HCDR1 comprising an amino acid sequence of SEQ ID NO: 17, a HCDR2 comprising an amino acid sequence of SEQ ID NO: 31, a HCDR3 comprising an amino acid sequence of SEQ ID NO: 45, a LCDR1 comprising an amino acid sequence of SEQ ID NO: 64, a LCDR2 comprising an amino acid sequence of SEQ ID NO: 69, and a LCDR3 comprising an amino acid sequence of SEQ ID NO: 74;
(b3) a HCDR1 comprising an amino acid sequence of SEQ ID NO: 18, a HCDR2 comprising an amino acid sequence of SEQ ID NO: 32, a HCDR3 comprising an amino acid sequence of SEQ ID NO: 46, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b4) a HCDR1 comprising an amino acid sequence of SEQ ID NO: 19, a HCDR2 comprising an amino acid sequence of SEQ ID NO: 33, a HCDR3 comprising an amino acid sequence of SEQ ID NO: 47, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b5) a HCDR1 comprising an amino acid sequence of SEQ ID NO: 19, a HCDR2 comprising an amino acid sequence of SEQ ID NO: 33, a HCDR3 comprising an amino acid sequence of SEQ ID NO: 47, a LCDR1 comprising an amino acid sequence of SEQ ID NO: 65, a LCDR2 comprising an amino acid sequence of SEQ ID NO: 70, and a LCDR3 comprising an amino acid sequence of SEQ ID NO: 75;
(b6) a HCDR1 comprising an amino acid sequence of SEQ ID NO: 20, a HCDR2 comprising an amino acid sequence of SEQ ID NO: 34, a HCDR3 comprising an amino acid sequence of SEQ ID NO: 48, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b7) a HCDR1 comprising an amino acid sequence of SEQ ID NO: 22, a HCDR2 comprising an amino acid sequence of SEQ ID NO: 36, a HCDR3 comprising an amino acid sequence of SEQ ID NO: 50, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b8) a HCDR1 comprising an amino acid sequence of SEQ ID NO: 23, a HCDR2 comprising an amino acid sequence of SEQ ID NO: 37, a HCDR3 comprising an amino acid sequence of SEQ ID NO: 51, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b9) a HCDR1 comprising an amino acid sequence of SEQ ID NO: 23, a HCDR2 comprising an amino acid sequence of SEQ ID NO: 37, a HCDR3 comprising an amino acid sequence of SEQ ID NO: 51, a LCDR1 comprising an amino acid sequence of SEQ ID NO: 66, a LCDR2 comprising an amino acid sequence of SEQ ID NO: 71, and a LCDR3 comprising an amino acid sequence of SEQ ID NO: 76;
(b10) a HCDR1 comprising an amino acid sequence of SEQ ID NO: 24, a HCDR2 comprising an amino acid sequence of SEQ ID NO: 38, a HCDR3 comprising an amino acid sequence of SEQ ID NO: 52, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b11) a HCDR1 comprising an amino acid sequence of SEQ ID NO: 25, a HCDR2 comprising an amino acid sequence of SEQ ID NO: 39, a HCDR3 comprising an amino acid sequence of SEQ ID NO: 53, a LCDR1 comprising an amino acid sequence of SEQ ID NO: 66, a LCDR2 comprising an amino acid sequence of SEQ ID NO: 71, and a LCDR3 comprising an amino acid sequence of SEQ ID NO: 76;
(b12) a HCDR1 comprising an amino acid sequence of SEQ ID NO: 26, a HCDR2 comprising an amino acid sequence of SEQ ID NO: 40, a HCDR3 comprising an amino acid sequence of SEQ ID NO: 54, a LCDR1 comprising an amino acid sequence of SEQ ID NO: 66, a LCDR2 comprising an amino acid sequence of SEQ ID NO: 71, and a LCDR3 comprising an amino acid sequence of SEQ ID NO: 76;
(b13) a HCDR1 comprising an amino acid sequence of SEQ ID NO: 26, a HCDR2 comprising an amino acid sequence of SEQ ID NO: 40, a HCDR3 comprising an amino acid sequence of SEQ ID NO: 54, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b14) a HCDR1 comprising an amino acid sequence of SEQ ID NO: 27, a HCDR2 comprising an amino acid sequence of SEQ ID NO: 41, a HCDR3 comprising an amino acid sequence of SEQ ID NO: 55, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b15) a HCDR1 comprising an amino acid sequence of SEQ ID NO: 28, a HCDR2 comprising an amino acid sequence of SEQ ID NO: 42, a HCDR3 comprising an amino acid sequence of SEQ ID NO: 56, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(c1) a heavy chain variable domain (VH) comprising an amino acid sequence that is at least 70%, 80% or 90% identical to SEQ ID NO: 2, and a light chain variable domain (VL) comprising an amino acid sequence that is at least 70%, 80% or 90% identical to SEQ ID NO: 58;
(c2) a heavy chain variable domain (VH) comprising an amino acid sequence that is at least 70%, 80% or 90% identical to SEQ ID NO: 3, and a light chain variable domain (VL) comprising an amino acid sequence that is at least 70%, 80% or 90% identical to SEQ ID NO: 59;
(c3) a heavy chain variable domain (VH) comprising an amino acid sequence that is at least 70%, 80% or 90% identical to SEQ ID NO: 4, and a light chain variable domain (VL) comprising an amino acid sequence that is at least 70%, 80% or 90% identical to SEQ ID NO: 58;
(c4) a heavy chain variable domain (VH) comprising an amino acid sequence that is at least 70%, 80% or 90% identical to SEQ ID NO: 5, and a light chain variable domain (VL) comprising an amino acid sequence that is at least 70%, 80% or 90% identical to SEQ ID NO: 58;
(c5) a heavy chain variable domain (VH) comprising an amino acid sequence that is at least 70%, 80% or 90% identical to SEQ ID NO: 5, and a light chain variable domain (VL) comprising an amino acid sequence that is at least 70%, 80% or 90% identical to SEQ ID NO: 60;
(c6) a heavy chain variable domain (VH) comprising an amino acid sequence that is at least 70%, 80% or 90% identical to SEQ ID NO: 6, and a light chain variable domain (VL) comprising an amino acid sequence that is at least 70%, 80% or 90% identical to SEQ ID NO: 58;
(c7) a heavy chain variable domain (VH) comprising an amino acid sequence that is at least 70%, 80% or 90% identical to SEQ ID NO: 8, and a light chain variable domain (VL) comprising an amino acid sequence that is at least 70%, 80% or 90% identical to SEQ ID NO: 58;
(c8) a heavy chain variable domain (VH) comprising an amino acid sequence that is at least 70%, 80% or 90% identical to SEQ ID NO: 9, and a light chain variable domain (VL) comprising an amino acid sequence that is at least 70%, 80% or 90% identical to SEQ ID NO: 58;
(c9) a heavy chain variable domain (VH) comprising an amino acid sequence that is at least 70%, 80% or 90% identical to SEQ ID NO: 9, and a light chain variable domain (VL) comprising an amino acid sequence that is at least 70%, 80% or 90% identical to SEQ ID NO: 61;
(c10) a heavy chain variable domain (VH) comprising an amino acid sequence that is at least 70%, 80% or 90% identical to SEQ ID NO: 10, and a light chain variable domain (VL) comprising an amino acid sequence that is at least 70%, 80% or 90% identical to SEQ ID NO: 58;
(c11) a heavy chain variable domain (VH) comprising an amino acid sequence that is at least 70%, 80% or 90% identical to SEQ ID NO: 11, and a light chain variable domain (VL) comprising an amino acid sequence that is at least 70%, 80% or 90% identical to SEQ ID NO: 61;
(c12) a heavy chain variable domain (VH) comprising an amino acid sequence that is at least 70%, 80% or 90% identical to SEQ ID NO: 12, and a light chain variable domain (VL) comprising an amino acid sequence that is at least 70%, 80% or 90% identical to SEQ ID NO: 61;
(c13) a heavy chain variable domain (VH) comprising an amino acid sequence that is at least 70%, 80% or 90% identical to SEQ ID NO: 12, and a light chain variable domain (VL) comprising an amino acid sequence that is at least 70%, 80% or 90% identical to SEQ ID NO: 58;
(c14) a heavy chain variable domain (VH) comprising an amino acid sequence that is at least 70%, 80% or 90% identical to SEQ ID NO: 13, and a light chain variable domain (VL) comprising an amino acid sequence that is at least 70%, 80% or 90% identical to SEQ ID NO: 58;
(c15) a heavy chain variable domain (VH) comprising an amino acid sequence that is at least 70%, 80% or 90% identical to SEQ ID NO: 14, and a light chain variable domain (VL) comprising an amino acid sequence that is at least 70%, 80% or 90% identical to SEQ ID NO: 58;
(d1) a heavy chain variable domain (VH) of SEQ ID NO: 2, and a light chain variable domain (VL) of SEQ ID NO: 58;
(d2) a heavy chain variable domain (VH) of SEQ ID NO: 3, and a light chain variable domain (VL) of SEQ ID NO: 59;
(d3) a heavy chain variable domain (VH) of SEQ ID NO: 4, and a light chain variable domain (VL) of SEQ ID NO: 58;
(d4) a heavy chain variable domain (VH) of SEQ ID NO: 5, and a light chain variable domain (VL) of SEQ ID NO: 58;
(d5) a heavy chain variable domain (VH) of SEQ ID NO: 5, and a light chain variable domain (VL) of SEQ ID NO: 60;
(d6) a heavy chain variable domain (VH) of SEQ ID NO: 6, and a light chain variable domain (VL) of SEQ ID NO: 58;
(d7) a heavy chain variable domain (VH) of SEQ ID NO: 8, and a light chain variable domain (VL) of SEQ ID NO: 58;
(d8) a heavy chain variable domain (VH) of SEQ ID NO: 9, and a light chain variable domain (VL) of SEQ ID NO: 58;
(d9) a heavy chain variable domain (VH) of SEQ ID NO: 9, and a light chain variable domain (VL) of SEQ ID NO: 61;
(d10) a heavy chain variable domain (VH) of SEQ ID NO: 10, and a light chain variable domain (VL) of SEQ ID NO: 58;
(d11) a heavy chain variable domain (VH) of SEQ ID NO: 11, and a light chain variable domain (VL) of SEQ ID NO: 61;
(d12) a heavy chain variable domain (VH) of SEQ ID NO: 12, and a light chain variable domain (VL) of SEQ ID NO: 61;
(d13) a heavy chain variable domain (VH) of SEQ ID NO: 12, and a light chain variable domain (VL) of SEQ ID NO: 58;
(d14) a heavy chain variable domain (VH) of SEQ ID NO: 13, and a light chain variable domain (VL) of SEQ ID NO: 58;
(d15) a heavy chain variable domain (VH) of SEQ ID NO: 14, and a light chain variable domain (VL) of SEQ ID NO: 58;

Whether a test antibody shares a common epitope with a certain antibody can be assessed based on competition between the two antibodies for the same epitope. The competition between antibodies can be detected by a cross-blocking assay or the like. For example, the competitive ELISA assay is a preferred cross-blocking assay. Specifically, in a cross-blocking assay, the CD137 protein used to coat the wells of a microtiter plate is pre-incubated in the presence or absence of a candidate competitor antibody, and then an anti-CD137 antibody of the present invention is added thereto. The amount of the anti-CD137 antibody of the present invention bound to the CD137 protein in the wells is indirectly correlated with the binding ability of a candidate competitor antibody (test antibody) that competes for the binding to the same epitope. That is, the greater the affinity of the test antibody for the same epitope, the lower the amount of the anti-CD137 antibody of the present invention bound to the CD137 protein-coated wells, and the higher the amount of the test antibody bound to the CD137 protein-coated wells.

The amount of the antibody bound to the wells can be readily determined by labeling the antibody in advance. For example, a biotin-labeled antibody can be measured using an avidin/peroxidase conjugate and an appropriate substrate. In particular, a cross-blocking assay that uses enzyme labels such as peroxidase is called a “competitive ELISA assay”. The antibody can be labeled with other labeling substances that enable detection or measurement. Specifically, radiolabels, fluorescent labels, and such are known.

Furthermore, when the test antibody has a constant region derived from a species different from that of the anti-CD137 antibody of the present invention, the amount of antibody bound to the wells can be measured by using a labeled antibody that recognizes the constant region of that antibody. Alternatively, if the antibodies are derived from the same species but belong to different classes, the amount of the antibodies bound to the wells can be measured using antibodies that distinguish individual classes.

If a candidate antibody can block binding of an anti-CD137 antibody by at least 20%, preferably by at least 20% to 50%, and even more preferably, by at least 50%, as compared to the binding activity obtained in a control experiment performed in the absence of the candidate competing antibody, the candidate competing antibody is either an antibody that binds substantially to the same epitope or an antibody that competes for binding to the same epitope as an anti-CD137 antibody of the present invention.

In another embodiment, the ability of a test antibody to competitively or cross competitively bind with another antibody can be appropriately determined by those skilled in the art using a standard binding assay such as BIAcore analysis or flow cytometry known in the art.

Methods for determining the spatial conformation of an epitope include, for example, X ray crystallography and two-dimensional nuclear magnetic resonance (see, Epitope Mapping Protocols in Methods in Molecular Biology, G. E. Morris (ed.), Vol. 66 (1996)).

Whether a test antibody shares a common epitope with a CD137 ligand can also be assessed based on competition between the test antibody and CD137 ligand for the same epitope. The competition between antibody and CD137 ligand can be detected by a cross-blocking assay or the like as mentioned above. In another embodiment, the ability of a test antibody to competitively or cross competitively bind with CD137 ligand can be appropriately determined by those skilled in the art using a standard binding assay such as BIAcore analysis or flow cytometry known in the art

In some embodiments, favorable examples of an antigen-binding molecule of the present invention include antigen-binding molecules that bind to the same epitope as the human CD137 epitope bound by the antibody selected from the group consisting of:

antibody that recognize a region comprising the SPCPPNSFSSAGGQRTCDICRQCKGVFRTRKECSSTSNAECDCTPGFHCLGAGCSMCEQDCKQGQELTKKG C sequence (SEQ ID NO: 154),

antibody that recognize a region comprising the DCTPGFHCLGAGCSMCEQDCKQGQELTKKGC sequence (SEQ ID NO: 149),

antibody that recognize a region comprising the LQDPCSNCPAGTFCDNNRNQICSPCPPNSFSSAGGQRTCDICRQCKGVFRTRKECSSTSNAEC sequence (SEQ ID NO: 152), and

antibody that recognize a region comprising the LQDPCSNCPAGTFCDNNRNQIC sequence (SEQ ID NO: 147) in the human CD137 protein.

Depending on the targeted cancer antigen, those skilled in the art can appropriately select a heavy chain variable region sequence and a light chain variable region sequence that bind to the cancer antigen for the heavy chain variable region and the light chain variable region to be included in the cancer-specific antigen-binding domain. When an epitope bound by an antigen-binding domain is contained in multiple different antigens, antigen-binding molecules containing the antigen-binding domain can bind to various antigens that have the epitope.

“Epitope” means an antigenic determinant in an antigen, and refers to an antigen site to which various binding domains in antigen-binding molecules disclosed herein bind. Thus, for example, an epitope can be defined according to its structure. Alternatively, the epitope may be defined according to the antigen-binding activity of an antigen-binding molecule that recognizes the epitope. When the antigen is a peptide or polypeptide, the epitope can be specified by the amino acid residues that form the epitope. Alternatively, when the epitope is a sugar chain, the epitope can be specified by its specific sugar chain structure.

A linear epitope is an epitope that contains an epitope whose primary amino acid sequence is recognized. Such a linear epitope typically contains at least three and most commonly at least five, for example, about 8 to 10 or 6 to 20 amino acids in its specific sequence.

In contrast to the linear epitope, “conformational epitope” is an epitope in which the primary amino acid sequence containing the epitope is not the only determinant of the recognized epitope (for example, the primary amino acid sequence of a conformational epitope is not necessarily recognized by an epitope-defining antibody). Conformational epitopes may contain a greater number of amino acids compared to linear epitopes. A conformational epitope-recognizing antibody recognizes the three-dimensional structure of a peptide or protein. For example, when a protein molecule folds and forms a three dimensional structure, amino acids and/or polypeptide main chains that form a conformational epitope become aligned, and the epitope is made recognizable by the antibody. Methods for determining epitope conformations include, for example, X ray crystallography, two-dimensional nuclear magnetic resonance spectroscopy, site-specific spin labeling, and electron paramagnetic resonance spectroscopy, but are not limited thereto. See, for example, Epitope Mapping Protocols in Methods in Molecular Biology (1996), Vol. 66, Morris (ed.).

Examples of a method for assessing the binding of an epitope in a cancer-specific antigen by a test antigen-binding molecule are shown below. According to the examples below, methods for assessing the binding of an epitope in a target antigen by another binding domain can also be appropriately conducted.

For example, whether a test antigen-binding molecule that comprises an antigen-binding domain for a cancer-specific antigen recognizes a linear epitope in the antigen molecule can be confirmed for example as mentioned below. For example, a linear peptide comprising an amino acid sequence forming the extracellular domain of a cancer-specific antigen is synthesized for the above purpose. The peptide can be synthesized chemically, or obtained by genetic engineering techniques using a region in a cDNA of a cancer-specific antigen encoding the amino acid sequence that corresponds to the extracellular domain. Then, a test antigen-binding molecule containing an antigen-binding domain for a cancer-specific antigen is assessed for its binding activity towards a linear peptide comprising the extracellular domain-constituting amino acid sequence. For example, an immobilized linear peptide can be used as an antigen to evaluate the binding activity of the antigen-binding molecule towards the peptide by ELISA. Alternatively, the binding activity towards a linear peptide can be assessed based on the level at which the linear peptide inhibits binding of the antigen-binding molecule to cancer-specific antigen-expressing cells. The binding activity of the antigen-binding molecule towards the linear peptide can be demonstrated by these tests.

Whether the above-mentioned test antigen-binding molecule containing an antigen-binding domain towards an antigen recognizes a conformational epitope can be confirmed as below. For example, an antigen-binding molecule that comprises an antigen-binding domain for a cancer-specific antigen strongly binds to cancer-specific antigen-expressing cells upon contact, but does not substantially bind to an immobilized linear peptide comprising an amino acid sequence forming the extracellular domain of the cancer-specific antigen. Herein, “does not substantially bind” means that the binding activity is 80% or less, generally 50% or less, preferably 30% or less, and particularly preferably 15% or less compared to the binding activity to antigen-expressing cells. of ELISA or fluorescence activated cell sorting (FACS) using antigen-expressing cells as antigen.

In the ELISA format, the binding activity of a test antigen-binding molecule comprising an antigen-binding domain towards antigen-expressing cells can be assessed quantitatively by comparing the levels of signals generated by enzymatic reaction. Specifically, a test antigen-binding molecule is added to an ELISA plate onto which antigen-expressing cells are immobilized. Then, the test antigen-binding molecule bound to the cells is detected using an enzyme-labeled antibody that recognizes the test antigen-binding molecule. Alternatively, when FACS is used, a dilution series of a test antigen-binding molecule is prepared, and the antibody-binding titer for antigen-expressing cells can be determined to compare the binding activity of the test antigen-binding molecule towards antigen-expressing cells.

The binding of a test antigen-binding molecule to an antigen expressed on the surface of cells suspended in buffer or the like can be detected using a flow cytometer. Known flow cytometers include, for example, the following devices:

FACSCanto™ II

FACSAria™

FACSArray™

FACSVantage™ SE

FACSCalibur™ (all are trade names of BD Biosciences)

EPICS ALTRA HyPerSort

Cytomics FC 500

EPICS XL-MCL ADC EPICS XL ADC

Cell Lab Quanta/Cell Lab Quanta SC (all are trade names of Beckman Coulter).

Suitable methods for assaying the binding activity of the above-mentioned test antigen-binding molecule comprising an antigen-binding domain towards an antigen include, for example, the method below. First, antigen-expressing cells are reacted with a test antigen-binding molecule, and then this is stained with an FITC-labeled secondary using FACSCalibur (BD). The fluorescence intensity obtained by analysis using the CELL QUEST Software (BD), i.e., the Geometric Mean value, reflects the quantity of antibody bound to the cells. That is, the binding activity of a test antigen-binding molecule, which is represented by the quantity of the test antigen-binding molecule bound, can be measured by determining the Geometric Mean value.

Whether a test antigen-binding molecule comprising an antigen-binding domain of the present invention shares a common epitope with another antigen-binding molecule can be assessed based on competition between the two molecules for the same epitope. The competition between antigen-binding molecules can be detected by a cross-blocking assay or the like. For example, the competitive ELISA assay is a preferred cross-blocking assay.

Specifically, in a cross-blocking assay, the antigen coating the wells of a microtiter plate is pre-incubated in the presence or absence of a candidate competitor antigen-binding molecule, and then a test antigen-binding molecule is added thereto. The quantity of test antigen-binding molecule bound to the antigen in the wells indirectly correlates with the binding ability of a candidate competitor antigen-binding molecule that competes for the binding to the same epitope. That is, the greater the affinity of the competitor antigen-binding molecule for the same epitope, the lower the binding activity of the test antigen-binding molecule towards the antigen-coated wells.

The quantity of the test antigen-binding molecule bound to the wells via the antigen can be readily determined by labeling the antigen-binding molecule in advance. For example, a biotin-labeled antigen-binding molecule can be measured using an avidin/peroxidase conjugate and appropriate substrate. In particular, a cross-blocking assay that uses enzyme labels such as peroxidase is called “competitive ELISA assay”. The antigen-binding molecule can also be labeled with other labeling substances that enable detection or measurement. Specifically, radiolabels, fluorescent labels, and such are known.

When the candidate competitor antigen-binding molecule can block the binding of a test antigen-binding molecule comprising an antigen-binding domain by at least 20%, preferably at least 20 to 50%, and more preferably at least 50% compared to the binding activity in a control experiment conducted in the absence of the competitor antigen-binding molecule, the test antigen-binding molecule is determined to substantially bind to the same epitope bound by the competitor antigen-binding molecule, or to compete for binding to the same epitope.

When the structure of an epitope bound by a test antigen-binding molecule comprising an antigen-binding domain of the present invention is already identified, whether the test and control antigen-binding molecules share a common epitope can be assessed by comparing the binding activities of the two antigen-binding molecules towards a peptide prepared by introducing amino acid mutations into the peptide forming the epitope.

As a method for measuring such binding activities, for example, the binding activities of test and control antigen-binding molecules towards a linear peptide into which a mutation is introduced are measured by comparison in the above ELISA format. Besides the ELISA methods, the binding activity towards the mutant peptide bound to a column can be determined by passing the test and control antigen-binding molecules through the column, and then quantifying the antigen-binding molecule eluted in the eluate. Methods for adsorbing a mutant peptide to a column, for example, in the form of a GST fusion peptide, are known.

Alternatively, when the identified epitope is a conformational epitope, whether test and control antigen-binding molecules share a common epitope can be assessed by the following method. First, cells expressing an antigen targeted by an antigen-binding domain and cells expressing an antigen having an epitope introduced with a mutation are prepared. The test and control antigen-binding molecules are added to a cell suspension prepared by suspending these cells in an appropriate buffer such as PBS. Then, the cell suspension is appropriately washed with a buffer, and an FITC-labeled antibody that can recognize the test and control antigen-binding molecules is added thereto. The fluorescence intensity and number of cells stained with the labeled antibody are determined using FACSCalibur (BD). The test and control antigen-binding molecules are appropriately diluted using a suitable buffer, and used at desired concentrations. For example, they may be used at a concentration within the range of 10 micro g/ml to 10 ng/ml. The fluorescence intensity determined by analysis using the CELL QUEST Software (BD), i.e., the Geometric Mean value, reflects the quantity of the labeled antibody bound to the cells. That is, the binding activities of the test and control antigen-binding molecules, which are represented by the quantity of the labeled antibody bound, can be measured by determining the Geometric Mean value.

In some embodiments, an antigen-binding molecule of the present invention comprises

(a) a heavy chain variable domain amino acid sequence comprising, at each of the following positions (all by Kabat numbering), one or more of the following amino acid residues indicated for that position:

A, D, E, I, G, K, L, M, N, R, T, W or Y at the amino acid position 26;

D, F, G, I, M or L, at the amino acid position 27;

D, E, F, G, H, I, K, L, M, N, P, Q, R, S, T, V, W or Y at the amino acid position 28;

F or W at the amino acid position 29;

A, D, E, F, G, H, I, K, L, M, N, P, Q, R, S, T, V, W or Y at the amino acid position 30;

F, I, N, R, S, T or V at the amino acid position 31;

A, H, I, K, L, N, Q, R, S, T or V at the amino acid position 32;

W at the amino acid position 33;

F, I, L, M or V at the amino acid position 34;

F, H, S, T, V or Y at the amino acid position 35;

E, F, H, I, K, L, M, N, Q, S, T, W or Y at the amino acid position 50;

I, K or V at the amino acid position 51;

K, M, R, or T at the amino acid position 52;

A, E, F, G, H, I, K, L, M, N, P, Q, R, S, V, W or Y at the amino acid position 52b;

A, D, E, F, G, H, I, K, L, M, N, P, Q, R, S, T, V, W or Y at the amino acid position 52c;

A, E, F, H, K, L, M, N, Q, R, S, T, V, W or Y at the amino acid position 53;

A, D, E, F, G, H, I, K, L, M, N, Q, R, S, T, V, W or Y at the amino acid position 54;

E, F, G, H, L, M, N, Q, W or Y at the amino acid position 55;

A, D, E, F, G, H, I, K, L, M, N, Q, R, S, T, V, W or Y at the amino acid position 56;

A, D, E, G, H, I, K, L, M, N, P, Q, R, S, T or V at the amino acid position 57;

A, F, H, K, N, P, R or Y at the amino acid position 58;

A, D, E, F, G, H, I, K, L, M, N, P, Q, R, S, T, V, W or Y at the amino acid position 59;

A, D, E, F, G, H, I, K, L, M, N, P, Q, R, S, T, V, W or Y at the amino acid position 60;

A, D, E, F, G, H, I, K, L, M, N, P, Q, R, S, T, V, W or Y at the amino acid position 61;

A, D, E, F, G, H, I, K, L, M, N, P, Q, R, S, T, V, W or Y at the amino acid position 62;

A, D, E, F, G, H, I, K, L, M, N, P, Q, R, S, T, V, W or Y at the amino acid position 63;

A, D, E, F, G, H, I, K, L, M, N, P, Q, R, S, T, V, W or Y at the amino acid position 64;
A, D, E, F, G, H, I, K, L, M, N, P, Q, R, S, T, V, W or Y at the amino acid position 65;
H or R at the amino acid position 93;
F, G, H, L, M, S, T, V or Y at the amino acid position 94;
I or V at the amino acid position 95;
F, H, I, K, L, M, T, V, W or Y at the amino acid position 96;
F, Y or W at the amino acid position 97;
A, F, G, H, I, K, L, M, N, Q, R, S, T, V, W or Y at the amino acid position 98;
A, F, G, H, I, K, L, M, N, P, Q, R, S, T, V, W or Y at the amino acid position 99;
A, D, E, F, G, H, I, K, L, M, N, P, Q, R, S, T, V, W or Y at the amino acid position 100;
A, D, E, F, G, H, I, K, L, M, N, P, Q, R, S, T, V, W or Y at the amino acid position 100a;
A, D, E, F, G, H, I, K, L, M, N, P, Q, R, S, T, V, W or Y at the amino acid position 100b;
A, D, E, F, G, H, I, K, L, M, N, P, Q, R, S, T, V, W or Y at the amino acid position 100c;
A, D, E, F, G, H, I, K, L, M, N, P, Q, R, S, T, V, W or Y at the amino acid position 100d;
A, D, E, F, G, H, I, K, L, M, P, Q, R, S, T, V, W or Y at the amino acid position 100e;
A, E, F, G, H, I, K, L, M, N, P, Q, R, S, T, V, W or Y at the amino acid position 100f;
A, E, F, G, H, I, K, L, M, N, P, Q, R, S, T, V, W or Y at the amino acid position 100g;
A, D, E, G, H, I, L, M, N, P, S, T or V at the amino acid position 100h;
A, D, E, F, G, H, I, K, L, M, N, P, Q, R, S, T, V, W or Y at the amino acid position 100i;
A, D, F, I, L, M, N, Q, S, T or V at the amino acid position 101;
A, D, E, F, G, H, I K, L, M, N, Q, R, S, T, V, W or Y at the amino acid position 102; and/or
(b) a light chain variable domain amino acid sequence comprising, at each of the following positions (all by Kabat numbering), one or more of the following amino acid residues indicated for that position:
A, D, F, G, H, I, K, L, M, N, Q, R, S, T, V, W or Y at the amino acid position 24;
A, G, N, P, S, T or V at the amino acid position 25;
A, D, E, F, G, I, K, L, M, N, Q, R, S, T or V at the amino acid position 26;
A, D, E, F, G, H, I, K, L, M, N, Q, R, S, T, V, W or Y at the amino acid position 27;
A, D, E, F, G, H, I, K, L, M, N, P, Q, R, S, T, V, W or Y at the amino acid position 27a;
A, I, L, M, P, T or V at the amino acid position 27b;
A, E, F, H, I, K, L, M, N, P, Q, R, T, W or Y at the amino acid position 27c;
A, E, G, H, I, K, L, M, N, P, Q, R, S, T, V, W or Y at the amino acid position 27d;
A, D, E, F, G, H, I, K, L, M, N, P, Q, R, S, T, V, W or Y at the amino acid position 27e;
G, N, S or T at the amino acid position 28;
A, F, G, H, K, L, M, N, Q, R, S, T, W or Y at the amino acid position 29;
A, F, G, H, I, K, L, M, N, Q, R, V, W or Y at the amino acid position 30;
I, L, Q, S, T or V at the amino acid position 31;
F, W or Y at the amino acid position 32;
A, F, H, L, M, Q or V at the amino acid position 33;
A, H or S at the amino acid position 34;
I, K, L, M or R at the amino acid position 50;
A, E, I, K, L, M, Q, R, S, T or V at the amino acid position 51;
A, D, E, F, G, H, I, K, L, M, N, Q, R, S, T, V, W or Y at the amino acid position 52;
A, E, F, G, H, K, L, M, N, P, Q, R, S, V, W or Y at the amino acid position 53;
A, D, E, F, G, H, I, K, L, M, N, P, Q, R, S, T, V, W or Y at the amino acid position 54;
A, D, E, F, G, H, I, K, L, M, N, P, Q, R, S, T, V or Y at the amino acid position 55;
A, D, E, F, G, H, I, K, L, M, N, P, Q, R, S, T, V, W or Y at the amino acid position 56;
A, G, K, S or Y at the amino acid position 89;
Q at the amino acid position 90;
G at the amino acid position 91;
A, D, H, K, N, Q, R, S or T at the amino acid position 92;
A, D, E, F, G, H, I, K, L, M, N, Q, R, S, T, V, W or Y at the amino acid position 93;
A, D, H, I, M, N, P, Q, R, S, T or V at the amino acid position 94;
P at the amino acid position 95;
F or Y at the amino acid position 96; and
A, D, E, G, H, I, K, L, M, N, Q, R, S, T or V at the amino acid position 97.

The antigen-binding molecule of the present invention can be produced by a method generally known to those skilled in the art. For example, the antibody can be prepared by a method given below, though the method for preparing the antibody of the present invention is not limited thereto. Many combinations of host cells and expression vectors are known in the art for antibody preparation by the transfer of isolated genes encoding polypeptides into appropriate hosts. All of these expression systems can be applied to the isolation of the antigen-binding molecule of the present invention. In the case of using eukaryotic cells as the host cells, animal cells, plant cells, or fungus cells can be appropriately used. Specifically, examples of the animal cells can include the following cells:

(1) mammalian cells such as CHO (Chinese hamster ovary cell line), COS (monkey kidney cell line), myeloma cells (Sp2/O, NS0, etc.), BHK (baby hamster kidney cell line), HEK293 (human embryonic kidney cell line with sheared adenovirus (Ad)5 DNA), PER.C6 cell (human embryonic retinal cell line transformed with the adenovirus type 5 (Ad5) E1A and E1B genes), Hela, and Vero (Current Protocols in Protein Science (May, 2001, Unit 5.9, Table 5.9.1));

(2) amphibian cells such as Xenopus oocytes; and
(3) insect cells such as sf9, sf21, and Tn5.
The antibody can also be prepared using E. coli (mAbs 2012 March-April; 4 (2): 217-225) or yeast (WO2000023579). The antibody prepared using E. coli is not glycosylated. On the other hand, the antibody prepared using yeast is glycosylated.

An antibody heavy chain-encoding DNA that encodes a heavy chain with one or more amino acid residues in a variable domain substituted by different amino acids of interest, and a DNA encoding a light chain of the antibody are expressed. The DNA that encodes a heavy chain or a light chain with one or more amino acid residues in a variable domain substituted by different amino acids of interest can be obtained, for example, by obtaining a DNA encoding an antibody variable domain prepared by a method known in the art against a certain antigen, and appropriately introducing substitution such that codons encoding the particular amino acids in the domain encode the different amino acids of interest.

Alternatively, a DNA encoding a protein in which one or more amino acid residues in an antibody variable domain prepared by a method known in the art against a certain antigen are substituted by different amino acids of interest may be designed in advance and chemically synthesized to obtain the DNA that encodes a heavy chain with one or more amino acid residues in a variable domain substituted by different amino acids of interest. The amino acid substitution site and the type of the substitution are not particularly limited. Examples of the region preferred for the amino acid alteration include solvent-exposed regions and loops in the variable region. Among others, CDR1, CDR2, CDR3, FR3, and loops are preferred. Specifically, Kabat numbering positions 31 to 35, 50 to 65, 71 to 74, and 95 to 102 in the H chain variable domain and Kabat numbering positions 24 to 34, 50 to 56, and 89 to 97 in the L chain variable domain are preferred. Kabat numbering positions 31, 52a to 61, 71 to 74, and 97 to 101 in the H chain variable domain and Kabat numbering positions 24 to 34, 51 to 56, and 89 to 96 in the L chain variable domain are more preferred.

The amino acid alteration is not limited to the substitution and may be deletion, addition, insertion, or modification, or a combination thereof.

The DNA that encodes a heavy chain with one or more amino acid residues in a variable domain substituted by different amino acids of interest can also be produced as separate partial DNAs. Examples of the combination of the partial DNAs include, but are not limited to: a DNA encoding a variable domain and a DNA encoding a constant domain; and a DNA encoding a Fab domain and a DNA encoding an Fc domain. Likewise, the light chain-encoding DNA can also be produced as separate partial DNAs.

These DNAs can be expressed by the following method: for example, a DNA encoding a heavy chain variable domain, together with a DNA encoding a heavy chain constant domain, is integrated to an expression vector to construct a heavy chain expression vector. Likewise, a DNA encoding a light chain variable domain, together with a DNA encoding a light chain constant domain, is integrated to an expression vector to construct a light chain expression vector. These heavy chain and light chain genes may be integrated to a single vector.

The DNA encoding the antibody of interest is integrated to expression vectors so as to be expressed under the control of expression control regions, for example, an enhancer and a promoter. Next, host cells are transformed with the resulting expression vectors and allowed to express antibodies. In this case, appropriate hosts and expression vectors can be used in combination.

Examples of the vectors include M13 series vectors, pUC series vectors, pBR322, pBluescript, and pCR-Script. In addition to these vectors, for example, pGEM-T, pDIRECT, or pT7 can also be used for the purpose of cDNA subcloning and excision.

Particularly, expression vectors are useful for using the vectors for the purpose of producing the antibody of the present invention. For example, when the host is E. coli such as JM109, DH5 alpha, HB101, or XL1-Blue, the expression vectors indispensably have a promoter that permits efficient expression in E. coli, for example, lacZ promoter (Ward et al., Nature (1989) 341, 544-546; and FASEB J. (1992) 6, 2422-2427, which are incorporated herein by reference in their entirety), araB promoter (Better et al., Science (1988) 240, 1041-1043, which is incorporated herein by reference in its entirety), or T7 promoter. Examples of such vectors include the vectors mentioned above as well as pGEX-5X-1 (manufactured by Pharmacia), “QIAexpress system” (manufactured by Qiagen N.V.), pEGFP, and pET (in this case, the host is preferably BL21 expressing T7 RNA polymerase).

The vectors may contain a signal sequence for polypeptide secretion. In the case of production in the periplasm of E. coli, pelB signal sequence (Lei, S. P. et al., J. Bacteriol. (1987) 169, 4397, which is incorporated herein by reference in its entirety) can be used as the signal sequence for polypeptide secretion. The vectors can be transferred to the host cells by use of, for example, a Lipofectin method, a calcium phosphate method, or a DEAE-dextran method.

In addition to the expression vectors for E. coli, examples of the vectors for producing the polypeptide of the present invention include mammal-derived expression vectors (e.g., pcDNA3 (manufactured by Invitrogen Corp.), pEGF-BOS (Nucleic Acids. Res. 1990, 18 (17), p. 5322, which is incorporated herein by reference in its entirety), pEF, and pCDM8), insect cell-derived expression vectors (e.g., “Bac-to-BAC baculovirus expression system” (manufactured by GIBCO BRL), and pBacPAK8), plant-derived expression vectors (e.g., pMH1 and pMH2), animal virus-derived expression vectors (e.g., pHSV, pMV, and pAdexLcw), retrovirus-derived expression vectors (e.g., pZlPneo), yeast-derived expression vectors (e.g., “Pichia Expression Kit” (manufactured by Invitrogen Corp.), pNV11, and SP-Q01), and Bacillus subtilis-derived expression vectors (e.g., pPL608 and pKTH50).

For the purpose of expression in animal cells such as CHO cells, COS cells, NIH3 T3 cells, or HEK293 cells, the vectors indispensably have a promoter necessary for intracellular expression, for example, SV40 promoter (Mulligan et al., Nature (1979) 277, 108, which is incorporated herein by reference in its entirety), MMTV-LTR promoter, EF1 alpha promoter (Mizushima et al., Nucleic Acids Res. (1990) 18, 5322, which is incorporated herein by reference in its entirety), CAG promoter (Gene. (1991) 108, 193, which is incorporated herein by reference in its entirety), or CMV promoter and, more preferably, have a gene for screening for transformed cells (e.g., a drug resistance gene that can work as a marker by a drug (neomycin, G418, etc.)). Examples of the vectors having such properties include pMAM, pDR2, pBK-RSV, pBK-CMV, pOPRSV, and pOP13. In addition, EBNA1 protein may be coexpressed therewith for the purpose of increasing the number of gene copies. In this case, vectors having a replication origin OriP are used (Biotechnol Bioeng. 2001 Oct. 20; 75 (2): 197-203; and Biotechnol Bioeng. 2005 Sep. 20; 91 (6): 670-7).

An exemplary method intended to stably express the gene and increase the number of intracellular gene copies involves transforming CHO cells deficient in nucleic acid synthesis pathway with vectors having a DHFR gene serving as a complement thereto (e.g., pCHOI) and using methotrexate (MTX) in the gene amplification. An exemplary method intended to transiently express the gene involves using COS cells having an SV40 T antigen gene on their chromosomes to transform the cells with vectors having a replication origin of SV40 (pcD, etc.). A replication origin derived from polyomavirus, adenovirus, bovine papillomavirus (BPV), or the like can also be used. In order to increase the number of gene copies in the host cell system, the expression vectors can contain a selective marker such as an aminoglycoside phosphotransferase (APH) gene, a thymidine kinase (TK) gene, an E. coli xanthine guanine phosphoribosyltransferase (Ecogpt) gene, or a dihydrofolate reductase (dhfr) gene.

The antibody can be recovered, for example, by culturing the transformed cells and then separating the antibody from within the molecule-transformed cells or from the culture solution thereof. The antibody can be separated and purified by appropriately using in combination methods such as centrifugation, ammonium sulfate fractionation, salting out, ultrafiltration, C1q, FcRn, protein A and protein G columns, affinity chromatography, ion-exchanged chromatography, and gel filtration chromatography.

The technique mentioned above, such as the knobs-into-holes technology (WO1996/027011; Ridgway J B et al., Protein Engineering (1996) 9, 617-621; and Merchant A M et al., Nature Biotechnology (1998) 16, 677-681) or the technique of suppressing the unintended association between H chains by the introduction of electric charge repulsion (WO2006/106905), can be applied to a method for efficiently preparing the multispecific antibody.

The present invention further provides a method for producing the antigen-binding molecule of the present invention and specifically provides a method for producing an antigen-binding molecule comprising: an antibody variable region that is capable of binding to two different antigens (first antigen and second antigen), but does not bind to CD3 and CD137 at the same time (this variable region is referred to as a first variable region); and a variable region binding to a third antigen different from CD3 and CD137 (this variable region is referred to as a second variable region), the method comprising the step of preparing an antigen-binding molecule library containing diverse amino acid sequences of the first variable region.

Examples thereof can include a production method comprising the following steps:

(i) preparing a library of antigen-binding molecules with at least one amino acid altered in their antibody variable regions each binding to CD3 or CD137, wherein the altered variable regions differ in at least one amino acid from each other;

(ii) selecting, from the prepared library, an antigen-binding molecule comprising a variable region that has binding activity against CD3 and CD137, but does not bind to CD3 and CD137 at the same time;

(iii) culturing a host cell comprising a nucleic acid encoding the variable region of the antigen-binding molecule selected in the step (ii), and a nucleic acid encoding a variable region of an antigen-binding molecule binding to the third antigen, to express an antigen-binding molecule comprising the antibody variable region that is capable of binding to CD3 and CD137, but does not bind to CD3 and CD137 at the same time, and the variable region binding to the third antigen; and

(iv) recovering the antigen-binding molecule from the host cell cultures.

In this production method, the step (ii) may be the following selection step:

(v) selecting, from the prepared library, an antigen-binding molecule comprising a variable region that has binding activity against CD3 and CD137, but does not bind to CD3 and CD137 each expressed on a different cell, at the same time.

The antigen-binding molecules used in the step (i) are not particularly limited as long as these molecules each comprise an antibody variable region. The antigen-binding molecules may be antibody fragments such as Fv, Fab, or Fab′ or may be Fc region-containing antibodies.

The amino acid to be altered is selected from, for example, amino acids whose alteration does not cancel the binding to the antigen, in the antibody variable region binding to CD3 or CD137.

In the present invention, one amino acid alteration may be used alone, or a plurality of amino acid alterations may be used in combination.

In the case of using a plurality of amino acid alterations in combination, the number of the alterations to be combined is not particularly limited and is, for example, 2 or more and 30 or less, preferably 2 or more and 25 or less, 2 or more and 22 or less, 2 or more and 20 or less, 2 or more and 15 or less, 2 or more and 10 or less, 2 or more and 5 or less, or 2 or more and 3 or less.

The plurality of amino acid alterations to be combined may be added to only the antibody heavy chain variable domain or light chain variable domain or may be appropriately distributed to both of the heavy chain variable domain and the light chain variable domain.

Examples of the region preferred for the amino acid alteration include solvent-exposed regions and loops in the variable region. Among others, CDR1, CDR2, CDR3, FR3, and loops are preferred. Specifically, Kabat numbering positions 31 to 35, 50 to 65, 71 to 74, and 95 to 102 in the H chain variable domain and Kabat numbering positions 24 to 34, 50 to 56, and 89 to 97 in the L chain variable domain are preferred. Kabat numbering positions 31, 52a to 61, 71 to 74, and 97 to 101 in the H chain variable domain and Kabat numbering positions 24 to 34, 51 to 56, and 89 to 96 in the L chain variable domain are more preferred.

The alteration of an amino acid residue also include: the random alteration of amino acids in the region mentioned above in the antibody variable region binding to CD3 or CD137; and the insertion of a peptide previously known to have binding activity against the CD3 or CD137, to the region mentioned above. The antigen-binding molecule of the present invention can be obtained by selecting a variable region that is capable of binding to CD3 and CD137, but cannot bind to these antigens at the same time, from among the antigen-binding molecules thus altered.

Whether the variable region is capable of binding to CD3 and CD137, but cannot bind to these antigens at the same time, and further, whether the variable region is capable of binding to both CD3 and CD137 at the same time when any one of CD3 and CD137 resides on a cell and the other antigen exists alone, both of the antigens each exist alone, or both of the antigens reside on the same cell, but cannot bind to these antigens each expressed on a different cell, at the same time, can also be confirmed according to the method mentioned above.

The present invention further provides a nucleic acid encoding the antigen-binding molecule of the present invention. The nucleic acid of the present invention may be in any form such as DNA or RNA.

The present invention further provides a vector comprising the nucleic acid of the present invention. The type of the vector can be appropriately selected by those skilled in the art according to host cells that receive the vector. For example, any of the vectors mentioned above can be used.

The present invention further relates to a host cell transformed with the vector of the present invention. The host cell can be appropriately selected by those skilled in the art. For example, any of the host cells mentioned above can be used.

The present invention also provides a pharmaceutical composition comprising the antigen-binding molecule of the present invention and a pharmaceutically acceptable carrier. The pharmaceutical composition of the present invention can be formulated according to a method known in the art by supplementing the antigen-binding molecule of the present invention with the pharmaceutically acceptable carrier. For example, the pharmaceutical composition can be used in the form of a parenteral injection of an aseptic solution or suspension with water or any other pharmaceutically acceptable solution. For example, the pharmaceutical composition may be formulated with the antigen-binding molecule mixed in a unit dosage form required for generally accepted pharmaceutical practice, in appropriate combination with pharmacologically acceptable carriers or media, specifically, sterilized water, physiological saline, plant oil, an emulsifier, a suspending agent, a surfactant, a stabilizer, a flavoring agent, an excipient, a vehicle, a preservative, a binder, etc. Specific examples of the carrier can include light anhydrous silicic acid, lactose, crystalline cellulose, mannitol, starch, carmellose calcium, carmellose sodium, hydroxypropylcellulose, hydroxypropylmethylcellulose, polyvinyl acetal diethylaminoacetate, polyvinylpyrrolidone, gelatin, medium-chain fatty acid triglyceride, polyoxyethylene hydrogenated castor oil 60, saccharide, carboxymethylcellulose, cornstarch, and inorganic salts. The amount of the active ingredient in such a preparation is determined such that an appropriate dose within the prescribed range can be achieved.

An aseptic composition for injection can be formulated according to conventional pharmaceutical practice using a vehicle such as injectable distilled water. Examples of aqueous solutions for injection include physiological saline, isotonic solutions containing glucose and other adjuvants, for example, D-sorbitol, D-mannose, D-mannitol, and sodium chloride. These solutions may be used in combination with an appropriate solubilizer, for example, an alcohol (specifically, ethanol) or a polyalcohol (e.g., propylene glycol and polyethylene glycol), or a nonionic surfactant, for example, polysorbate 80(TM) or HCO-50.

Examples of oily solutions include sesame oil and soybean oil. These solutions may be used in combination with benzyl benzoate or benzyl alcohol as a solubilizer. The solutions may be further mixed with a buffer (e.g., a phosphate buffer solution and a sodium acetate buffer solution), a soothing agent (e.g., procaine hydrochloride), a stabilizer (e.g., benzyl alcohol and phenol), and an antioxidant. The injection solutions thus prepared are usually charged into appropriate ampules. The pharmaceutical composition of the present invention is preferably administered parenterally. Specific examples of its dosage forms include injections, intranasal administration agents, transpulmonary administration agents, and percutaneous administration agents. Examples of the injections include intravenous injection, intramuscular injection, intraperitoneal injection, and subcutaneous injection, through which the pharmaceutical composition can be administered systemically or locally.

The administration method can be appropriately selected depending on the age and symptoms of a patient. The dose of a pharmaceutical composition containing a polypeptide or a polynucleotide encoding the polypeptide can be selected within a range of, for example, 0.0001 to 1000 mg/kg of body weight per dose. Alternatively, the dose can be selected within a range of, for example, 0.001 to 100000 mg/body of a patient, though the dose is not necessarily limited to these numeric values. Although the dose and the administration method vary depending on the weight, age, symptoms, etc. of a patient, those skilled in the art can appropriately select the dose and the method.

The present invention also provides a method for treating cancer, comprising the step of administering the antigen-binding molecule of the present invention, the antigen-binding molecule of the present invention for use in the treatment of cancer, use of the antigen-binding molecule of the present invention in the production of a therapeutic agent for cancer, and a process for producing a therapeutic agent for cancer, comprising the step of using the antigen-binding molecule of the present invention.

The three-letter codes and corresponding one-letter codes of amino acids used herein are defined as follows: alanine: Ala and A, arginine: Arg and R, asparagine: Asn and N, aspartic acid: Asp and D, cysteine: Cys and C, glutamine: Gln and Q, glutamic acid: Glu and E, glycine: Gly and G, histidine: His and H, isoleucine: Ile and I, leucine: Leu and L, lysine: Lys and K, methionine: Met and M, phenylalanine: Phe and F, proline: Pro and P, serine: Ser and S, threonine: Thr and T, tryptophan: Trp and W, tyrosine: Tyr and Y, and valine: Val and V.

Those skilled in the art should understand that one of or any combination of two or more of the aspects described herein is also included in the present invention unless a technical contradiction arises on the basis of the technical common sense of those skilled in the art.

All references cited herein are incorporated herein by reference in their entirety.

The present invention will be further illustrated with reference to Examples below. However, the present invention is not intended to be limited by Examples below.

### EXAMPLES

### [Example 1] Affinity Matured Variant Screening Derived from Parental Dual-Fab H183L072 for Improvement in In Vitro Cytotoxicity on Tumor Cells

1.1 Sequence of Affinity Matured Variants

To increase the binding affinity of parental Dual-Fab H183L072 (Heavy chain: SEQ ID NO: 1; Light chain: SEQ ID NO: 57), more than 1,000 Dual-Fab variants were generated using H183L072 as a template by introduce single or multiple mutations on variable region. Antibodies are expressed Expi293 (Invitrogen) and purified by Protein A purification followed by gel filtration, if gel filtration is necessary. The sequences of 15 represented variants with multiple mutations are listed in Table 1.1 and 1.2 and binding kinetics are evaluated in the Example 1.2.2 at 25 degrees C. and/or 37 degrees C. using Biacore T200 instrument (GE Healthcare) described below. Fold of affinity changes towards human CD137 and human CD3 by single mutations on variable regions are listed in Table 1.3.

TABLE 1.1a
SEQ ID NOs of human CD3 and CD137 antigen used for
affinity measurements

| Antigen name | SEQ ID NO |
|---|---|
| Human CD3eg linker | 84 |
| Human CD137 ECD | 201 |

TABLE 1.1b
Names and SEQ ID NOs of antibodies, variable
regions including VH, VL and CDRs 1, 2 and 3

| Ab | VHR | VLR | | VHR_ | VHR_ | VHR_ | | VLR_ | VLR_ | VLR_ |
|---|---|---|---|---|---|---|---|---|---|---|
| name | name | name | VHR | CDR1 | CDR2 | CDR3 | VLR | CDR1 | CDR2 | CDR3 |
| H183/L072 | dBBDu183H | dBBDu072L | 001 | 015 | 029 | 043 | 057 | 062 | 067 | 072 |
| H0868L0581 | dBBDu183H0868 | dBBDu072L0581 | 002 | 016 | 030 | 044 | 058 | 063 | 068 | 073 |
| H1550L0918 | dBBDu183H1550 | dBBDu072L0918 | 003 | 017 | 031 | 045 | 059 | 064 | 069 | 074 |
| H1571L0581 | dBBDu183H1571 | dBBDu072L0581 | 004 | 018 | 032 | 046 | 058 | 063 | 068 | 073 |
| H1610L0581 | dBBDu183H1610 | dBBDu072L0581 | 005 | 019 | 033 | 047 | 058 | 063 | 068 | 073 |
| H1610L0939 | dBBDu183H1610 | dBBDu072L0939 | 005 | 019 | 033 | 047 | 060 | 065 | 070 | 075 |
| H1643L0581 | dBBDu183H1643 | dBBDu072L0581 | 006 | 020 | 034 | 048 | 058 | 063 | 068 | 073 |
| H1647L0581 | dBBDu183H1647 | dBBDu072L0581 | 008 | 022 | 036 | 050 | 058 | 063 | 068 | 073 |
| H1649L0581 | dBBDu183H1649 | dBBDu072L0581 | 009 | 023 | 037 | 051 | 058 | 063 | 068 | 073 |
| H1649L0943 | dBBDu183H1649 | dBBDu072L0943 | 009 | 023 | 037 | 051 | 061 | 066 | 071 | 076 |
| H1651L0581 | dBBDu183H1651 | dBBDu072L0581 | 010 | 024 | 038 | 052 | 058 | 063 | 068 | 073 |
| H1652L0943 | dBBDu183H1652 | dBBDu072L0943 | 011 | 025 | 039 | 053 | 061 | 066 | 071 | 076 |
| H1673L0943 | dBBDu183H1673 | dBBDu072L0943 | 012 | 026 | 040 | 054 | 061 | 066 | 071 | 076 |
| H1673L0581 | dBBDu183H1673 | dBBDu072L0581 | 012 | 026 | 040 | 054 | 058 | 063 | 068 | 073 |
| H2591L0581 | dBBDu183H2591 | dBBDu072L0581 | 013 | 027 | 041 | 055 | 058 | 063 | 068 | 073 |
| H2594L0581 | dBBDu183H2594 | dBBDu072L0581 | 014 | 028 | 042 | 056 | 058 | 063 | 068 | 073 |
| CD3ϵ | CD3ϵVH | CD3ϵVL | 077 | | | | 078 | | | |
| CD137 | CD137VH | CD137VL | 079 | | | | 080 | | | |

TABLE 1.2a
Amino acid sequences of antigens

| | SEQ | |
|---|---|---|
| Antigen | ID | |
| name | NO | Amino Acid Sequence |
| Human | 84 | QDGNEEMGGITQTPYKVSISGTTVILTCPQYPGSEIL |
| CD3eg | | WQHNDKNIGGDEDDKNIGSDEDHLSLKEFSELEQSGY |
| linker | | YVCYPRGSKPEDANFYLYLRARVGSADDAKKDAAKKD |
| | | DAKKDDAKKDGSQSIKGNHLVKVYDYQEDGSVLLTCD |
| | | AEAKNITWFKDGKMIGFLTEDKKKWNLGSNAKDPRGM |
| | | YQCKGSQNKSKPLQVYYRMDYKDDDDK |
| Human | 201 | LQDPCSNCPAGTFCDNNRNQICSPCPPNSFSSAGGQR |
| CD137 | | TCDICRQCKGVFRTRKECSSTSNAECDCTPGFHCLGA |
| ECD | | GCSMCEQDCKQGCIELTKKGCKDCCFGTFNDQKRGIC |
| | | RPWINCSLDGKSVLVNGTKERDVVCGPSPADLSPGAS |
| | | SVTPPAPAREPGHSPQHHHHHHGGGGSGLNDIFEAQK |
| | | IEWHE |

TABLE 1.2b
Amino acid sequences of variable regions and CDRs 1, 2 and 3

| | SEQ | |
|---|---|---|
| | ID | |
| Name of VHR, VLR, CDR | NO | Amino Acid Sequence |
| dBBDu183H | 001 | QVQLVESGGGLVQPGRSLRLSCAASGFTFSNAWMHWVRQAPGK |
| | | GLEWVAQIKDKGNAYAAYYAPSVKGRFTISRDDSKNSIYLQMN |
| | | SLKTEDTAVYYCHYVHYASASTVLPAFGVDAWGQGTIVTVSS |
| dBBDu183H0868 | 002 | QVQLVESGGGLVQPGRSLRLSCAASGFKFSNVWMHWVRQAPGK |
| | | GLEWVAQIKDKYNAYAAYYAPSVKGRFTISRDDSKNSIYLQMN |
| | | SLKTEDTAVYYCHYVHYASASTLLPAFGVDAWGQGTTVIVSS |
| dBBDu183H1550 | 003 | QVQLVESGGGLVQPGRSLRLSCAASGFKESNVWMHWVRQAPGK |
| | | GLEWVAQIKDKYNAYAAYYAPSVKGRFTISRDDSKNSIYLQMN |
| | | SLKTEDTAVYYCHYIHYASASTLLPAFGVDAWGQGTIVTVSS |
| dB8Du183H1571 | 004 | QVQLVESGGGLVQPGRSLRLSCAASGFKFSNVWFHWVRQAPGK |
| | | GLEWVAQIKDKYNAYATYYAPSVKGRFTISRDDSKNSIYLQMN |
| | | SLKTEDTAVYYCHYVHYASASTLLPAFGVDAWGQGTTVTVSS |
| dBBDu183H1610 | 005 | QVQLVESGGGLVQPGRSLRLSCAASGFVFSNVWMHWVRQAPGK |
| | | GLEWVAQIKDKWNAYAAYYAPSVKGRFTISRDDSKNSIYLQMN |
| | | SLKTEDTAVYYCHYIHYASASTLLPAEGIDAWGQGTTVTVSS |
| dBBDu183H643 | 006 | QVQLVESGGGLVQPGRSLRLSCAASGFKESNVWFHWVRQAPGK |
| | | GLEWVAQIKDYYNAYAAYYAPSVKGRFTISRDDSKNSIYLQMN |
| | | SLKTEDTAVYYCHYVHYASASTLLPAEGVDAWGQGTTVTVSS |
| dBBDu183H1647 | 008 | QVQLVESGGGLVQPGRSLRLSCAASGFKFSNTWFHWVRQAPGK |
| | | GLEWVAQIKDYYNDYAAYYAPSVKGRFTISRDDSKNSIYLQMN |
| | | SLKTEDTAVYYCHYVHYASASTLLPAEGVDAWGQGTTVTVSS |
| dBBDu183H1649 | 009 | QVQLVESGGGLVQPGRSLRLSCAASGFVFSNVWFHWVRQAPGK |
| | | GLEWVAQIKDKYNAYADYYAPSVKERFTISRDDSKNSIYLQMN |
| | | SLKTEDTAVYYCHYVHYASASTLLPAEGVDAWGQGTTVIVSS |
| dBBDu183H1651 | 010 | QVQLVESGGGLVQPGRSLRLSCAASGFVFSNVWFHWVROAPGK |
| | | GLEWVAQIKDKYNAYADYYAPSVEGRFTISRDDSKNSIYLQMN |
| | | SLKTEDTAVYYCHYVHYASASTLLPAEGVDAWGQGTTVTVSS |
| dBBDu183H1652 | 011 | QVQLVESGGGLVQPGRSLRLSCAASGFVFSNVWFHWVRQAPGK |
| | | GLEWVAQIKDYYNAYADYYAPSVEGRFTISRDDSKNSIYLQMN |
| | | SLKTEDTAVYYCHYVHYASASTLLPAEGVDAWGQGTTVTVSS |
| dBBDu183H1673 | 012 | QVQLVESGGGLVQPGRSLRLSCAASGFVFSNVWFHWVRQAPGK |
| | | GLEWVAQIKDKWNAYADYYAPSVKERFTISRDDSKNSIYLQMN |
| | | SLKTEDTAVYYCHYIHYASASTLLPAEGIDAWGQGTIVTVSS |
| dBBDu183H2591 | 013 | QVQLVESGGGLVQPGRSLRLSCAASGFKFSNVWFHWVRQAPGK |
| | | GLEWVAQIKDYYNAYAGYYHPSVKGRFTISRDDSKNSIYLQMN |
| | | SLKTEDTAVYYCHYVHYAAASTLLPAEGVDAWGQGTTVTVSS |
| dBBDu183H2594 | 014 | QVQLVESGGGLVQPGRSLRLSCAASGFKFSNVWFHWVRQAPGK |
| | | GLEWVAQIKDYYNAYAGYYHPSVKGRFTISRODSKNSIYLQMN |
| | | SLKTEDTAVYYCHYVHYAAASQLLPAEGVDAWGQGTTVTVSS |
| dBBDu183H_VHR_CDR1 | 015 | NAWMH |
| dBBDu183H0868_VHR_CDR1 | 016 | NVWMH |
| dBBDu183H1550_VHR_CDR1 | 017 | NVWMH |
| dBBDu183H1571_VHR_CDR1 | 018 | NVWFH |
| dBBDu183H1610_VHR_CDR1 | 019 | NVWMH |
| dBBDu183H1643_VHR_CDR1 | 020 | NVWFH |
| dBBDu183H1647_VHR_CDR1 | 022 | NTWFH |
| dBBDu183H1649_VHR_CDR1 | 023 | NVWFH |
| dBBDu183H1651_VHR_CDR1 | 024 | NVWFH |
| dBBDu183H1652_VHR_CDR1 | 025 | NVWFH |
| dBBDu183H1673_VHR_CDR1 | 026 | NVWFH |
| dBBDu183H2591_VHR_CDR1 | 027 | NVWFH |
| dBBDu183H2594_VHR_CDR1 | 028 | NVWFH |
| dBBDu183H_VHR_CDR2 | 029 | QIKDKGNAYAAYYAPSVKG |
| dBBDu183H0868_VHR_CDR2 | 030 | QIKDKYNAYAAYYAPSVKG |
| dBBDu183H1550_VHR_CDR2 | 031 | QIKDKYNAYAAYYAPSVKG |
| dBBDu183H1571_VHR_CDR2 | 032 | QIKDKYNAYATYYAPSVKG |
| dBBDu183H1610_VHR_CDR2 | 033 | QIKDKWNAYAAYYAPSVKG |
| dBBDu183H1643_VHR_CDR2 | 034 | QIKDYYNAYAAYYAPSVKG |
| dBBDu183H1647_VHR_CDR2 | 036 | QIKDYYNDYAAYYAPSVKG |
| dBBDu183H1649_VHR_CDR2 | 037 | QIKDKYNAYADYYAPSVKE |
| dBBDu183H1651_VHR_CDR2 | 038 | QIKDKYNAYADYYAPSVEG |
| dBBDu183H1652_VHR_CDR2 | 039 | QIKDYYNAYADYYAPSVEG |
| dBBDu183H1673_VHR_CDR2 | 040 | QIKDKWNAYADYYAPSVKE |
| dBBDu183H2591_VHR_CDR2 | 041 | QIKDYYNAYAGYYHPSVKG |
| dBBDu183H2594_VHR_CDR2 | 042 | QIKDYYNAYAGYYHPSVKG |
| dBBDu183H_VHR_CDR3 | 043 | VHYASASTVLPAFGVDA |
| dBBDu183H0868_VHR_CDR3 | 044 | VHYASASTLLPAFGVDA |
| dBBDu183H1550_VHR_CDR3 | 045 | IHYASASTLLPAFGVDA |
| dBBDu183H1571_VHR_CDR3 | 046 | VHYASASTLLPAFGVDA |
| dBBDu183H1610_VHR_CDR3 | 047 | IHYASASTLLPAEGIDA |
| dBBDu183H1643_VHR_CDR3 | 048 | VHYASASTLLPAEGVDA |
| dBBDu183H1647_VHR_CDR3 | 050 | VHYASASTLLPAEGVDA |
| dBBDu183H1649_VHR_CDR3 | 051 | VHYASASTLLPAEGVDA |
| dBBDu183H1651_VHR_CDR3 | 052 | VHYASASTLLPAEGVDA |
| dBBDu183H1652_VHR_CDR3 | 053 | VHYASASTLLPAEGVDA |
| dBBDu183H1673_VHR_CDR3 | 054 | IHYASASTLLPAEGIDA |
| dBBDu183H2591_VHR_CDR3 | 055 | VHYAAASTLLPAEGVDA |
| dBBDu183H2594_VHR_CDR3 | 056 | VHYAAASQLLPAEGVDA |
| dBBDu072L | 057 | DIVMTQSPLSLPVTPGEPASISCQASQELVHMNRNTYLHWYQQ |
| | | KPGQAPRLLIYKVSNRFPGVPDRFSGSGSGTDFTLKISRVEAE |
| | | DVGVYYCAQGTSVPFTFGQGTKLEIK |
| dBBDu072L0581 | 058 | DIVMTQSPLSLPVTPGEPASISCQPSQEVVHMNRNTYLHWYQQ |
| | | KPGQAPRLLIYKVSNRFPGVPDRFSGSGSGTDFTLKISRVEAE |
| | | DVGVYYCAQGTSHPFTFGQGTKLEIK |
| dBBDu072L0918 | 059 | DIVMTQSPLSLPVTPGEPASISCQPSQEVVHMNNVVYLHWYQQ |
| | | KPGQAPRLLIYKVSNRFPGVPDRFSGSGSGTDFTLKISRVEAE |
| | | DVGVYYCAQGTSHPFTFGQGTKLEIK |
| dBBDu072L0939 | 060 | DIVMTQSPLSLPVTPGEPASISCQPSQEVVHMNRNTYLHWYQQ |
| | | KPGQAPRLLIYKVSNVFPGVPDRFSGSGSGTDFTLKISRVEAE |
| | | DVGVYYCAQGTHHPFTFGQGTKLEIK |
| dBBDu072L0943 | 061 | DIVMTQSPLSLPVTPGEPASISCQPSEEVVHMNRNTYLHWYQQ |
| | | KPGQAPRLLIYKVSNLFPGVPDRFSGSGSGTDFTLKISRVEAE |
| | | DVGVYYCAQGTHHPFTFGQGTKLEIK |
| dBBDu072L_VLR_CDR1 | 062 | QASQELVHMNRNTYLH |
| dBBDu072L0581_VLR_CDR1 | 063 | QPSQEVVHMNRNTYLH |
| dB8Du072L0918_VLR_CDR1 | 064 | QPSQEVVHMNNVVYLH |
| dBBDu072L0939_VLR_CDR1 | 065 | QPSQEVVHMNRNTYLH |
| dBBDu072L0943_VLR_CDR1 | 066 | QPSEEVVHMNRNTYLH |
| dBBDu072L_VLR_CDR2 | 067 | KVSNRFP |
| dBBDu072L0581_VLR_CDR2 | 068 | KVSNRFP |
| dBBDu072L0918_VLR_CDR2 | 069 | KVSNRFP |
| dBBDu072L0939_VLR_CDR2 | 070 | KVSNVFP |
| dBBDu072L0943_VLR_CDR2 | 071 | KVSNLFP |
| dBBDu072L_VLR_CDR3 | 072 | AQGTSVPFT |
| dBBDu072L0581_VLR_CDR3 | 073 | AQGTSHPFT |
| dBBDu072L0918_VLR_CDR3 | 074 | AQGTSHPFT |
| dBBDu072L0939_VLR_CDR3 | 075 | AQGTHHPFT |
| dBBDu072L0943_VLR_CDR3 | 076 | AQGTHHPFT |
| CD3εVH | 077 | QVQLVESGGGVVQPGGSLRLSCAASGFTFSNAWMHWVRQAPGK |
| | | GLEWVAQIKDKSQNYATYVAESVKGRFTISRADSKNSIYLQMN |
| | | SLKTEDTAVYYCRYVHYAAGYGVDIWGQGTTVTVSS |
| CD3εVL | 078 | DIVMTQSPLSLPVTPGEPASISCRSSQPLVHSNRNTYLHWYQQ |
| | | KPGQAPRLLIYKVSNRFSGVPDRFSGSGSGTDFTLKISRVEAE |
| | | DVGVYYCGQGTQVPYTFGQGTKLEIK |
| CD137VH | 079 | QVQLQQWGAGLLKPSETLSLTCAVYGGSFSGYYWSWIRQSPEK |
| | | GLEWIGEINHGGYVTYNPSLESRVTISVDTSKNQFSLKLSSVT |
| | | AADTAVYYCARDYGPGNYDWYFDLWGRGTLVTVSS |
| CD137VL | 080 | EIVLTQSPATLSLSPGERATLSCRASQSVSSYLAWYQQKPGQA |
| | | PRLLIYDASNRATGIPARFSGSGSGTDFTLTISSLEPEDFAVY |
| | | YCQQRSNWPPALTFGGGTKVEIK |

TABLE 1.3a
Fold changes of affinty for CD137 by single mutations in the heavy chain variable region

| All | | Kabat Numbering | | | | | | | | | | | | | | | | | | | | | | | |
|---|---|---|---|---|---|---|---|---|---|---|---|---|---|---|---|---|---|---|---|---|---|---|---|---|---|
| (without | | 26 | 27 | 28 | 29 | 30 | 31 | 32 | 33 | 34 | 35 | 50 | 51 | 52 | 52a | 52b | 52c | 53 | 54 | 55 | 56 | 57 | 58 | 59 | 60 |
| original & | | Template Sequence Name dBBDu183H | | | | | | | | | | | | | | | | | | | | | | | |
| Cys) | | G | F | T | F | S | N | A | W | M | H | Q | I | K | D | K | G | N | A | Y | A | A | Y | Y | A |
| Ala | A | 0.4 | 0.3 | 0.4 | 0.0 | 1.2 | 0.3 | | 0.0 | 0.0 | 0.0 | 0.0 | 0.1 | 0.0 | 0.0 | 1.0 | 0.8 | 0.0 | | 0.3 | | | 0.2 | 0.7 | |
| Ile | I | 0.3 | 0.7 | 0.8 | 0.3 | 0.6 | 0.3 | 2.2 | 0.0 | 1.4 | 0.0 | 0.8 | 0.0 | 0.0 | 0.0 | 0.8 | 0.7 | 0.0 | 0.5 | 0.0 | 0.2 | 0.1 | 0.2 | 0.7 | 0.7 |
| Leu | L | 0.3 | 0.5 | 0.9 | 0.3 | 0.5 | 0.3 | 1.2 | 0.0 | 1.8 | 0.0 | 0.8 | 0.1 | 0.1 | 0.0 | 0.5 | 0.8 | 0.0 | 1.0 | 0.3 | 0.5 | 0.0 | 0.4 | 1.1 | 1.1 |
| Met | M | 0.3 | 09 | 0.7 | 0.3 | 0.8 | 0.0 | 1.6 | 0.0 | | 0.0 | 1.5 | 0.2 | 0.3 | 0.0 | 0.6 | 0.6 | 0.0 | 1.0 | 0.4 | 0.7 | 0.7 | 0.6 | 1.1 | 1.0 |
| Pro | P | 0.1 | 0.3 | 0.5 | 0.1 | 0.9 | 0.4 | 0.0 | 0.0 | 0.3 | 0.0 | 0.0 | 0.0 | 0.0 | 0.0 | 0.4 | 0.4 | 0.0 | 0.0 | 0.0 | 0.2 | 1.0 | 0.2 | 0.3 | 0.1 |
| Val | V | 0.0 | 0.3 | 1.2 | 0.1 | 0.8 | 0.2 | 3.7 | 0.0 | 1.0 | 0.5 | 0.3 | 1.0 | 0.0 | 0.0 | 0.6 | 0.7 | 0.0 | 0.5 | 0.0 | 0.2 | 0.5 | 0.2 | 0.8 | 0.9 |
| Gly | G | | 0.4 | 0.3 | 0.0 | 0.9 | 0.3 | 0.1 | 0.0 | 0.0 | 0.0 | 0.0 | 0.1 | 0.0 | 0.0 | 0.9 | | 0.0 | 1.0 | 0.3 | 0.6 | 0.9 | 0.2 | 0.9 | 0.8 |
| Asn | N | 0.5 | 0.3 | 0.0 | 0.0 | 0.6 | | 0.3 | 0.0 | 0.0 | 0.4 | 0.5 | 0.2 | 0.2 | 0.0 | 0.2 | 0.9 | 0.0 | 1.1 | 0.7 | 0.7 | 0.3 | 0.3 | 0.8 | 1.3 |
| Gln | Q | 0.4 | 0.2 | 0.5 | 0.0 | 0.7 | 0.2 | 1.6 | 0.0 | 0.0 | 0.0 | | 0.0 | 0.3 | 0.0 | 0.8 | 0.7 | 0.0 | 1.0 | 0.2 | 0.8 | 0.9 | 0.4 | 0.8 | 1.1 |
| Ser | S | 0.4 | 0.3 | 0.0 | 0.0 | | 0.3 | 0.9 | 0.0 | 0.0 | 0.0 | 0.8 | 0.4 | 0.0 | 0.0 | 0.8 | 1.3 | 0.0 | 0.9 | 0.0 | 0.7 | 0.8 | 0.3 | 0.8 | 1.0 |
| Thr | T | 0.5 | 0.3 | | 0.1 | 0.9 | 0.5 | 3.9 | 0.0 | 0.0 | 0.5 | 2.0 | 0.3 | 0.0 | 0.0 | 0.1 | 1.0 | 0.0 | 0.3 | 0.0 | 0.4 | 0.6 | 0.3 | 0.8 | 1.3 |
| Asp | D | 0.8 | 0.5 | 0.0 | 0.0 | 0.4 | 0.0 | 0.0 | 0.0 | 0.0 | 0.0 | 0.3 | 0.0 | 0.0 | | 0.0 | 0.6 | 0.0 | 1.1 | 0.4 | 0.3 | 1.2 | 0.1 | 0.8 | 0.6 |
| Glu | E | 0.5 | 0.2 | 0.3 | 0.0 | 0.6 | 0.0 | 0.4 | 0.0 | 0.0 | 0.0 | 0.5 | 0.0 | 0.0 | 0.0 | 0.5 | 0.8 | 0.0 | 1.0 | 0.4 | 0.5 | 0.9 | 0.3 | 0.7 | 0.8 |
| His | H | 0.4 | 0.3 | 1.1 | 0.0 | 0.6 | 0.4 | 0.2 | 0.0 | 0.1 | | 0.6 | 0.0 | 0.0 | 0.0 | 0.7 | 1.3 | 0.0 | 1.0 | 0.4 | 0.6 | 0.5 | 0.3 | 0.8 | 1.3 |
| Lys | K | 0.0 | 0.2 | 1.1 | 0.0 | 0.7 | 0.0 | 1.0 | 0.0 | 0.0 | 0.0 | 0.8 | 0.1 | | 0.0 | | 0.2 | 0.0 | 0.9 | 0.2 | 0.8 | 0.9 | 0.3 | 1.0 | 1.1 |
| Arg | R | 0.3 | 0.2 | 1.5 | 0.0 | 0.7 | 0.0 | 0.3 | 0.0 | 0.0 | 0.0 | 0.0 | 0.0 | 0.2 | 0.0 | 1.2 | 0.2 | 0.0 | 0.9 | 0.0 | 0.8 | 0.5 | 0.4 | 0.9 | 1.2 |
| Phe | F | 0.3 | | 1.3 | | 0.7 | 0.2 | 0.1 | 0.0 | 2.5 | 4.3 | 2.6 | 0.0 | 0.0 | 0.0 | 0.7 | 1.7 | 0.0 | 0.9 | 0.5 | 0.0 | 0.1 | 0.9 | 1.5 | 1.2 |
| Trp | W | 0.3 | 0.3 | 0.8 | 0.1 | 0.5 | 0.3 | 0.0 | | 0.0 | 0.0 | 1.7 | 0.0 | 0.0 | 0.0 | 0.9 | 2.0 | 0.0 | 0.8 | 0.0 | 0.3 | 0.0 | 0.2 | 1.5 | 0.9 |
| Tyr | Y | 0.3 | 0.4 | 1.1 | 0.2 | 0.5 | 0.0 | 0.0 | 0.0 | 0.0 | 3.9 | 3.1 | 0.0 | 0.0 | 0.0 | 1.0 | 2.0 | 0.0 | 0.9 | | 0.3 | 0.0 | | | 1.2 |
| All | | Kabat Numbering | | | | | | | | | | | | | | | | | | | | | | | |
| (without | | 61 | 62 | 63 | 64 | 65 | 93 | 94 | 95 | 96 | 97 | 98 | 99 | 100 | 100a | 100b | 100c | 100d | 100e | 100f | 100g | 100h | 100i | 101 | 102 |
| original | | Template Sequence Name dBBDu183H | | | | | | | | | | | | | | | | | | | | | | | |
| & Cys) | | P | S | V | K | G | H | Y | V | H | Y | A | S | A | S | T | V | L | P | A | F | G | V | D | A |
| Ala | A | 0.8 | 0.7 | 0.9 | 1.2 | 1.2 | 0.0 | 0.0 | 0.0 | 0.0 | 0.0 | | 1.4 | | 0.0 | 0.0 | 0.4 | 0.8 | 0.5 | | 0.5 | 0.0 | 0.0 | 2.0 | |
| Ile | I | 0.5 | 0.7 | 1.1 | 1.1 | 1.3 | 0.0 | 0.0 | 0.3 | 0.5 | 0.0 | 0.0 | 0.0 | 0.0 | 0.0 | 0.0 | 2.5 | 0.0 | 0.0 | 0.0 | 1.0 | 0.0 | 1.1 | 0.0 | 0.0 |
| Leu | L | 0.6 | 0.9 | 0.9 | 1.1 | 1.3 | 0.0 | 0.0 | 0.2 | 0.8 | 0.0 | 0.0 | 0.3 | 0.0 | 0.0 | 0.1 | 3.3 | | 0.0 | 0.0 | 1.2 | 0.0 | 0.1 | 0.0 | 0.9 |
| Met | M | 0.6 | 0.8 | 0.8 | 1.1 | 1.2 | 0.0 | 3.2 | 0.0 | 1.4 | 0.0 | 0.0 | 0.8 | 0.7 | 0.0 | 0.5 | 1.3 | 0.9 | 0.0 | 0.0 | 0.6 | 0.0 | 0.0 | 0.0 | 0.0 |
| Pro | P | | 0.7 | 1.0 | 0.7 | 1.3 | 0.0 | 0.0 | 0.0 | 0.0 | 0.0 | 0.0 | 0.0 | 0.3 | 0.0 | 0.0 | 0.0 | 0.0 | | 0.3 | 0.0 | 0.0 | 0.0 | 0.0 | 0.0 |
| Val | V | 0.6 | 0.9 | | 1.0 | 1.2 | 0.0 | 0.0 | | 0.8 | 0.0 | 0.0 | 0.0 | 0.0 | 0.0 | 0.0 | | 0.5 | 0.0 | 0.0 | 0.5 | 0.0 | | 0.0 | 0.0 |
| Gly | G | 1.0 | 1.0 | 1.3 | 1.1 | | 0.0 | 0.0 | 0.0 | 0.0 | 0.0 | 0.0 | 1.0 | 0.7 | 0.0 | 0.0 | 0.0 | 0.0 | 0.4 | 0.6 | 0.0 | | 0.0 | 0.0 | 1.6 |
| Asn | N | 1.2 | 0.9 | 0.9 | 1.0 | 1.2 | 0.1 | 0.1 | 0.0 | 0.0 | 0.0 | 0.0 | 0.0 | 0.4 | 0.0 | 0.1 | 0.0 | 0.0 | 0.2 | 0.0 | 0.0 | 0.0 | 0.0 | 0.7 | 0.0 |
| Gln | Q | 0.6 | 1.0 | 1.3 | 1.1 | 1.2 | 0.0 | 0.3 | 0.0 | 0.0 | 0.0 | 0.0 | 0.6 | 0.6 | 0.0 | 0.3 | 1.4 | 0.5 | 0.4 | 0.0 | 0.8 | 0.0 | 0.0 | 1.7 | 0.0 |
| Ser | S | 1.1 | | 0.9 | 1.2 | 1.2 | 0.0 | 1.4 | 0.0 | 0.0 | 0.0 | 0.3 | | 0.0 | | 1.0 | 0.5 | 0.5 | 0.4 | 0.7 | 0.4 | 0.0 | 0.0 | 3.3 | 0.9 |
| Thr | T | 1.4 | 1.0 | 0.9 | 1.1 | 1.3 | 0.0 | 0.6 | 0.0 | 0.5 | 0.0 | 0.0 | 0.6 | 0.0 | 0.0 | | 0.5 | 0.0 | 0.2 | 0.0 | 0.7 | 0.0 | 0.2 | 3.3 | 0.0 |
| Asp | D | 0.7 | 0.9 | 0.9 | 1.1 | 1.3 | 0.0 | 0.2 | 0.0 | 0.0 | 0.0 | 0.0 | 0.0 | 0.0 | 0.0 | 0.0 | 0.0 | 0.0 | 0.3 | 0.0 | 0.0 | 0.0 | 0.0 | | 0.0 |
| Glu | E | 0.7 | 0.8 | 0.9 | 1.2 | 1.3 | 0.0 | 0.0 | 0.0 | 0.0 | 0.0 | 0.3 | 0.0 | 0.5 | 0.0 | 0.0 | 0.5 | 0.4 | 0.8 | 0.0 | 1.9 | 0.0 | 0.0 | 2.2 | 0.0 |
| His | H | 0.9 | 0.9 | 1.0 | 1.1 | 1.2 | | 0.0 | 0.0 | | 0.0 | 0.0 | 1.5 | 0.0 | 0.0 | 0.0 | 0.5 | 0.0 | 0.1 | 1.0 | 0.7 | 0.0 | 0.0 | 0.0 | 0.0 |
| Lys | K | 0.8 | 0.9 | 0.8 | | 1.1 | 0.0 | 0.0 | 0.0 | 0.0 | 0.0 | 0.0 | 1.2 | 1.1 | 0.0 | 0.0 | 0.5 | 0.7 | 0.3 | 0.0 | 0.0 | 0.0 | 0.0 | 0.0 | 0.0 |
| Arg | R | 1.0 | 1.1 | 1.0 | 1.1 | 1.1 | 0.0 | 0.0 | 0.0 | 0.0 | 0.0 | 0.0 | 1.1 | 0.5 | 0.0 | 0.0 | 0.7 | 0.0 | 0.3 | 0.0 | 0.0 | 0.0 | 0.0 | 0.0 | 0.0 |
| Phe | F | 0.7 | 0.5 | 0.9 | 1.1 | 1.3 | 0.0 | 1.8 | 0.0 | 1.4 | 1.8 | 0.0 | 0.0 | 0.0 | 0.0 | 0.0 | 0.0 | 0.0 | 0.0 | 0.0 | | 0.0 | 0.0 | 0.0 | 0.0 |
| Trp | W | 0.8 | 0.8 | 1.0 | 1.2 | 1.3 | 0.0 | 0.0 | 0.0 | 1.6 | 0.8 | 0.0 | 0.0 | 0.0 | 0.0 | 0.0 | 0.6 | 0.2 | 0.0 | 0.0 | 0.6 | 0.0 | 0.0 | 0.0 | 0.0 |
| Tyr | Y | 0.7 | 0.7 | 0.0 | 1.1 | 1.3 | 0.0 | | 0.0 | 3.3 | | 0.0 | 0.5 | 0.0 | 0.0 | 0.0 | 0.6 | 0.0 | 0.0 | 0.0 | 1.0 | 0.0 | 0.0 | 0.0 | 0.0 |

TABLE 1.3b
Fold changes of affinity for CD137 by single mutations in the light chain varible region

| All | | Kabat numbering | | | | | | | | | | | | | | | |
|---|---|---|---|---|---|---|---|---|---|---|---|---|---|---|---|---|---|
| (without | | 24 | 25 | 26 | 27 | 27a | 27b | 27c | 27d | 27e | 28 | 29 | 30 | 31 | 32 | 33 | 34 |
| original & | | Temple Sequence Name dBBDu072L | | | | | | | | | | | | | | | |
| Cys) | | Q | A | S | Q | E | L | V | H | M | N | R | N | T | Y | L | H |
| Ala | A | 1.0 | | 0.8 | 1.0 | 0.9 | 0.4 | 0.5 | 0.7 | 1.0 | 0.4 | 0.8 | 0.7 | 0.4 | 0.1 | 0.9 | 0.0 |
| Ile | I | 0.9 | 0.2 | 0.2 | 0.7 | 0.8 | 1.1 | 1.0 | 0.8 | 0.9 | 0.3 | 0.3 | 0.3 | 0.9 | 0.0 | 0.1 | 0.0 |
| Leu | L | 0.9 | 0.1 | 0.3 | 0.8 | 0.9 | | 0.8 | 0.6 | 0.8 | 0.3 | 0.9 | 0.3 | 0.5 | 0.2 | | 0.1 |
| Met | M | 1.0 | 0.1 | 0.6 | 0.9 | 0.9 | 0.4 | 0.9 | 0.6 | | 0.4 | 0.9 | 1.8 | 0.3 | 0.0 | 0.9 | 0.0 |
| Pro | P | 0.0 | 1.4 | 0.2 | 0.1 | 0.8 | 0.8 | 0.5 | 0.8 | 1.0 | 0.4 | 0.2 | 0.4 | 0.4 | 0.0 | 0.2 | 0.0 |
| Val | V | 0.9 | 0.5 | 0.3 | 0.8 | 0.9 | 1.1 | | 0.7 | 0.9 | 0.3 | 0.3 | 0.7 | 1.0 | 0.0 | 0.6 | 0.0 |
| Gly | G | 1.0 | 0.7 | 0.9 | 0.9 | 1.0 | 0.1 | 0.4 | 0.6 | 0.7 | 0.5 | 1.1 | 0.9 | 0.0 | 0.1 | 0.1 | 0.0 |
| Asn | N | 0.9 | 0.2 | 0.7 | 0.8 | 0.9 | 0.0 | 0.5 | 0.5 | 0.8 | | 0.9 | | 0.0 | 0.2 | 0.1 | 0.0 |
| Gln | Q | | 0.1 | 0.7 | | 1.1 | 0.2 | 0.8 | 0.6 | 1.0 | 0.4 | 0.9 | 1.0 | 0.5 | 0.2 | 1.4 | 0.0 |
| Ser | S | 1.0 | 1.1 | | 0.8 | 0.6 | 0.3 | 0.3 | 0.6 | 0.9 | 0.5 | 0.9 | 0.2 | 0.4 | 0.0 | 0.0 | 0.0 |
| Thr | T | 1.0 | 0.7 | 1.0 | 0.9 | 0.5 | 0.4 | 0.7 | 0.6 | 0.9 | 0.5 | 0.8 | 0.3 | | 0.0 | 0.2 | 0.0 |
| Asp | D | 0.9 | 0.0 | 0.8 | 0.9 | 1.1 | 0.2 | 0.3 | 0.3 | 0.7 | 0.0 | 0.3 | 0.1 | 0.1 | 0.0 | 0.0 | 0.0 |
| Glu | E | 0.0 | 0.0 | 0.8 | 1.0 | | 0.2 | 0.7 | 0.6 | 0.9 | 0.2 | 0.4 | 0.1 | 0.4 | 0.0 | 0.1 | 0.0 |
| His | H | 1.0 | 0.0 | 0.2 | 0.9 | 1.1 | 0.3 | 0.6 | | 0.8 | 0.4 | 0.8 | 1.0 | 0.0 | 0.1 | 0.9 | 0.0 |
| Lys | K | 1.0 | 0.0 | 0.5 | 0.9 | 0.9 | 0.3 | 0.9 | 0.6 | 1.1 | 0.3 | 1.1 | 0.6 | 0.4 | 0.0 | 0.0 | 0.0 |
| Arg | R | 0.9 | 0.0 | 0.3 | 0.9 | 0.9 | 0.0 | 0.9 | 0.6 | 1.0 | 0.4 | | 0.5 | 0.4 | 0.0 | 0.0 | 0.0 |
| Phe | F | 0.9 | 0.0 | 0.2 | 0.6 | 1.1 | 0.3 | 0.6 | 0.4 | 0.7 | 0.4 | 0.7 | 0.9 | 0.2 | 1.7 | 0.3 | 0.0 |
| Trp | W | 1.0 | 0.0 | 0.1 | 0.5 | 1.0 | 0.3 | 0.7 | 0.5 | 0.7 | 0.3 | 0.6 | 0.9 | 0.1 | 0.7 | 0.1 | 0.0 |
| Tyr | Y | 0.9 | 0.0 | 0.2 | 0.7 | 1.1 | 0.0 | 0.7 | 0.5 | 0.7 | 0.4 | 0.6 | 0.6 | 0.1 | | 0.1 | 0.0 |
| All | | Kabat numbering | | | | | | | | | | | | | | | |
| (without | | 50 | 51 | 52 | 53 | 54 | 55 | 56 | 89 | 90 | 91 | 92 | 93 | 94 | 95 | 96 | 97 |
| original & | | Temple Sequence Name dBBDu072L | | | | | | | | | | | | | | | |
| Cys) | | K | V | S | N | R | F | P | A | Q | G | T | S | V | P | F | T |
| Ala | A | 0.1 | 0.8 | 1.0 | 0.7 | 0.9 | 0.0 | 0.1 | | 0.0 | 0.4 | 0.5 | 0.9 | 0.1 | 0.0 | 0.0 | 0.6 |
| Ile | I | 2.5 | 0.6 | 1.4 | 0.2 | 1.0 | 0.0 | 0.1 | 0.0 | 0.0 | 0.0 | 0.1 | 1.1 | 0.7 | 0.0 | 0.0 | 1.3 |
| Leu | L | 1.0 | 1.7 | 1.2 | 0.2 | 0.9 | 0.2 | 0.1 | 0.0 | 0.0 | 0.0 | 0.2 | 1.3 | 0.3 | 0.0 | 0.0 | 0.3 |
| Met | M | 2.9 | 1.0 | 1.1 | 0.1 | 0.5 | 0.2 | 0.2 | 0.0 | 0.0 | 0.0 | 0.4 | 1.0 | 0.7 | 0.0 | 0.0 | 1.1 |
| Pro | P | 0.0 | 0.0 | 0.3 | 0.0 | 0.9 | 0.0 | | 0.0 | 0.0 | 0.0 | 0.0 | 0.0 | 0.1 | | 0.0 | 0.0 |
| Val | V | 0.3 | | 1.3 | 0.5 | 1.3 | 0.0 | 0.0 | 0.0 | 0.0 | 0.0 | 0.3 | 1.1 | | 0.0 | 0.0 | 1.0 |
| Gly | G | 0.0 | 0.4 | 0.8 | 0.6 | 0.9 | 0.0 | 0.1 | 0.5 | 0.0 | | 0.4 | 0.4 | 0.0 | 0.0 | 0.0 | 0.6 |
| Asn | N | 0.4 | 0.3 | 1.0 | | 0.06 | 0.0 | 0.2 | 0.0 | 0.0 | 0.0 | 0.0 | 1.0 | 0.2 | 0.0 | 0.0 | 0.5 |
| Gln | Q | 0.2 | 0.8 | 1.0 | 0.4 | 0.8 | 0.0 | 0.2 | 0.0 | | 0.0 | 0.0 | 1.1 | 0.8 | 0.0 | 0.0 | 0.7 |
| Ser | S | 0.0 | 0.6 | | 0.5 | 0.9 | 0.0 | 0.1 | 0.0 | 0.0 | 0.0 | 0.0 | | 0.0 | 0.0 | 0.0 | 0.8 |
| Thr | T | 0.2 | 0.6 | 1.0 | 0.0 | 2.5 | 0.0 | 0.2 | 0.0 | 0.0 | 0.0 | 0.0 | 1.1 | 0.4 | 0.0 | 0.0 | |
| Asp | D | 0.0 | 0.0 | 0.8 | 0.3 | 0.3 | 0.0 | 0.2 | 0.0 | 0.0 | 0.0 | 0.0 | 0.7 | 0.0 | 0.0 | 0.0 | 0.0 |
| Glu | E | 0.0 | 0.6 | 1.0 | 0.2 | 0.6 | 0.0 | 0.1 | 0.0 | 0.0 | 0.0 | 0.0 | 1.0 | 0.0 | 0.0 | 0.0 | 0.4 |
| His | H | 0.0 | 0.6 | 1.2 | 0.5 | 0.6 | 0.0 | 0.0 | 0.0 | 0.2 | 0.0 | 0.0 | 1.3 | 1.8 | 0.0 | 0.0 | 0.6 |
| Lys | K | | 1.4 | 1.2 | 0.2 | 1.0 | 0.0 | 0.0 | 0.0 | 0.0 | 0.0 | 0.0 | 1.1 | 0.1 | 0.0 | 0.0 | 1.1 |
| Arg | R | 1.7 | 1.4 | 1.0 | 0.3 | 0.0 | 0.0 | 0.0 | 0.0 | 0.0 | 0.0 | 0.0 | 1.0 | 0.4 | 0.0 | 0.0 | 0.9 |
| Phe | F | 0.1 | 0.2 | 1.2 | 0.3 | 0.3 | | 0.0 | 0.0 | 0.0 | 0.0 | 0.0 | 1.0 | 0.0 | 0.0 | | 0.3 |
| Trp | W | 0.0 | 0.1 | 1.0 | 0.0 | 0.4 | 0.2 | 0.0 | 0.0 | 0.0 | 0.0 | 0.0 | 1.2 | 0.0 | 0.0 | 0.0 | 0.1 |
| Tyr | Y | 0.0 | 0.2 | 1.2 | 0.3 | 0.2 | 0.0 | 0.0 | 0.0 | 0.0 | 0.0 | 0.0 | 0.8 | 0.1 | 0.0 | 0.6 | 0.3 |

TABLE 1.3c
Fold changes of affinity for CD3 by single mutations in the heavy chain varible region

| All | | Kabat Numbering | | | | | | | | | | | | | | | | | | | | | | | |
|---|---|---|---|---|---|---|---|---|---|---|---|---|---|---|---|---|---|---|---|---|---|---|---|---|---|
| (without | | 26 | 27 | 28 | 29 | 30 | 31 | 32 | 33 | 34 | 35 | 50 | 51 | 52 | 52a | 52b | 52c | 53 | 54 | 55 | 56 | 57 | 58 | 59 | 60 |
| original & | | Template Sequence Name dBBDu183H | | | | | | | | | | | | | | | | | | | | | | | |
| Cys) | | G | F | T | F | S | N | A | W | M | H | Q | I | K | D | K | G | N | A | Y | A | A | Y | Y | A |
| Ala | A | 0.5 | 0.2 | 0.0 | 0.2 | 0.8 | 0.4 | | 0.0 | 0.0 | 0.0 | 0.0 | 0.0 | 0.0 | 0.0 | 0.7 | 1.6 | 0.7 | | 0.4 | | | 0.6 | 1.0 | |
| Ile | I | 0.5 | 0.6 | 1.1 | 0.3 | 0.8 | 0.5 | 0.1 | 0.0 | 0.6 | 0.0 | 0.4 | | 0.0 | 0.0 | 0.8 | 1.1 | 0.4 | 0.7 | 0.0 | 0.6 | 0.6 | 0.3 | 0.9 | 0.6 |
| Leu | L | 0.6 | 0.4 | 0.9 | 0.4 | 0.8 | 0.2 | 0.0 | 0.0 | 0.7 | 0.0 | 0.2 | 0.0 | 0.0 | 0.0 | 0.6 | 1.5 | 0.6 | 1.1 | 0.5 | 0.7 | 0.5 | 0.4 | 1.0 | 1.2 |
| Met | M | 0.6 | 0.5 | 0.8 | 0.4 | 0.7 | 0.2 | 0.0 | 0.0 | | 0.0 | 0.1 | 0.3 | 0.6 | 0.0 | 0.5 | 1.4 | 0.5 | 1.0 | 0.6 | 1.0 | 0.9 | 0.4 | 1.0 | 1.0 |
| Pro | P | 0.1 | 0.0 | 0.7 | 0.2 | 0.3 | 0.1 | 0.0 | 0.0 | 0.0 | 0.0 | 0.0 | 0.0 | 0.0 | 0.0 | 0.5 | 1.3 | 0.0 | 0.0 | 0.0 | 0.3 | 0.9 | 0.5 | 0.7 | 0.5 |
| Val | V | 0.0 | 0.3 | 1.0 | 0.0 | 0.8 | 0.5 | 0.9 | 0.0 | 0.1 | 0.0 | 0.0 | 1.1 | 0.0 | 0.0 | 0.6 | 1.5 | 0.5 | 0.7 | 0.3 | 0.7 | 0.9 | 0.3 | 1.2 | 1.0 |
| Gly | G | | 0.5 | 0.7 | 0.0 | 1.1 | 0.3 | 0.3 | 0.0 | 0.0 | 0.0 | 0.0 | 0.0 | 0.0 | 0.2 | 0.8 | | 0.4 | 1.0 | 0.6 | 0.7 | 0.7 | 0.3 | 1.1 | 1.9 |
| Asn | N | 0.4 | 0.4 | 0.8 | 0.0 | 0.7 | | 0.5 | 0.0 | 0.0 | 0.0 | 0.0 | 0.0 | 0.0 | 0.1 | 0.5 | 1.2 | | 1.1 | 0.7 | 0.9 | 0.7 | 0.5 | 1.2 | 1.0 |
| Gln | Q | 0.4 | 0.0 | 0.0 | 0.2 | 0.8 | 0.2 | 0.4 | 0.0 | 0.0 | 0.0 | | 0.0 | 0.3 | 0.0 | 0.7 | 1.9 | 0.5 | 1.1 | 0.5 | 1.0 | 1.1 | 0.3 | 1.1 | 1.1 |
| Ser | S | 0.2 | 0.0 | 1.2 | 0.1 | | 0.5 | 0.4 | 0.0 | 0.0 | 0.5 | 0.0 | 0.4 | 0.0 | 0.0 | 0.7 | 1.0 | 0.8 | 1.0 | 0.0 | 0.9 | 1.1 | 0.4 | 1.1 | 1.3 |
| Thr | T | 0.4 | 0.0 | | 0.2 | 1.1 | 0.6 | 0.0 | 0.0 | 0.0 | 0.0 | 0.0 | 0.2 | 0.5 | 0.0 | 0.3 | 0.9 | 0.6 | 0.9 | 0.0 | 0.7 | 1.3 | 0.2 | 1.2 | 0.9 |
| Asp | D | 0.2 | 0.0 | 0.6 | 0.0 | 1.0 | 0.1 | 0.0 | 0.0 | 0.0 | 0.0 | 0.0 | 0.0 | 0.0 | | 0.0 | 1.1 | 0.4 | 0.9 | 0.4 | 0.5 | 1.0 | 0.0 | 1.1 | 1.3 |
| Glu | E | 0.6 | 0.0 | 0.6 | 0.1 | 0.6 | 0.0 | 0.1 | 0.0 | 0.0 | 0.0 | 0.0 | 0.0 | 0.0 | 0.0 | 0.5 | 1.2 | 0.5 | 1.0 | 0.6 | 0.6 | 1.0 | 0.2 | 1.1 | 1.1 |
| His | H | 0.0 | 0.1 | 0.9 | 0.0 | 1.0 | 0.3 | 0.5 | 0.0 | 0.0 | | 0.3 | 0.0 | 0.0 | 0.3 | 0.7 | 1.7 | 1.1 | 1.1 | 0.7 | 0.9 | 0.9 | 0.6 | 1.4 | 1.1 |
| Lys | K | 0.6 | 0.3 | 2.7 | 0.1 | 0.9 | 0.4 | 0.5 | 0.0 | 0.0 | 0.0 | 0.0 | 1.0 | | 0.0 | | 1.6 | 0.6 | 1.1 | 0.4 | 1.3 | 1.5 | 0.6 | 1.3 | 0.9 |
| Arg | R | 0.5 | 0.2 | 2.9 | 0.0 | 0.8 | 0.5 | 0.6 | 0.0 | 0.0 | 0.0 | 0.0 | 0.0 | 0.8 | 0.0 | 1.2 | 1.4 | 0.8 | 1.1 | 0.4 | 1.3 | 1.3 | 0.5 | 1.3 | 1.0 |
| Phe | F | 0.4 | | 1.0 | | 1.0 | 0.5 | 0.0 | 0.4 | 0.1 | 0.0 | 0.3 | 0.0 | 0.0 | 0.0 | 0.4 | 2.0 | 0.8 | 1.2 | 0.8 | 0.6 | 0.4 | 1.4 | 0.6 | 0.7 |
| Trp | W | 0.6 | 0.0 | 0.9 | 0.5 | 1.3 | 0.4 | 0.1 | | 0.0 | 0.0 | 0.0 | 0.0 | 0.0 | 0.0 | 0.6 | 1.5 | 1.2 | 1.1 | 0.5 | 0.8 | 0.4 | 0.0 | 0.6 | 1.0 |
| Tyr | Y | 0.6 | 0.0 | 1.5 | 0.4 | 1.2 | 0.3 | 0.0 | 0.1 | 0.0 | 0.0 | 0.9 | 0.0 | 0.0 | 0.0 | 0.7 | 1.5 | 0.9 | 1.0 | | 0.7 | 0.4 | | | 0.7 |
| All | | Kabat Numbering | | | | | | | | | | | | | | | | | | | | | | | |
| (without | | 61 | 62 | 63 | 64 | 65 | 93 | 94 | 95 | 96 | 97 | 98 | 99 | 100 | 100a | 100b | 100c | 100d | 100e | 100f | 100g | 100h | 100i | 101 | 102 |
| original & | | Template Sequence Name dBBDu183H | | | | | | | | | | | | | | | | | | | | | | | |
| Cys) | | P | S | V | K | G | H | Y | V | H | Y | A | S | A | S | T | V | L | P | A | F | G | V | D | A |
| Ala | A | 1.9 | 1.4 | 1.1 | 1.0 | 1.1 | 0.0 | 0.4 | 0.0 | 0.3 | 0.0 | | 1.0 | | 0.8 | 1.1 | 1.1 | 1.2 | 1.2 | | 1.7 | 4.8 | 4.6 | 0.8 | |
| Ile | I | 1.8 | 1.4 | 1.4 | 1.0 | 1.0 | 0.0 | 0.0 | 7.5 | 0.0 | 0.0 | 4.6 | 0.7 | 1.4 | 1.0 | 1.4 | 1.0 | 1.2 | 1.4 | 1.0 | 1.2 | 2.6 | 0.8 | 0.6 | 0.9 |
| Leu | L | 1.8 | 1.5 | 1.6 | 1.1 | 0.9 | 0.0 | 0.8 | 0.0 | 0.0 | 0.0 | 3.7 | 0.8 | 1.2 | 1.1 | 1.1 | 1.2 | | 1.3 | 1.2 | 1.3 | 0.8 | 1.8 | 2.3 | 0.7 |
| Met | M | 1.8 | 1.3 | 1.4 | 1.1 | 0.9 | 0.0 | 0.0 | 0.3 | 0.1 | 0.0 | 1.0 | 0.9 | 1.0 | 0.8 | 1.0 | 1.2 | 1.0 | 1.8 | 1.3 | 1.4 | 4.2 | 5.6 | 0.7 | 1.0 |
| Pro | P | | 1.3 | 1.2 | 1.6 | 0.9 | 0.0 | 0.0 | 0.0 | 0.0 | 0.0 | 0.2 | 0.6 | 1.3 | 1.1 | 1.1 | 1.0 | 1.4 | | 0.6 | 1.8 | 0.6 | 1.2 | 0.0 | 0.0 |
| Val | V | 1.8 | 1.4 | | 1.1 | 1.2 | 0.0 | 2.6 | | 0.0 | 0.0 | 4.7 | 0.8 | 1.2 | 1.0 | 1.2 | | 1.0 | 1.5 | 1.0 | 1.2 | 3.4 | | 0.5 | 1.0 |
| Gly | G | 1.5 | 1.4 | 1.0 | 1.0 | | 0.0 | 1.1 | 0.0 | 0.2 | 0.0 | 1.4 | 1.0 | 1.4 | 1.2 | 1.1 | 1.5 | 2.0 | 1.6 | 1.5 | 1.2 | | 1.8 | 0.4 | 1.0 |
| Asn | N | 1.5 | 1.4 | 1.3 | 1.0 | 0.9 | 0.0 | 0.0 | 0.0 | 0.0 | 0.0 | 1.5 | 0.6 | 0.8 | 0.8 | 1.0 | 1.0 | 1.2 | 1.3 | 1.3 | 0.9 | 0.8 | 5.0 | 1.1 | 1.5 |
| Gln | Q | 1.8 | 1.3 | 1.2 | 1.0 | 1.0 | 0.0 | 0.4 | 0.0 | 0.3 | 0.0 | 0.7 | 1.0 | 0.8 | 0.5 | 1.1 | 1.4 | 1.1 | 1.5 | 1.2 | 1.3 | 0.4 | 3.6 | 0.0 | 1.3 |
| Ser | S | 1.3 | | 1.1 | 1.0 | 1.0 | 0.0 | 0.0 | 0.0 | 0.2 | 0.0 | 1.0 | | 1.1 | | 1.5 | 1.1 | 1.2 | 1.2 | 1.7 | 1.4 | 1.1 | 1.9 | 0.6 | 1.9 |
| Thr | T | 1.0 | 1.3 | 1.2 | 1.1 | 1.0 | 0.0 | 0.3 | 0.0 | 0.0 | 0.0 | 2.0 | 0.9 | 0.9 | 0.9 | | 1.0 | 1.3 | 1.4 | 1.6 | 1.3 | 2.3 | 1.0 | 0.2 | 0.5 |
| Asp | D | 1.9 | 1.2 | 1.3 | 1.0 | 1.0 | 0.0 | 0.0 | 0.0 | 0.0 | 0.0 | 0.4 | 0.3 | 0.5 | 0.6 | 0.6 | 0.8 | 1.0 | 0.7 | 0.4 | 0.3 | 0.5 | 2.4 | | 1.3 |
| Glu | E | 1.8 | 1.2 | 1.1 | 1.0 | 1.0 | 0.0 | 0.0 | 0.0 | 0.0 | 0.0 | 0.3 | 0.3 | 0.5 | 0.6 | 0.6 | 0.8 | 0.7 | 1.0 | 0.5 | 0.5 | 0.5 | 1.4 | 0.0 | 1.1 |
| His | H | 1.8 | 1.3 | 1.0 | 1.0 | 0.9 | | 0.5 | 0.0 | | 0.0 | 5.9 | 0.7 | 1.2 | 1.1 | 1.2 | 1.2 | 1.3 | 1.5 | 1.2 | 1.8 | 1.5 | 2.2 | 0.4 | 3.6 |
| Lys | K | 1.7 | 1.4 | 1.3 | | 1.1 | 0.2 | 0.0 | 0.0 | 0.6 | 0.0 | 4.6 | 1.8 | 1.9 | 0.9 | 1.3 | 1.2 | 1.8 | 2.1 | 1.4 | 1.7 | 0.3 | 2.4 | 0.0 | 1.5 |
| Arg | R | 1.6 | 1.5 | 1.2 | 0.9 | 1.0 | 2.4 | 0.3 | 0.0 | 0.3 | 0.0 | 2.7 | 2.0 | 2.1 | 0.6 | 1.8 | 1.5 | 1.8 | 2.2 | 1.6 | 3.4 | 0.4 | 4.0 | 0.0 | 1.0 |
| Phe | F | 1.8 | 1.9 | 1.1 | 1.3 | 0.9 | 0.0 | 0.4 | 0.0 | 0.0 | 0.0 | 12.1 | 1.0 | 1.4 | 1.6 | 1.2 | 1.8 | 1.3 | 1.9 | 1.1 | | 0.4 | 3.2 | 2.5 | 2.0 |
| Trp | W | 1.8 | 2.0 | 0.8 | 1.0 | 1.0 | 0.0 | 0.4 | 0.0 | 0.0 | 0.2 | 7.0 | 0.9 | 1.9 | 1.6 | 1.8 | 1.5 | 1.7 | 1.7 | 0.7 | 0.9 | 0.0 | 4.9 | 0.0 | 3.3 |
| Tyr | Y | 1.9 | 1.5 | 1.1 | 1.0 | 1.0 | 0.0 | | 0.0 | 0.0 | | 25.1 | 0.8 | 1.7 | 1.2 | 1.8 | 1.3 | 1.4 | 2.0 | 1.5 | 1.2 | 0.0 | 3.0 | 0.0 | 2.5 |

TABLE 1.3d
Fold changes of affinity for CD3 by single mutations
in the light chain variable legion

| | | Kabat Numbering | | | | | | | | | | | | | | | |
|---|---|---|---|---|---|---|---|---|---|---|---|---|---|---|---|---|---|
| | | 24 | 25 | 26 | 27 | 27a | 27b | 27c | 27d | 27e | 28 | 29 | 30 | 31 | 32 | 33 | 34 |
| All (without | | Template Sequence Name dBBDu072L | | | | | | | | | | | | | | | |
| original & Cys) | | Q | A | S | Q | E | L | V | H | M | N | R | N | T | Y | L | H |
| Ala | A | 1.1 | 0.0 | 0.8 | 1.2 | 1.2 | 0.7 | 0.4 | 0.0 | 1.3 | 0.0 | 0.6 | 0.3 | 0.2 | 0.0 | 0.6 | 0.6 |
| Ile | I | 1.2 | 0.3 | 0.5 | 0.8 | 1.1 | 0.5 | 1.0 | 0.0 | 0.9 | 0.0 | 0.2 | 0.5 | 0.7 | 0.0 | 0.0 | 0.0 |
| Leu | L | 1.2 | 0.0 | 0.5 | 1.0 | 1.2 | | 0.8 | 0.0 | 1.0 | 0.0 | 0.8 | 0.5 | 0.0 | 0.0 | | 0.0 |
| Met | M | 1.1 | 0.0 | 0.6 | 1.2 | 1.2 | 0.7 | 1.1 | 0.0 | | 0.0 | 0.7 | 1.6 | 0.0 | 0.0 | 1.1 | 0.0 |
| Pro | P | 0.0 | 1.2 | 0.2 | 0.0 | 1.0 | 1.8 | 0.0 | 0.0 | 1.2 | 0.0 | 0.0 | 0.0 | 0.0 | 0.0 | 0.0 | 0.0 |
| Val | V | 1.2 | 0.9 | 0.6 | 0.9 | 1.0 | 1.7 | | 0.0 | 0.8 | 0.0 | 0.3 | 0.5 | 0.7 | 0.0 | 0.4 | 0.0 |
| Gly | G | 1.2 | 0.9 | 0.9 | 1.0 | 1.4 | 0.0 | 0.0 | 0.0 | 0.9 | 0.0 | 1.2 | 0.0 | 0.0 | 0.0 | 0.0 | 0.3 |
| Asn | N | 1.1 | 0.5 | 0.9 | 1.0 | 1.1 | 0.0 | 0.3 | 0.0 | 0.9 | | 0.3 | | 0.0 | 0.0 | 0.0 | 0.2 |
| Gln | Q | | 0.0 | 0.8 | | 1.2 | 0.0 | 1.0 | 0.0 | 1.1 | 0.0 | 0.7 | 1.2 | 0.4 | 0.0 | 1.7 | 0.0 |
| Ser | S | 1.1 | 1.3 | | 1.1 | 1.3 | 0.0 | 0.0 | 0.0 | 1.2 | 0.0 | 0.8 | 0.0 | 0.5 | 0.0 | 0.0 | 0.5 |
| Thr | T | 1.1 | 1.1 | 1.4 | 1.0 | 1.3 | 0.6 | 0.8 | 0.0 | 1.2 | 0.0 | 0.8 | 0.1 | | 0.0 | 0.3 | 0.4 |
| Asp | D | 1.2 | 0.0 | 0.8 | 1.1 | 1.1 | 0.0 | 0.1 | 0.0 | 0.8 | 0.0 | 0.3 | 0.1 | 0.0 | 0.0 | 0.0 | 0.0 |
| Glu | E | 0.0 | 0.0 | 0.7 | 1.1 | | 0.0 | 0.7 | 0.0 | 1.0 | 0.0 | 0.3 | 0.3 | 0.4 | 0.0 | 0.0 | 0.0 |
| His | H | 1.1 | 0.0 | 0.4 | 1.2 | 1.4 | 0.0 | 0.3 | | 0.9 | 0.0 | 0.8 | 0.8 | 0.1 | 0.0 | 1.4 | |
| Lys | K | 1.0 | 0.0 | 0.7 | 0.8 | 1.1 | 0.0 | 1.1 | 0.0 | 1.4 | 0.0 | 1.1 | 1.3 | 0.4 | 0.0 | 0.0 | 0.4 |
| Arg | R | 1.0 | 0.0 | 0.5 | 1.0 | 1.2 | 0.0 | 1.2 | 0.0 | 1.4 | 0.0 | | 1.5 | 0.4 | 0.0 | 0.0 | 0.0 |
| Phe | F | 0.9 | 0.0 | 0.6 | 0.9 | 1.1 | 0.3 | 0.5 | 0.2 | 0.9 | 0.0 | 0.6 | 0.9 | 0.0 | 0.0 | 1.1 | 0.0 |
| Trp | W | 1.1 | 0.0 | 0.3 | 0.7 | 1.1 | 0.0 | 0.3 | 0.0 | 1.0 | 0.0 | 0.5 | 0.9 | 0.0 | 0.0 | 0.0 | 0.0 |
| Tyr | Y | 1.0 | 0.0 | 0.4 | 0.9 | 1.2 | 0.0 | 0.5 | 0.0 | 0.9 | 0.0 | 0.5 | 0.8 | 0.0 | | 0.0 | 0.0 |

Kabat numbering

| | | 50 | 51 | 52 | 53 | 54 | 55 | 56 | 89 | 90 | 91 | 92 | 93 | 94 | 95 | 96 | 97 |
|---|---|---|---|---|---|---|---|---|---|---|---|---|---|---|---|---|---|
| All (without | | Template Sequence Name dBBDu072L | | | | | | | | | | | | | | | |
| original & Cys) | | K | V | S | N | R | F | P | A | Q | G | T | S | V | P | F | T |
| Ala | A | 0.0 | 0.6 | 1.2 | 0.6 | 1.0 | 7.2 | 4.1 | | 0.0 | 0.0 | 0.1 | 1.1 | 1.0 | 0.0 | 0.0 | 0.8 |
| Ile | I | 0.0 | 1.4 | 1.5 | 0.0 | 1.3 | 3.4 | 3.1 | 0.0 | 0.0 | 0.0 | 0.0 | 0.8 | 0.8 | 0.0 | 0.0 | 0.5 |
| Leu | L | 0.0 | 0.6 | 1.6 | 0.6 | 1.3 | 2.9 | 3.1 | 0.0 | 0.0 | 0.0 | 0.3 | 0.9 | 0.3 | 0.0 | 0.0 | 0.4 |
| Met | M | 0.0 | 0.5 | 1.4 | 0.9 | 1.7 | 3.6 | 3.9 | 0.1 | 0.0 | 0.0 | 0.3 | 1.0 | 0.8 | 0.0 | 0.0 | 0.7 |
| Pro | P | 0.0 | 0.0 | 0.0 | 0.6 | 0.9 | 8.1 | | 0.0 | 0.0 | 0.0 | 0.0 | 0.0 | 1.5 | | 0.0 | 0.0 |
| Val | V | 0.0 | | 1.6 | 0.3 | 1.1 | 4.4 | 3.4 | 0.4 | 0.0 | 0.0 | 0.4 | 0.7 | | 0.0 | 0.0 | 0.6 |
| Gly | G | 0.0 | 0.4 | 1.4 | 0.6 | 1.1 | 8.1 | 3.7 | 0.7 | 0.0 | | 0.2 | 0.8 | 0.0 | 0.0 | 0.0 | 1.0 |
| Asn | N | 0.0 | 0.0 | 1.5 | | 1.0 | 3.9 | 3.3 | 0.0 | 0.0 | 0.0 | 0.2 | 0.8 | 0.9 | 0.0 | 0.0 | 0.5 |
| Gln | Q | 0.0 | 0.5 | 1.4 | 0.8 | 1.2 | 2.9 | 3.7 | 0.2 | | 0.0 | 0.0 | 1.0 | 1.0 | 0.0 | 0.0 | 0.5 |
| Ser | S | 0.0 | 0.3 | | 0.9 | 1.2 | 6.6 | 4.1 | 0.6 | 0.0 | 0.0 | 0.5 | | 0.7 | 0.0 | 0.0 | 1.1 |
| Thr | T | 0.0 | 0.2 | 1.3 | 0.4 | 0.8 | 4.2 | 3.9 | 0.0 | 0.0 | 0.0 | | 1.0 | 1.3 | 0.0 | 0.0 | |
| Asp | D | 0.0 | 0.0 | 1.0 | 0.2 | 0.5 | 6.1 | 3.0 | 0.0 | 0.0 | 0.0 | 0.0 | 0.7 | 0.5 | 0.0 | 0.0 | 0.6 |
| Glu | E | 0.0 | 0.4 | 1.0 | 0.5 | 0.8 | 3.4 | 3.4 | 0.0 | 0.4 | 0.0 | 0.0 | 1.0 | 0.4 | 0.0 | 0.0 | 0.6 |
| His | H | 0.0 | 0.2 | 1.7 | 1.2 | 1.9 | 7.4 | 2.9 | 0.0 | 0.1 | 0.0 | 0.2 | 0.9 | 0.8 | 0.0 | 0.0 | 0.5 |
| Lys | K | | 0.8 | 1.9 | 1.4 | 1.7 | 8.3 | 5.6 | 1.1 | 0.0 | 0.0 | 0.8 | 1.0 | 0.4 | 0.0 | 0.0 | 0.6 |
| Arg | R | 0.0 | 0.9 | 1.8 | 1.8 | | 4.0 | 5.5 | 0.0 | 0.0 | 0.0 | 0.5 | 1.0 | 0.5 | 0.0 | 0.0 | 0.4 |
| Phe | F | 0.0 | 0.0 | 1.6 | 0.7 | 1.5 | | 3.3 | 0.2 | 0.0 | 0.0 | 0.3 | 1.0 | 0.2 | 0.0 | | 0.3 |
| Trp | W | 0.0 | 0.0 | 1.4 | 0.7 | 1.4 | 0.2 | 3.0 | 0.1 | 0.0 | 0.0 | 0.0 | 1.1 | 0.0 | 0.0 | 0.0 | 0.0 |
| Tyr | Y | 0.0 | 0.0 | 1.5 | 0.7 | 1.6 | 10.6 | 2.7 | 0.5 | 0.0 | 0.0 | 0.0 | 0.7 | 0.2 | 0.0 | 0.4 | 0.2 |

In Tables 1.3a to 1.3 d, mutated positions according to Kabat numbering and original amino acids at the respective positions are shown in the top two rows. The values represent fold changes of affinity when each of the mutations shown in the leftmost column was introduced into each position.

1.2. Binding Kinetics Information of Affinity Matured Variants

1.2.1 Expression and Purification of Human CD3 and CD137

The gamma and epsilon subunits of the human CD3 complex (human CD3 eg linker) were linked by a 29-mer linker and a Flag-tag was fused to the C-terminal end of the gamma subunit (SEQ ID NO: 84, Table 1.1a and 1.2a). This construct was expressed transiently using FreeStyle293F cell line (Thermo Fisher). Conditioned media expressing human CD3 eg linker was concentrated using a column packed with Q HP resins (GE healthcare) then applied to FLAG-tag affinity chromatography. Fractions containing human CD3 eg linker were collected and subsequently subjected to a Superdex 200 gel filtration column (GE healthcare) equilibrated with 1×D-PBS. Fractions containing human CD3 eg linker were then pooled and stored at −80 degrees C.

Human CD137 extracellular domain (ECD) (SEQ ID NO: 201, Table 1.1a and 1.2a) with hexahistidine (His-tag) and biotin acceptor peptide (BAP) on its C-terminus was expressed transiently using FreeStyle293F cell line (Thermo Fisher). Conditioned media expressing human CD137 ECD was applied to a HisTrap HP column (GE healthcare) and eluted with buffer containing imidazole (Nacalai). Fractions containing human CD137 ECD were collected and subsequently subjected to a Superdex 200 gel filtration column (GE healthcare) equilibrated with 1×D-PBS. Fractions containing human CD137 ECD were then pooled and stored at −80 degrees C.

1.2.2 Affinity Measurement Towards Human CD3 and CD137

Binding affinity of Dual-Fab antibodies (Dual-Ig) to human CD3 were assessed at 25 degrees C. using Biacore T200 instrument (GE Healthcare). Anti-human Fc (GE Healthcare) was immobilized onto all flow cells of a CM4 sensor chip using amine coupling kit (GE Healthcare). Antibodies were captured onto the anti-Fc sensor surfaces, then recombinant human CD3 or CD137 was injected over the flow cell. All antibodies and analytes were prepared in ACES pH 7.4 containing 20 mM ACES, 150 mM NaCl, 0.05% Tween 20, 0.005% NaN3. Sensor surface was regenerated each cycle with 3M MgCl2. Binding affinity were determined by processing and fitting the data to 1:1 binding model using Biacore T200 Evaluation software, version 2.0 (GE Healthcare). CD137 binding affinity assay was conducted in same condition except assay temperature was set at 37 degrees C. Binding affinity of Dual-Fab antibodies to recombinant human CD3 & CD137 are shown in Table 1.4.

TABLE 1.4

| | CD3 | | | CD137 | | |
|---|---|---|---|---|---|---|
| | Kon (M-1 · s-1) | Koff (s-1) | KD (M) | Kon (M-1 · s-1) | Koff (s-1) | KD (M) |
| H183/L072 | 3.54E+04 | 1.20E−02 | 3.40E−07 | 3.47E+03 | 1.96E−02 | 5.66E−06 |
| H0868L0581 | 1.23E+05 | 1.94E−02 | 1.57E−07 | 1.22E+04 | 1.36E−03 | 1.11E−07 |
| H1550L0918 | 7.20E+04 | 3.16E−03 | 4.38E−08 | 1.09E+04 | 5.79E−03 | 5.30E−07 |
| H1571L0581 | 1.42E+05 | 1.56E−02 | 1.10E−07 | 1.21E+04 | 1.05E−03 | 8.68E−08 |
| H1610L0581 | 6.80E+04 | 1.42E−03 | 2.09E−08 | 1.07E+04 | 1.10E−03 | 1.03E−07 |
| H1610L0939 | 5.00E+04 | 2.53E−03 | 5.07E−08 | 1.30E+04 | 8.01E−04 | 6.18E−08 |
| H1643L0581 | 9.46E+04 | 2.51E−02 | 2.65E−07 | 1.23E+04 | 6.06E−04 | 4.94E−08 |
| H1647L0581 | 4.43E+04 | 1.01E−01 | 2.28E−06 | 9.98E+03 | 6.47E−04 | 6.48E−08 |
| H1649L0581 | 7.50E+04 | 3.36E−02 | 4.49E−07 | 1.29E+04 | 5.53E−04 | 4.28E−08 |
| H1649L0943 | 6.10E+04 | 4,81E−02 | 7.89E−07 | 1.43E+04 | 4.68E−04 | 3.28E−08 |
| H1651L0581 | 7.18E+04 | 3.71E−02 | 5.17E−07 | 1.40E+04 | 6.03E−04 | 4.32E−08 |
| H1652L0943 | 6.23E+04 | 6.36E−02 | 1.02E−06 | 1.29E+04 | 4.70E−04 | 3.64E−08 |
| H1673L0581 | 7.96E+04 | 1.06E−03 | 1.33E−08 | 1.19E+04 | 9.60E−04 | 8.04E−08 |
| H1673L0943 | 5.50E+04 | 1.16E−03 | 2.10E−08 | 1.22E+04 | 7.22E−04 | 5.91E−08 |
| H2591L0581 | 1.02E+05 | 5.35E−02 | 5.25E−07 | 2.04E+04 | 7.42E−04 | 3.63E−08 |
| H2594L0581 | 9.83E+04 | 5.84E−02 | 5.93E−07 | 2.09E+04 | 1.63E−03 | 7.81E−08 |

1.3. Bi-Specific and Tri-Specific Antibody Preparation

To evaluate the efficacy of Dual-Ig variants, bi-specific or tri-specific antibodies were generated with one arm recognising tumor antigen and the other recognizing effector cells, predominantly T-cells. Anti-GPC3 (Heavy chain: SEQ ID NO: 206; Light chain: SEQ ID NO: 207) targeting tumor antigen glypican-3 (GPC3) or negative control, Keyhole Limpet Hemocyanin (KLH) (herein termed as Ctrl) antibodies were used as anti-target binding arms while antibodies described in Example 1.1 and 1.2 were generated using Fab-arm exchange (FAE) according to a method described in (Proc Natl Acad Sci USA. 2013 Mar. 26; 110(13): 5145-5150). The molecular format of bi-specific or tri-specific antibodies are the same format as a conventional IgG. For example, GPC3/H1643L581 is a tri-specific antibody that is able to bind GPC3, CD3, and CD137. To identify which Dual-Ig tri-specific variants described in Example 1.1 contributes to improved cytotoxicity attributed to CD137 activity, GPC3/CD3 epsilon, a bi-specific antibody (Table 1.1) that is able to bind GPC3 and CD3 was included as a control. All antibodies generated comprises a silent Fc with attenuated affinity for Fc gamma receptor.

### [Example 2] Evaluation of In Vitro Cytotoxicity by Affinity Matured Variants Derived from Parental Dual-Fab H183L072 on Tumor Cells

2.1. Assessment of CD3 Agonistic Activity of Affinity Matured Variants In Vitro

To evaluate CD3 agonistic activity as a result of affinity maturation, NFAT-luc2 Jurkat luciferase assay is conducted. Briefly, 4×10³ cells/well SK-pca60 cells (reference Example 13) which express human GPC3 on the cell membrane, was used as target cells and co-cultured with 2.0×10⁴ cells/well of NFAT-luc2 Jurkat cells (E:T ratio 5) for 24 hours in the presence of 0.02, 0.2 and 2 nM of tri-specific antibodies.

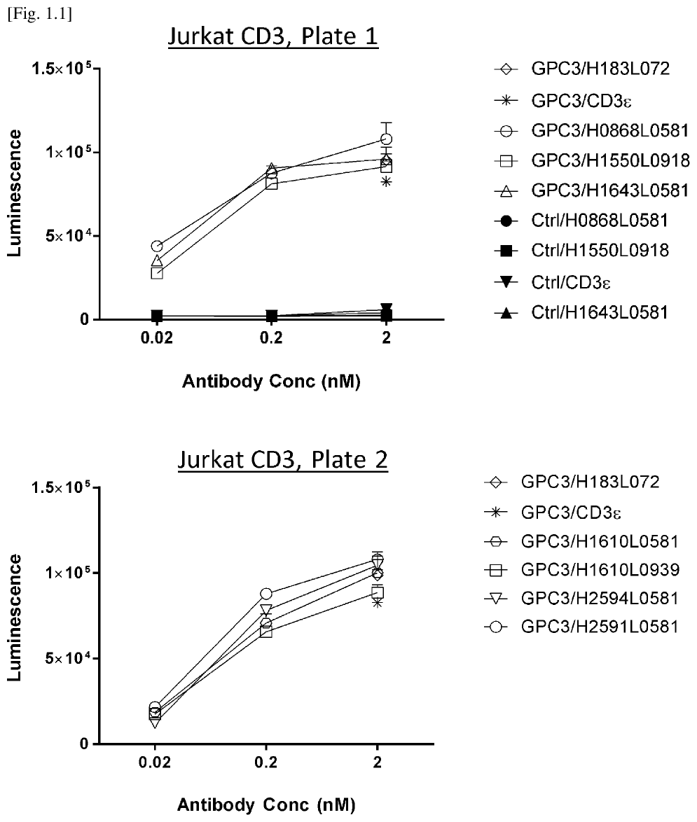

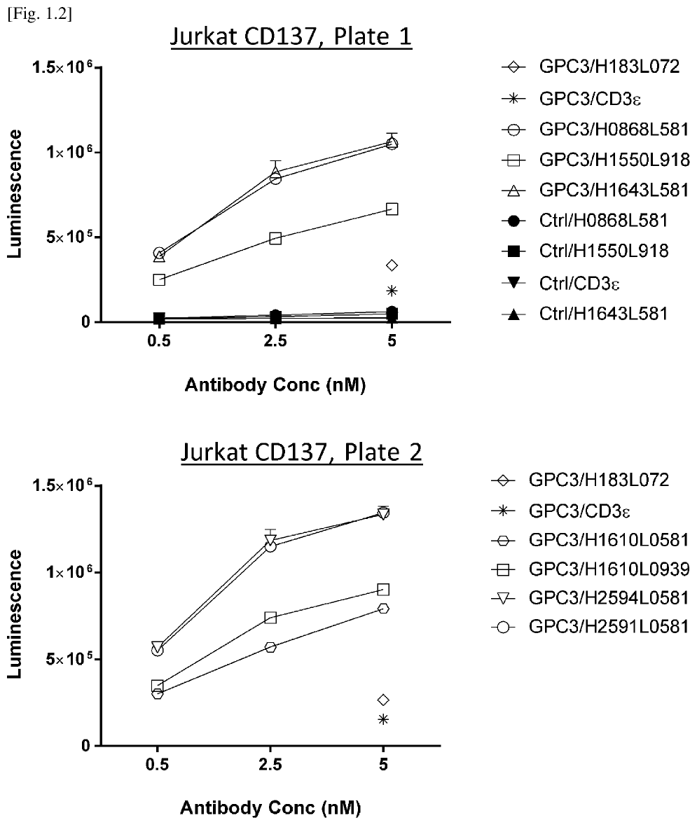

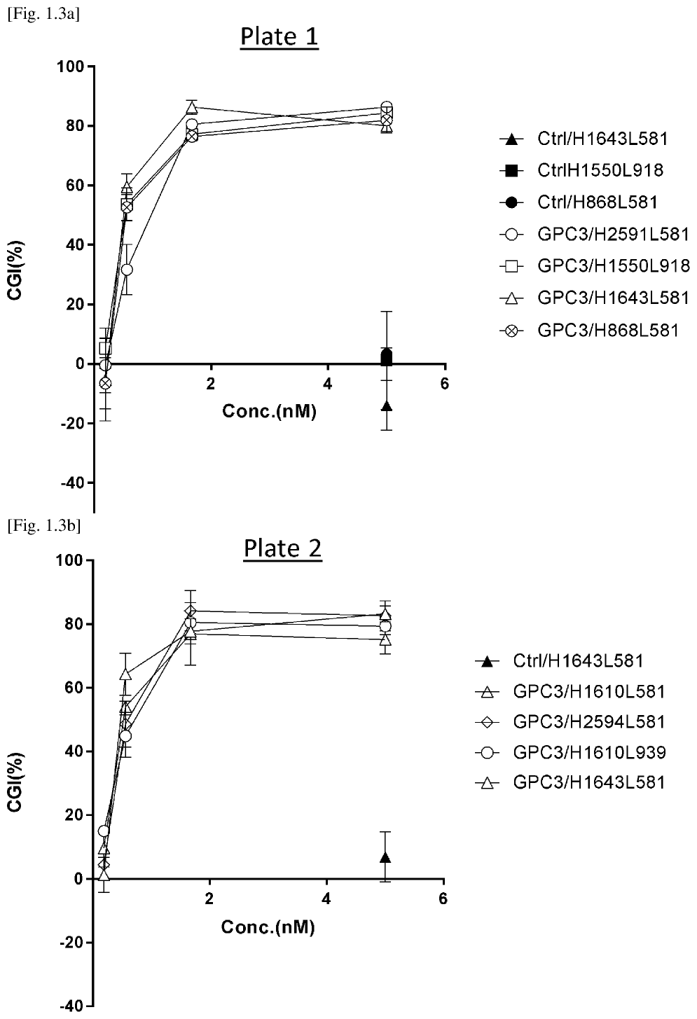

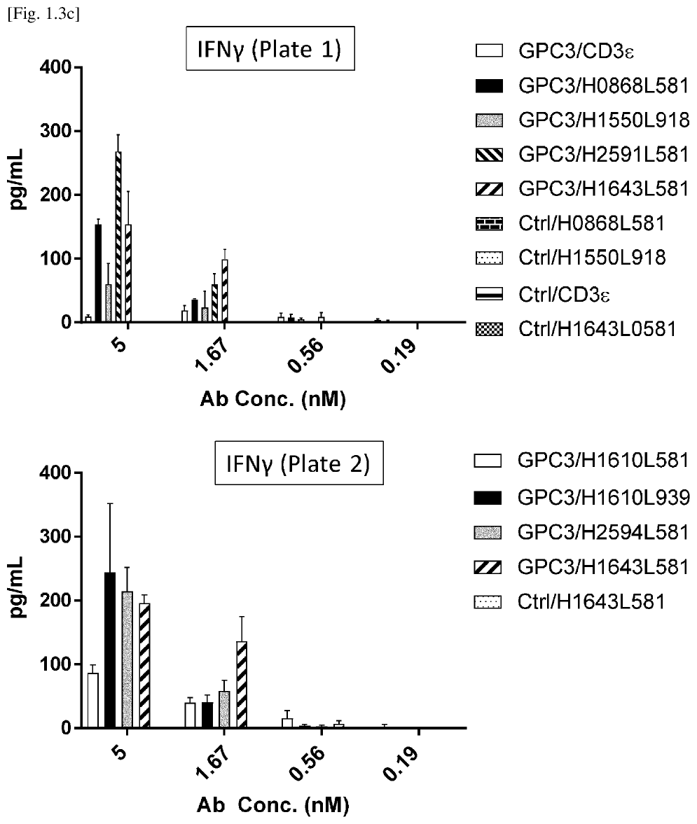

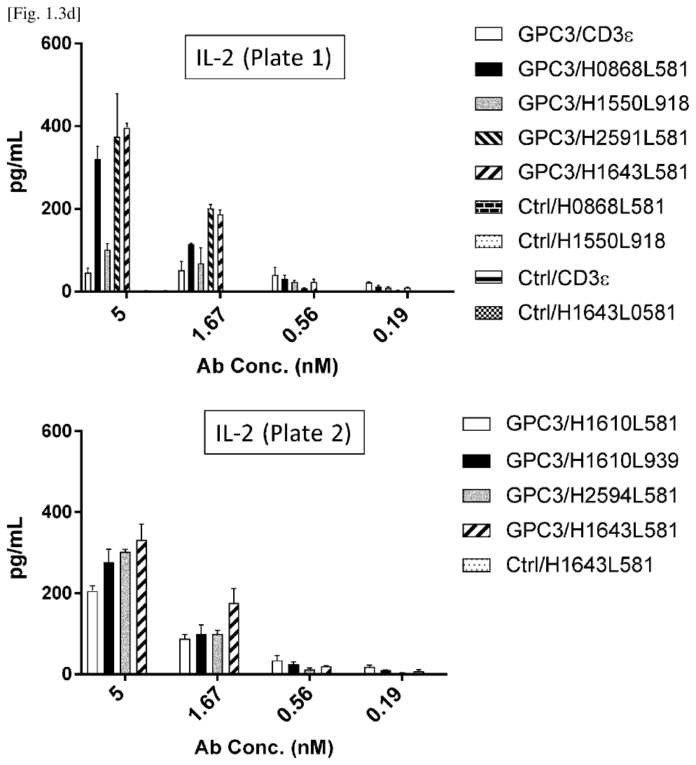

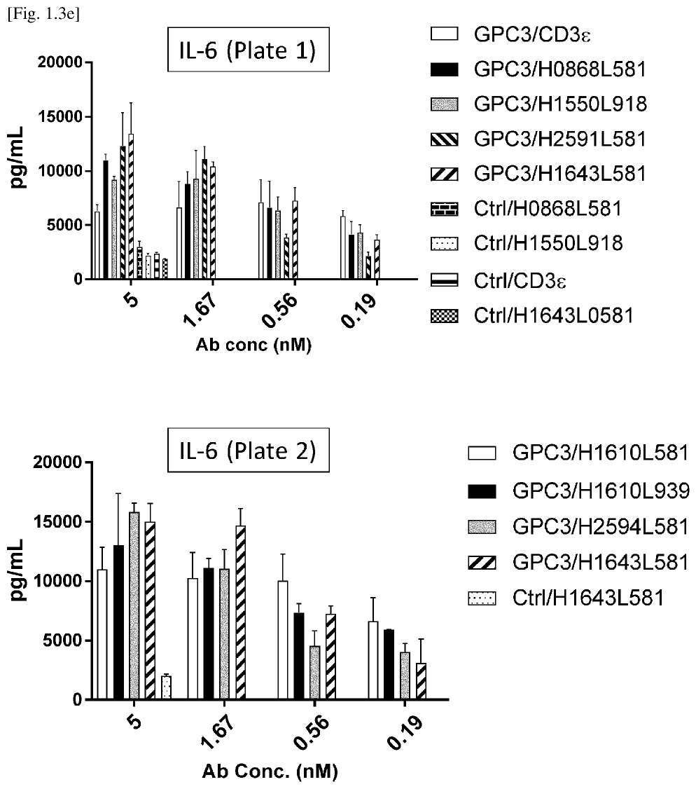

Variants were divided into plate 1 in FIG. 1.1 upper panel and plate 2 in FIG. 1.1 lower panel. 24 hours later, luciferase activity was detected with Bio-Glo luciferase assay system (Promega, G7940) according to manufacturer's instructions. Luminescence (units) was detected using GloMax(registered trademark) Explorer System (Promega #GM3500) and captured values were plotted using Graphpad Prism 7. Parental tri-specific antibody GPC3/H183L072 and bi-specific antibody GPC3/CD3 epsilon were included at 2 nM concentration. FIG. 1.1 showed that most variants have similar CD3 agonist activity. Particularly at 2 nM, variants have similar activity as parental H183L072. FIG. 1.1 upper panel showed that all variants in Plate 1 has similar CD3 agonistic activity. FIG. 1.1 lower panel showed that H1610L939 have slightly weaker CD3 agonist activity while H2591L581 has the strongest CD3 agonistic activity amongst the variants in plate 2.

2.2. Assessment of CD137 Agonistic Activity of Affinity Matured Variants In Vitro

To evaluate which antibody variant could result in strong CD137 agonistic activity as a result of affinity maturation, the GloResponse™ NF-kappa B-Luc2/CD137 Jurkat cell line (Promega #CS196004) as effector cells while similar to above, SK-pca60 cell line (Reference Example 13) was used as target cells. Both 4.0×10³ cells/well SK-pca60 cells (target cells) and 2.0×10⁴ cells/well NF-kappa B-Luc2/CD137 Jurkat (Effector cells) were added on the each well of white-bottomed, 96-well assay plate (Costar, 3917) at E:T ratio of 5. Antibodies were added to each well at 0.5 nM, 2.5 nM and 5 nM concentration incubated at 37 degrees Celsius, 5% CO2 at 37 degrees Celsius for 5 hours. The expressed Luciferase was detected with Bio-Glo luciferase assay system (Promega, G7940) according to Manufacturer's instructions. Luminescence (units) was detected using GloMax(registered trademark) Explorer System (Promega #GM3500) and captured values were plotted using Graphpad Prism 7.

In FIG. 1.2, antibody variants were divided into plate 1 (FIG. 1.2 upper panel) and plate 2 (FIG. 1.2 lower panel) All variants in both plates have detectable CD137 agonistic activity compared to GPC3/CD3 epsilon, which is used as a negative control. Parental antibody before affinity maturation, GPC3/H183L072 was also used as a control in both plates. In FIG. 1.2, all variants showed stronger CD137 agonistic antibody than the parental antibody GPC3/H183L072 after affinity maturation for CD137 binding. Accordingly, GPC3/H1643L581 and GPC3/H868L581 in plate 1 (FIG. 1.2 upper panel) and GPC3/H2594L581 and GPC3/H2591L581 in plate 2 (FIG. 1.2 lower panel) were the top variants that resulted in stronger CD137 agonistic activity. Whereas variants such as GPC3/H1550L918 in plate 1 and GPC3/H1610L581 and GPC3/H1610L939 in plate 2 showed weaker CD137 activity.

Taken together, FIGS. 1.1 and 1.2 show that GPC3/H1643L581, GPC3/H868L581 in plate 1 and GPC3/H2591L581 in plate 2 appear to have similarly strong activity in Jurkat cells whereas GPC3/H1610L939 has weaker activity amongst the variants.

2.3. Evaluation of In Vitro Cytotoxicity of Affinity Matured Variants

In order to extend the observations of CD3, CD137 activation to in vitro cytotoxicity, affinity matured variants described earlier were subjected to evaluation of T-cell dependent cytotoxicity (TDCC) activity on SK-pca60 cells using human peripheral blood mononuclear cells.

2.3.1. Preparation of Frozen Human PBMC

Cryovials containing PBMCs purchased commercially (STEMCELL Technologies.) were placed in the water bath at 37 degrees C. to thaw cells. Cells were then dispensed into a 15 mL falcon tube containing 9 mL of media (media used to culture target cells). Cell suspension was then subjected to centrifugation at 1,200 rpm for 5 minutes at room temperature. The supernatant was aspirated gently and fresh warmed medium was added for resuspension and used as the human PBMC solution.

2.3.2. Measurement of TDCC Activity Using Anti-GPC3 Affinity Matured Dual-Fab Tri-Specific Antibodies

Cytotoxic activity was assessed by observing the rate of tumor cell growth inhibition using xCELLigence Real-Time Cell Analyzer (Roche Diagnostics) in the presence of PBMCs. FIG. 1.3 shows the TDCC activity of anti-GPC3 affinity matured Dual-Fab tri-specific antibodies. SK-pca60 cell line was used as target cells. Target cells were detached from the dish and cells were plated into E-plate 96 (Roche Diagnostics) in aliquots of 100 micro L/well by adjusting the cells to 3.5×10³ cells/well, and measurement of cell growth was initiated using the xCELLigence Real-Time Cell Analyzer. 24 hours later, the plate was removed and 50 micro L of the respective antibodies prepared at each concentration (3-fold serial dilutions starting from 5 nM i.e., 0.19, 0.56, 1.67 and 5 nM) were added to the plate. After 15 minutes of reaction at room temperature, 50 micro L of the fresh human PBMC solution prepared in (Example 2.3.1) was added in effector: target ratio of 0.5 (i.e. 1.75×10³ cells/well) and measurement of cell growth was resumed using xCELLigence Real-Time Cell Analyzer. The reaction was carried out under the conditions of 5% carbon dioxide gas at 37 degrees C. As CD137 signaling enhances T-cell survival and prevents activation induced cell death, TDCC assay was conducted at a low E:T ratio. An extended period of time may be required to observe superior cytotoxicity contributed by CD137 activation. As such, approximately 120 hours after the addition of PBMCs, Cell Growth Inhibition (CGI) rate (%) was determined using the equation below. The Cell Index Value obtained from xCELLigence Real-Time Cell Analyzer used in the calculation was a normalized value where the Cell Index value immediately at the time point before antibody addition was defined as 1.

Cell Growth Inhibition rate (%)=(A−B)×100/(A−1)

A represents the mean value of Cell Index values in wells without antibody addition (containing only target cells and human PBMCs), and B represents the mean value of the Cell Index values of target wells. The examinations were performed in triplicates.

Affinity matured variants were divided into 2 plates as with above examples with GPC3/H1643L581 as an internal plate control for reference in FIG. 1.3. Although most variants show similar TDCC activity, it can be observed that H1643L581 showed relatively stronger TDCC activity at lower concentration of 0.56 nM and 1.67 nM in both plates among the variants. FIG. 1.3a showed that GPC3/H2591L581 is relatively weaker while FIG. 1.3b showed that GPC3/H1610L939 is relatively weaker at 0.56 nM concentration.

2.3.3. Measurement of Cytokine Release Using Anti-GPC3 Affinity Matured Dual-Fab Tri-Specific Antibodies

To further confirm in vitro potency of antibodies, they were also evaluated for cytokine release. Supernatant from TDCC assay similarly conducted in Example 2.3.2 at 48h were harvested and evaluated for the presence of cytokines. Since most antibodies show similar CD3 agonistic activity as GPC3/CD3 epsilon in FIG. 1.1, GPC3/CD3 epsilon was added to this assay to evaluate cytokine release as a result of synergistic activity with CD137. Similarly, GPC3/H1643L581 was used as an internal plate control. Total cytokine release was evaluated using cytometric bead array (CBA) Human Th1/T2 Cytokine kit II (BD Biosciences #551809). IFN gamma (FIG. 1.3c), IL-2 (FIG. 1.3d) and IL-6 (FIG. 1.3e) were evaluated.

As shown in FIGS. 1.3c and 1.3d, GPC3/H2591L581 and GPC3/H1643L581 are the top 2 variants that resulted in high IFN gamma and IL-2 at 5 nM and 1.67 nM in Plate 1. In plate 2, GPC3/H1610L939, GPC3/H2594L581 and GPC3/H1643L581 shows relatively strong cytokine release at 5 nM. However, only GPC3/H1643L581 shows stronger cytokine release at 1.67 nM. As for IL-6 levels shown in FIG. 1.3e, all variants showed similar levels to GPC3/CD3 epsilon in plate 1 except for GPC3/H2591L581 which showed lower levels of IL-6 at 0.56 nM and 0.19 nM. Similarly in plate 2, all variants show similar cytokine release levels as GPC3/H1643L581. Taken together, Dual Fab variants can show improved IFN gamma and IL-2 compared to GPC3/CD3 epsilon without increasing IL-6 levels significantly.

Taken together, affinity matured variants show stronger CD137 agonistic activity which can elicit TDCC activity corresponding to cytokine release. Particularly, variants showed improved IFN gamma and IL-2 levels relative to GPC3/CD3 epsilon.

### [Example 3] Evaluation of Off-Target Cytotoxicity of GPC3/CD3/Human CD137 (2+1)

Tri-specific antibodies and Anti-GPC3/Dual (1+1) Tri-specific antibodies.

3.1. Preparation of Anti-GPC3/CD137×CD3 (2+1) Trispecific Antibodies

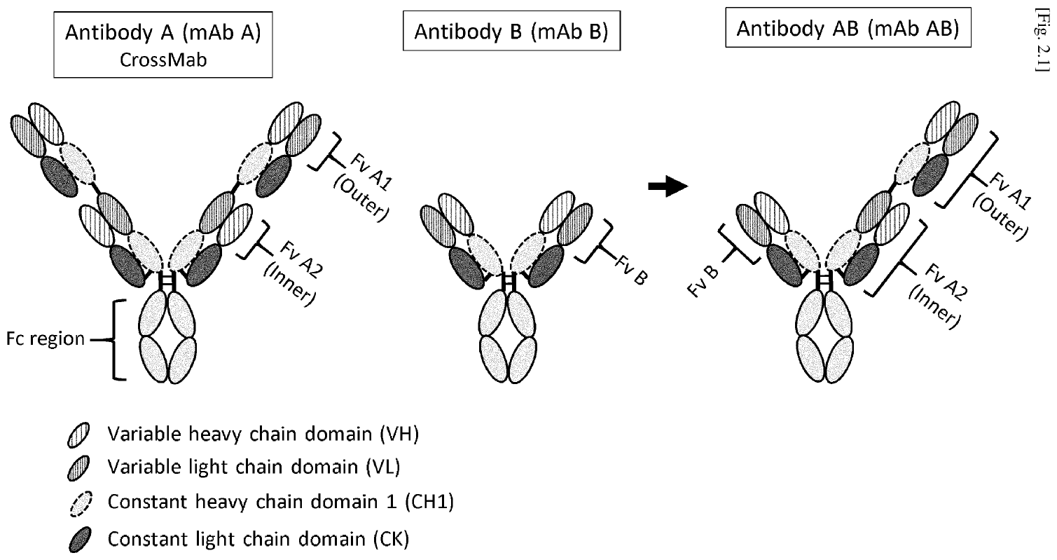

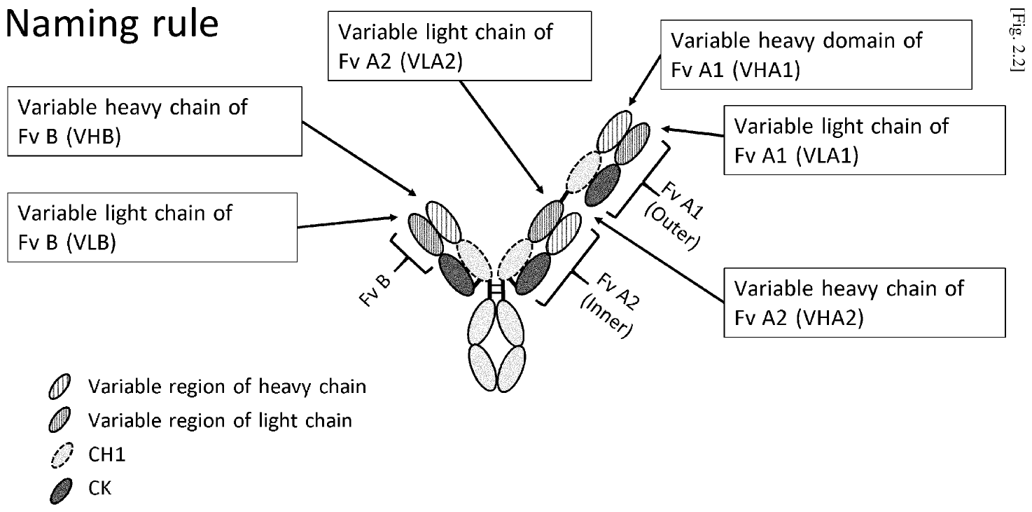

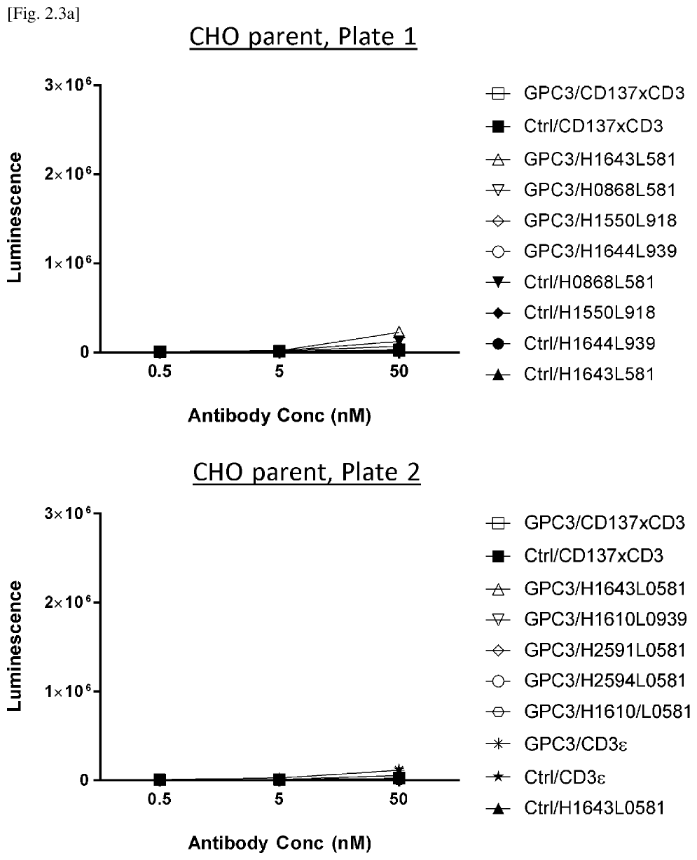

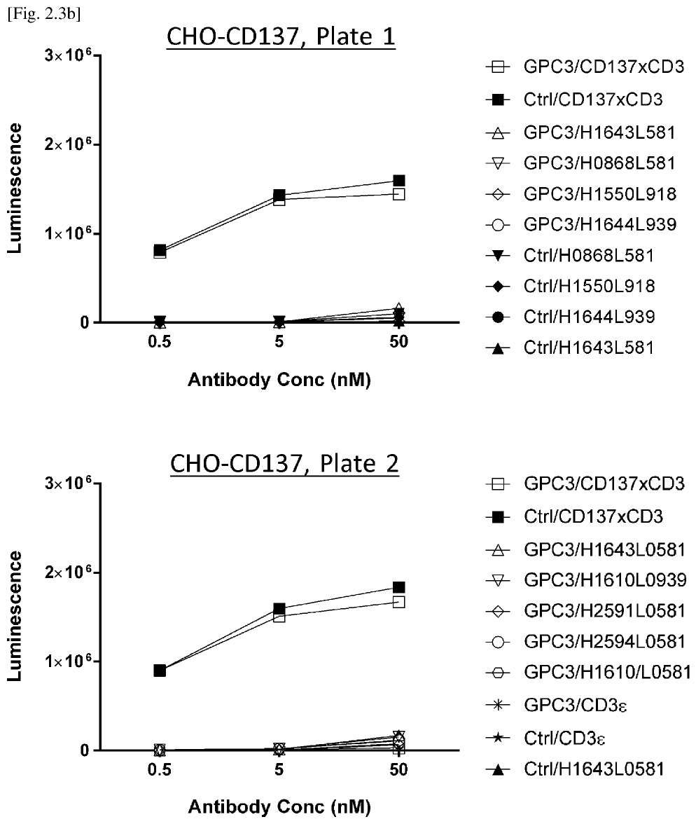

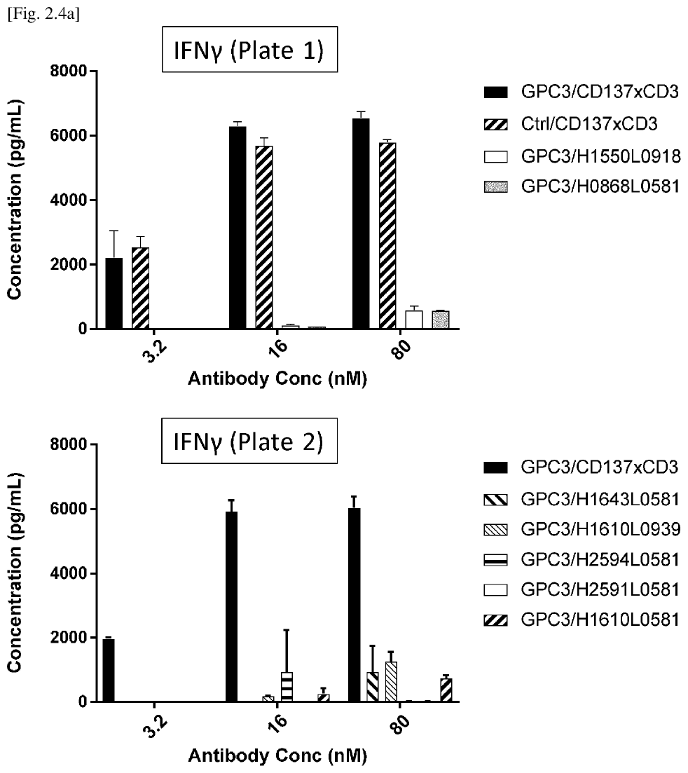

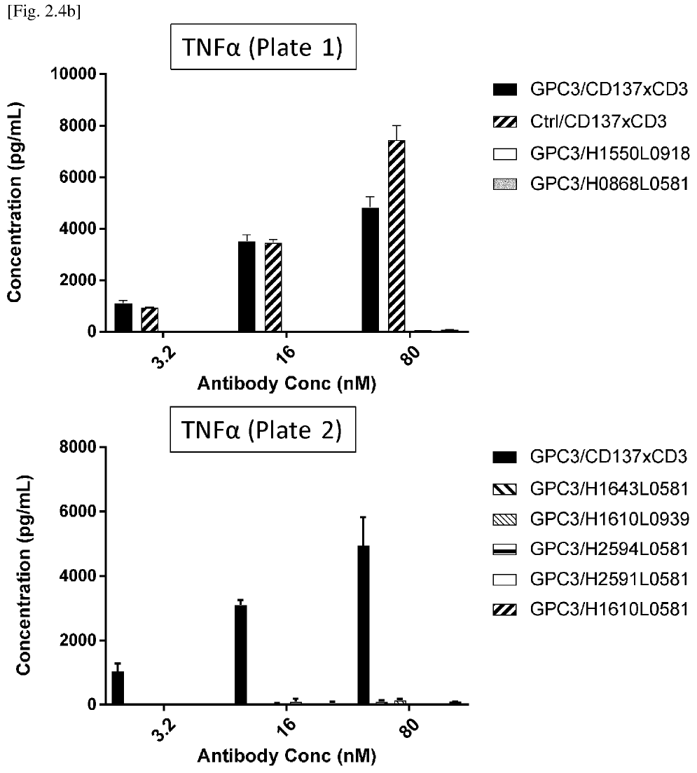

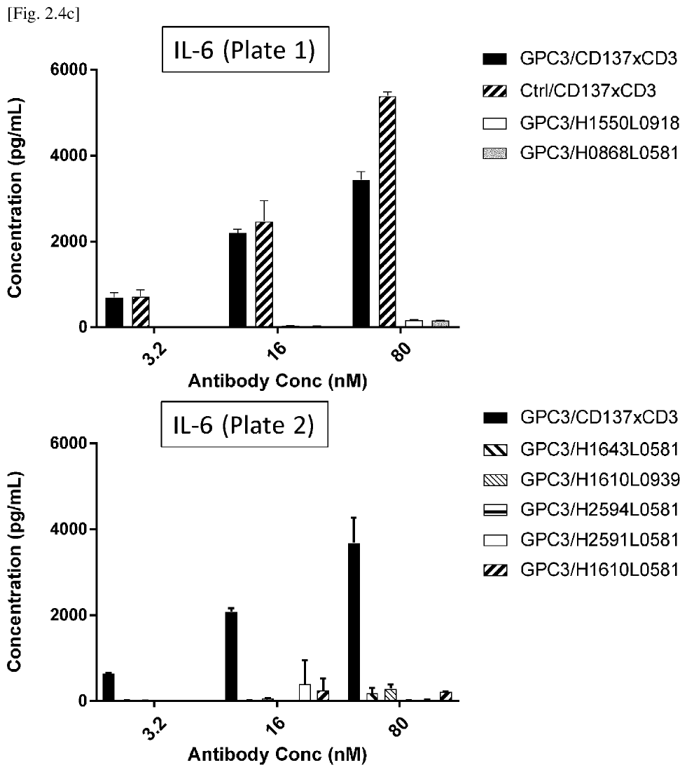

To investigate target independent cytotoxicity and cytokine release, tri-specific antibodies were generated by utilizing CrossMab and FAE technology (FIGS. 2.1 and 2.2). Tetravalent IgG-like molecule, Antibody A (mAb A) which of each arm has two binding domains resulting in four binding domains in one molecule was generated with CrossMab as mentioned above. Bivalent IgG, Antibody B (mAb B) is the same format as a conventional IgG. Fc region of both mAb A and mAb B is Fc gamma R silent with attenuated affinity for Fc gamma receptor, deglycosylated and applicable for FAE. Six tri-specific antibodies were constructed. The target antigen of each Fv region in six trispecific antibodies is shown in Table 2.1. The naming rule of each of binding domain of mAb A, mAb B, and mAb AB are shown in FIG. 2.2. The pair of mAb A and mAb B to generate the respective tri-specific antibodies, mAb AB, and their SEQ ID NOs are shown in Table 2.2 and Table 2.2. Antibody CD3 D(2)_i121 which was described in WO2005/035584A1 (abbreviated as AN121) was used as anti-CD3 antibody. Tri-specific antibodies described in Table 2 were expressed and purified by the method described above.

TABLE 2.1
Target of each arm of antibodies

| Name of mAb AB | Fv A1 | Fv A2 | Fv B |
|---|---|---|---|
| GPC3/CD137 × CD3 | Anti-CD137 | Anti-CD3ε | Anti-GPC3 |
| Ctrl/CD137 × CD3 | Anti-CD137 | Anti-CD3ε | Ctrl |

TABLE 2.2
SEQ ID NO of each variable sequence of antibody described in Table 2.1

| | Name of mAb | VHAl | VLA1 | VHA2 | VLA2 | Name of mAb | VHB | VLB |
|---|---|---|---|---|---|---|---|---|
| Name of | A to generate | (SEQ ID | (SEQ ID | (SEQ ID | (SEQ ID | B to generate | (SEQ ID | (SEQ ID |
| mAb AB | mAb AB | NO.) | NO.) | NO.) | NO.) | mAb AB | NO.) | NO.) |
| GPC3/CD137xCD3 | CD137xCD3 | 202 | 203 | 204 | 205 | GPC3 | 206 | 207 |
| CtrI/CD137xCtrl | CD137xCtrl | 202 | 203 | Ctrl | Ctrl | Ctrl | Ctrl | Ctrl |

TABLE 2.3
Amino acid sequence of variable region of
antibody described in Table 2.1 and 2.2

| VH/VL | |
|---|---|
| name | Amino Acid Sequence |
| CD137VH | QVQLQQWGAGLLKPSETLSLTCAVYGGSFSGYYWSWIRQS |
| (SEQ ID | PEKGLEWIGEINHGGYVTYNPSLESRVTISVDTSKNQFSL |
| NO: 202) | KLSSVTAADTAVYYCARDYGPGNYDWYFDLWGRGTLVTVS |
| | S |
| CD137VL | EIVLTQSPATLSISPGERATLSCRASQSVSSYLAWYQQKP |
| (SEQ ID | GQAPRWYDASNRATGIPARFSGSGSGTDFTLTISSLEPED |
| NO: 203) | FAVYYCQQRSNWPPALTFGGGTKVEIK |
| CD3VH | QVQLVESGGGLVQPGRSLRLSCAASGFTFSNAWMHWVRQA |
| (SEQ ID | PGKGLEWVAQIKDRANSYNTYYAESVKGRFTISRDDSKNS |
| NO: 204) | IYLQMNSLKTEDTAVYYCRYVHYTTYAGSSFSYGVDAWGQ |
| | GTTVTVSS |
| CD3VL | DIVMTQSPLSLPVTPGEPASISCRSSQPLVHSNRNTYLHW |
| (SEQ ID | YQQKPGQAPRLLIYKVSNRFSGVPDRFSGSGSGTDFTLKI |
| NO: 205) | SRVEAEDVGVYYCGQGTQVPYTFGQGTKLEIK |
| GPC3VH | QVQLVQSGAEVKKPGASVIVSCKASGYTFTDYEMHWIRQP |
| (SEQ ID | PGEGLEWIGAIDGPTPDTAYSEKFKGRVTLTADKSTSTAY |
| NO: 206) | MELSSLTSEDTAVYYCTRFYSYTYWGQGTLVTVSS |
| GPV3VL | DIVMTQSPISLPVTPGEPASISCRSSQPLVHSNRNTYLHW |
| (SEQ ID | YQQKPGQAPRLLIYKVSNRFSGVPDRFSGSGSGTDFTLKI |
| NO: 207) | SRVEAEDVGVYYCGQGTQVPYTFGQGTKLEIK |

3.2. Evaluation of the Binding of GPC3/CD137×CD3 Trispecific Antibodies

Binding affinity of trispecific antibodies to human CD3 and CD137 were assessed at 37 degrees C. using Biacore T200 instrument (GE Healthcare). Anti-human Fc antibody (GE Healthcare) was immobilized onto all flow cells of a CM4 sensor chip using amine coupling kit (GE Healthcare). Antibodies were captured onto the anti-Fc sensor surfaces, then recombinant human CD3 or CD137 was injected over the flow cell. All antibodies and analytes were prepared in ACES pH 7.4 containing 20 mM ACES, 150 mM NaCl, 0.05% Tween 20, 0.005% NaN3. Sensor surface was regenerated each cycle with 3M MgCl₂. Binding affinity was determined by processing and fitting the data to 1:1 binding model using Biacore T200 Evaluation software, version 2.0 (GE Healthcare).

Binding affinity of tri-specific antibodies to recombinant human CD3 and CD137 is shown in Table 2.4.

TABLE 2.4
Binding affinity of trispecific antibodies described in Table 2.1 for human CD137 or CD3
measured by Biacore

| | CD137 | | | CD3 | | |
|---|---|---|---|---|---|---|
| Ab name | ka (M⁻¹s⁻¹ ) | kd (s⁻¹) | KD (M) | ka (M⁻¹s⁻¹) | kd (s⁻¹) | KD (M) |
| GPC3/CD137xCD3 | 5.47E+05 | 2.06E−02 | 3.77E−08 | 8.18E+04 | 1.61E−03 | 1.97E−08 |
| Ctrl/CD137xCD3 | 5.48E+05 | 1.82E−02 | 3.31E−08 | 8.24E+04 | 1.52E−03 | 1.85E−08 |

3.3. Assessment of Off Target Cytotoxicity to Human CD137 Expression Cells of GPC3/CD137×CD3 Tri-Specific Antibodies and Anti-GPC3/Dual-Fab Tri-Specific Antibodies.

Tri-specific antibodies, GPC3/CD137×CD3, GPC3/Ctrl×CD3 in 2+1 format or GPC3/H183L072 in 1+1 format derived from parental Dual-Fab H183L072 resulted in dose-dependent activation of Jurkat cells in the presence of target cells, SK-pca60 expressing GPC3 (Reference Example 15-5; FIG. 28). It was also shown that only trispecific format in 2+1 format resulted in Jurkat cell activation in the presence CHO-expressing hCD137 but not tri-specific format in 1+1 format using GPC3/H183L072 (Reference Example 15-6; FIG. 29). This suggested that 2+1 format could potentially result in tumor antigen independent activation of T cells.

To investigate if affinity maturation of H183L072 may result in potential off-target cytotoxicity, affinity matured variants were subjected to the same evaluation, comparing against tri-specific 2+1 antibody format where hCD3 expressing Jurkat cells are co-cultured with hCD137 expressing CHO cells. 5.0×10³ cells/well of hCD137 expressing CHO (FIG. 2.3b) or parental CHO (FIG. 2.3a) were co-cultured with 2.5×10⁴ NFAT-luc2 Jurkat cells for 24 hours in the presence of 0.5, 5 and 50 nM of tri-specific antibodies. FIG. 2.3a showed no non-specific activation of Jurkat cells by all tri-specific antibodies when co-cultured with parental CHO cells. However, it was observed that both GPC3/CD137×CD3 and Ctrl/CD137×CD3 can activate Jurkat cells in the presence of hCD137 expressing CHO cells. Affinity matured variants in 1+1 format did not result in activation of Jurkat cells when co-cultured with hCD137 expressing CHO cells. Taken together, this suggests that trispecific format GPC3/CD137×CD3 can result in Jurkat cell activation independent of target or tumor antigen binding, giving rise to off-target cytotoxicity unlike that of GPC3/Dual (1+1) format even after affinity maturation of CD137 binding.

3.4. Assessment of Off Target Cytokine Release of GPC3/CD137×CD3 Tri-Specific Antibodies and GPC3/Dual-Fab Trispecific Antibodies from PBMCs

Comparison of tri-specific formats for off-target toxicity was also assessed using human PBMC solution. Briefly, 2.0×10⁵ PBMCs prepared as described in Example 2.3.1 were incubated with 80, 16 and 3.2 nM of tri-specific antibodies in the absence of target cells for 48 hours. As IL-2 was not detected by any antibodies, IL-6, IFN gamma and TNF alpha levels in the supernatant are shown in FIG. 2.4a to 2.4c. Measurement of cytokine release was conducted similarly to that described in Example 2.3.3. Similar to Example 2, affinity matured variants were divided into 2 plates. As shown in FIG. 2.4, GPC3/CD137×CD3 but not anti-GPC3/Dual-Fab resulted in IFN gamma (FIG. 2.4a), TNF alpha (FIG. 2.4b), and IL-6 (FIG. 2.4c) release from PBMCs. These results suggest that GPC3/CD137×CD3 tri-specific format resulted in non-specific activation of PBMCs in the absence of target cells. Finally, the data showed that Dual-Fab tri-specific 1+1 format can confer target-specific effector cell activation without off-target toxicity.

### [Example 4] Evaluation of In Vivo Efficacy of GPC3/CD3 Epsilon Bispecific Antibodies and Anti-GPC3/Dual-Fab (1+1) Trispecific Antibodies

4.1. Anti-GPC3/Dual-Fab, GPC3/CD3 Epsilon and GPC3/CD137 Bi-Specific Antibody Preparation

Antibodies for in vivo efficacy studies were generated as described in Example 1.3. In addition to anti-GPC3/Dual-Fab and GPC3/CD3 epsilon used in Example 1, antiCD137 antibody was generated as bivalent form as with antibodies generated (Table 1.1) in Example 1.1 before FAE was conducted in Example 1.3 to obtain GPC3/CD137. As for humanized huNOG mouse studies, antibodies comprise of human Fc with attenuated affinity for Fc gamma receptor. Whereas for CD137/CD3 double humanized mice studies, antibodies comprise of mouse Fc with attenuated affinity for Fc gamma receptor.

4.2. Generation of CD137/CD3 Double Humanized Mouse

Human CD137 knock-in (KI) mouse strain was generated by replacing mouse endogenous Cd137 genomic region with human CD137 genomic sequence using mouse embryonic stem cells. Human CD3 EDG-replaced mouse was established as a strain in which all three components of the CD3 complex—CD3e, CD3d, and CD3g—are replaced with their human counterparts, CD3E, CD3D, and CD3G (Scientific Rep. 2018; 8: 46960). CD137/CD3 double humanized mouse strain was established by crossbreeding the human CD137 KI mice with the human CD3EDG-replaced mice.

4.3. Preparation of LLC1/hGPC3 Cell Line

The mouse cancer cell line LL/2(LLC1) (ATCC) were transfected with pCXND3-hGPC3 and performed single cell clone isolation with 500 micro g/ml G418. Selected clone (LLC1/hGPC3) were confirmed the expression of hGPC3.

4.4. Assessment of In Vivo Efficacy of Anti-GPC3/Dual-Fab Tri-Specific Antibodies with hCD3/hCD137 Mice

Antibodies prepared in Example 4.1 were evaluated for their in vivo efficacy using tumor-bearing models.

For in vivo efficacy evaluation, CD3/CD137 double humanized mice established in Example 4.2, which is called as “hCD3/hCD137 mice” hereafter, were used. LLC1/hGPC3 cells which have stable expression of human GPC3 were transplanted into the hCD3/hCD137 mice, and the hCD3/hCD137 mice with confirmed tumor formation were treated by administration of the GPC3/H1643L0581, GPC3/CD137, or GPC3/CD3 epsilon antibodies.

More specifically, in drug efficacy tests of the GPC3/H1643L0581 using the LLC1/hGPC3 model, the tests below were performed. LLC1/hGPC3 (1×10⁶ cells) were transplanted into the inguinal subcutaneous region of hCD3/hCD137 mice. The day of transplantation was defined as day 0. On the days 9 after the transplantation, the mice were randomized into groups according to their body weight and tumor size. On the day of randomization, the GPC3/H1643L0581, GPC3/CD137, or GPC3/CD3 epsilon antibody were administered intravenously through the caudate vein at 6 mg/kg. The combination therapy group were treated with 6 mg/kg of GPC3/CD3 epsilon and 6 mg/kg of GPC3/CD137 antibodies. The antibodies were administered only once. Tumor volume and body weight were measured with anti tumor testing system (ANTES version 7.0.0.0) every 3-4 days.

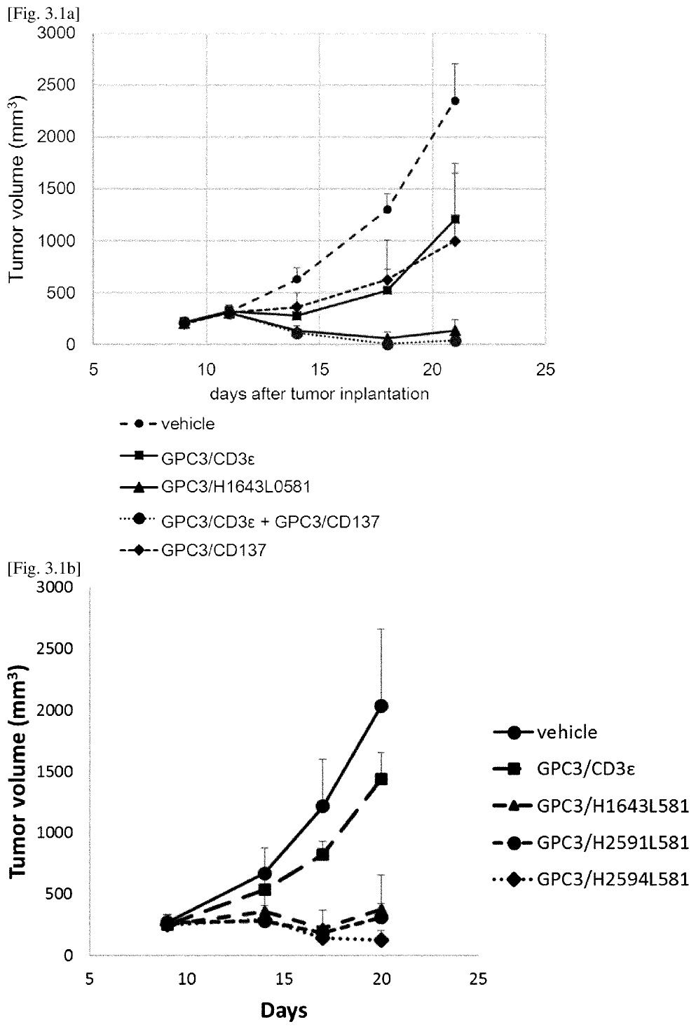

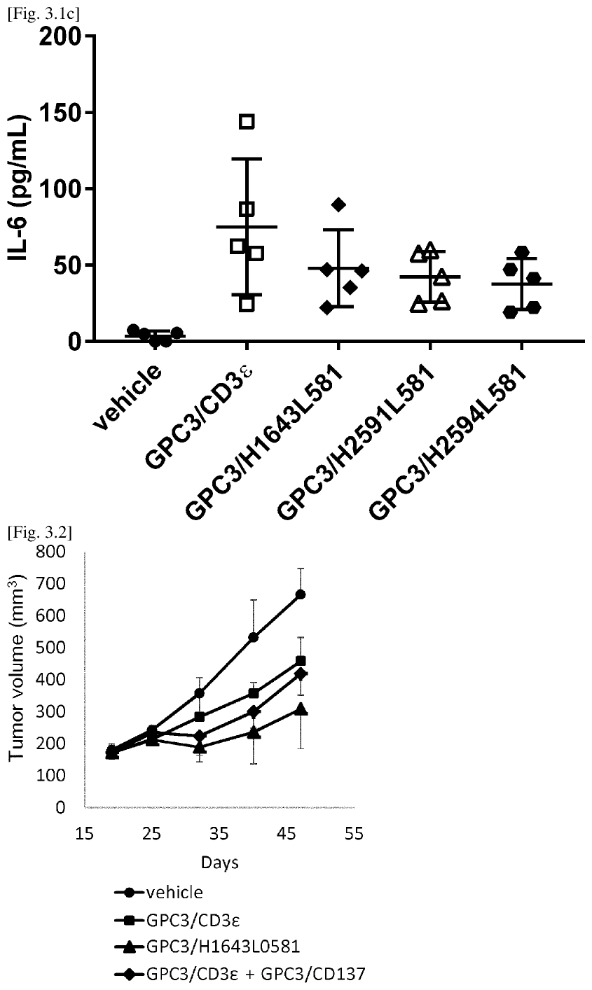

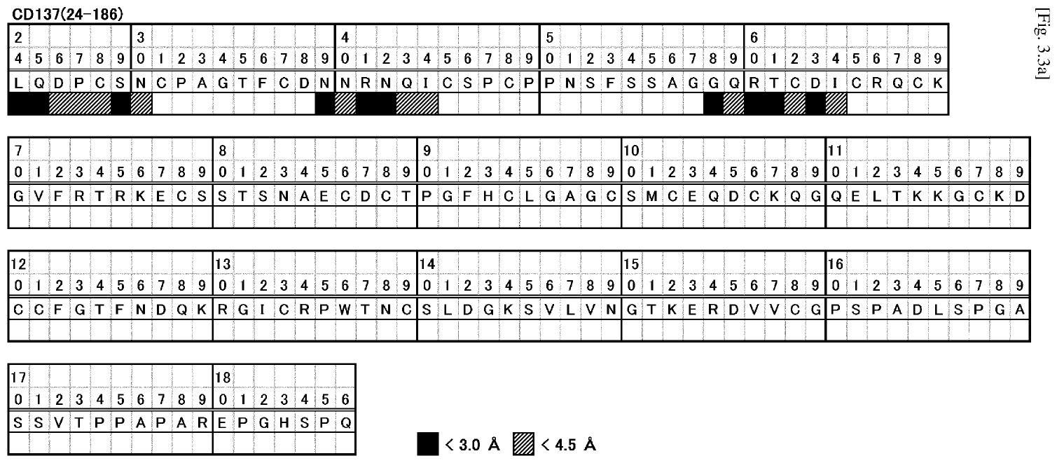

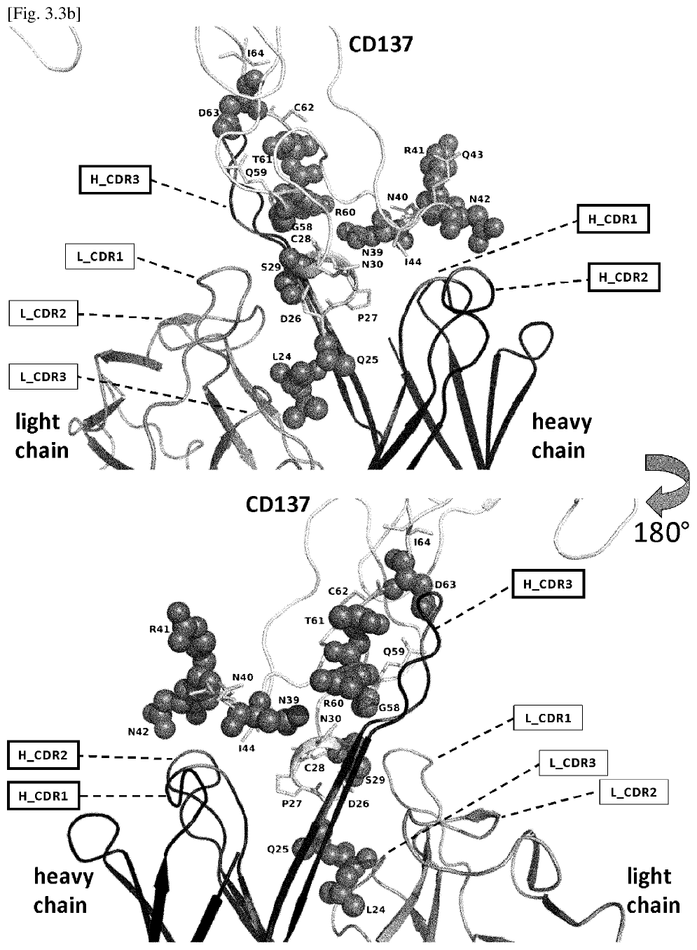

As a result, anti-tumor activities were more clearly observed in GPC3/H1643L0581 group than GPC3/CD3 epsilon group and GPC3/CD137 group (FIG. 3.1a).

In another in vivo efficacy evaluation, LLC/hGPC3 cells were transplanted into the right flank of hCD3/hCD137 mice. On day 9, the mice were randomized into groups on the basis of their tumor volume and body weight, and injected i.v. with vehicle or antibodies prepared in Example 4.1. Tumor volume was measured twice per week. For IL-6 analysis, mice were bled at 2h after treatment. Plasma samples were analyzed with Bio-Plea Pro Mouse Cytokine Th1 Panel according to the manufacture's protocol. As shown in FIGS. 3.1b and 3.1c, GPC3/Dual group showed stronger anti-tumor activity and less IL-6 production compared to GPC3/CD3 epsilon group.

4.5. Assessment of In Vivo Efficacy of Anti-GPC3/Dual-Fab Tri-Specific Antibodies with HuNOG Mice

The anti-tumor activity of anti-GPC3/Dual-Fab antibody, GPC3/CD3 epsilon bispecific antibody and GPC3/CD137 bi-specific antibody prepared in Example 4.1 were tested in a human hepatic sk-pca-13a cancer model. The GPC3/CD3 epsilon bi-specific antibody was also tested in combination with the GPC3/CD137 bi-specific antibody. Sk-pca-13a cells were subcutaneously transplanted to NOG humanized mice. In order to obtain the sk-pca-13a cell line, the human GPC3 gene was integrated into the chromosome of the human liver adenocarcinoma cell line SK-HEP-1 (ATCC No. HTB-52) by a method well known to those skilled in the art.

NOG female mice were purchased from In-Vivo Science. For humanization, mice were sub lethally irradiated followed 1 day later by injection of 100,000 human cord blood cells (ALLCELLS). Sixteen weeks later, sk-pca-13a cells (1×10⁷ cells) were mixed with Matrigel™ Basement Membrane Matrix (Corning) and transplanted to the right flank of humanized NOG mice. The day of transplantation was defined as day 0. On day 19, the mice were randomized on the basis of tumor volume and body weight, and injected i.v. with either vehicle (PBS containing 0.05% Tween), 5 mg/kg GPC3/CD3 epsilon, 5 mg/kg GPC3/H1643L0581, or combination of 5 mg/kg GPC3/CD3 epsilon and 5 mg/kg GPC3/CD137.

As a result, anti-GPC3/Dual-Fab (GPC3/H1643L0581) showed greater anti-tumor activity than GPC3/CD3 epsilon (FIG. 3.2).

### [Example 5] X-Ray Crystal Structure Analysis of H0868L0581/hCD137 Complex

5.1. Preparation of Antibody for Co-Crystal Analysis

H0868L581 was selected for co-crystal analysis with hCD137 protein. The bivalent antibody was transiently transfected and expressed using an Expi293 Expression system (Thermo Fisher Scientific). Culture supernatants were harvested and antibodies were purified from the supernatants using MabSelect SuRe affinity chromatography (GE Healthcare) followed by gel filtration chromatography using Superdex200 (GE Healthcare).

5.2. Expression and Purification of Extracellular Domain (24-186) of Human CD137

Extracellular domain of human CD137 fused to Fc via Factor Xa cleavable linker (CD137-FFc, SEQ ID NO: 81) was expressed in the HEK293 Cell in the presence of kifunensine. The CD137-FFc from culture medium was purified by affinity chromatography (HiTrap MabSelect SuRe column, GE Healthcare) and size exclusion chromatography (HiLoad 16/600 Superdex 200 pg column, GE healthcare). Fc was cleaved with Factor Xa and the resultant CD137 extracellular domain was further purified with tandemly connected gel filtration column (HiLoad 16/600 Superdex 200 pg, GE healthcare) and Protein A column (HiTrap MabSelect SuRe 1 ml, GE Healthcare) and subsequently purified using Benzamidine Sepharose resin (GE Healthcare). Fractions containing CD137 extracellular domain were pooled and stored at −80 degrees C.

5.3. Preparation of Fab Fragment of H0868L0581 and Anti-CD137 Control Antibody

Antibodies for crystal structure analysis were transiently transfected and expressed using an Expi293 Expression system (Thermo Fisher Scientific). Culture supernatants were harvested and antibodies were purified from the supernatants using MabSelect SuRe affinity chromatography (GE Healthcare) followed by gel filtration chromatography using Superdex200 (GE Healthcare). Fab fragments of H0868L0581 and known anti-CD137 control antibody (called as 137Ctrl hereafter, Heavy chain SEQ ID NO: 82, Light chain SEQ ID NO: 83) were prepared by the conventional method using limited digestion with Lys-C(Roche), followed by loading onto a protein A column (MabSlect SuRe, GE Healthcare) to remove Fc fragments, a cation exchange column (HiTrap SP HP, GE Healthcare), and a gel filtration column (Superdex200 16/60, GE Healthcare). Fractions containing Fab fragment were pooled and stored at −80 degrees C.

5.4. Preparation of H0868L0581 Fab, 137Ctrl and Human CD137 Complex

Purified CD137 was mixed with GST-tag fused Endoglycosidase F1(in-house) for deglycosylation, followed by purification of CD137 using gel filtration column (HiLoad 16/600 Superdex 200 pg, GE healthcare) and Protein A column (HiTrap MabSelect SuRe 1 ml, GE Healthcare). Purified CD137 was mixed with H0868L0581 Fab. The complex was purified by gel filtration column (Superdex 200 Increase 10/300 GL, GE healthcare) and subsequently purified H0868L0581 Fab and CD137 complex was mixed with 137Ctrl. The ternary complex was purified by gel filtration chromatography (Superdex200 10/300 increase, GE Healthcare) using a column equilibrated with 25 mM HEPES pH 7.3, 100 mM NaCl.

5.5. Crystallization

The purified complexes were concentrated to about 10 mg/mL, and crystallization was carried out by the sitting drop vapor diffusion method at 21 degrees C. The reservoir solution consisted of 0.1M Tris hydrochloride pH8.5, 25.0% v/v Polyethylene glycol monomethyl ether 550.

5.6. Data Collection and Structure Determination

X-ray diffraction data were measured by X06SA at SLS. During the measurement, the crystal was constantly placed in a nitrogen stream at −178 degrees C. to maintain it in a frozen state, and a total of 1440 X-ray diffraction images were collected using an Eiger X16M (DECTRIS) attached to a beam line, while rotating the crystal 0.25 degrees at a time. Determining the cell parameters, indexing the diffraction spots, and processing the diffraction data obtained from the diffraction images were performed using the autoPROC program (Acta. Cryst. 2011, D67: 293-302), XDS Package (Acta. Cryst. 2010, D66: 125-132), and AIMLESS (Acta. Cryst. 2013, D69: 1204-1214), and finally the diffraction intensity data up to 3.705 angstrom resolution was obtained. The crystallography data statistics are shown in Table 2.5.

The structure was determined by molecular replacement with the program Phaser (J. Appl. Cryst. 2007, 40: 658-674). The search model was derived from the published crystal structure (PDB code: 4NKI and 6MI2). A model was built with the Coot program (Acta Cryst. 2010, D66: 486-501) and refined with the program Refmac5 (Acta Cryst. 2011, D67: 355-367) and PHENIX (Acta Cryst. 2010, D66: 213-221). The crystallographic reliability factor (R) for the diffraction intensity data from 77.585-3.705 angstrom was 22.33%, with a Free R value of 27.50%. The structure refinement statistics are shown in Table 2.5.

TABLE 2.5
X-ray data collection and refinement statistics
Data collection

| Space group | C2 |
|---|---|
| Unit Cell | |
| a,b,c (Å) | 233.795, 74.019, 81.986 |
| α,β,γ (°) | 90.000, 108.858, 90.000 |
| Resolution (Å) | 77.585-3.705 |
| Total reflections | 99,488 |
| Unique reflections | 14,221 |
| Completeness (highest resolution shell) (%) | 99.2 (99.7) |
| R_{merge} ^{a} (highest resolution shell) | 0.161 (1.052) |

Refinement

| | |
|---|---|
| Resolution (Å) | 48.822-3.705 |
| Reflections | 14,195 |
| R factor ^{b} (R_{free} ^{c}) (%) | 22.33 (27.50) |
| rms deviation from ideal | |
| Bond lengths (Å) | 0.003 |
| Bond angles (°) | 0.618 |

^{a}; R_{merge} = ΣhklΣj|Ij (hkl) − [symbol] I (hkl) [symbol] |/ΣhklΣj|Ij (hkl)|, where Ij (hkl) and [symbol] I (hkl) [symbol] are the intensity of measurement j and the mean intensity for the reflection with indices hkl, respectively.
^{b}; R factor = Σhkl|F_{calc}(hkl)| − |F_{obs} (hkl)|/Σhkl|F_{obs} (hkl)|, where F_{obs} and F_{calc} are the observed and calculated structure factor amplitudes, respectively.
^{c}; R_{free} is calculated with 5% of the reflection randomly set aside.

5.7. Identification of the Interaction Sites of H0868L0581 Fab and CD137

The crystal structure of the ternary complex of H0868L0581 Fab, 137Ctrl and CD137 was determined at 3.705 angstrom resolution. In Figure. 3.3a and 3.3 b, the epitope of the H0868L0581 Fab contact region is mapped in the CD137 amino acid sequence and in the crystal structure, respectively. The epitope includes the amino acid residues of CD137 that contain one or more atoms located within 4.5 angstrom distance from any part of the H0868L0581 Fab in the crystal structure. In addition, the epitope within 3.0 angstrom is highlighted in FIGS. 3.3a and 3.3b.

As shown in FIGS. 3.3a and 3.3b, the crystal structure showed that the L24-N30 in CRD1 of CD137 bound in a pocket formed between Heavy chain and Light chain of H0868L0581 Fab, particularly L24-S29 are deeply buried in a manner that the N-terminus of CD137 is oriented toward the depth of the pocket. In addition, N39-I44 in CRD1 and G58-I64 in CRD2 in CD137 were recognized by Heavy chain CDRs of H0868L0581 Fab. CRD is the name of domains divided by the structure formed by Cys-Cys called CRD reference as described in WO2015/156268.

We identified anti-human CD137 antibody which recognize the N-terminus region, especially L24-N30, of human CD137, and also identified that the antibody against this region can activate CD137 on cells.

### [Reference Example 1] Obtainment of Fab Domain Binding to CD3Epsilon and Human CD137 from Dual Fab Phage Display Library

1.1. Construction of Heavy Chain Phage Display Library with GLS3000 Light Chain

The antibody library fragments synthesized in Reference Example 12 was used to construct the dual Fab library for phage display. The dual library was prepared as a library in which H chains are diversified as shown in Reference Example 12 while L chains are fixed to the original sequence GLS3000 (SEQ ID NO: 85). The H chain library sequences derived from CE115HA000 by adding the V11L/L78I mutation to FR (framework) and further diversifying CDRs as shown in Table 27 (in Reference Example 12) were entrusted to the DNA synthesizing company DNA2.0, Inc. to obtain antibody library fragments (DNA fragments). The obtained antibody library fragments were inserted to phagemids for phage display amplified by PCR. GLS3000 was selected as L chains. The constructed phagemids for phage display were transferred to E. coli by electroporation to prepare E. coli harboring the antibody library fragments.

Phage library displaying Fab domain were produced from the E. coli harboring the constructed phagemids by infection of helper phage M13KO7TC/FkpA which code FkpA chaperone gene and then incubate in the presence of 0.002% arabinose at 25 degrees Celsius (this phage library named as DA library) or 0.02% arabinose at 20 degrees Celsius (this phage library named as DX library) for overnight. M13KO7TC is a helper phage which has an insert of the trypsin cleavage sequence between the N2 domain and the CT domain of the pIII protein on the helper phage (see National Publication of International Patent Application No. 2002-514413). Introduction of insert gene into M13KO7TC gene have been already disclosed elsewhere (see National Publication of International Patent Application No. WO2015046554).

1.2. Obtainment of Fab Domain Binding to CD3Epsilon and Human CD137 with Double Round Selection

Fab domains binding to CD3 epsilon and human CD137 were identified from the dual Fab library constructed in Reference Example 1.1. Biotin-labeled CD3 epsilon peptide antigen (amino acid sequence: SEQ ID NO: 86), CD3 epsilon peptide antigen biotin-labeled through disulfide-bond linker (FIG. 4, called C3 NP1-27; amino acid sequence: SEQ ID NO: 194, synthesized by Genscript), biotin-labeled human CD137 fused to human IgG1 Fc fragment (named as human CD137-Fc) and SS-biotinylated human CD137 fused to human IgG1 Fc fragment (named as ss-human CD137-Fc) was used as an antigen. ss-human CD137-Fc was prepared by using EZ-Link Sulfo-NHS—SS-Biotinylation Kit (PIERCE, Cat. No. 21445) to human CD137 fused to human IgG1 Fc fragment. Biotinylation was conducted in accordance with the instruction manual.

Phages were produced from the E. coli harboring the constructed phagemids for phage display. 2.5 M NaCl/10% PEG was added to the culture solution of the E. coli that had produced phages, and a pool of the phages thus precipitated was diluted with TBS to obtain a phage library solution. Next, BSA (final concentration: 4%) was added to the phage library solution. The panning method was performed with reference to a general panning method using antigens immobilized on magnetic beads (J. Immunol. Methods. (2008) 332 (1-2), 2-9; J. Immunol. Methods. (2001) 247 (1-2), 191-203; Biotechnol. Prog. (2002) 18 (2) 212-20; and Mol. Cell Proteomics (2003) 2 (2), 61-9). The magnetic beads used were NeutrAvidin coated beads (Sera-Mag SpeedBeads NeutrAvidin-coated) or Streptavidin coated beads (Dynabeads M-280 Streptavidin). To eliminate antibodies displaying phage which bind to magnetic beads itself or human IgG1 Fc region, subtraction for magnetic beads and biotin labeled human Fc was conducted.

Specifically, Phage solution was mixed with 250 pmol of human CD137-Fc and 4 nmol of free human IgG1 Fc domain and incubated at room temperature for 60 minutes. Magnetic beads was blocked by 2% skim-milk/TBS with free Streptavidin (Roche) at room temperature for 60 minutes or more and washed three times with TBS, and then mixed with incubated phage solution. After incubation at room temperature for 15 minutes, the beads were washed three-times with TBST (TBS containing 0.1% Tween 20; TBS was available from Takara Bio Inc.) and then further washed twice with 1 mL of TBS. 5micro L of 100 mg/mL Trypsin and 495 micro L of TBS were added and incubated at room temperature for 15 minutes, immediately after which the beads were separated using a magnetic stand to recover phage solution. The E. coli strain was infected by the phages through the gentle spinner culture of the strain at 37 degrees C. for 1 hour. The infected E. coli was inoculated to a plate of 225 mm×225 mm. Next, phages were recovered from the culture solution of the inoculated E. coli to prepare a phage library solution.

In this panning round 1 procedure antibody displaying phages which bind to human CD137 was concentrated. In the 2^{nd} round of panning, 250 pmol of ss-human CD137-Fc was used as biotin-labeled antigen and wash was conducted three-times with TBST and then two-times with TBS. Elution was conducted with 25 mM DTT at room temperature for 15 minutes and then digested by Trypsin.

In the 3^{rd} round and 6^{th} round of panning, 62.5 pmol of C3 NP1-27 was used as biotin-labeled antigen and wash was conducted three-times with TBST and then two-times with TBS. Elution was conducted with 25 mM DTT at room temperature for 15 minutes and then digested by Trypsin.

In the 4^{th}, 5^{th} and 7^{th} round of panning, 62.5 pmol of ss-human CD137-Fc was used as biotin-labeled antigen and wash was conducted three-times with TBST and then two-times with TBS. Elution was conducted with 25 mM DTT at room temperature for 15 minutes and then digested by Trypsin.

1.3. Binding of Fab Domain Displayed by Phage to CD3Epsilon or Human CD137

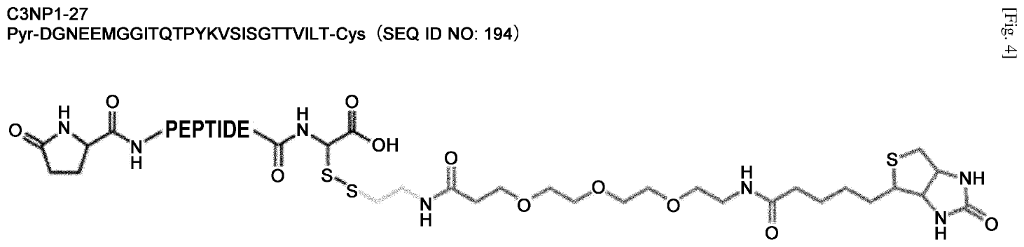

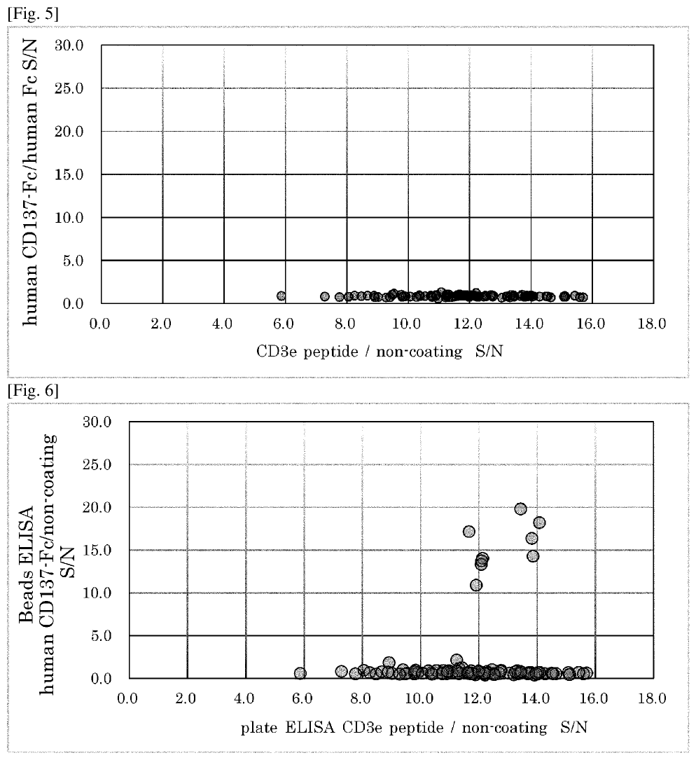

A phage-containing culture supernatant was recovered according to a general method (Methods Mol. Biol. (2002) 178, 133-145) from each 96 single colony of the E. coli obtained by the method described above. The phage-containing culture supernatant was subjected to ELISA by the following procedures: Streptavidin-coated Microplate (384well, greiner, Cat #781990) was coated overnight at 4 degrees C. or at room temperature for 1 hour with 10 micro L of TBS containing the biotin-labeled antigen (biotin-labeled CD3 epsilon peptide or biotin-labeled human CD137-Fc). Each well of the plate was washed with TBST to remove unbound antigens. Then, the well was blocked with 80 micro L of TBS/2% skim milk for 1 hour or longer. After removal of TBS/2% skim milk, the prepared culture supernatant was added to each well, and the plate was left standing at room temperature for 1 hour so that the phage-displayed antibody bound to the antigen contained in each well. Each well was washed with TBST, and HRP/Anti M13 (GE Healthcare 27-9421-01) were then added to each well. The plate was incubated for 1 hour. After washing with TBST, TMB single solution (ZYMED Laboratories, Inc.) was added to the well. The chromogenic reaction of the solution in each well was terminated by the addition of sulfuric acid. Then, the developed color was assayed on the basis of absorbance at 450 nm. The results are shown in FIG. 5.

As shown in FIG. 5, all clones showed binding to human CD3 epsilon but did not show binding to human CD137 even though panning procedure to human CD137 was conducted 5-times. It might depend on the less sensitivity of this phage ELISA analysis with Streptavidin-coated Microplate so phage ELISA with Streptavidin coated beads was also conducted.

1.4. Binding of Fab Domain Displayed by Phage to Human CD137 (Phage Beads ELISA)

First, Streptavidin-coated magnetic beads MyOne-T1 beads was washed three-times with blocking buffer including 0.5× block Ace, 0.02% Tween and 0.05% ProClin 300 and then blocked with this blocking buffer at room temperature for 60 minutes or more. After washing once with TBST, 0.625 pmol of ss-human CD137-Fc was added to magnetic beads and incubated at room temperature for 10 minutes or more and then magnetic beads were applied to each well of 96well plate (Corning, 3792 black round bottom PS plate). 12.5 micro L each of the Fab displaying phage solution with 12.5 micro L of TBS was added to the wells, and the plate was allowed to stand at room temperature for 30 minutes to allow each Fab to bind to biotin-labeled antigen in each well. After that each well was washed with TBST. Anti-M13(p8) Fab-HRP diluted with blocking buffer including 0.5× block Ace, 0.02% Tween and 0.05% ProClin 300 was added to each well. The plate was incubated for 10 minutes. After washing 3-times with TBST, LumiPhos-HRP (Lumigen) was added to each well. 2 minutes later the fluorescence of each well was detected. The measurement results are shown in FIG. 6.

Some clones showed obvious binding to human CD137. This result showed that some Fab domains which bind to both human CD3 epsilon and CD137 were also obtained from this designed library with phage display panning strategy. Nonetheless the binding to human CD137 was still weak compared to CD3 epsilon peptide. The VH fragment of each human CD137 binding clones were amplified by PCR using primers specifically binding to the phagemid vector (SEQ ID NOs: 196 and 197) and the DNA sequences were analyzed. The result showed all binding clones have same VH sequence, it meant only one Fab clone showed binding to both human CD137 and CD3 epsilon. To improve this, double round selection was also applied to phage display strategy in next experiment.

### [Reference Example 2] Obtainment of Fab Domain Binding to CD3Epsilon and Human CD137 from Dual Fab Phage Display Library with Double Round Selection Method

2.1. Construction of Heavy Chain Phage Display Library with GLS3000 Light Chain

Phage library displaying Fab domain were produced from the E. coli harboring the constructed phagemids by infection of helper phage M13KO7TC/FkpA which code FkpA chaperone (SEQ ID NO: 91) and then incubate in the presence of 0.002% arabinose at 25 degrees Celsius (this phage library named as DA library) or 0.02% arabinose at 20 degrees Celsius (this phage library named as DX library) for overnight. M13KO7TC is a helper phage which has an insert of the trypsin cleavage sequence between the N2 domain and the CT domain of the pIII protein on the helper phage (see Japanese Patent Application Kohyo Publication No. 2002-514413). Introduction of insert gene into M13KO7TC gene have been already disclosed elsewhere (see WO2015/046554).

2.2. Obtainment of Fab Domain Binding to CD3Epsilon and Human CD137 with Double Round Selection

Fab domains binding to CD3 epsilon and human CD137 were identified from the dual Fab library constructed in Reference Example 2.1. Biotin-labeled CD3 epsilon peptide antigen (amino acid sequence: SEQ ID NO: 86), CD3 epsilon peptide antigen biotin-labeled through disulfide-bond linker (C3 NP1-27: SEQ ID NO: 194) and biotin-labeled human CD137 fused to human IgG1 Fc fragment (named as human CD137-Fc) was used as an antigen.

To produce much more Fab domain binding to human CD137 and CD3 epsilon, double round selection was also applied for phage display panning at panning round 2 and subsequent round.

Phages were produced from the E. coli harboring the constructed phagemids for phage display. 2.5 M NaCl/10% PEG was added to the culture solution of the E. coli that had produced phages, and a pool of the phages thus precipitated was diluted with TBS to obtain a phage library solution. Next, BSA (final concentration: 4%) was added to the phage library solution. The panning method was performed with reference to a general panning method using antigens immobilized on magnetic beads (J. Immunol. Methods. (2008) 332 (1-2), 2-9; J. Immunol. Methods. (2001) 247 (1-2), 191-203; Biotechnol. Prog. (2002) 18 (2) 212-20; and Mol. Cell Proteomics (2003) 2 (2), 61-9). The magnetic beads used were NeutrAvidin coated beads (Sera-Mag SpeedBeads NeutrAvidin-coated) or Streptavidin coated beads (Dynabeads M-280 Streptavidin). To eliminate antibodies displaying phage which bind to magnetic beads itself or human IgG1 Fc region, subtraction for magnetic beads and biotin labeled human Fc was conducted.

Specifically, at panning round1, magnetic beads was blocked by 2% skim-milk/TBS at room temperature for 60 minutes or more and washed three times with TBS. Phage solution of DA library or DX library were added to blocked magnetic beads and incubated at room temperature for 60 minutes or more, then supernatant was recovered. 500 pmol of biotin labeled human IgG1 Fc was added to new magnetic beads and incubated at room temperature for 15 minutes and then add 2% skim-milk/TBS. After blocking at room temperature for 60 minutes or more, magnetic beads was washed three times with TBS. Recovered phage solution were added to blocked magnetic beads and incubated at room temperature for 60 minutes or more, then supernatant was recovered. 500 pmol of the biotin-labeled CD137-Fc was added to new magnetic beads and incubated at room temperature for 15 minutes and then add 2% skim-milk/TBS. After blocking at room temperature for 60 minutes or more, magnetic beads was washed three times with TBS. Recovered phage solution were added to blocked magnetic beads and 8 nmol of free human IgG1

Fc domain was also added, and then incubated at room temperature for 60 minutes. The beads were washed twice with TBST (TBS containing 0.1% Tween 20; TBS was available from Takara Bio Inc.) and then further washed once with 1 mL of TBS. After addition of 0.5 mL of 1 mg/mL trypsin, the beads were suspended at room temperature for 15 minutes, immediately after which the beads were separated using a magnetic stand to recover a phage solution. The recovered phage solution was added to an E. coli strain ER2738 in a logarithmic growth phase (0D600: 0.4-0.5). The E. coli strain was infected by the phages through the gentle spinner culture of the strain at 37 degrees C. for 1 hour. The infected E. coli was inoculated to a plate of 225 mm×225 mm. Next, phages were recovered from the culture solution of the inoculated E. coli to prepare a phage library solution.

In this panning round1 procedure antibody displaying phages which bind to human CD137 was concentrated so from next round of panning procedure double round selection was conducted to recover antibody displaying phages which bind to both CD3 epsilon and human CD137.

Specifically, at panning round2, magnetic beads was blocked by 2% skim-milk/TBS at room temperature for 60 minutes or more and washed three times with TBS. Phage solution were added to blocked magnetic beads and incubated at room temperature for 60 minutes or more, then supernatant was recovered. 500 pmol of biotin labeled human IgG1 Fc was added to new magnetic beads and incubated at room temperature for 15 minutes and then add 2% skim-milk/TBS. After blocking at room temperature for 60 minutes or more, magnetic beads was washed three times with TBS. Recovered phage solution were added to blocked magnetic beads and incubated at room temperature for 60 minutes or more, then supernatant was recovered. 500 pmol of the biotin-labeled CD137-Fc was added to new magnetic beads and incubated at room temperature for 15 minutes and then add 2% skim-milk/TBS.

After blocking at room temperature for 60 minutes or more, magnetic beads was washed three times with TBS. Recovered phage solution were added to blocked magnetic beads and then incubated at room temperature for 60 minutes. The beads were washed three times with TBST (TBS containing 0.1% Tween 20; TBS was available from Takara Bio Inc.) and then further washed twice with 1 mL of TBS. FabRICATOR(IdeS, protease for hinge region of IgG, GENOVIS)(named as IdeS elution campaign) was used to recover antibody displaying phages. In that procedure, 10 units/micro L Fabricator 20 micro L with 80 micro L TBS buffer was added and beads were suspended at 37 degrees Celsius for 30 minutes, immediately after which the beads were separated using a magnetic stand to recover phage solution.

In this 1^{st} cycle of panning procedure antibody displaying phages which bind to human CD137 was concentrated so then move on to 2^{nd} cycle panning procedure to recover antibody displaying phages which also bind to CD3 epsilon before phage infection and amplification. 500 pmol of the biotin-labeled CD3 epsilon was added to new magnetic beads and incubated at room temperature for 15 minutes and then add 2% skim-milk/TBS. After blocking at room temperature for 60 minutes or more, magnetic beads was washed three times with TBS. Recovered phage solution, 50 micro L of TBS and 250 micro L of 8% BSA blocking buffer were added to blocked magnetic beads and then incubated at 37 degrees Celsius for 30 minutes, at room temperature for 60 minutes, 4 degrees Celsius for overnight and then at room temperature for 60 minutes to transfer antibody displaying phage from human CD137 to CD3 epsilon.

The beads were washed three times with TBST (TBS containing 0.1% Tween 20; TBS was available from Takara Bio Inc.) and then further washed twice with 1 mL of TBS. The beads supplemented with 0.5 mL of 1 mg/mL trypsin were suspended at room temperature for 15 minutes, immediately after which the beads were separated using a magnetic stand to recover a phage solution. The phages recovered from the trypsin-treated phage solution were added to an E. coli strain ER2738 in a logarithmic growth phase (0D600: 0.4-0.7). The E. coli strain was infected by the phages through the gentle spinner culture of the strain at 37 degrees C. for 1 hour. The infected E. coli was inoculated to a plate of 225 mm×225 mm. Next, phages were recovered from the culture solution of the inoculated E. coli to recover a phage library solution.

In the third and fourth round of panning, wash number increased to fifth with TBST and then twice with TBS. In 2^{nd} cycle of double round selection, C3 NP1-27 antigen was used instead of biotin labeled CD3 epsilon peptide antigen, and elution was conducted by DTT solution to cleave the disulfide bond between CD3 epsilon peptide and biotin. Precisely, after washing with TBS twice, 500 micro L of 25 mM DTT solution was added and beads were suspended at room temperature for 15 minutes, immediately after which the beads were separated using a magnetic stand to recover phage solution. 0.5 mL of 1 mg/mL trypsin were added to recovered phage solution and incubated at room temperature for 15 minutes

2.3. Binding of IgG Having Obtained Fab Domain to Human CD137 and Cynomolgus Monkey CD137

96 clones were picked from each panning output pools of DA and DX library at round3 and round4 and their VH gene sequence were analyzed. Twenty-nine VH sequence was obtained so all of them were converted into IgG format. The VH fragments of each clones were amplified by PCR using primers specifically binding to the phagemid vector (SEQ ID NOs: 196 and 197). The amplified VH fragment was integrated into an animal expression plasmid which have already had human IgG1 CH1-Fc region. The prepared plasmids were used for expression in animal cells by the method of Reference Example 9. GLS3000 was used as Light chain and its expression plasmid was prepared as shown in Reference Example 12.2).

The prepared antibodies were subjected to ELISA to evaluate their binding capacity to human CD137 (SEQ ID NO: 195) and cynomolgus monkey (called as cyno) CD137 (SEQ ID NO: 92). FIG. 7 shows the amino acids sequence difference between human and cynomolgus monkey CD137. There are 8 different residues among them.

First, 20 micro g of Streptavidin-coated magnetic beads MyOne-T1 beads was washed three-times with blocking buffer including 0.5× block Ace, 0.02% Tween and 0.05% ProClin 300 and then blocked with this blocking buffer at room temperature for 60 minutes or more. After washing once with TBST, magnetic beads were applied to each well of white round bottom PS plate (Corning, 3605) and 0.625 pmol of biotin labeled human CD137-Fc, biotin labeled cyno CD137-Fc or biotin labeled human Fc was added to magnetic beads and incubated at room temperature for 15 minutes or more. After washing once with TBST, 25 micro L each of the 50 ng/micro L purified IgG was added to the wells, and the plate was allowed to stand at room temperature for one hour to allow each IgG to bind to biotin-labeled antigen in each well.

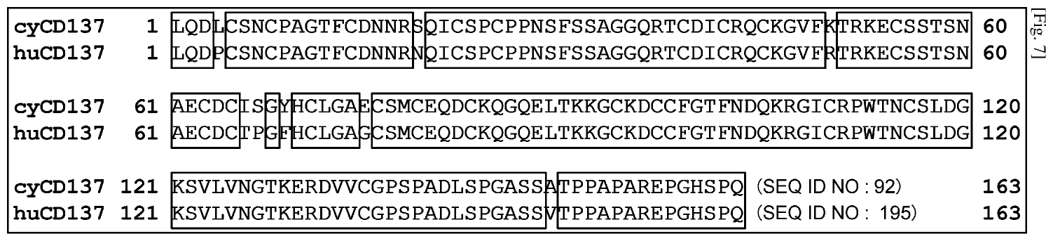

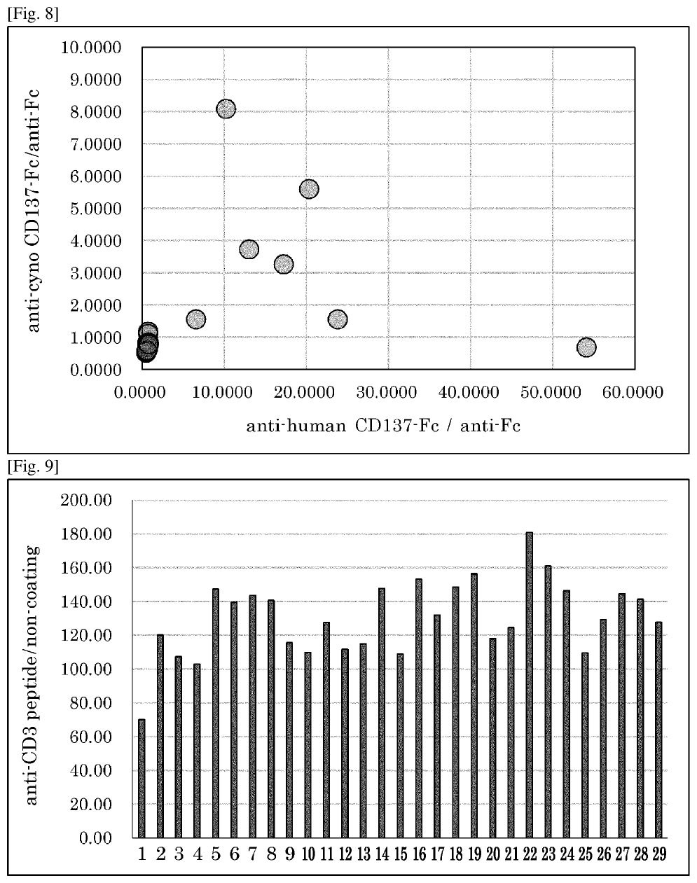

After that each well was washed with TBST. Goat anti-human kappa Light chain alkaline phosphatase conjugate (BETHYL, A80-115AP) diluted with TBS was added to each well. The plate was incubated for one hour. After washing with TBST, each sample were transferred to 96well plate (Corning, 3792 black round bottom PS plate) and APS-5 (Lumigen) was added to each well. 2 minutes later the fluorescence of each well was detected. The measurement results are shown in Table 3 and FIG. 8. Among them, clones DXDU01_3 #094, DXDU01_3 #072, DADU01_3 #018, DADU01_3 #002, DXDU01_3 #019 and DXDU01_3 #051 showed binding to both human and cyno CD137. On the other hand, DADU01_3 #001, which showed strongest binding to human CD137, did not show binding to cyno CD137.

TABLE 3

| | | | | S/N | | |
|---|---|---|---|---|---|---|
| | RLU | | | ratio | | |
| | | human | cyno | human | cyno | SEQ |
| | | CD137- | CD137- | CD137- | CD137- | ID |
| | Fc | Fc | Fc | Fc/Fc | Fc/Fc | NO |
| DADU01_3#031 | 2122 | 1633 | 1783 | 0.7696 | 0.8402 | |
| DXDU01_3#053 | 1935 | 1469 | 1555 | 0.7592 | 0.8036 | |
| DADU01_3#006 | 3202 | 1842 | 1886 | 0.5753 | 0.5890 | |
| DXDU01_3#035 | 2005 | 1424 | 1484 | 0.7102 | 0.7401 | |
| DXDU01_3#064 | 1826 | 1369 | 2150 | 0.7497 | 1.1774 | |
| DADU01_3#036 | 1960 | 1491 | 2173 | 0.7607 | 1.1087 | |
| DXDU01_3#043 | 2311 | 1533 | 1919 | 0.6633 | 0.8304 | |
| DXDU01_3#094 | 2367 | 24241 | 19145 | 10.2412 | 8.0883 | 97 |
| DADU01_3#003 | 2349 | 1596 | 1658 | 0.6794 | 0.7058 | |
| DADU01_3#051 | 2276 | 1595 | 1534 | 0.7008 | 0.6740 | |
| DADU01_4#089 | 3578 | 1970 | 1894 | 0.5506 | 0.5293 | |
| DADU01_3#013 | 2770 | 1707 | 1710 | 0.6162 | 0.6173 | |
| DXDU01_3#049 | 2586 | 1559 | 1578 | 0.6029 | 0.6102 | |
| DXDU01_3#072 | 2148 | 14137 | 3348 | 6.5815 | 1.5587 | 98 |
| DADU01_3#042 | 2570 | 1779 | 1600 | 0.6922 | 0.6226 | |
| DADU01_3#020 | 1970 | 1640 | 1641 | 0.8325 | 0.8330 | |
| DADU01_3#050 | 2246 | 1785 | 1689 | 0.7947 | 0.7520 | |
| DADU01_3#018 | 1899 | 32770 | 6205 | 17.2565 | 3.2675 | 99 |
| DADU01_3#002 | 1924 | 39141 | 10775 | 20.3436 | 5.6003 | 100 |
| DADU01_3#058 | 1931 | 1461 | 1363 | 0.7566 | 0.7059 | |
| DADU01_3#078 | 1689 | 1374 | 1326 | 0.8135 | 0.7851 | |
| DADU01_3#044 | 1992 | 1647 | 1606 | 0.8268 | 0.8062 | |
| DXDU01_3#019 | 3264 | 77805 | 5093 | 23.8373 | 1.5604 | 101 |
| DADU01_3#001 | 1760 | 95262 | 1209 | 54.1261 | 0.6869 | 102 |
| DADU01_3#071 | 3389 | 1927 | 1860 | 0.5686 | 0.5488 | |
| DADU01_3#024 | 3131 | 1783 | 1763 | 0.5695 | 0.5631 | |
| DXDU01_3#051 | 2914 | 38065 | 10870 | 13.0628 | 3.7303 | 103 |
| DADU01_3#004 | 3053 | 1918 | 1802 | 0.6282 | 0.5902 | |
| DADU01_3#045 | 1988 | 1662 | 1573 | 0.8360 | 0.7912 | |

2.4. Binding of IgG Having Obtained Fab Domain to Human CD3Epsilon

Each antibodies were also subjected to ELISA to evaluate their binding capacity to CD3 epsilon.

First, a MyOne-T1 streptavidin beads were mixed with 0.625 pmol of biotin-labeled CD3 epsilon and incubated at room temperature for 10 minutes, then blocking buffer including 0.5× block Ace, 0.02% Tween and 0.05% ProClin 300/TBS was added to block the magnetic beads. Mixed solution was dispended to each well of 96well plate (Corning, 3792 black round bottom PS plate) and incubated at room temperature for 60 minutes or more. After that magnetic beads were washed by TBS once, 100 ng of purified IgG was added to the magnetic beads in each well, and the plate was allowed to stand at room temperature for one hour to allow each IgG to bind to biotin-labeled antigen in each well.

After that each well was washed with TBST, Goat anti-human kappa Light chain alkaline phosphatase conjugate (BETHYL, A80-115AP) diluted with TBS was added to each well. The plate was incubated for one hour. After washing with TBST, APS-5 (Lumigen) was added to each well. 2 minutes later the fluorescence of each well was detected. The measurement results are shown in Table 4 and FIG. 9. All clones showed obvious binding to CD3 epsilon peptide. These data proves the Fab domain which bind to both CD3 epsilon, human CD137 and cyno CD137 could be efficiently obtained by designed Dual Fab antibody phage display library with double round selection procedure with higher hit-rate than with conventional phage display panning procedure conducted in Reference Example 1.

TABLE 4

| | | | S/N ratio |
|---|---|---|---|
| | RLU | | CD3 peptide/ |
| | Non coating | CD3 peptide | non coating |
| DADU01_3#031 | 1505 | 142935 | 70.13 |
| DXDU01_3#053 | 2082 | 148836 | 120.32 |
| DADU01_3#006 | 3843 | 127079 | 107.42 |
| DXDU01_3#035 | 3302 | 119726 | 103.03 |
| DXDU01_3#064 | 3901 | 171861 | 147.52 |
| DADU01_3#036 | 1562 | 159897 | 139.65 |
| DXDU01_3#043 | 1147 | 168793 | 143.65 |
| DXDU01_3#094 | 2473 | 164780 | 140.72 |
| DADU01_3#003 | 3104 | 151738 | 115.65 |
| DADU01_3#051 | 2489 | 135224 | 109.85 |
| DADU01_4#089 | 1366 | 150267 | 127.67 |
| DADU01_3#013 | 4688 | 136821 | 111.78 |
| DXDU01_3#049 | 3205 | 141259 | 114.94 |
| DXDU01_3#072 | 2168 | 176615 | 147.67 |
| DADU01_3#042 | 4271 | 135203 | 108.86 |
| DADU01_3#020 | 1454 | 197301 | 153.18 |
| DADU01_3#050 | 1564 | 166509 | 132.05 |
| DADU01_3#018 | 2293 | 181896 | 148.73 |
| DADU01_3#002 | 2954 | 173838 | 156.47 |
| DADU01_3#058 | 2618 | 136587 | 118.05 |
| DADU01_3#078 | 1754 | 146653 | 124.49 |
| DADU01_3#044 | 1091 | 196612 | 180.88 |
| DXDU01_3#019 | 1919 | 190761 | 161.12 |
| DADU01_3#001 | 1840 | 198383 | 146.41 |
| DADU01_3#071 | 4237 | 144562 | 109.60 |
| DADU01_3#024 | 3782 | 152018 | 129.38 |
| DXDU01_3#051 | 1904 | 169289 | 144.69 |
| DADU01_3#004 | 2310 | 166261 | 141.26 |
| DADU01_3#045 | 1730 | 154444 | 127.85 |

2.5. Evaluation of binding of IgG having obtained Fab domain to CD3 epsilon and human CD137 at same time

Six antibodies (DXDU01_3 #094(#094), DADU01_3 #018(#018), DADU01_3 #002(#002), DXDU01_3 #019(#019), DXDU01_3 #051(#051) and DADU01_3 #001(#001 or dBBDu_126)) were selected to evaluate further. An anti-human CD137 antibody (SEQ ID NO: 93 for the Heavy chain and SEQ ID NO: 94 for the Light chain) described in WO2005/035584A1 (abbreviated as B) was used as a control antibody. Purified antibodies were subjected to ELISA to evaluate their binding capacity to CD3 epsilon and human CD137 at same time.

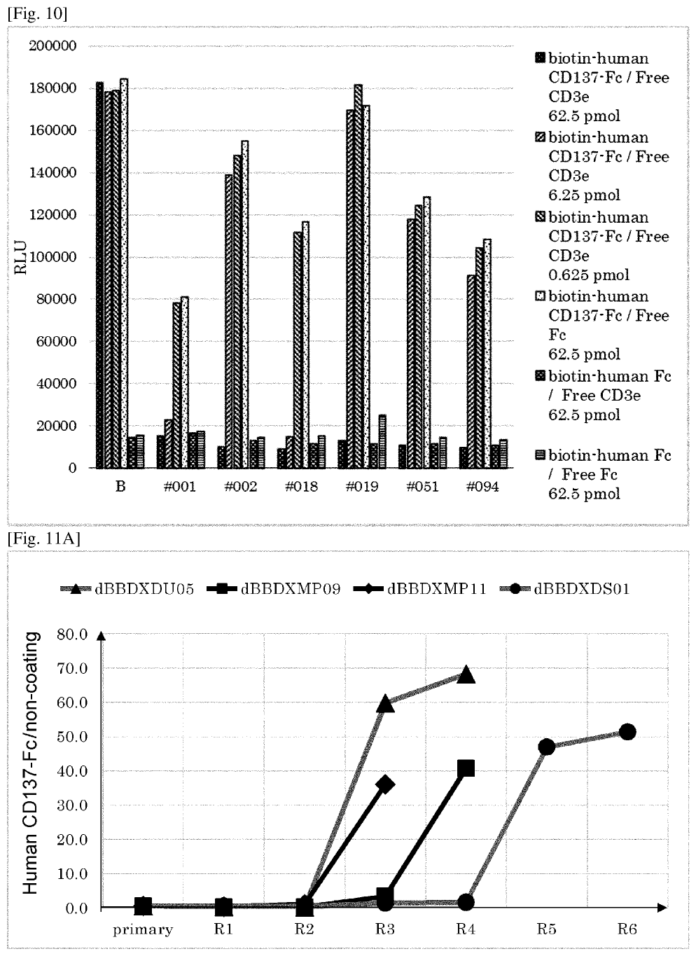

First, a MyOne-T1 streptavidin beads were mixed with 0.625 pmol of biotin-labeled human CD137-Fc or biotin-labeled human Fc and incubated at room temperature for 10 minutes, then 2% skim-milk/TBS was added to block the magnetic beads. Mixed solution was dispended to each well of 96well plate (Corning, 3792 black round bottom PS plate) and incubated at room temperature for 60 minutes or more. After that magnetic beads were washed by TBS once. 100 ng of purified IgG was mixed with 62.5, 6.25 or 0.625 pmol of free CD3 epsilon peptide or 62.5 pmol of free human Fc or TBS and then added to the magnetic beads in each well, and the plate was allowed to stand at room temperature for one hour to allow each IgG to bind to biotin-labeled antigen in each well. After that each well was washed with TBST. Goat anti-human kappa Light chain alkaline phosphatase conjugate (BETHYL, A80-115AP) diluted with TBS was added to each well. The plate was incubated for one hour. After washing with TBST, APS-5 (Lumigen) was added to each well. 2 minutes later the fluorescence of each well was detected. The measurement results are shown in FIG. 10 and Table 5.

TABLE 5

| | biotin-human CD137-Fc | | |
|---|---|---|---|
| | Free CD3e | Free Fc | |
| | 62.5 pmol | 62.5 pmol | Signal decrease |
| B | 182548 | 184279 | 0.94% |
| #001 | 15125 | 80997 | 81.33% |
| #002 | 9966 | 154791 | 93.56% |
| #018 | 9024 | 116919 | 92.28% |
| #019 | 12850 | 171835 | 92.52% |
| #051 | 10804 | 128260 | 91.58% |
| #094 | 9664 | 108313 | 91.08% |

Inhibition of binding to human CD137-Fc by free CD3 epsilon peptide was observed in all tested antibodies but not in control anti-CD137 antibody, and inhibition was not observed by free Fc domain. This results demonstrates those obtained antibodies could not bind to human CD137-Fc in the presence of CD3 epsilon peptide, in other words, these antibody do not bind to human CD137 and CD3 epsilon at same time. So it was proved that Fab domains which can bind to two different antigen, CD137 and CD3 epsilon, but not bind to at same time were successfully obtained with designed library and phage display double round selection.

### [Reference Example 3] Obtainment of Fab Domain Binding to CD3Epsilon, Human CD137 and Cyno CD137 from Dual Fab Library with Double Round Alternative Selection or Quadruple Round Selection

3.1. Panning Strategy to Improve the Efficiency to Obtain Fab Domain Binding to Cyno CD137

Fab domain binding to CD3 epsilon, human CD137 and cyno CD137 were successfully obtained in Reference Example 2, but binding to cyno CD137 was weaker than to human CD137. One of the considerable strategy to improve it is alternative panning with double round selection, in which different antigens would be used in different panning rounds. By this method selection pressure to both CD3 epsilon, human CD137 and cyno CD137 could be put on dual Fab library in each round with favorable antigen combination, CD3 epsilon with human CD137, CD3 epsilon with cyno CD137 or human CD137 with cyno CD137. And another strategy to improve it is the triple or quadruple round selection in which we can use all necessary antigens in one panning round.

In the double round selection procedure in Reference Example 2, over-night incubation was used to make antibody displaying phage transfer from 1^{st} antigen to 2^{nd} antigen. This methods worked well, but when affinity to 1^{st} antigen is stronger than to 2^{nd} antigen, transfer may be hardly occur (for example when 1^{st} antigen was CD3 epsilon in this dual library). To deal with this, elution of binding phage with base solution was also conducted. The campaign names and conditions of each panning procedure are described in Table 6.

Fab domains binding to CD3 epsilon, human CD137 and cyno CD137 were identified from the dual Fab library constructed in Reference Example 1.1. Biotin-labeled CD3 epsilon peptide antigen (amino acid sequence: SEQ ID NO: 86, CD3 epsilon peptide antigen biotin-labeled through disulfide-bond linker (C3 NP1-27; amino acid sequence: SEQ ID NO: 194), heterodimer of biotin-labeled human CD3 epsilon fused to human IgG1 Fc fragment and biotin-labeled human CD3 delta fused to human IgG1 Fc fragment (named as CD3 ed-Fc, amino acid sequence: SEQ ID NO: 95, 96), biotin-labeled human CD137 fused to human IgG1 Fc fragment (named as human CD137-Fc), biotin-labeled cynomolgus monkey CD137 fused to human IgG1 Fc fragment (named as cyno CD137-Fc) and biotin-labeled cynomolgus monkey CD137 (named as cyno CD137) was used as an antigen.

TABLE 6

| Campaign | | | Cycle 1 | | Cycle 2 | | Cycle 3 | | Cycle 4 | |
|---|---|---|---|---|---|---|---|---|---|---|
| Name | Round | panning | Antigen | Elution | Antigen | Elution | Antigen | Elution | Antigen | Elution |
| DU05 | Round 1 | Double | human CD137-Fc | IdeS | C3NP1-27 | DTT | | | | |
| | Round 2 | Double | cyno CD137-Fc | IdeS | C3NP1-27 | DTT | | | | |
| | Round 3 | Double | human CD137-Fc | IdeS | C3NP1-27 | DTT | | | | |
| | Round 4 | Double | cyno CD137-Fc | IdeS | C3NP1-27 | DTT | | | | |
| MP09 | Round 1 | Double | cyno CD137-Fc | IdeS | CD3ed-Fc | IdeS | | | | |
| | Round 2 | Double | human CD137-Fc | IdeS | cyno CD137-Fc | Trypsin | | | | |
| | Round 3 | Quadraple | human CD137-Fc | IdeS | CD3ed-Fc | IdeS | cyno CD137-Fc | IdeS | CD3ed-Fc | IdeS |
| | Round 4 | Quadraple | cyno CD137-Fc | IdeS | CD3ed-Fc | IdeS | human CD137-Fc | IdeS | CD3ed-Fc | IdeS |
| MP11 | Round 1 | Double | cyno CD137-Fc | IdeS | CD3ed-Fc | IdeS | | | | |
| | Round 2 | Quadraple | human CD137-Fc | IdeS | CD3ed-Fc | IdeS | cyno CD137-Fc | IdeS | CD3ed-Fc | IdeS |
| | Round 3 | Quadraple | cyno CD137-Fc | IdeS | CD3ed-Fc | IdeS | human CD137-Fc | IdeS | CD3ed-Fc | IdeS |
| DS01 | Round 1 | Single | human CD137-Fc | Trypsin | | | | | | |
| | Round 2 | Double | CD3 peptide | TEA | human CD137-Fc | Trypsin | | | | |
| | Round 3 | Double | CD3 peptide | TEA | human CD137-Fc | Trypsin | | | | |
| | Round 4 | Double | CD3 peptide | TEA | cyno CD137-Fc | Trypsin | | | | |
| | Round 5 | Double | CD3 peptide | TEA | human CD137-Fc | Trypsin | | | | |
| | Round 6 | Double | CD3 peptide | TEA | cyno CD137-Fc | Trypsin | | | | |

3.2. Obtainment of Fab Domain Binding to CD3Epsilon, Human CD137 and Cyno CD137 with Double Round Selection and Alternative Panning

Panning condition named as campaign DUOS was conducted to obtain Fab domain binding to CD3 epsilon, human CD137 and cyno CD137 with double round selection and alternative panning as shown in Table 6.

Human CD137-Fc was used in even-numbered round and cyno CD137-Fc was used in odd-numbered round. Detailed panning procedure of double round selection was as same as it shown in Reference Example 2. In DUOS campaign, double round selection was conducted since the 1^{st} round of panning.

3.3. Obtainment of Fab Domain Binding to CD3Epsilon, Human CD137 and Cyno CD137 with Base-Elution Double Round Selection and Alternative Panning

In previous double round selection with different antigens shown in Reference Example 2, antibody displaying phages were eluted as the complex with its 1^{st} antigen because IdeS or DTT cleaved the linker region between antigen and biotin, so 1^{st} antigen were also brought to the 2^{nd} cycle of double round selection and compete with 2^{nd} antigen. To suppress the carry-in of 1^{st} antigen, elution with base buffer, which induce dissociation of binding antibodies from antigen and is very popular method in conventional phage display panning, was also conducted (name as campaign DS01). Detailed panning procedure of panning round1 was as same as it shown in Reference Example 2. In round1, conventional panning with biotin labeled human CD137-Fc was conducted.

In panning round1 Fab displaying phages which bind to human CD137 were accumulated so from panning round2 base-elution double round selection was conducted to obtain Fab domain which bind to CD3 epsilon, human CD137 and cyno CD137.

Specifically, at panning round2, magnetic beads was blocked by 2% skim-milk/TBS at room temperature for 60 minutes or more and washed three times with TBS. Phage solution were added to blocked magnetic beads and incubated at room temperature for 60 minutes or more, then supernatant was recovered. 500 pmol of biotin labeled human IgG1 Fc was added to new magnetic beads and incubated at room temperature for 15 minutes and then add 2% skim-milk/TBS. After blocking at room temperature for 60 minutes or more, magnetic beads was washed three times with TBS. Recovered phage solution were added to blocked magnetic beads and incubated at room temperature for 60 minutes or more, then supernatant was recovered. 500 pmol of the biotin-labeled CD3 epsilon peptide was added to new magnetic beads and incubated at room temperature for 15 minutes and then add 2% skim-milk/TBS.

After blocking at room temperature for 60 minutes or more, magnetic beads was washed three times with TBS. Recovered phage solution were added to blocked magnetic beads and then incubated at room temperature for 60 minutes. The beads were washed three times with TBST (TBS containing 0.1% Tween 20; TBS was available from Takara Bio Inc.) and then further washed twice with 1 mL of TBS. 0.1 M Triethylamine (TEA, Wako 202-02646) was used to recover antibody displaying phages. In that procedure, 500 micro L of 0.1 M TEA was added and beads were suspended at room temperature for 10 minutes, immediately after which the beads were separated using a magnetic stand to recover phage solution. 100 micro L of 1M Tris-HCl (pH 7.5) was added to neutralize phage solution for 15 minutes.

In this 1^{st} cycle of panning procedure antibody displaying phages which bind to CD3 epsilon was concentrated so then move on to 2nd cycle panning procedure to recover antibody displaying phages which also bind to CD137 before phage infection and amplification. 500 pmol of the biotin-labeled human CD137-Fc was added to new magnetic beads and incubated at room temperature for 15 minutes and then add 2% skim-milk/TBS. After blocking at room temperature for 60 minutes or more, magnetic beads was washed three times with TBS. Recovered phage solution, 50 micro L of TBS and 250 micro L of 8% BSA blocking buffer were added to blocked magnetic beads and then incubated at room temperature for 60 minutes.

The beads were washed three times with TBST (TBS containing 0.1% Tween 20; TBS was available from Takara Bio Inc.) and then further washed twice with 1 mL of TBS. The beads supplemented with 0.5 mL of 1 mg/mL trypsin were suspended at room temperature for 15 minutes, immediately after which the beads were separated using a magnetic stand to recover a phage solution. The phages recovered from the trypsin-treated phage solution were added to an E. coli strain ER2738 in a logarithmic growth phase (0D600: 0.4-0.7). The E. coli strain was infected by the phages through the gentle spinner culture of the strain at 37 degrees C. for 1 hour. The infected E. coli was inoculated to a plate of 225 mm×225 mm. Next, phages were recovered from the culture solution of the inoculated E. coli to recover a phage library solution.

In the 2^{nd} cycle of double round selection in fourth and sixth round of panning, biotin labeled cyno CD137-Fc was used instead of biotin labeled human CD137-Fc. Through panning round4 to round6, 250 pmol of biotin labeled human or cyno CD137-Fc was used in the 2^{nd} cycle of double round selection.

3.4. Obtainment of Fab Domain Binding to CD3Epsilon, Human CD137 and Cyno CD137 with Quadruple Round Selection

In previous double round selection only two different antigens could be used in the panning one round. To break through this limitation, quadruple round selection was also conducted (name as campaign MP09 and MP11, shown in Table 6).

In panning round1 of both MP09 and MP11 and panning round2 of MP09, double round selection was conducted.

Specifically, magnetic beads was blocked by 2% skim-milk/TBS at room temperature for 60 minutes or more and washed three times with TBS. Phage solution were added to blocked magnetic beads and incubated at room temperature for 60 minutes or more, then supernatant was recovered. 500 pmol of biotin labeled human IgG1 Fc was added to new magnetic beads and incubated at room temperature for 15 minutes and then add 2% skim-milk/TBS. After blocking at room temperature for 60 minutes or more, magnetic beads was washed three times with TBS. Recovered phage solution were added to blocked magnetic beads and incubated at room temperature for 60 minutes or more, then supernatant was recovered. 268 pmol of the biotin-labeled cyno CD137-Fc was added to new magnetic beads and incubated at room temperature for 15 minutes and then add 2% skim-milk/TBS.

After blocking at room temperature for 60 minutes or more, magnetic beads was washed three times with TBS. Recovered phage solution were added to blocked magnetic beads and then incubated at room temperature for 60 minutes. The beads were washed three times with TBST (TBS containing 0.1% Tween 20; TBS was available from Takara Bio Inc.) and then further washed twice with 1 mL of TBS. FabRICATOR (IdeS, protease for hinge region of IgG, GENOVIS)(named as IdeS elution campaign) was used to recover antibody displaying phages. In that procedure, 10 units/micro L Fabricator 20 micro L with 80 micro L TBS buffer was added and beads were suspended at 37 degrees Celsius for 30 minutes, immediately after which the beads were separated using a magnetic stand to recover phage solution.

In this 1^{st} cycle of panning procedure antibody displaying phages which bind to cyno CD137 was concentrated so then move on to 2nd cycle panning procedure to recover antibody displaying phages which also bind to CD3 epsilon before phage infection and amplification. To remove IdeS protease from phage solution, 40 micro L of helper phage M13KO7 (1.2E+13 pfu) and 200 micro L of 10% PEG-2.5M NaCl was added and a pool of the phages thus precipitated was diluted with TBS to obtain a phage library solution. 500 pmol of the biotin-labeled CD3 ed-Fc was added to new magnetic beads and incubated at room temperature for 15 minutes and then add 2% skim-milk/TBS. After blocking at room temperature for 60 minutes or more, magnetic beads was washed three times with TBS. Recovered phage solution and 500 micro L of 8% BSA blocking buffer were added to blocked magnetic beads and then incubated at room temperature for 60 minutes.

The beads were washed three times with TBST (TBS containing 0.1% Tween 20; TBS was available from Takara Bio Inc.) and then further washed twice with 1 mL of TBS. 10 units/micro L Fabricator 20 micro L with 80 micro L TBS buffer was added and beads were suspended at 37 degrees Celsius for 30 minutes, immediately after which the beads were separated using a magnetic stand to recover phage solution. 5 micro L of 100 mg/mL trypsin and 395 micro L of TBS were added and incubated at room temperature for 15 minutes. The phages recovered from the trypsin-treated phage solution were added to an E. coli strain ER2738 in a logarithmic growth phase (0D600: 0.4-0.7). The E. coli strain was infected by the phages through the gentle spinner culture of the strain at 37 degrees C. for 1 hour. The infected E. coli was inoculated to a plate of 225 mm×225 mm. Next, phages were recovered from the culture solution of the inoculated E. coli to recover a phage library solution.

In the second round of panning campaign of MP09, biotin-labeled human CD137-Fc was used as 1^{st} cycle panning antigen and biotin-labeled cyno CD137 with elution by Trypsin was used as 2^{nd} cycle panning antigen as shown in Table 6.

Quadruple panning was conducted in panning round3 and round4 of MP09 campaign and panning round2 and round3 of MP11 campaign.

In panning round3 of MP09 and round2 of MP11 campaign, magnetic beads was blocked by 2% skim-milk/TBS at room temperature for 60 minutes or more and washed three times with TBS. Phage solution were added to blocked magnetic beads and incubated at room temperature for 60 minutes or more, then supernatant was recovered. 500 pmol of biotin labeled human IgG1 Fc was added to new magnetic beads and incubated at room temperature for 15 minutes and then add 2% skim-milk/TBS. After blocking at room temperature for 60 minutes or more, magnetic beads was washed three times with TBS. Recovered phage solution were added to blocked magnetic beads and incubated at room temperature for 60 minutes or more, then supernatant was recovered. 250 pmol of the biotin-labeled human CD137-Fc was added to new magnetic beads and incubated at room temperature for 15 minutes and then add 2% skim-milk/TBS.

After blocking at room temperature for 60 minutes or more, magnetic beads was washed three times with TBS. Recovered phage solution were added to blocked magnetic beads and then incubated at room temperature for 60 minutes. The beads were washed three times with TBST (TBS containing 0.1% Tween 20; TBS was available from Takara Bio Inc.) and then further washed twice with 1 mL of TBS. FabRICATOR (IdeS, protease for hinge region of IgG, GENOVIS) (named as IdeS elution campaign) was used to recover antibody displaying phages. In that procedure, 10 units/micro L Fabricator 20 micro L with 80 micro L TBS buffer was added and beads were suspended at 37 degrees Celsius for 30 minutes, immediately after which the beads were separated using a magnetic stand to recover phage solution.

To remove IdeS protease from phage solution, 40 micro L of helper phage M13KO7 (1.2E+13 pfu) and 200 micro L of 10% PEG-2.5M NaCl was added and a pool of the phages thus precipitated was diluted with TBS to obtain a phage library solution. 250 pmol of the biotin-labeled CD3 ed-Fc was added to new magnetic beads and incubated at room temperature for 15 minutes and then add 2% skim-milk/TBS. After blocking at room temperature for 60 minutes or more, magnetic beads was washed three times with TBS. Recovered phage solution and 500 micro L of 8% BSA blocking buffer were added to blocked magnetic beads and then incubated at room temperature for 60 minutes. The beads were washed three times with TBST (TBS containing 0.1% Tween 20; TBS was available from Takara Bio Inc.) and then further washed twice with 1 mL of TBS. 10 units/micro L Fabricator 20 micro L with 80 micro L TBS buffer was added and beads were suspended at 37 degrees Celsius for 30 minutes, immediately after which the beads were separated using a magnetic stand to recover phage solution.

In 3^{rd} cycle of quadruple round selection, 40 micro L of helper phage M13KO7 (1.2E+13 pfu) and 200 micro L of 10% PEG-2.5M NaCl was added and a pool of the phages thus precipitated was diluted with TBS to obtain a phage library solution. 250 pmol of the biotin-labeled cyno CD137-Fc was added to new magnetic beads and incubated at room temperature for 15 minutes and then add 2% skim-milk/TBS. After blocking at room temperature for 60 minutes or more, magnetic beads was washed three times with TBS. Recovered phage solution and 500 micro L of 8% BSA blocking buffer were added to blocked magnetic beads and then incubated at room temperature for 60 minutes. The beads were washed three times with TBST (TBS containing 0.1% Tween 20; TBS was available from Takara Bio Inc.) and then further washed twice with 1 mL of TBS. 10 units/micro L Fabricator 20 micro L with 80 micro L TBS buffer was added and beads were suspended at 37 degrees Celsius for 30 minutes, immediately after which the beads were separated using a magnetic stand to recover phage solution.

In 4^{th} cycle of quadruple round selection, 40 micro L of helper phage M13KO7 (1.2E+13 pfu) and 200 micro L of 10% PEG-2.5M NaCl was added and a pool of the phages thus precipitated was diluted with TBS to obtain a phage library solution. 500 pmol of the biotin-labeled CD3 ed-Fc was added to new magnetic beads and incubated at room temperature for 15 minutes and then add 2% skim-milk/TBS. After blocking at room temperature for 60 minutes or more, magnetic beads was washed three times with TBS. Recovered phage solution and 500 micro L of 8% BSA blocking buffer were added to blocked magnetic beads and then incubated at room temperature for 60 minutes.

The beads were washed three times with TBST (TBS containing 0.1% Tween 20; TBS was available from Takara Bio Inc.) and then further washed twice with 1 mL of TBS. 10 units/micro L Fabricator 20 micro L with 80 micro L TBS buffer was added and beads were suspended at 37 degrees Celsius for 30 minutes, immediately after which the beads were separated using a magnetic stand to recover phage solution. 5 micro L of 100 mg/mL trypsin and 395 micro L of TBS were added and incubated at room temperature for 15 minutes. The phages recovered from the trypsin-treated phage solution were added to an E. coli strain ER2738 in a logarithmic growth phase (0D600: 0.4-0.7). The E. coli strain was infected by the phages through the gentle spinner culture of the strain at 37 degrees C. for 1 hour. The infected E. coli was inoculated to a plate of 225 mm×225 mm. Next, phages were recovered from the culture solution of the inoculated E. coli to recover a phage library solution.

In panning round4 of MP09 and round3 of MP11 campaign, biotin labeled human CD137-Fc was used as 1^{st} cycle antigen and biotin labeled cyno CD137-Fc was used as 3^{rd} cycle antigen.

3.5. Binding of Fab Domain Displayed by Phage to Human and Cyno CD137 (Phage ELISA)

Fab displaying phage solution were prepared through panning procedure in Reference Example 3.2, 3.3 and 3.4. First, 20 micro g of Streptavidin-coated magnetic beads MyOne-T1 beads was washed three-times with blocking buffer including 0.4% block Ace, 1% BSA, 0.02% Tween and 0.05% ProClin 300 and then blocked with this blocking buffer at room temperature for 60 minutes or more. After washing once with TBST, magnetic beads were applied to each well of 96well plate (Corning, 3792 black round bottom PS plate) and 0.625 pmol of biotin labeled human CD137-Fc, biotin labeled cyno CD137-Fc or biotin labeled CD3 epsilon peptide was added to magnetic beads and incubated at room temperature for 15 minutes or more.

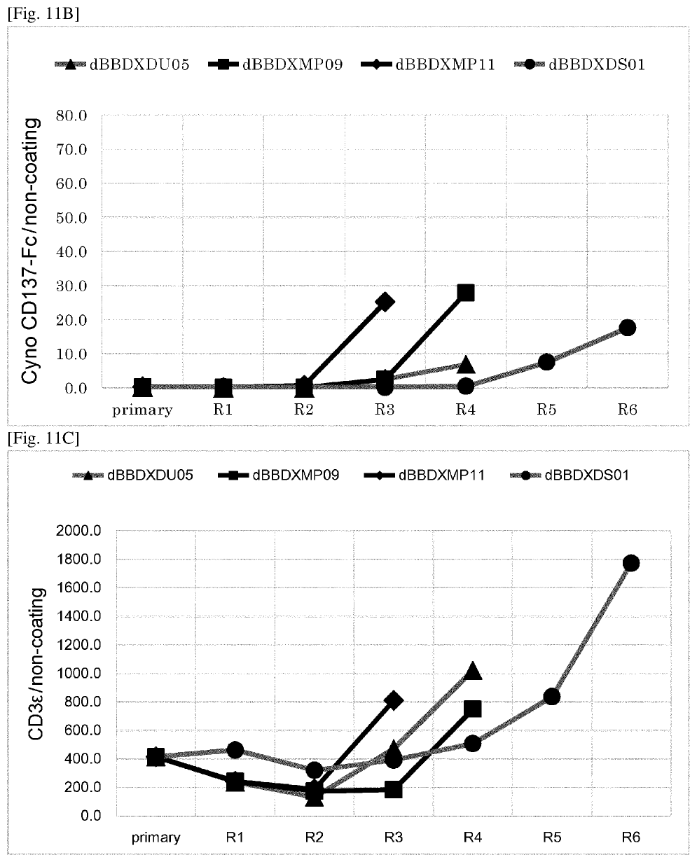

After washing once with TBST, 250 nL each of the Fab displaying phage solution with 24.75 micro L of TBS was added to the wells, and the plate was allowed to stand at room temperature for one hour to allow each Fab to bind to biotin-labeled antigen in each well. After that each well was washed with TBST. Anti-M13(p8) Fab-HRP diluted with TBS was added to each well. The plate was incubated for 10 minutes. After washing with TBST, LumiPhos-HRP (Lumigen) was added to each well. 2 minutes later the fluorescence of each well was detected. The measurement results are shown in FIG. 11.

The binding to each antigens, human CD137, cyno CD137 and CD3 epsilon, were observed in each panning output phage solution. This result showed that double round selection with base elution worked as well as previous double round selection with IdeS elution method, and that double round selection with alternative panning also worked well to obtain Fab domain which bind to three different antigens. Nonetheless the binding to cyno CD137 was still weak compared to human CD137 although these methods collect Fab domains which bind to three different antigens. On the other hand, in MP09 or MP11 campaign, the binding to CD3 epsilon, human CD137 and cyno CD137 were observed at same round point and their binding to cyno CD137 was higher than other campaign. This result demonstrated that quadruple round selection can concentrate Fab domain which bind to three different antigens more efficiently.

3.6. Preparation of IgG Having Obtained Fab Domain

96 clones were picked from each panning output pools and their VH gene sequence were analyzed. Thirty-two clones were selected because their VH sequence were appeared more than twice among all analyzed pools. Their VH gene were amplified by PCR and converted into IgG format. The VH fragments of each clones were amplified by PCR using primers specifically binding to the H chain in the library (SEQ ID NOs: 196 and 197). The amplified VH fragment was integrated into an animal expression plasmid which have already had human IgG1 CH1-Fc region. The prepared plasmids were used for expression in animal cells by the method of Reference Example 9. These sample were called as clone converted IgG. GLS3000 was used as Light chain.

VH genes of each panning output pools were also converted into IgG format. Phagemid vector library were prepared from the E. coli of each panning output pools DUOS, DS01 and MP11, and digested with NheI and SalI restriction enzyme to extract VH genes directly. The extracted VH fragments were integrated into an animal expression plasmid which have already had human IgG1 CH1-Fc region. The prepared plasmids were introduced into E. coli and 192 or 288 colonies were picked from each panning output pools and their VH sequence were analyzed. In MP09 and 11 campaign, clones which had different VH sequences were picked up as possible. The prepared plasmids from each E. coli colonies were used for expression in animal cells by the method of Reference Example 9. These sample were called as bulk converted IgG. GLS3000 was used as Light chain.

3.7. Assessment of the Obtained Antibodies for their CD3Epsilon, Human CD137 and Cyno CD137 Binding Activity

The prepared bulk converted IgG antibodies were subjected to ELISA to evaluate their binding capacity to CD3 epsilon, human CD137 and cyno CD137.

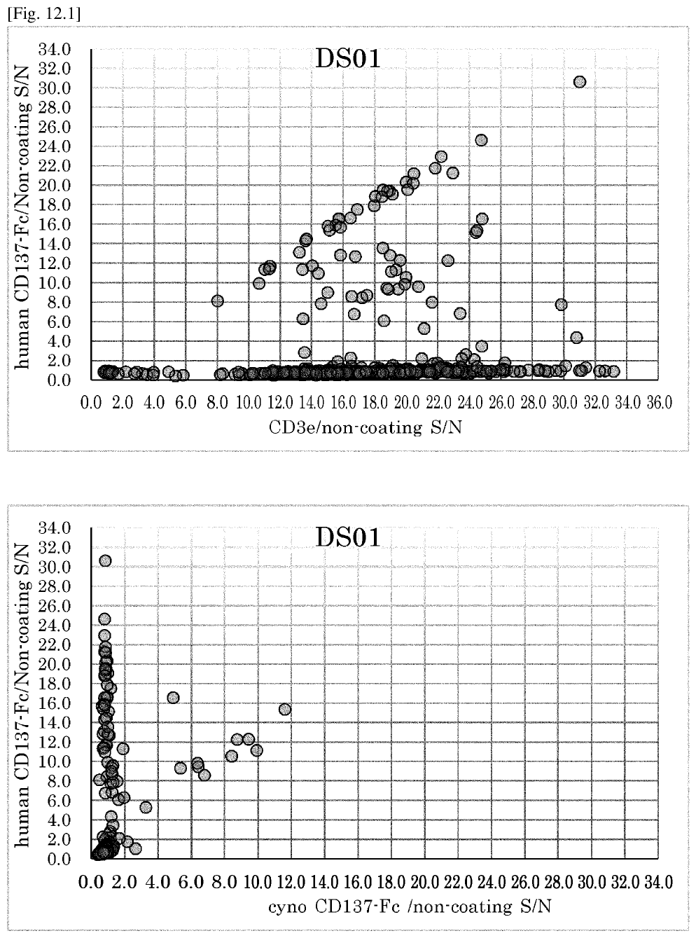

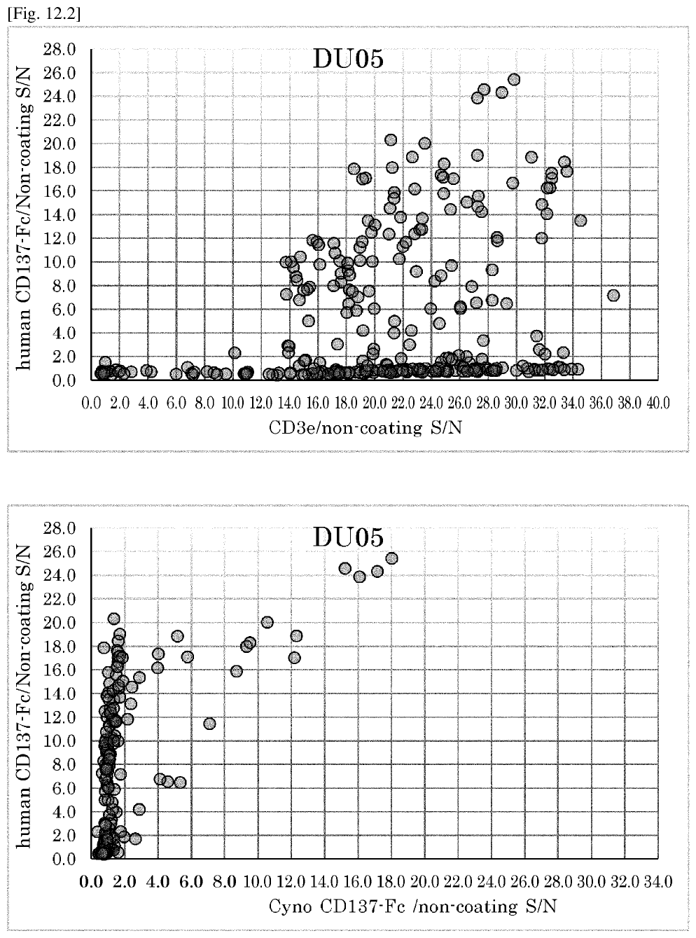

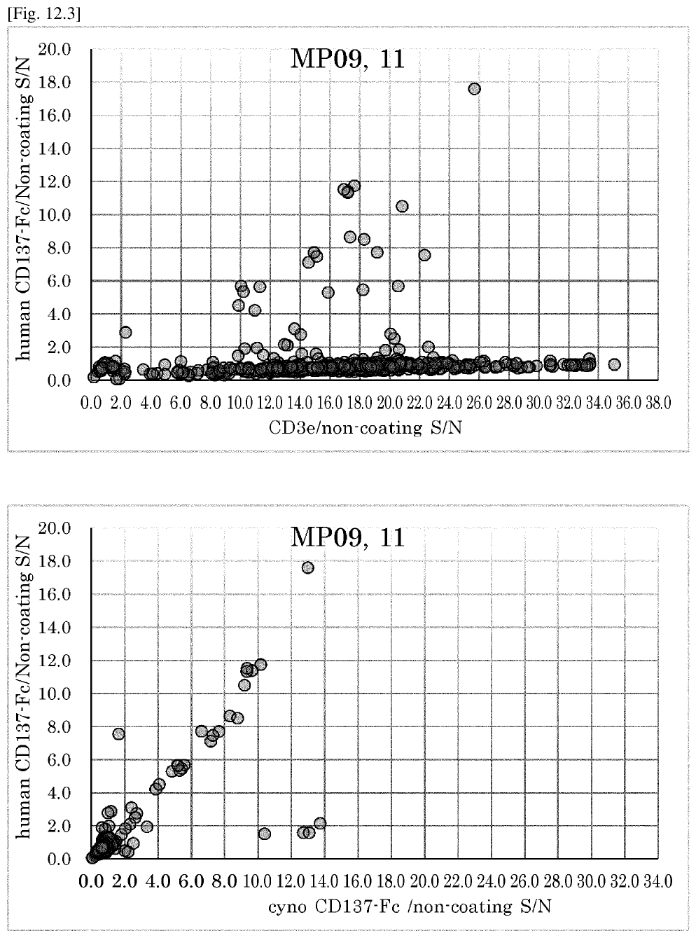

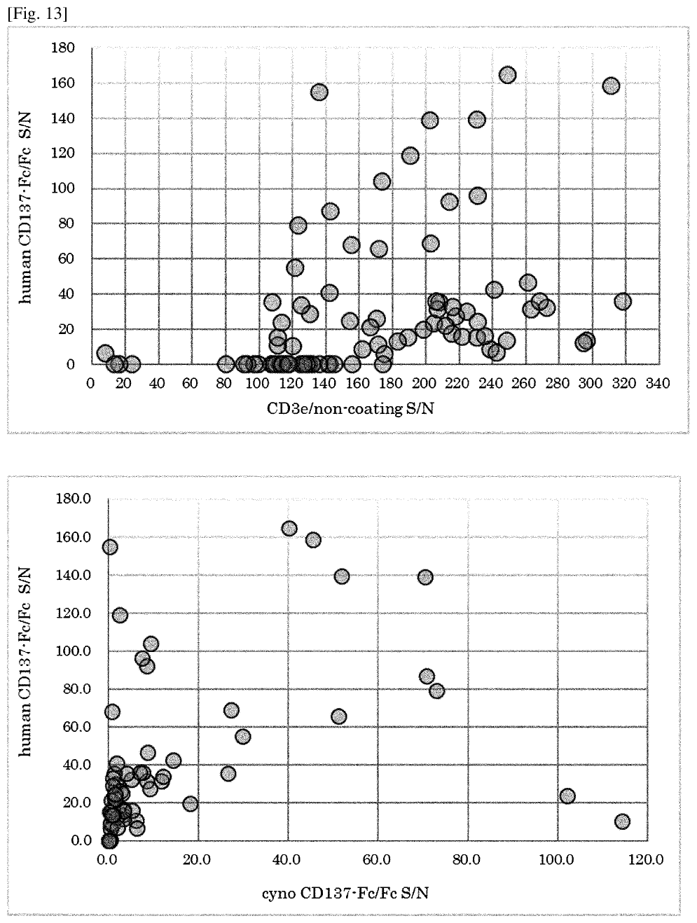

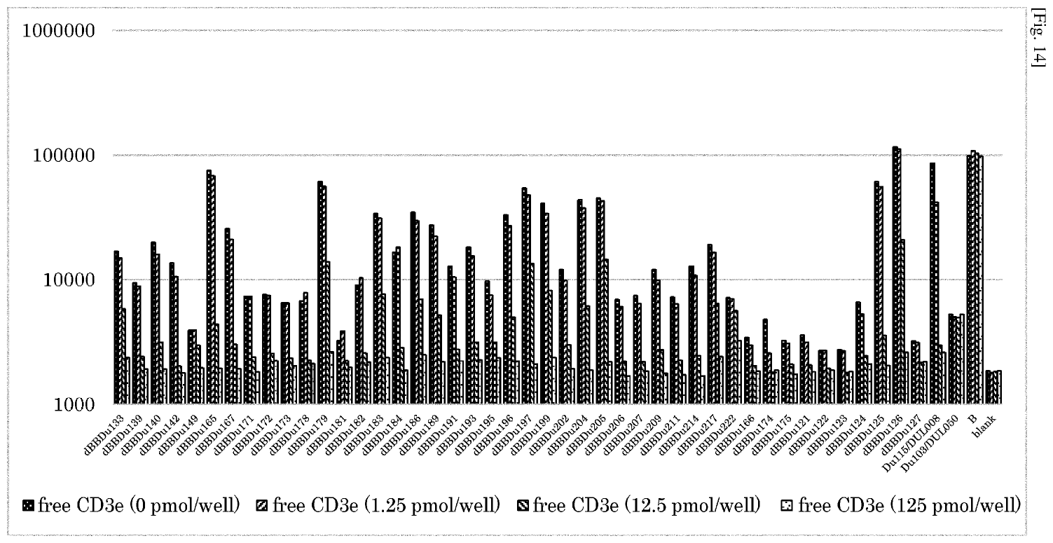

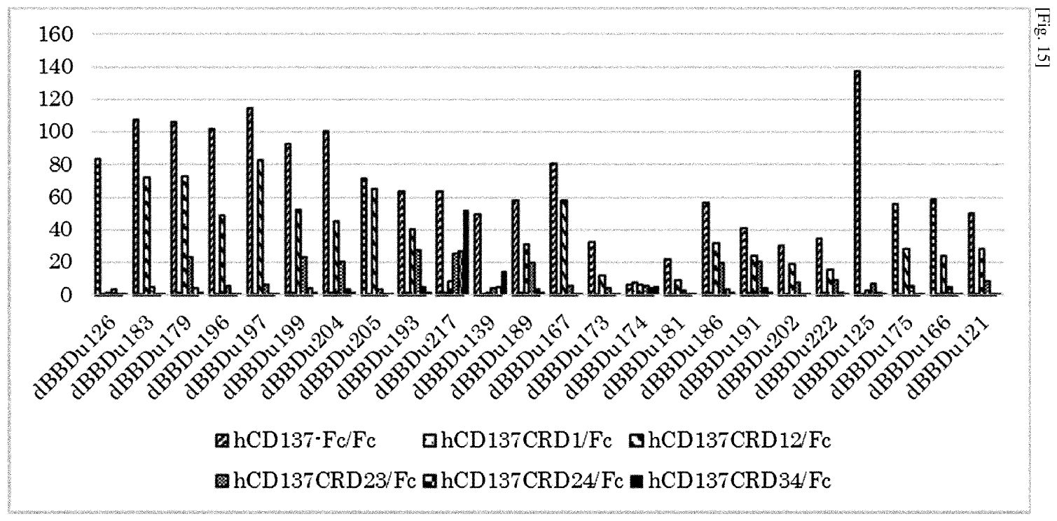

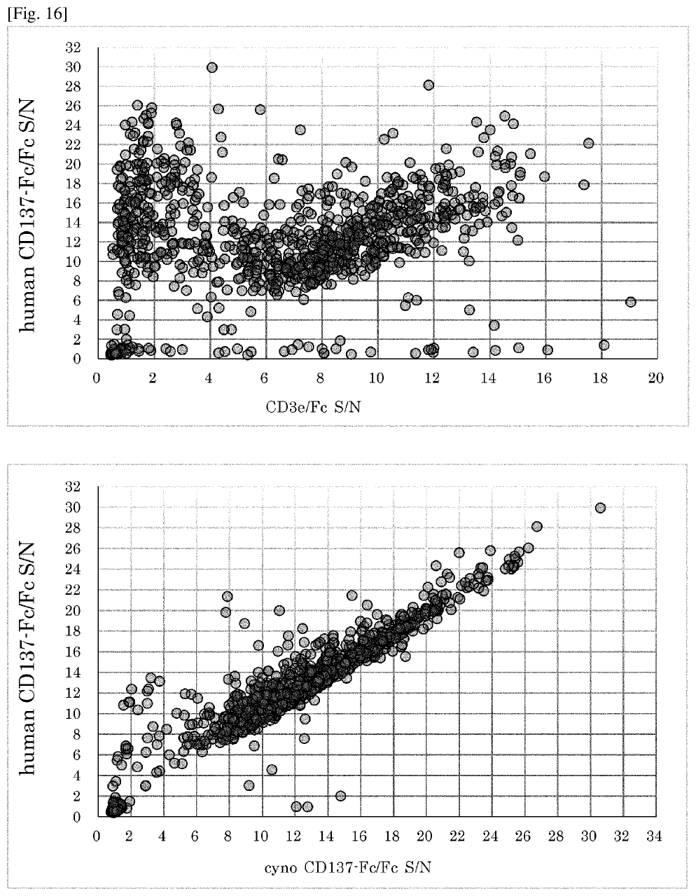

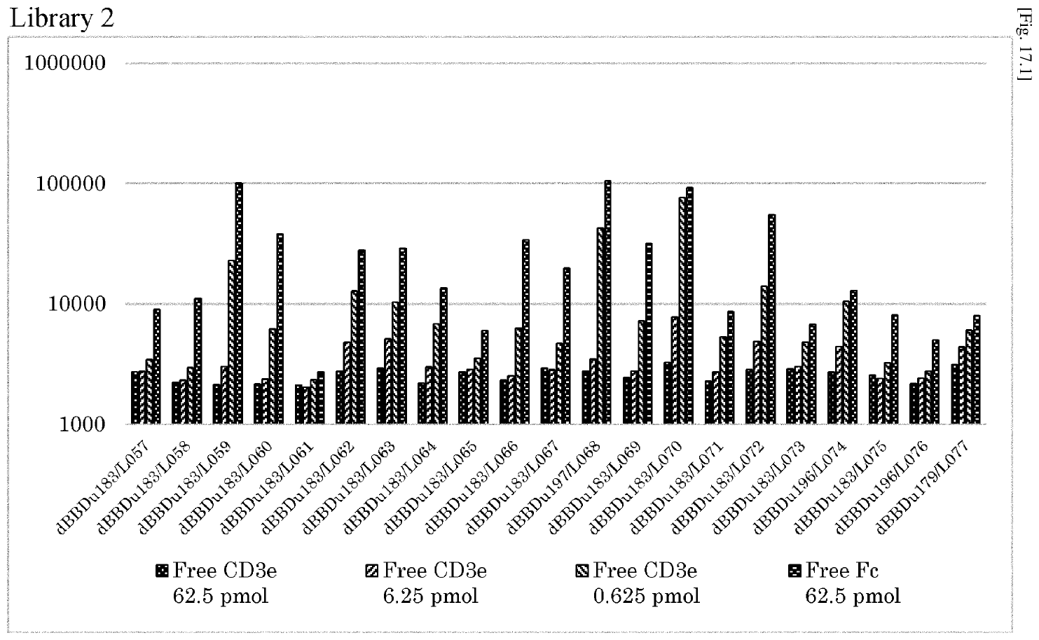

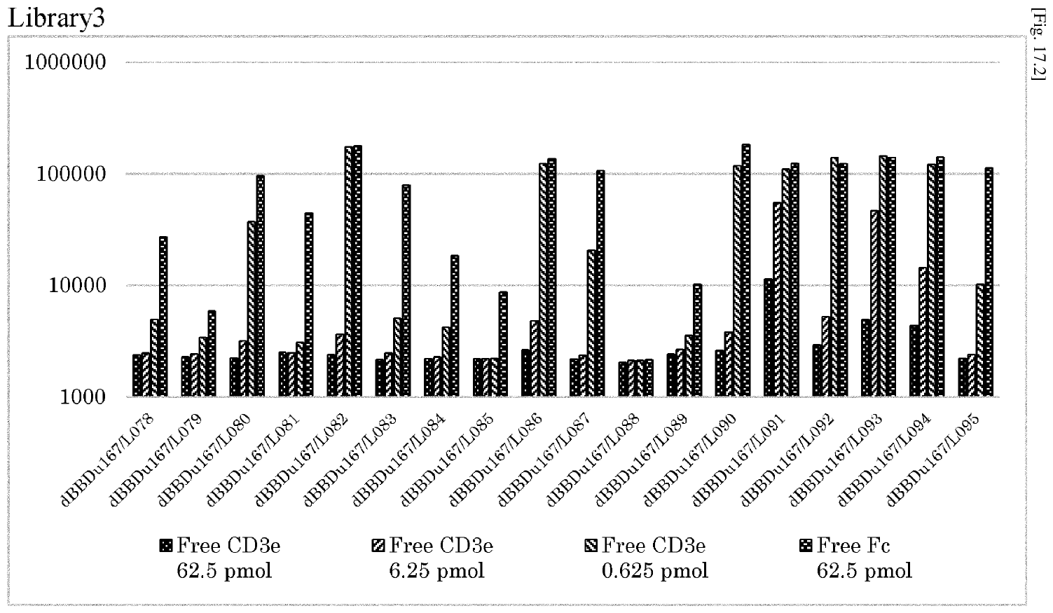

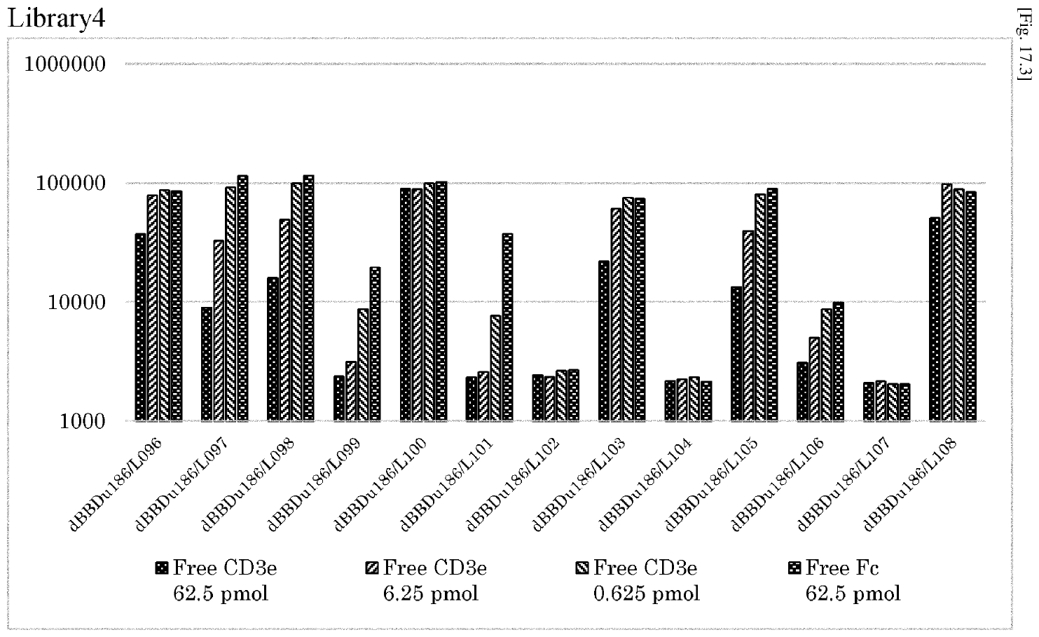

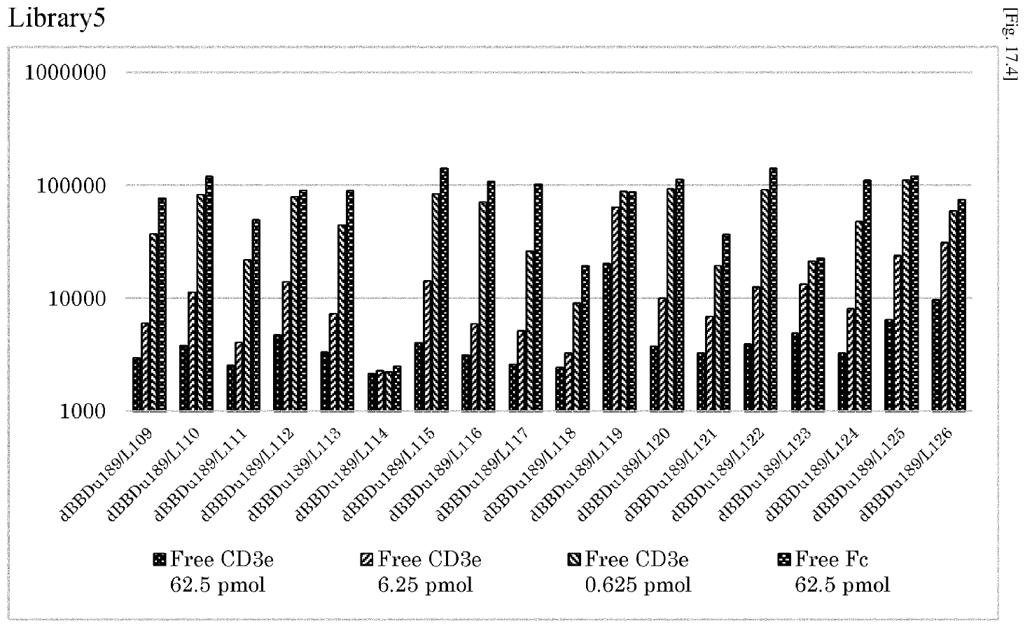

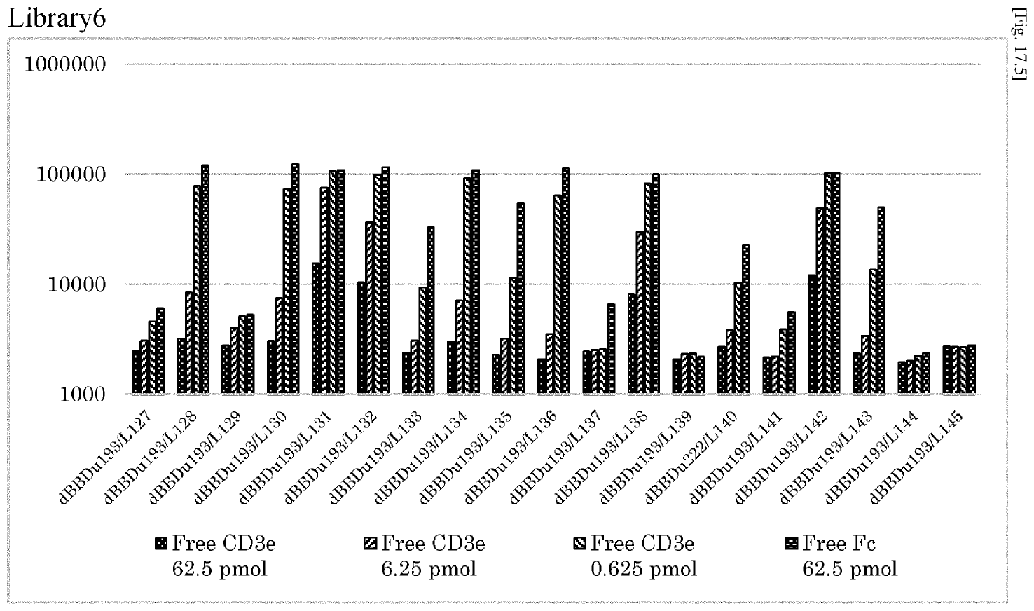

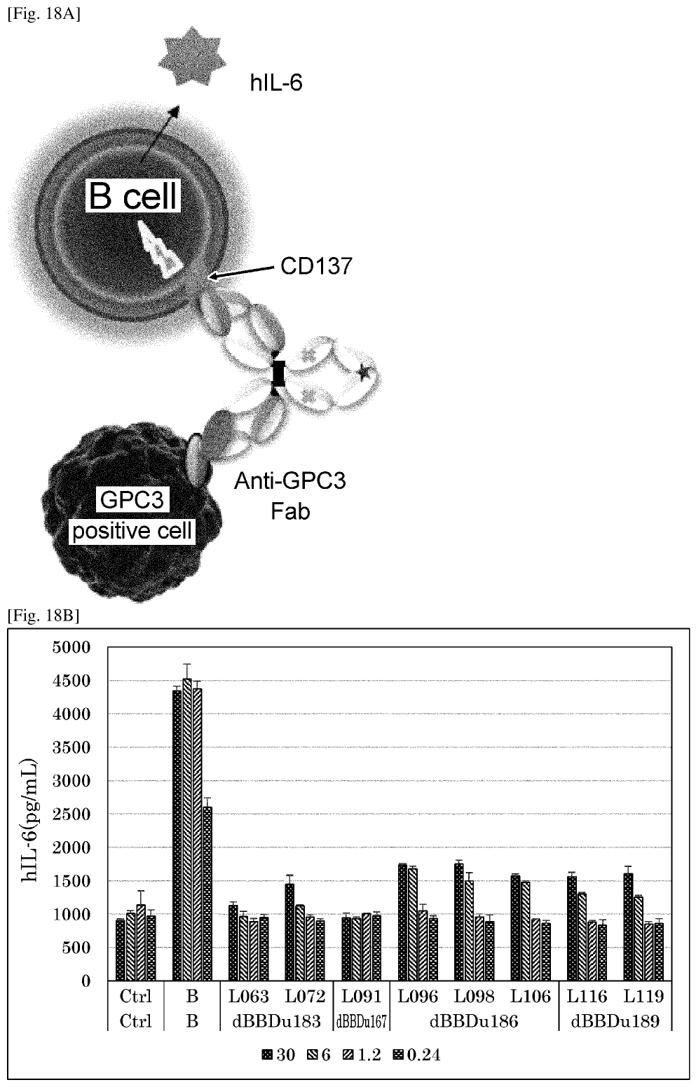

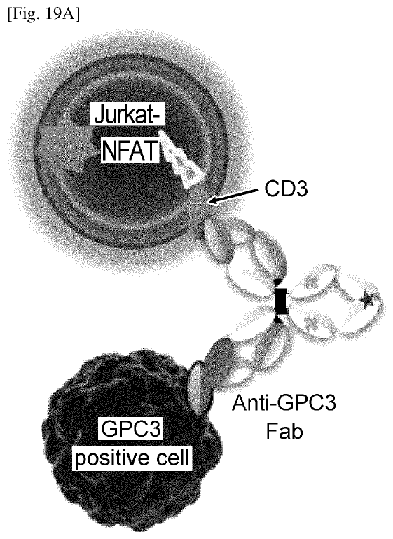

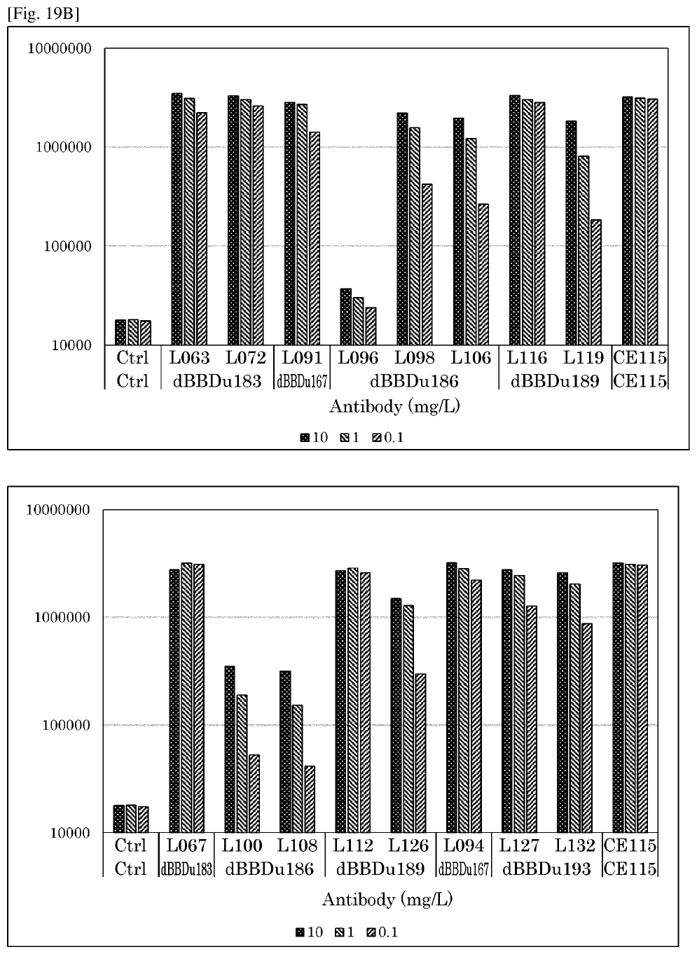

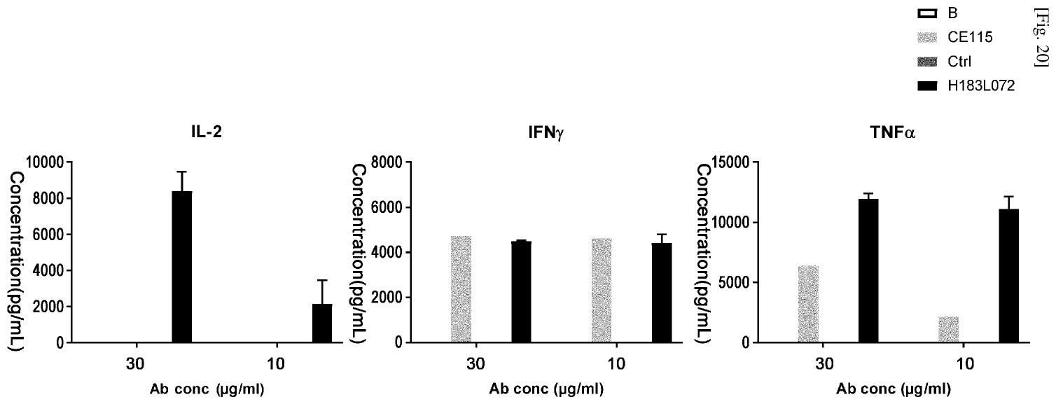

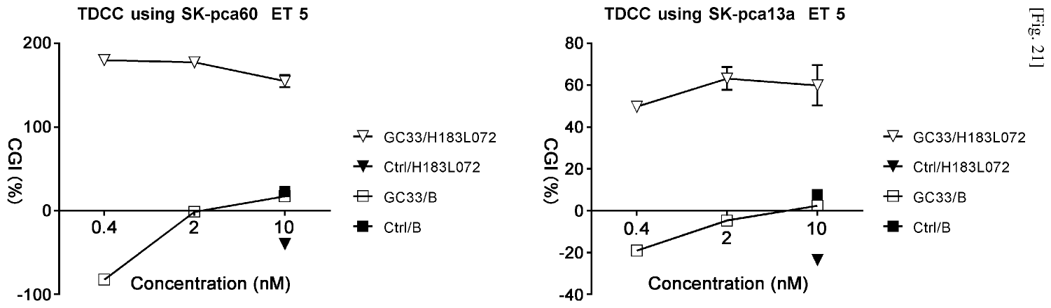

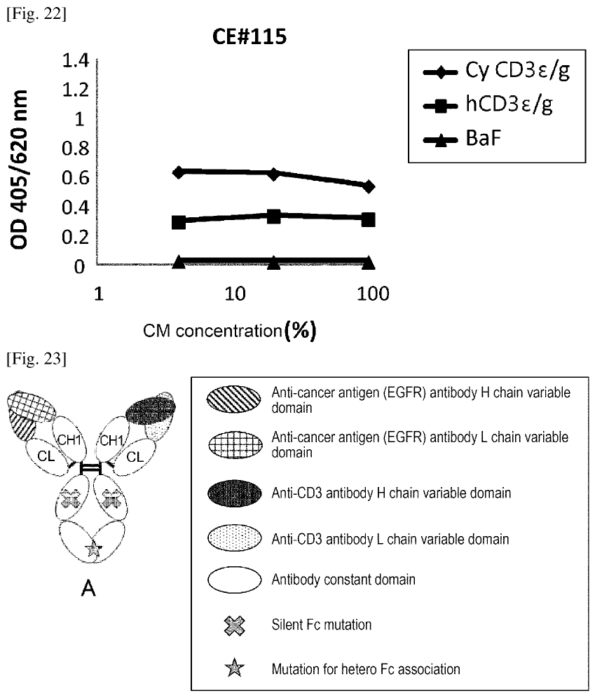

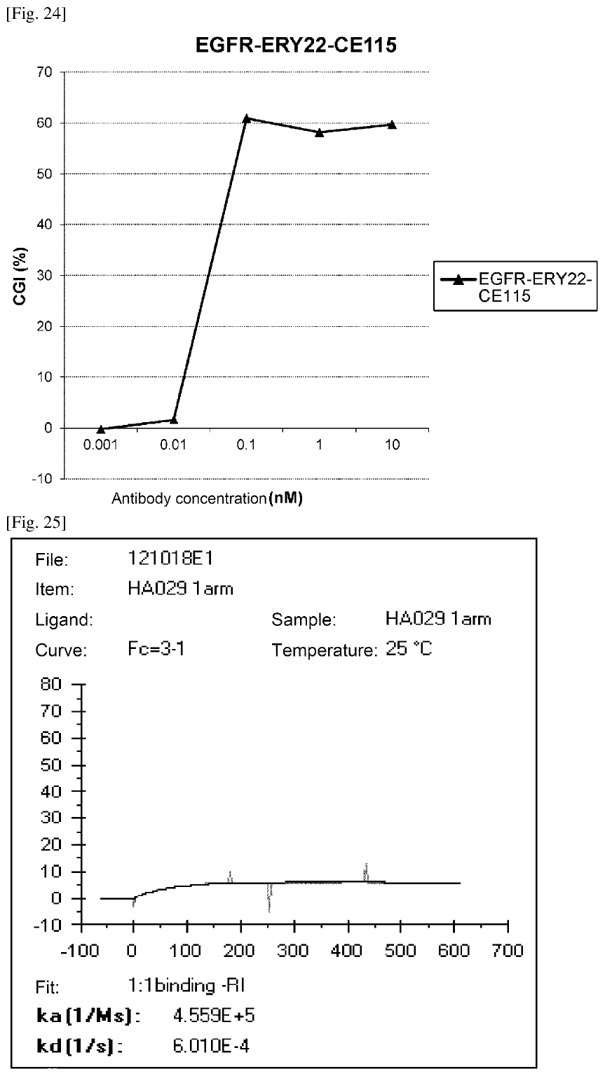

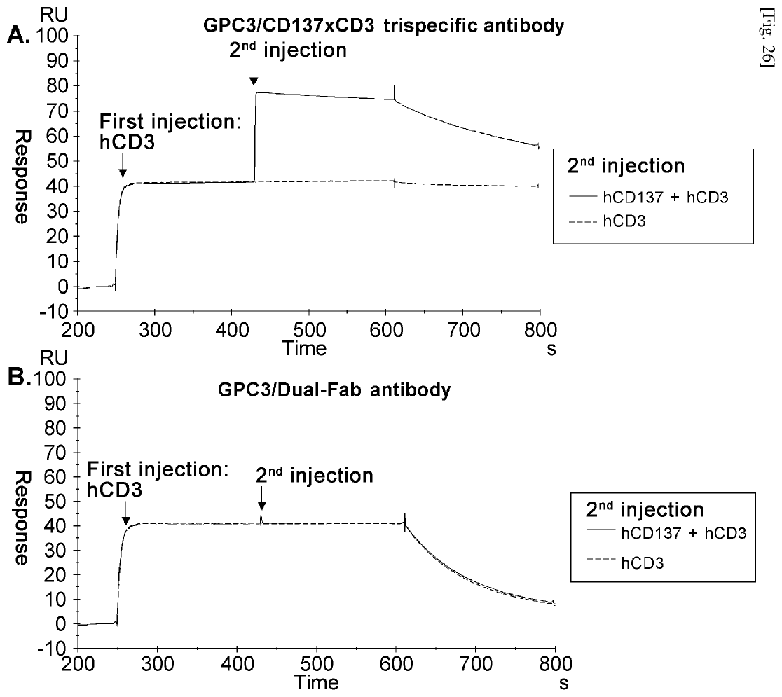

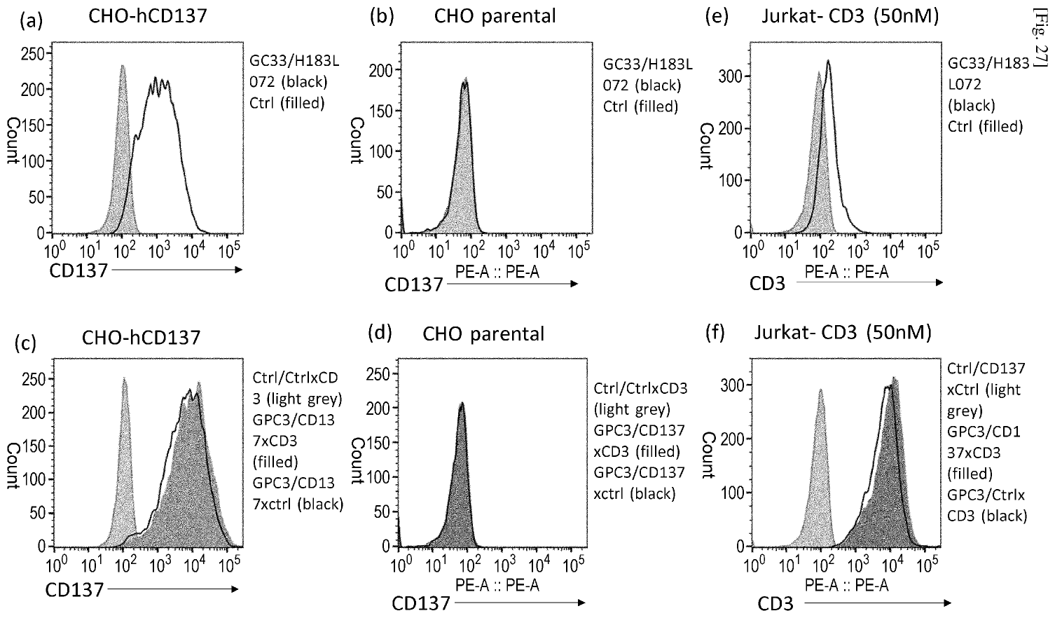

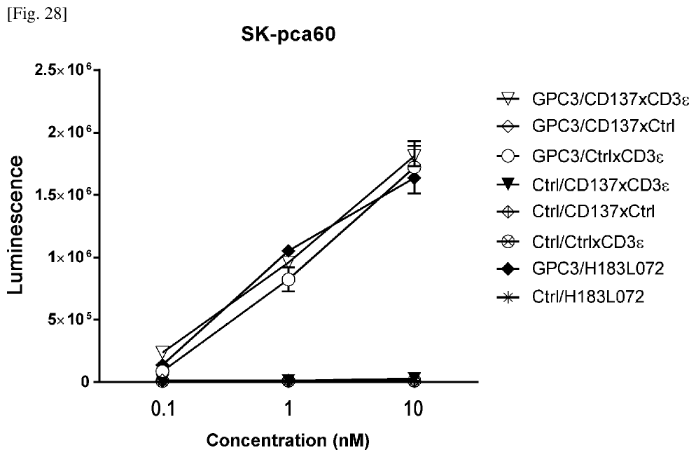

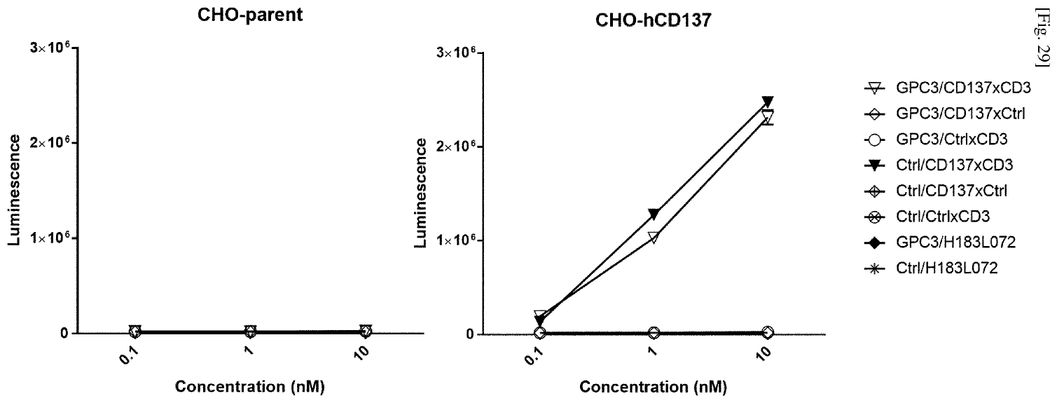

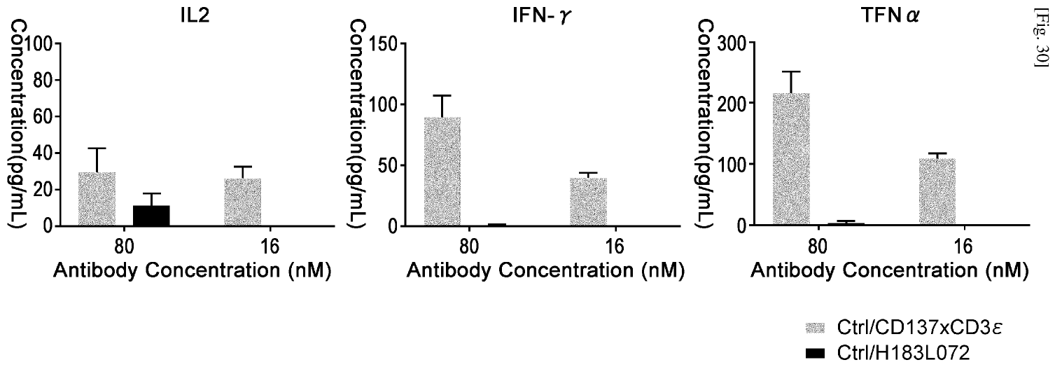

First, a Streptavidin-coated microplate (384 well, Greiner) was coated with 20 micro L of TBS containing biotin-labeled CD3 epsilon peptide, biotin labeled human CD137-Fc or biotin labeled cyno CD137-Fc at room temperature for one or more hours. After removing biotin-labeled antigen that are not bound to the plate by washing each well of the plate with TBST, the wells were blocked with 20 micro L of Blocking Buffer (2% skim milk/TBS) for one or more hours. Blocking Buffer was removed from each well. 20 micro L each of the IgG containing mammalian cell supernatant twice diluted with 2% Skim milk/TBS were added to the wells, and the plate was allowed to stand at room temperature for one hour to allow each IgG to bind to biotin-labeled antigen in each well. After that each well was washed with TBST. Goat anti-human kappa Light chain alkaline phosphatase conjugate (BETHYL, A80-115AP) diluted with TBS was added to each well. The plate was incubated for one hour. After washing with TBST, the chromogenic reaction of the solution in each well added with Blue Phos Microwell Phosphatase Substrate System (KPL) was terminated by adding Blue Phos Stop Solution (KPL). Then, the color development was measured by absorbance at 615 nm. The measurement results are shown in FIG. 12.

Many IgG clones which showed binding to both CD3 epsilon, human CD137 and cyno CD137 were obtained from each panning procedure so it proves that both double round selection with alternative panning, double selection with base elution and quadruple round selection were all worked as expected. Especially, Most of all clones from quadruple round selection which bound to human CD137 showed equality level of binding to cyno-CD137 compared to other two panning conditions. In those panning conditions it was likely to be obtained less clones which showed binding to both CD3 epsilon and human CD137, it mainly because clones which had same VH sequences each other were not picked up on purpose as possible in this campaign. Fifty-four clones which showed better binding to each protein and had different VH sequences each other were selected and evaluated further.

3.8. Assessment of the Purified IgG Antibodies for their CD3Epsilon, Human CD137 and Cyno CD137 Binding Activity

The binding capability of purified IgG antibodies were evaluated. Thirty-two clone converted IgGs in Reference Example 3.5 and fifty-four bulk converted IgGs which was selected in Reference Example 3.6 were used.

First, 20 micro g of Streptavidin-coated magnetic beads MyOne-T1 beads was washed three-times with blocking buffer including 0.4% block Ace, 1% BSA, 0.02% Tween and 0.05% ProClin 300 and then blocked with this blocking buffer at room temperature for 60 minutes or more. After washing once with TBST, magnetic beads were applied to each well of white round bottom PS plate (Corning, 3605) and 0.625 pmol of biotin labeled CD3 epsilon peptide, 2.5 pmol of biotin labeled human CD137-Fc, 2.5 pmol of biotin labeled cyno CD137-Fc or 0.625 pmol of biotin labeled human Fc was added to magnetic beads and incubated at room temperature for 15 minutes or more.

After washing once with TBST, 25 micro L each of the 50 ng/micro L purified IgG was added to the wells, and the plate was allowed to stand at room temperature for one hour to allow each IgG to bind to biotin-labeled antigen in each well. After that each well was washed with TBST. Goat anti-human kappa Light chain alkaline phosphatase conjugate (BETHYL, A80-115AP) diluted with TBS was added to each well. The plate was incubated for one hour. After washing with TBST, each sample were transferred to 96well plate (Corning, 3792 black round bottom PS plate) and APS-5 (Lumigen) was added to each well. 2 minutes later the fluorescence of each well was detected. The measurement results are shown in FIG. 13. Many clones showed equal level of binding to both human and cyno CD137 and also showed binding to CD3 epsilon.

3.9. Evaluation of Binding of IgG Having Obtained Fab Domain to CD3Epsilon and Human CD137 at Same Time

Thirty-seven antibodies which showed obvious binding to both CD3 epsilon, human CD137 and cyno CD137 in Reference Example 3.7 were selected to evaluate further. Seven antibodies obtained in Reference Example 2.3 were also evaluated (these 7 clones were renamed as in Table 7). Purified antibodies were subjected to ELISA to evaluate their binding capacity to CD3 epsilon and human CD137 at same time. Anti-human CD137 antibody named as B described in Reference Example 2.5 was used as control antibody.

TABLE 7

| Old name | New name |
|---|---|
| DXDU01_3_#094 | dBBDu121 |
| DXDU01_3_#072 | dBBDu122 |
| DADU01_3_#018 | dBBDu123 |
| DADU01_3_#002 | dBBDu124 |
| DXDU01_3_#019 | dBBDu125 |
| DADU01_3_#001 | dBBDu126 |
| DXDU01_3_#051 | dBBDu127 |

First, 20 micro g of Streptavidin-coated magnetic beads MyOne-T1 beads was washed three-times with blocking buffer including 0.4% block Ace, 1% BSA, 0.02% Tween and 0.05% ProClin 300 and then blocked with this blocking buffer at room temperature for 60 minutes or more. After washing once with TBST, magnetic beads were applied to each well of black round bottom PS plate (Corning, 3792). 1.25 pmol of biotin-labeled human CD137-Fc was added and incubated at room temperature for 10 minute. After that magnetic beads were washed by TBS once. 1250 ng of purified IgG was mixed with 125, 12.5 or 1.25 pmol of free CD3 epsilon peptide or TBS and then added to the magnetic beads in each well, and the plate was allowed to stand at room temperature for one hour to allow each IgG to bind to biotin-labeled antigen in each well. After that each well was washed with TBST. Goat anti-human kappa Light chain alkaline phosphatase conjugate (BETHYL, A80-115AP) diluted with TBS was added to each well. The plate was incubated for 10 minutes. After washing with TBST, APS-5 (Lumigen) was added to each well. 2 minutes later the fluorescence of each well was detected. The measurement results are shown in FIG. 14 and Table 8.

TABLE 8

| | biotin-human CD137-Fc | | |
|---|---|---|---|
| | free CD3e | | |
| | (pmol/well) | | Signal |
| | 0 | 125 | decrease |
| dBBDu133 | 16927 | 2373 | 85.98% |
| dBBDu139 | 9436 | 1924 | 79.61% |
| dBBDu140 | 19960 | 1923 | 90.37% |
| dBBDu142 | 13665 | 1786 | 86.93% |
| dBBDu149 | 3915 | 1962 | 49.89% |
| dBBDu165 | 75488 | 1954 | 97.41% |
| dBBDu167 | 25731 | 1937 | 92.47% |
| dBBDu171 | 7394 | 1819 | 75.40% |
| dBBDu172 | 7589 | 2241 | 70.47% |
| dBBDu173 | 6544 | 2041 | 68.81% |
| dBBDu178 | 6777 | 2126 | 68.63% |
| dBBDu179 | 61009 | 2625 | 95.70% |
| dBBDu181 | 3241 | 1990 | 38.60% |
| dBBDu182 | 9081 | 2178 | 76.02% |
| dBBDu183 | 34000 | 2369 | 93.03% |
| dBBDu184 | 16701 | 1888 | 88.70% |
| dBBDu186 | 34783 | 2497 | 92.82% |
| dBBDu189 | 27434 | 2193 | 92.01% |
| dBBDu191 | 12863 | 2230 | 82.66% |
| dBBDu193 | 18193 | 2278 | 87.48% |
| dBBDu195 | 9715 | 2361 | 75.70% |
| dBBDu196 | 33099 | 2222 | 93.29% |
| dBBDu197 | 54367 | 2111 | 96.12% |
| dBBDu199 | 40880 | 2372 | 94.20% |
| dBBDu202 | 12055 | 1930 | 83.99% |
| dBBDu204 | 43663 | 1879 | 95.70% |
| dBBDu205 | 45191 | 2194 | 95.15% |
| dBBDu206 | 6967 | 1697 | 75.64% |
| dBBDu207 | 7466 | 1844 | 75.30% |
| dBBDu209 | 12051 | 1779 | 85.24% |
| dBBDu211 | 7284 | 1732 | 76.22% |
| dBBDu214 | 12852 | 1701 | 86.76% |
| dBBDu217 | 19093 | 2416 | 87.35% |
| dBBDu222 | 7188 | 3236 | 54.98% |
| dBBDu166 | 3437 | 1844 | 46.35% |
| dBBDu174 | 4804 | 1884 | 60.78% |
| dBBDu175 | 3257 | 1755 | 46.12% |
| dBBDu121 | 3609 | 1826 | 49.40% |
| dBBDu122 | 2698 | 1882 | 30.24% |
| dBBDu123 | 2746 | 1840 | 32.99% |
| dBBDu124 | 6621 | 2116 | 68.04% |
| dBBDu125 | 61364 | 2058 | 96.65% |
| dBBDu126 | 116289 | 2613 | 97.75% |
| dBBDu127 | 3232 | 2198 | 31.99% |
| Du115/DUL008 | 86183 | 2620 | 96.96% |
| Du103/DUL050 | 5273 | 5297 | −0.46% |
| B | 99359 | 98110 | 1.26% |
| blank | 1860 | 1850 | 0.54% |

The binding to human CD137 of all tested clones except for control anti-CD137 antibody B was inhibited by excess amount of free CD3 epsilon peptide, it demonstrated that obtained antibodies with dual Fab library did not bind to CD3 epsilon and human CD137 at same time.

3.10. Evaluation of the Human CD137 Epitope of IgGs Having Obtained Fab Domain to CD3Epsilon and Human CD137

Twenty-one antibodies in Reference Example 3.8 were selected to evaluate further (Table 10). Purified antibodies were subjected to ELISA to evaluate their binding epitope of human CD137.

To analyze the epitope, a fusion protein of the fragmented human CD137 and the Fc region of an antibody that domain divided by the structure formed by Cys-Cys called CRD reference (Table 9) as described in WO2015/156268. Fragmented human CD137-Fc fusion protein to include the amino acid sequence shown in Table 9, the respective gene fragments by PCR from a polynucleotide encoding the full-length human CD137-Fc fusion protein (SEQ ID NO: 90) were incorporated into a plasmid vector for expression in animal cells by methods known to those skilled in the art. Fragmented human CD137-Fc fusion protein was purified as an antibody by the method described in WO2015/156268.

TABLE 9

| Name of the | | Domains | |
|---|---|---|---|
| fragmented | Amino acid sequence of the | that are | SEQ ID |
| human CD137 | fragmented human CD137 | included | NO |
| Full length | LQDPCSNCPAGTFCDNNRNQICSPCPPNSFSSA | CRD1, 2, | 90 |
| | GGQRTCDICRQCKGVFRTRKECSSTSNAECDCT | 3, 4 | |
| | PGFHCLGAGCSMCEQDCKQGQELTKKGCKDCCA | | |
| | FGTFNDQKRGICRPWTNCSLDGKSVLVNGTKER | | |
| | DVVCGPSPADLSPGASSVTPPAPAREPGHSPQ | | |
| CRD1 | LQDPCSNCPAGTFCDNNRNQIC | CRD1 | 147 |
| CRD2 | SPCPPNSFSSAGGQRTCDICRQCKGVFRTRKEC | CRD2 | 148 |
| | SSTSNAEC | | |
| CRD3 | DCTPGFHCLGAGCSMCEQDCKQGQELTKKGC | CRD3 | 149 |
| CRD4 | KDCCFGTFNDQKRGICRPWTNCSLDGKSVLVNG | CRD4 | 150 |
| | TKERDVVCGPSPADLSPGASSVTPPAPAREPGH | | |
| | SPQ | | |
| CRD1-3 | LQDPCSNCPAGTFCDNNRNQICSPCPPNSFSSA | CRD1, 2, | 151 |
| | GGQRTCDICRQCKGVFRTRKECSSTSNAECDCT | 3 | |
| | PGFHCLGAGCSMCEQDCKQGQELTKKGC | | |
| CRD1-2 | LQDPCSNCPAGTFCDNNRNQICSPCPPNSFSSA | CRD1, 2 | 152 |
| | GGQRTCDICRQCKGVFRTRKECSSTSNAEC | | |
| CRD2-4 | SPCPPNSFSSAGGQRTCDICRQCKGVFRTRKEC | CRD2, 3, | 153 |
| | SSTSNAECDCTPGFHCLGAGCSMCEQDCKQGQE | 4 | |
| | LTKKGCKDCCFGTFNDQKRGICRPWTNCSLDGK | | |
| | SVLVNGTKERDVVCGPSPADLSPGASSVTPPAP | | |
| | AREPGHSPQ | | |
| CRD2-3 | SPCPPNSFSSAGGQRTCDICRQCKGVFRTRKEC | CRD2, 3 | 154 |
| | SSTSNAECDCTPGFHCLGAGCSMCEQDCKQGQE | | |
| | LTKKGC | | |
| CRD3-4 | DCTPGFHCLGAGCSMCEQDCKQGQELTKKGCKD | CRD3, 4 | 155 |
| | CCFGTFNDQKRGICRPWTNCSLDGKSVLVNGTK | | |
| | ERDVVCGPSPADLSPGASSVTPPAPAREPGHSP | | |
| | Q | | |

First, 20 micro g of Streptavidin-coated magnetic beads MyOne-T1 beads was washed three-times with blocking buffer including 0.4% block Ace, 1% BSA, 0.02% Tween and 0.05% ProClin 300 and then blocked with this blocking buffer at room temperature for 60 minutes or more. After washing once with TBST, magnetic beads were applied to each well of black round bottom PS plate (Corning, 3792). 1.25 pmol of biotin-labeled human CD137-Fc, human CD137 domain1-Fc, human CD137 domain1/2-Fc, human CD137 domain2/3-Fc, human CD137 domain2/3/4-Fc, human CD137 domain3/4-Fc and human Fc was added and incubated at room temperature for 10 minute. After that magnetic beads were washed by TBS once. 1250 ng of purified IgG was added to the magnetic beads in each well, and the plate was allowed to stand at room temperature for one hour to allow each IgG to bind to biotin-labeled antigen in each well. After that each well was washed with TBST. Goat anti-human kappa Light chain alkaline phosphatase conjugate (BETHYL, A80-115AP) diluted with TBS was added to each well. The plate was incubated for 10 minutes. After washing with TBST, APS-5 (Lumigen) was added to each well. 2 minutes later the fluorescence of each well was detected. The measurement results are shown in FIG. 15.

Each clones recognized different epitope domain of human CD137. Antibodies which recognize only domain1/2 (e.g. dBBDu183, dBBDu205), both domain1/2 and domain2/3 (e.g. dBBDu193, dBBDu_202, dBBDu222), both domain2/3, 2/3/4 and 3/4 (e.g. dBBDu139, dBBDu217), broadly human CD137 domains (dBBDu174) and which do not bind to each separated human CD137 domains (e.g. dBBDu126). This result demonstrates many dual binding antibodies to several human CD137 epitopes can be obtained with this designed library and double round selection procedure.

The CD137-binding epitope region of dBBDu126 cannot be decided by this ELISA assay, but it can be guessed that it will recognize position(s) in which human and cynomolgus monkey have different residues because dBBDu126 cannot cross-react with cyno CD137 as described in Reference Example 2.3. As shown in FIG. 7, there are 8 different position between human and cyno, and 75E (75G in human) was identified as occasion which interfere the binding of dBBDu126 to cyno CD137 by the binding assay to cyno CD137/human CD137 hybrid molecules and the crystal structure analysis of binding complex. Crystal structure also reveal dBBDu126 mainly recognize CRD3 region of human CD137.

TABLE 10

| Clone name | SEQ ID NO |
|---|---|
| dBBDu126 | 102 |
| dBBDu183 | 104 |
| dBBDu179 | 105 |
| dBBDu196 | 106 |
| dBBDu197 | 107 |
| dBBDu199 | 108 |
| dBBDu204 | 109 |
| dBBDu205 | 110 |
| dBBDu193 | 111 |
| dBBDu217 | 112 |
| dBBDu139 | 113 |
| dBBDu189 | 114 |
| dBBDu167 | 115 |
| dBBDu173 | 116 |
| dBBDu174 | 117 |
| dBBDu181 | 118 |
| dBBDu186 | 119 |
| dBBDu191 | 120 |
| dBBDu202 | 121 |
| dBBDu222 | 122 |
| dBBDu125 | 101 |

### [Reference Example 4] Affinity Maturation of Antibody Domain Binding to CD3 Epsilon and Human CD137 from Dual Fab Library with Designed Light Chain Library

4.1. Construction of Light Chain Library with Obtained Heavy Chain

Many antibodies which bind to both CD3 epsilon and human CD137 were obtained in Reference Example 3, but their affinity to human CD137 were still weak so affinity maturation to improve their affinity was conducted.

Thirteen VH sequences, dBBDu_179, 183, 196, 197, 199, 204, 205, 167, 186, 189, 191, 193 and 222 were selected for affinity maturation. In those, dBBDu_179, 183, 196, 197, 199, 204 and 205 have same CDR3 sequence and different CDR1 or 2 sequences so these 7 phagemids were mixed to produce Light chain Fab library. dBBDu_191, 193 and 222 three phagemids were also mixed to produce Light chain Fab library although they had different CDR3 sequences. The list of light chain library was shown in Table 11.

TABLE 11

| Library name | VH |
|---|---|
| Library 2 | dBBDu_179, 183, 196, 197, 199, 204, 205 |
| Library 3 | dBBDu_167 |
| Library 4 | dBBDu_186 |
| Library 5 | dBBDu_189 |
| Library 6 | dBBDu_191, 193, 222 |

The synthesized antibody VL library fragments described in Reference Example 12 were amplified by PCR method with the primers of SEQ ID NO: 198 and 199. Amplified VL fragments were digested by SfiI and KpnI restriction enzyme and introduced into phagemid vectors which had each thirteen VH fragments. The constructed phagemids for phage display were transferred to E. coli by electroporation to prepare E. coli harboring the antibody library fragments.

Phage library displaying Fab domain were produced from the E. coli harboring the constructed phagemids by infection of helper phage M13KO7TC/FkpA which code FkpA chaperone gene and then incubation with 0.002% arabinose at 25 degrees Celsius for overnight. M13KO7TC is a helper phage which has an insert of the trypsin cleavage sequence between the N2 domain and the CT domain of the pIII protein on the helper phage (see Japanese Patent Application Kohyo Publication No. 2002-514413). Introduction of insert gene into M13KO7TC gene have been already disclosed elsewhere (see WO2015/046554).

4.2. Obtainment of Fab Domain Binding to CD3Epsilon and Human CD137 with Double Round Selection

Fab domains binding to CD3 epsilon, human CD137 and cyno CD137 were identified from the dual Fab library constructed in Reference Example 4.1. CD3 epsilon peptide antigen biotin-labeled through disulfide-bond linker(C3 NP1-27), biotin-labeled human CD137 fused to human IgG1 Fc fragment (named as human CD137-Fc) and biotin-labeled cynomolgus monkey CD137 fused to human IgG1 Fc fragment (named as cyno CD137-Fc) was used as an antigen.

Phages were produced from the E. coli harboring the constructed phagemids for phage display. 2.5 M NaCl/10% PEG was added to the culture solution of the E. coli that had produced phages, and a pool of the phages thus precipitated was diluted with TBS to obtain a phage library solution. Next, BSA (final concentration: 4%) was added to the phage library solution. The panning method was performed with reference to a general panning method using antigens immobilized on magnetic beads (J. Immunol. Methods. (2008) 332 (1-2), 2-9; J. Immunol. Methods. (2001) 247 (1-2), 191-203; Biotechnol. Prog. (2002) 18 (2) 212-20; and Mol. Cell Proteomics (2003) 2 (2), 61-9).

The magnetic beads used were NeutrAvidin coated beads (Sera-Mag SpeedBeads NeutrAvidin-coated) or Streptavidin coated beads (Dynabeads M-280 Streptavidin).

Specifically, Phage solution was mixed with 100 pmol of human CD137-Fc and 4 nmol of free human IgG1 Fc domain and incubated at room temperature for 60 minutes. Magnetic beads was blocked by 2% skim-milk/TBS with free Streptavidin (Roche) at room temperature for 60 minutes or more and washed three times with TBS, and then mixed with incubated phage solution. After incubation at room temperature for 15 minutes, the beads were washed three-times with TBST (TBS containing 0.1% Tween 20; TBS was available from Takara Bio Inc.) for 10 minutes and then further washed twice with 1 mL of TBS for 10 minutes. FabRICATOR(IdeS, protease for hinge region of IgG, GENOVIS)(named as IdeS elution campaign) was used to recover antibody displaying phages.

In that procedure, 10 units/micro L Fabricator 20 micro L with 80 micro L TBS buffer was added and beads were suspended at 37 degrees Celsius for 30 minutes, immediately after which the beads were separated using a magnetic stand to recover phage solution. 5 micro L of 100 mg/mL Trypsin and 400 micro L of TBS were added and incubated at room temperature for 15 minutes. The recovered phage solution was added to an E. coli strain ER2738 in a logarithmic growth phase (0D600: 0.4-0.5). The E. coli strain was infected by the phages through the gentle spinner culture of the strain at 37 degrees C. for 1 hour. The infected E. coli was inoculated to a plate of 225 mm×225 mm. Next, phages were recovered from the culture solution of the inoculated E. coli to prepare a phage library solution.

In this panning round1 procedure antibody displaying phages which bind to human CD137 was concentrated. In the 2^{nd} round of panning, 160 pmol of C3 NP1-27 was used as biotin-labeled antigen and wash was conducted seven-times with TBST for 2 minutes and then three-times with TBS for 2 minutes. Elution was conducted with 25 mM DTT at room temperature for 15 minutes and then digested by Trypsin.

In the 3^{rd} round of panning, 16 or 80 pmol of biotin-labeled cyno CD137-Fc were used as antigen and wash was conducted seven-times with TBST for 10 minutes and then three-times with TBS for 10 minutes. Elution was conducted with IdeS as same as round1.

In the 4^{th} round of panning, 16 or 80 pmol of biotin labeled human CD137-Fc were used as antigen and wash was conducted seven-times with TBST for 10 minutes and then three-times with TBS for 10 minutes. Elution was conducted with IdeS as same as round1.

4.3. Binding of IgG Having Obtained Fab Domain to Human CD137 and Cyno CD137

Fab genes of each panning output pools were converted into IgG format. The prepared mammalian expression plasmids were introduced into E. coli and 96 colonies were picked from each panning output pools and their VH and VL sequence were analyzed. Most of VH sequence in Library 2 had concentrated to dBBDu_183 and most of VH sequence in Library 6 had concentrated to dBBDu_193, respectively. The prepared plasmids from each E. coli colonies were used for expression in animal cells by the method of Reference Example 9.

The prepared IgG antibodies were subjected to ELISA to evaluate their binding capacity to CD3 epsilon, human CD137 and cyno CD137.

First, a Streptavidin-coated microplate (384 well, Greiner) was coated with 20 micro L of TBS containing biotin-labeled CD3 epsilon peptide, biotin labeled human CD137-Fc or biotin labeled cyno CD137-Fc at room temperature for one or more hours. After removing biotin-labeled antigen that are not bound to the plate by washing each well of the plate with TBST, the wells were blocked with 20 micro L of Blocking Buffer (2% skim milk/TBS) for one or more hours. Blocking Buffer was removed from each well. 20 micro L each of the 10 ng/micro L IgG containing mammalian cell supernatant twice diluted with 1% Skim milk/TBS were added to the wells, and the plate was allowed to stand at room temperature for one hour to allow each IgG to bind to biotin-labeled antigen in each well. After that each well was washed with TBST. Goat anti-human kappa Light chain alkaline phosphatase conjugate (BETHYL, A80-115AP) diluted with TBS was added to each well. The plate was incubated for one hour. After washing with TBST, the chromogenic reaction of the solution in each well added with Blue Phos Microwell Phosphatase Substrate System (KPL) was terminated by adding Blue Phos Stop Solution (KPL). Then, the color development was measured by absorbance at 615 nm. The measurement results are shown in FIG. 16.

Many IgG clones which showed binding to both CD3 epsilon, human CD137 and cyno CD137 were obtained from each panning procedure. Ninety-six clones which showed better binding were selected and evaluated further.

4.4. Evaluation of Binding of IgG Having Obtained Fab Domain to CD3Epsilon and Human CD137 at Same Time

Ninety-six antibodies which showed obvious binding to both CD3 epsilon, human CD137 and cyno CD137 in Reference Example 4.3 were selected to evaluate further. Purified antibodies were subjected to ELISA to evaluate their binding capacity to CD3 epsilon and human CD137 at same time.

First, 20 micro g of Streptavidin-coated magnetic beads MyOne-T1 beads was washed three-times with blocking buffer including 0.5× block Ace, 0.02% Tween and 0.05% ProClin 300 and then blocked with this blocking buffer at room temperature for 60 minutes or more. After washing once with TBST, magnetic beads were applied to each well of black round bottom PS plate (Corning, 3792). 0.625 pmol of biotin-labeled human CD137-Fc was added and incubated at room temperature for 10 minute.

After that magnetic beads were washed by TBS once. 250 ng of purified IgG was mixed with 62.5, 6.25 or 0.625 pmol of free CD3 epsilon or 62.5 pmol of free human IgG1 Fc domain and then added to the magnetic beads in each well, and the plate was allowed to stand at room temperature for one hour to allow each IgG to bind to biotin-labeled antigen in each well. After that each well was washed with TBST. Goat anti-human kappa Light chain alkaline phosphatase conjugate (BETHYL, A80-115AP) diluted with TBS was added to each well. The plate was incubated for 10 minutes. After washing with TBST, APS-5 (Lumigen) was added to each well. 2 minutes later the fluorescence of each well was detected. The measurement results are shown in FIG. 17 and Table 12. The binding to human CD137 of most tested clones was inhibited by excess amount of free CD3 epsilon peptide, it demonstrated that obtained antibodies with dual Fab library did not bind to CD3 epsilon and human CD137 at same time.

TABLE 12

| | biotin-human CD137-Fc | | |
|---|---|---|---|
| | Free CD3e | Free Fc | Signal |
| | 62.5 pmol | 62.5 pmol | decrease |
| dBBDu183/L057 | 2732 | 9025 | 69.73% |
| dBBDu183/L058 | 2225 | 11115 | 79.98% |
| dBBDu183/L059 | 2134 | 100126 | 97.87% |
| dBBDu183/L060 | 2169 | 37723 | 94.25% |
| dBBDu183/L061 | 2118 | 2723 | 22.22% |
| dBBDu183/L062 | 2777 | 27880 | 90.04% |
| dBBDu183/L063 | 2943 | 28858 | 89.80% |
| dBBDu183/L064 | 2206 | 13474 | 83.63% |
| dBBDu183/L065 | 2725 | 6024 | 54.76% |
| dBBDu183/L066 | 2325 | 34020 | 93.17% |
| dBBDu183/L067 | 2936 | 19722 | 85.11% |
| dBBDu197/L068 | 2786 | 105219 | 97.35% |
| dBBDu183/L069 | 2463 | 31769 | 92.25% |
| dBBDu183/L070 | 3267 | 92395 | 96.46% |
| dBBDu183/L071 | 2297 | 8670 | 73.51% |
| dBBDu183/L072 | 2840 | 54764 | 94.81% |
| dBBDu183/L073 | 2876 | 6724 | 57.23% |
| dBBDu196/L074 | 2724 | 12891 | 78.87% |
| dBBDu183/L075 | 2568 | 8029 | 68.02% |
| dBBDu196/L076 | 2188 | 5037 | 56.56% |
| dBBDu179/L077 | 3147 | 8018 | 60.75% |
| dBBDu167/L078 | 2378 | 27120 | 91.23% |
| dBBDu167/L079 | 2269 | 5869 | 61.34% |
| dBBDu167/L080 | 2236 | 95870 | 97.67% |
| dBBDu167/L081 | 2508 | 44240 | 94.33% |
| dBBDu167/L082 | 2398 | 177750 | 98.65% |
| dBBDu167/L083 | 2164 | 78935 | 97.26% |
| dBBDu167/L084 | 2182 | 18392 | 88.14% |
| dBBDu167/L085 | 2202 | 8724 | 74.76% |
| dBBDu167/L086 | 2627 | 135762 | 98.06% |
| dBBDu167/L087 | 2168 | 106703 | 97.97% |
| dBBDu167/L088 | 2040 | 2163 | 5.69% |
| dBBDu167/L089 | 2424 | 10161 | 76.14% |
| dBBDu167/L090 | 2595 | 181795 | 98.57% |
| dBBDu167/L091 | 11345 | 124409 | 90.88% |
| dBBDu167/L092 | 2924 | 123122 | 97.63% |
| dBBDu167/L093 | 4934 | 139388 | 96.46% |
| dBBDu167/L094 | 4374 | 140938 | 96.90% |
| dBBDu167/L095 | 2207 | 112225 | 98.03% |
| dBBDu186/L096 | 37273 | 84887 | 56.09% |
| dBBDu186/L097 | 9006 | 114399 | 92.13% |
| dBBDu186/L098 | 15908 | 114905 | 86.16% |
| dBBDu186/L099 | 2367 | 19583 | 87.91% |
| dBBDu186/L100 | 88856 | 102097 | 12.97% |
| dBBDu186/L101 | 2340 | 37392 | 93.74% |
| dBBDu186/L102 | 2427 | 2685 | 9.61% |
| dBBDu186/L103 | 21977 | 74203 | 70.38% |
| dBBDu186/L104 | 2165 | 2145 | −0.93% |
| dBBDu186/L105 | 13426 | 89231 | 84.95% |
| dBBDu186/L106 | 3088 | 9857 | 68.67% |
| dBBDu186/L107 | 2104 | 2047 | −2.78% |
| dBBDu186/L108 | 50796 | 83558 | 39.21% |
| dBBDu189/L109 | 3000 | 76770 | 96.09% |
| dBBDu189/L110 | 3836 | 119618 | 96.79% |
| dBBDu189/L111 | 2568 | 49623 | 94.82% |
| dBBDu189/L112 | 4768 | 91051 | 94.76% |
| dBBDu189/L113 | 3357 | 89648 | 96.26% |
| dBBDu189/L114 | 2158 | 2512 | 14.09% |
| dBBDu189/L115 | 4058 | 141183 | 97.13% |
| dBBDu189/L116 | 3149 | 109316 | 97.12% |
| dBBDu189/L117 | 2625 | 102489 | 97.44% |
| dBBDu189/L118 | 2446 | 19372 | 87.37% |
| dBBDu189/L119 | 20377 | 88058 | 76.86% |
| dBBDu189/L120 | 3778 | 113755 | 96.68% |
| dBBDu189/L121 | 3300 | 37197 | 91.13% |
| dBBDu189/L122 | 3949 | 141349 | 97.21% |
| dBBDu189/L123 | 4950 | 22574 | 78.07% |
| dBBDu189/L124 | 3282 | 111075 | 97.05% |
| dBBDu189/L125 | 6494 | 121498 | 94.66% |
| dBBDu189/L126 | 9750 | 75082 | 87.01% |
| dBBDu193/L127 | 2471 | 6084 | 59.39% |
| dBBDu193/L128 | 3197 | 120777 | 97.35% |
| dBBDu193/L129 | 2773 | 5310 | 47.78% |
| dBBDu193/L130 | 3055 | 124130 | 97.54% |
| dBBDu193/L131 | 15481 | 109233 | 85.83% |
| dBBDu193/L132 | 10414 | 115982 | 91.02% |
| dBBDu193/L133 | 2388 | 33076 | 92.78% |
| dBBDu193/L134 | 3046 | 109154 | 97.21% |
| dBBDu193/L135 | 2284 | 54304 | 95.79% |
| dBBDu193/L136 | 2092 | 113254 | 98.15% |
| dBBDu193/L137 | 2458 | 6602 | 62.77% |
| dBBDu193/L138 | 8165 | 100690 | 91.89% |
| dBBDu193/L139 | 2077 | 2190 | 5.16% |
| dBBDu222/L140 | 2721 | 22972 | 88.16% |
| dBBDu193/L141 | 2166 | 5582 | 61.20% |
| dBBDu193/L142 | 12085 | 103522 | 88.33% |
| dBBDu193/L143 | 2338 | 50082 | 95.33% |
| dBBDu193/L144 | 1952 | 2366 | 17.50% |
| dBBDu193/L145 | 2739 | 2820 | 2.87% |

4.5. Evaluation of Affinity of IgG Having Obtained Fab Domain to CD3Epsilon, Human CD137 and Cyno CD137

The binding of each IgG obtained in the Reference Example 4.4 to human CD3 ed, human CD137 and cyno CD137 was confirmed using Biacore T200. Sixteen antibodies were selected by the results in Reference Example 4.4. Sensor chip CM3 (GE Healthcare) was immobilized with an appropriate amount of sure protein A (GE Healthcare) by amine coupling. The selected antibodies were captured by the chip to allow interaction to human CD3 ed, human CD137 and cyno CD137 as an antigen. The running buffer used was 20 mmol/1 ACES, 150 mmol/1 NaCl, 0.05% (w/v) Tween20, pH 7.4. All measurements were carried out at 25 degrees C. The antigens were diluted using the running buffer.

Regarding human CD137, the selected antibodies were assessed for its binding at antigen concentrations of 4000, 1000, 250, 62.5, and 15.6 nM. Diluted antigen solutions and the running buffer which is the blank were loaded at a flow rate of 30 micro L/min for 180 seconds to allow each concentration of the antigen to interact with the antibody captured on the sensor chip. Then, running buffer was run at a flow rate of 30 micro L/min for 300 seconds and dissociation of the antigen from the antibody was observed. Next, to regenerate the sensor chip, 10 mmol/L glycine-HCl, pH 1.5 was loaded at a flow rate of 30 micro L/min for 10 seconds and 50 mmol/L NaOH was loaded at a flow rate 30 micro L/min for 10 seconds.

Regarding cyno CD137, the selected antibodies were assessed for its binding at antigen concentrations of 4000, 1000 and 250 nM. Diluted antigen solutions and the running buffer which is the blank were loaded at a flow rate of 30 micro L/min for 180 seconds to allow each of the antigens to interact with the antibody captured on the sensor chip. Then, running buffer was run at a flow rate of 30 micro L/min for 300 seconds and dissociation of the antigen from the antibody was observed. Next, to regenerate the sensor chip, 10 mmol/L glycine-HCl, pH 1.5 was loaded at a flow rate of 30 micro L/min for 10 seconds and 50 mmol/L NaOH was loaded at a flow rate 30 micro L/min for 10 seconds.

Regarding human CD3 ed, the selected antibodies were assessed for its binding at antigen concentrations of 1000, 250, and 62.5 nM. Diluted antigen solutions and the running buffer which is the blank were loaded at a flow rate of 30 micro L/min for 120 seconds to allow each of the antigens to interact with the antibody captured on the sensor chip. Then, running buffer was run at a flow rate of 30 micro L/min for 180 seconds and dissociation of the antigen from the antibody was observed. Next, to regenerate the sensor chip, 10 mmol/L glycine-HCl, pH 1.5 was loaded at a flow rate of 30 micro L/min for 30 seconds and 50 mmol/L NaOH was loaded at a flow rate 30 micro L/min for 30 seconds.

Kinetic parameters such as the association rate constant ka (1/Ms) and the dissociation rate constant kd (1/s) were calculated based on the sensorgrams obtained by the measurements. The dissociation constant KD (M) was calculated from these constants. Each parameter was calculated using the Biacore T200 Evaluation Software (GE Healthcare). The results are shown in Table 13.

TABLE 13

| Hch | | SEQ ID | human CD137 | | |
|---|---|---|---|---|---|
| Name | Lch name | NO | ka (1/Ms) | kd (1/s) | KD (M) |
| dBBDu_183 | dBBDu_L063 | 123 | 2.05E+03 | 3.58E−03 | 1.74E−06 |
| dBBDu_183 | dBBDu_L072 | 124 | 1.76E+03 | 4.25E−03 | 2.41E−06 |
| dBBDu_167 | dBBDu_L091 | 125 | 2.72E+03 | 1.85E−02 | 6.79E−06 |
| dBBDu_186 | dBBDu_L096 | 126 | 2.46E+02 | 5.58E−04 | 2.27E−06 |
| dBBDu_186 | dBBDu_L098 | 127 | 2.31E+02 | 5.34E−04 | 2.31E−06 |
| dBBDu_186 | dBBDu_L106 | 128 | 1.30E+02 | 4.47E−04 | 3.44E−06 |
| dBBDu_189 | dBBDu_L116 | 129 | 7.07E+02 | 2.91E−03 | 4.12E−06 |
| dBBDu_189 | dBBDu_L119 | 130 | 1.48E+02 | 4.02E−04 | 2.71E−06 |
| dBBDu_183 | dBBDu_L067 | 131 | 1.38E+03 | 4.51E−03 | 3.26E−06 |
| dBBDu_186 | dBBDu_L100 | 132 | 3.91E+02 | 7.46E−04 | 1.91E−06 |
| dBBDu_186 | dBBDu_L108 | 133 | 3.35E+02 | 8.10E−04 | 2.41E−06 |
| dBBDu_189 | dBBDu_L112 | 134 | 1.18E+03 | 3.13E−03 | 2.66E−06 |
| dBBDu_189 | dBBDu_L126 | 135 | 1.34E+03 | 6.88E−04 | 5.13E−07 |
| dBBDu_167 | dBBDu.L094 | 136 | 1.21E+03 | 1.02E−02 | 8.43E−06 |
| dBBDu_193 | dBBDu.L127 | 137 | 4.40E+02 | 1.45E−03 | 3.30E−06 |
| dBBDu_193 | dBBDu.L132 | 138 | 4.71E+02 | 2.11E−03 | 4.48E−06 |
| | Lch | SEQ ID | cyno CD137 | | |
| Hch Name | name | NO | ka (1/Ms) | kd (1/s) | KD (M) |
| dBBDu_183 | dBBDu_L063 | 123 | 1.47E+03 | 4.57E−03 | 3.12E−06 |
| dBBDu_183 | dBBDu_L072 | 124 | 1.22E+03 | 5.93E−03 | 4.87E−06 |
| dBBDu_167 | dBBDu_L091 | 125 | 2.43E+03 | 1.01E−02 | 4.17E−06 |
| dBBDu_186 | dBBDu_L096 | 126 | 1.09E+01 | 2.23E−03 | 2.05E−04 |
| dBBDu_186 | dBBDu_L098 | 127 | 8.84E+00 | 1.19E−03 | 1.34E−04 |
| dBBDu_186 | dBBDu_L106 | 128 | 2.05E+01 | 1.26E−03 | 6.13E−05 |
| dBBDu_189 | dBBDu_L116 | 129 | 7.44E+02 | 8.23E−03 | 1.11E−05 |
| dBBDu_189 | dBBDu_L119 | 130 | 3.42E+01 | 1.22E−03 | 3.57E−05 |
| dBBDu_183 | dBBDu_L067 | 131 | 1.31E+03 | 8.13E−03 | 6.20E−06 |
| dBBDu_186 | dBBDu_L100 | 132 | 2.95E+01 | 2.08E−03 | 7.04E−05 |
| dBBDu_186 | dBBDu_L108 | 133 | 2.25E+02 | 3.61E−03 | 1.61E−05 |
| dBBDu_189 | dBBDu_L112 | 134 | 4.98E+03 | 2.86E−02 | 5.76E−06 |
| dBBDu_189 | dBBDu_L126 | 135 | 8.07E+02 | 2.47E−03 | 3.06E−06 |
| dBBDu_167 | dBBDu.L094 | 136 | 1.08E+04 | 7.48E−02 | 6.92E−06 |
| dBBDu_193 | dBBDu.L127 | 137 | 1.12E+02 | 3.16E−03 | 2.81E−05 |
| dBBDu_193 | dBBDu.L132 | 138 | 8.06E+00 | 6.10E−03 | 7.57E−04 |
| | | SEQ ID | human GD3ed | | |
| Hch Name | Lch name | NO | ka (1/Ms) | kd (1/s) | KD (M) |
| dBBDu_183 | dBBDu_L063 | 123 | 5.69E+04 | 1.57E−02 | 2.76E−07 |
| dBBDu_183 | dBBDu_L072 | 124 | 3.61E+04 | 7.85E−03 | 2.17E−07 |
| dBBDu_167 | dBBDu_L091 | 125 | 5.24E+04 | 2.16E−02 | 4.13E−07 |
| dBBDu_186 | dBBDu_L096 | 126 | 1.12E+04 | 1.02E−01 | 9.11E−06 |
| dBBDu_186 | dBBDu_L098 | 127 | 1.11E+04 | 2.09E−02 | 1.88E−06 |
| dBBDu_186 | dBBDu_L106 | 128 | 1.03E+04 | 3.18E−02 | 3.09E−06 |
| dBBDu_189 | dBBDu_L116 | 129 | 2.08E+04 | 4.34E−03 | 2.09E−07 |
| dBBDu_189 | dBBDu_L119 | 130 | 1.25E+04 | 2.58E−02 | 2.06E−06 |
| dBBDu_183 | dBBDu_L067 | 131 | 8.89E+04 | 1.93E−02 | 2.17E−07 |
| dBBDu_186 | dBBDu_L100 | 132 | 1.62E+04 | 5.46E−02 | 3.36E−06 |
| dBBDu_186 | dBBDu_L108 | 133 | 1.36E+04 | 4.08E−02 | 3.01E−06 |
| dBBDu_189 | dBBDu_L112 | 134 | 3.03E+04 | 1.00E−02 | 3.31E−07 |
| dBBDu_189 | dBBDu_L126 | 135 | 1.09E+04 | 2.81E−02 | 2.57E−06 |
| dBBDu_167 | dBBDu.L094 | 136 | 6.02E+04 | 2.10E−02 | 3.49E−07 |
| dBBDu_193 | dBBDu.L127 | 137 | 1.26E+04 | 1.91E−02 | 1.51E−06 |
| dBBDu_193 | dBBDu.L132 | 138 | 9.89E+03 | 2.01E−02 | 2.03E−06 |

### [Reference Example 5] Preparation of Anti-Human GPC3/Dual-Fab Trispecific Antibodies and Assessment of their Human CD137 Agonist Activities

5.1. Preparation of Anti-Human GPC3/Anti-Human CD137 Bispecific Antibodies and Anti-Human GPC3/Dual-Fab Trispecific Antibodies

The anti-human GPC3/anti-human CD137 bispecific antibodies and the anti-human GPC3/Dual-Fab Trispecific antibodies carrying human IgG1 constant regions were produced by the following procedure. Genes encoding an anti-human CD137 antibody (SEQ ID NO: 93 for the H chain, and SEQ ID NO: 94 for the L chain) described in WO2005/035584A1 (abbreviated as B) was used as a control antibody. The anti-human GPC3 side of the antibodies shared the heavy-chain variable region H0000 (SEQ ID NO: 139) and light-chain variable region GL4 (SEQ ID NO: 140).

Sixteen dual-Ig Fab described in Reference Example 4 and Table 13 was used as candidate dual-Ig antibody. For these molecules, the CrossMab technique reported by Schaefer et al. (Schaefer, Proc. Natl. Acad. Sci., 2011, 108, 11187-11192) was used to regulate the association between the H and L chains and efficiently obtain the bispecific antibodies. More specifically, these molecules were produced by exchanging the VH and VL domains of Fab against human GPC3. For promotion of heterologous association, the Knobs-into-Holes technology was used for the constant region of the antibody H chain. The Knobs-into-Holes technology is a technique that enables preparation of heterodimerized antibodies of interest through promotion of the heterodimerization of H chains by substituting an amino acid side chain present in the CH3 region of one of the H chains with a larger side chain (Knob) and substituting an amino acid side chain in the CH3 region of the other H chain with a smaller side chains (Hole) so that the knob will be placed into the hole (Burmeister, Nature, 1994, 372, 379-383).

Hereinafter, the constant region into which the Knob modification has been introduced will be indicated as Kn, and the constant region into which the Hole modification has been introduced will be indicated as H1. Furthermore, the modifications described in WO2011/108714 were used to reduce the Fc gamma binding. Specifically, modifications of substituting Ala for the amino acids at positions 234, 235, and 297 (EU numbering) were introduced. Gly at position 446 and Lys at position 447 (EU numbering) were removed from the C termini of the antibody H chains. A histidine tag was added to the C terminus of the Kn Fc region, and a FLAG tag was added to the C terminus of H1 Fc region. The anti-human GPC3 H chains prepared by introducing the above-mentioned modifications were GC33(2)H-G1dKnHS (SEQ ID NO: 141). The anti-human CD137 H chains prepared were BVH-G1dHIFS(SEQ ID NO: 142). The antibody L chains GC33(2)L-k0 (SEQ ID NO: 143) and BVL-k0 (SEQ ID NO: 144) were commonly used on the anti-human GPC3 side and the anti-CD137 side, respectively. The H chains and L chains of Dual antibodies are also shown in Table 13. The VH of each dual antibody clones were fused to G1dHIFS (SEQ ID NO: 156) CH region and the VL of each dual antibody clones were fused to k0 (SEQ ID NO: 157) CL region, respectively, as same as BVH-G1dHIFS and BVL-k0. The antibodies having the combinations shown in Table 15 were expressed to obtain the bispecific antibodies of interest. An antibody having received irrelevant was used as control (abbreviated as Ctrl). These antibodies were expressed by transient expression in FreeStyle293 cells (Invitrogen) and purified according to “Reference Example 9”.

5.2. Assessment of the In Vitro GPC3-Dependent CD137 Agonist Effect of Anti-Human GPC3/Dual-Fab Trispecific Antibodies

The agonistic activity for human CD137 was evaluated on the basis of the cytokine production using ELISA kit (R&D systems, DY206). In order to avoid the effect of CD3 epsilon binding domain of the anti-human GPC3/Dual-Fab antibodies, the B cell strain HDLM-2 was used, which did not express the CD3 epsilon neither GPC3, but express CD137 constitutively. The HDLM-2 was suspended in 20% FBS-containing RPMI-1640 medium at a density of 8×10⁵ cells/ml. The mouse cancer cell strain CT26-GPC3 which expressed GPC3 (Reference Example 13) was suspended in the same medium at a density of 4×10⁵ cells/ml. The same volume of each cell suspension was mixed, the mixed cell suspension was seeded into the 96-well plate at a volume of 200 micro 1/well. The anti-GPC3/Ctrl antibodies, the anti-GPC3/anti-CD137 antibodies, and eight anti-GPC3/Dual-Fab antibodies prepared in Reference Example 5.1 were added at 30 micro g/ml, 6 micro g/ml, 1.2 micro g/ml, 0.24 micro g/ml each. The cells were cultured under the condition of 37 degrees C. and 5% CO2 for 3 days. The culture supernatant was collected, and the concentration of human IL-6 contained in the supernatant was measured with Human IL-6 DuoSet ELISA (R&D systems, DY206) to assess the HDLM-2 activation. ELISA was performed by following the instructions provided by the kit manufacturer (R&D systems).

As a result (FIG. 18 and Table 14), seven of eight anti-GPC3/Dual-Fab antibodies showed the activation of IL-6 production of HDLM-2 as well as antiGPC3/anti-CD137 antibodies depending on antibody concentration. In Table 14, agonistic activity compared to Ctrl means the increase level of hIL-6 secretion beyond the background level in the presence of Ctrl. Based on this result, it was thought that these Dual-Fab antibodies have the agonistic activity on human CD137.

TABLE 14

| | | | Agonistic activity | | Agonistic activity | |
|---|---|---|---|---|---|---|
| | hIL-6 (pg/mL) | | compared to B | | compared to Ctrl | |
| | Antibody (μg/mL) | | | | | |
| | 30 | 6 | 30 | 6 | 30 | 6 |
| Ctrl | 906.060814 | 1012.42048 | | | 0.00% | 0.00% |
| B | 4344.80386 | 4524.76696 | 100.00% | 100.00% | 379.53% | 346.93% |
| L063 | 1129.89262 | 967.744207 | 6.51% | −1.27% | 24.70% | −4.41% |
| L072 | 1447.54151 | 1125.01544 | 15.75% | 3.21% | 59.76% | 11.12% |
| L091 | 944.057133 | 934.684418 | 1.10% | −2.21% | 4.19% | −7.68% |
| L096 | 1736.82678 | 1681.25602 | 24.16% | 19.04% | 91.69% | 66.06% |
| L098 | 1753.61596 | 1501.11166 | 24.65% | 13.91% | 93.54% | 48.27% |
| L106 | 1573.01967 | 1476.44391 | 19.40% | 13.21% | 73.61% | 45.83% |
| L116 | 1566.84383 | 1303.26238 | 19.22% | 8.28% | 72.93% | 28.73% |
| L119 | 1606.92382 | 1255.50299 | 20.38% | 6.92% | 77.35% | 24.01% |

### [Reference Example 6] Assessment of the Human CD3Epsilon Agonist Activities of Anti-Human GPC3/Dual-Fab Trispecific Antibodies

6.1. Preparation of Anti-Human GPC3/Anti-Human CD3Epsilon Bispecific Antibodies and Anti-Human GPC3/Dual-Fab Trispecific Antibodies

The anti-human GPC3/Ctrl bispecific antibodies and the anti-human GPC3/Dual-Fab Trispecific antibodies carrying human IgG1 constant regions were produced in Reference Example 5.1, and the anti-human GPC3/anti-human CD3 epsilon bispecific antibody was also prepared as same construct. CE115 VH (SEQ ID NO:145) and CE115 VL (SEQ ID NO:146) produced in Reference Example 10 was used for anti-human CD3 epsilon antibody Heavy chain and Light chain. The antibodies having the combinations shown in Table 15. These antibodies were expressed by transient expression in FreeStyle293 cells (Invitrogen) and purified according to “Reference Example 9”.

TABLE 15

| Antibody name | Hch gene1 | Lch gene1 | Hch gene1 | Lch gene1 |
|---|---|---|---|---|
| GPC3 ERY22-B | GC33(2)H-G1dKnHS | GC33(2)L-k0 | BVH-G1dHIFS | BVL-k0 |
| GPC3 ERY22-dBBDu_183/L063 | GC33(2)H-G1dKnHS | GC33(2)L-k0 | dBBDu_183VH-G1dHIFS | L063VL-k0 |
| GPC3 ERY22-dBBDu_183/L072 | GC33(2)H-G1dKnHS | GC33(2)L-k0 | dBBDu_183VH-G1dHIFS | L072VL-k0 |
| GPC3 ERY22-dBBDu_167/L091 | GC33(2)H-G1dKnHS | GC33(2)L-k0 | dBBDu_167VH-G1dHIFS | L091VL-k0 |
| GPC3 ERY22-dBBDu_186/L096 | GC33(2)H-G1dKnHS | GC33(2)L-k0 | dBBDu_186VH-G1dHIFS | L096VL-k0 |
| GPC3 ERY22-dBBDu_186/L098 | GC33(2)H-G1dKnHS | GC33(2)L-k0 | dBBDu_186VH-G1dHIFS | L098VL-k0 |
| GPC3 ERY22-dBBDu_186/L106 | GC33(2)H-G1dKnHS | GC33(2)L-k0 | dBBDu_186VH-G1dHIFS | L106VL-k0 |
| GPC3 ERY22-dBBDu_189/L116 | GC33(2)H-G1dKnHS | GC33(2)L-k0 | dBBDu_189VH-G1dHIFS | L116VL-k0 |
| GPC3 ERY22-dBBDu_189/L119 | GC33(2)H-G1dKnHS | GC33(2)L-k0 | dBBDu_189VH-G1dHIFS | L119VL-k0 |
| GPC3 ERY22-dBBDu_183/L067 | GC33(2)H-G1dKnHS | GC33(2)L-k0 | dBBDu_183VH-G1dHIFS | L067VL-k0 |
| GPC3 ERY22-dBBDu_186/L100 | GC33(2)H-G1dKnHS | GC33(2)L-k0 | dBBDu_186VH-G1dHIFS | L100VL-k0 |
| GPC3 ERY22-dBBDu_186/L108 | GC33(2)H-G1dKnHS | GC33(2)L-k0 | dBBDu_186VH-G1dHIFS | L108VL-k0 |
| GPC3 ERY22-dBBDu_189/L112 | GC33(2)H-G1dKnHS | GC33(2)L-k0 | dBBDu_189VH-G1dHIFS | L112VL-k0 |
| GPC3 ERY22-dBBDu_189/L126 | GC33(2)H-G1dKnHS | GC33(2)L-k0 | dBBDu_189VH-G1dHIFS | L126VL-k0 |
| GPC3 ERY22-dBBDu_167/L094 | GC33(2)H-G1dKnHS | GC33(2)L-k0 | dBBDu_167VH-G1dHIFS | L094VL-k0 |
| GPC3 ERY22-dBBDu_193/L127 | GC33(2)H-G1dKnHS | GC33(2)L-k0 | dBBDu_193VH-G1dHIFS | L127VL-k0 |
| GPC3 ERY22-dBBDu_193/L132 | GC33(2)H-G1dKnHS | GC33(2)L-k0 | dBBDu_193VH-G1dHIFS | L132VL-k0 |
| GPC3 ERY22-CE115 | GC33(2)H-G1dKnHS | GC33(2)L-k0 | CE115VH-G1dHIFS | CE115VL-k0 |
| GPC3 ERY22-Ctrl | GC33(2)H-G1dKnHS | GC33(2)L-k0 | CtrlVH-G1dHIFS | CtrlVL-k0 |

6.2. Assessment of the In Vitro GPC3-Dependent CD3 Agonist Effect of Anti-Human GPC3/Dual-Fab Trispecific Antibodies

The agonistic activity to human CD3 was evaluated by using GloResponse™ NFAT-luc2 Jurkat Cell Line (Promega, CS #176401) as effector cell. Jurkat cell is an immortalized cell line of human T lymphocyte cells derived from human acute T cell leukemia and it expresses human CD3 on itself. In NFAT luc2_jurkat cell, the expression of Luciferase was induced by the signal from CD3 activation. SK-pca60 cell line which express human GPC3 on the cell membrane (Reference Example 13) was used as target cell.

Both 5.00E+03 SK-pca60 cells (target cells) and 3.00E+04 NFAT-luc2 Jurkat Cells (Effector cells) were added on the each well of white-bottomed, 96-well assay plate (Costar, 3917), and then 10 micro L of each antibodies with 0.1, 1 or 10 mg/L concentration were added on each well and incubated in the presence of 5% CO2 at 37 degrees Celsius for 24 hours. The expressed Luciferase was detected with Bio-Glo luciferase assay system (Promega, G7940) in accordance with the attached instruction. 2104 EnVIsion was used for detection. The result was shown in FIG. 19.

Most Dual Fab clones showed obvious CD3 epsilon agonist activity and some of them showed equal level of activity with CE115 anti-human CD3 epsilon antibody. It demonstrated that addition of CD137 binding activity to Dual-Fab domain did not induce loss of CD3 epsilon agonist activity and that Dual-Fab domain showed not only binding to two different antigen, human CD3 epsilon and CD137 but also the agonist activity of both human CD3 epsilon and CD137 by only one domain.

Some Dual-Fab domain with Heavy chain dBBDu_186 showed weaker CD3 epsilon agonist activity than others. These antibodies also showed weaker affinity to human CD3 epsilon in biacore analysis in Reference Example 4.5. It demonstrates that the CD3 epsilon agonist activity of Dual-Fab from this Dual Fab library only depends on its affinity to human CD3 epsilon, it means the CD3 epsilon agonist activity was retained in this library design.

### [Reference Example 7] Assessment of the Human CD3Epsilon/Human CD137 Synergistic Activities of Dual-Fab Antibodies in PBMC T Cell Cytokine Release Assay

7.1. Antibody Preparation

Anti-CD137 antibodies described in WO2005/035584A1 (abbreviated as B), Ctrl antibodies described in Reference Example 5.1 and anti-CD3 epsilon CE115 antibody, described in Reference Example 7 were used as single antigen specific controls. Dual-Fab, H183L072 (Heavy chain: SEQ ID NO: 104, Light chain: SEQ ID NO: 124) described in Table 13 was selected for further evaluation and was expressed by transient expression in FreeStyle293 cells (Invitrogen) and purified according to “Reference Example 9”.

7.2. PBMC T Cell Assay

In order to investigate the synergistic effect of Dual-Fab antibody on CD3 epsilon and CD137 activation, total cytokine release was evaluated using cytometric bead array (CBA) Human Th1/T2 Cytokine kit II (BD Biosciences #551809). Relevant to CD137 activation, IL-2 (Interleukin-2), IFN gamma (Interferon gamma) and TNF alpha (Tumor Necrosis Factor-alpha) were evaluated from T cells were isolated from frozen human peripheral blood mononuclear cells (PBMC) purchased frozen (STEMCELL).

7.2.1. Preparation of Frozen Human PBMC and Isolation of T Cells

Cryovials containing PBMCs were placed in the water bath at 37 degrees C. to thaw cells. Cells were then dispensed into a 15 mL falcon tube containing 9 mL of media (media used to culture target cells). Cell suspension was then subjected to centrifugation at 1,200 rpm for 5 minutes at room temperature. The supernatant was aspirated gently and fresh warmed medium was added for resuspension and used as the human PBMC solution. T cells were isolated using Dynabeads Untouched Human T cell kit (Invitrogen #11344D) following manufacturer's instructions.

7.2.2. Cytokine Release Assay

30 micro g/mL and 10 micro g/mL of antibodies prepared in Reference Example 7.1 were coated on maxisorp 96-well plate (Thermofisher #442404) overnight. 1.00E+05 T cells were added to each well containing antibodies and incubated at 37 degrees C. for 72 hours. Plates were centrifuged at 1,200 rpm for 5 minutes and supernatant was collected. CBA was performed according to manufacturer's instructions and the results are shown in FIG. 20.

Only dual-Fab, H183L072 antibody showed IL-2 secretion by T cells. Neither antiCD137(B) nor anti-CD3 epsilon antibody (CE115) alone could result in induction of IL-2 from T cells. In addition, anti-CD137 antibody alone did not result in detection of any cytokine. As compared to anti-CD3 epsilon antibody, Dual-Fab antibody resulted in increased levels of TNF alpha and similar secretion of IFN gamma. These results suggest that dual-Fab antibody could elicit synergistic activation of both CD3 epsilon and CD137 for functional activation of T cells.

### [Reference Example 8] Assessment of the Cytotoxicity of Anti-GPC3/Dual-Fab Trispecific Antibodies

8.1. Anti-GPC3/Dual-Fab and Anti-GPC3/CD137 Bi-Specific Antibody Preparation

Anti-GPC3 or Ctrl antibodies described in Reference Example 6 and Dual-Fab (H183L072) or anti-CD137 antibodies were used to generate four antibodies, AntiGPC3/dual-Fab, anti-GPC3/CD137, Ctrl/H183L072, and Ctrl/CD137 antibodies using Fab-arm exchange (FAE) according to a method described in (Proc Natl Acad Sci USA. 2013 Mar. 26; 110(13): 5145-5150). The molecular format of all four antibodies is the same format as a conventional IgG. Anti-GPC3/H183L072 is tri-specific antibody that is able to bind GPC3, CD3, and CD137, anti-GPC3/CD137 is bi-specific antibody that is able to bind GPC3 and CD137, and Ctrl/H183L072, and Ctrl/CD137 were used as control. All four antibodies generated consist of a silent Fc with attenuated affinity for Fc gamma receptor.

8.2. T-Cell Dependent Cellular Cytotoxicity (TDCC) Assay

Cytotoxic activity was assessed by the rate of cell growth inhibition using xCELLigence Real-Time Cell Analyzer (Roche Diagnostics) as described in Reference Example 10.5.2. 1.00E+04 SK-pca60 or SK-pca13a, both transfectant cell lines expressing GPC3 were used as target(abbreviated as T) cells (Reference Examples 13 and 10 respectively) and co-cultured with 5.00E+04 frozen human PBMCs effector(abbreviated as E) cells that were prepared as described in Reference Example 7.2.1. It means 5-fold amount of effector cells were added on tumor cells, so it is described here as ET 5. Anti-GPC3/H183L072 antibodies and GPC3/CD137 antibodies were added at 0.4, 5 and 10 nM while Ctrl/H183L072 antibodies and Ctrl/CD137 antibodies were added at 10 nM each well. Measurement of cytotoxic activity was conducted similarly as described in Reference Example 10.5.2. The reaction was carried out under the conditions of 5% carbon dioxide gas at 37 degrees C. 72 hours after the addition of PBMCs, Cell Growth Inhibition (CGI) rate (%) was determined using the equation described in Reference Example 10.5.2 and plotted in the graph as shown in FIG. 21. Anti-GPC3/H183L072 dual-Fab antibody which showed CD3 activation on Jurkat cells in Reference Example 6.2 but not Control/H183L072 dual-Fab antibody which did not show CD3 activation and anti-GPC3/CD137 antibody resulted in strong cytotoxic activity of GPC3-expressing cells at all concentrations in both target cell lines, suggesting that Dual-Fab tri-specific antibodies can result in cytotoxic activity.

### [Reference Example 9] Preparation of Antibody Expression Vector and Expression and Purification of Antibody

Amino acid substitution or IgG conversion was carried out by a method generally known to those skilled in the art using QuikChange Site-Directed Mutagenesis Kit (Stratagene Corp.), PCR, or In fusion Advantage PCR cloning kit (Takara Bio Inc.), etc., to construct expression vectors. The obtained expression vectors were sequenced by a method generally known to those skilled in the art. The prepared plasmids were transiently transferred to human embryonic kidney cancer cell-derived HEK293H line (Invitrogen Corp.) or FreeStyle 293 cells (Invitrogen Corp.) to express antibodies. Each antibody was purified from the obtained culture supernatant by a method generally known to those skilled in the art using rProtein A Sepharose™ Fast Flow (GE Healthcare Japan Corp.). As for the concentration of the purified antibody, the absorbance was measured at 280 nm using a spectrophotometer, and the antibody concentration was calculated by use of an extinction coefficient calculated from the obtained value by PACE (Protein Science 1995; 4: 2411-2423).

### [Reference Example 10] Preparation of Anti-Human and Anti-Cynomolgus Monkey CD3Epsilon Antibody CE115

10.1. Preparation of Hybridoma Using Rat Immunized with Cell Expressing Human CD3 and Cell Expressing Cynomolgus Monkey CD3

Each SD rat (female, 6 weeks old at the start of immunization, Charles River Laboratories Japan, Inc.) was immunized with Ba/F3 cells expressing human CD3 epsilon gamma or cynomolgus monkey CD3 epsilon gamma as follows: at day 0 (the priming date was defined as day 0), 5×10⁷ Ba/F3 cells expressing human CD3 epsilon gamma were intraperitoneally administered together with a Freund complete adjuvant (Difco Laboratories, Inc.) to the rat. At day 14, 5×10⁷ Ba/F3 cells expressing cynomolgus monkey CD3 epsilon gamma were intraperitoneally administered thereto together with a Freund incomplete adjuvant (Difco Laboratories, Inc.). Then, 5×10⁷ Ba/F3 cells expressing human CD3 epsilon gamma and Ba/F3 cells expressing cynomolgus monkey CD3 epsilon gamma were intraperitoneally administered thereto a total of four times every other week in an alternate manner. One week after (at day 49) the final administration of CD3 epsilon gamma, Ba/F3 cells expressing human CD3 epsilon gamma were intravenously administered thereto as a booster. Three days thereafter, the spleen cells of the rat were fused with mouse myeloma cells SP2/0 according to a routine method using PEG1500 (Roche Diagnostics K.K.). Fusion cells, i.e., hybridomas, were cultured in an RPMI1640 medium containing 10% FBS (hereinafter, referred to as 10% FBS/RPMI1640).

On the day after the fusion, (1) the fusion cells were suspended in a semifluid medium (Stemcell Technologies, Inc.). The hybridomas were selectively cultured and also colonized.

Nine or ten days after the fusion, hybridoma colonies were picked up and inoculated at 1 colony/well to a 96-well plate containing a HAT selective medium (10% FBS/RPMI1640, 2 vol % HAT 50×concentrate (Sumitomo Dainippon Pharma Co., Ltd.), and 5 vol % BM-Condimed H1 (Roche Diagnostics K.K.)). After 3- to 4-day culture, the culture supernatant in each well was recovered, and the rat IgG concentration in the culture supernatant was measured. The culture supernatant confirmed to contain rat IgG was screened for a clone producing an antibody specifically binding to human CD3 epsilon gamma by cell-ELISA using attached Ba/F3 cells expressing human CD3 epsilon gamma or attached Ba/F3 cells expressing no human CD3 epsilon gamma (FIG. 22). The clone was also evaluated for cross reactivity with monkey CD3 epsilon gamma by cell-ELISA using attached Ba/F3 cells expressing cynomolgus monkey CD3 epsilon gamma (FIG. 22).

10.2. Preparation of Anti-Human and Anti-Monkey CD3Epsilon Chimeric Antibody

Total RNA was extracted from each hybridoma cell using RNeasy Mini Kits (Qiagen N.V.), and cDNA was synthesized using SMART RACE cDNA Amplification Kit (BD Biosciences). The prepared cDNA was used in PCR to insert the antibody variable region gene to a cloning vector. The nucleotide sequence of each DNA fragment was determined using BigDye Terminator Cycle Sequencing Kit (Applied Biosystems, Inc.) and a DNA sequencer ABI PRISM 3700 DNA Sequencer (Applied Biosystems, Inc.) according to the method described in the instruction manual included therein. CDRs and FRs of the CE115 H chain variable domain (SEQ ID NO: 162) and the CE115 L chain variable domain (SEQ ID NO: 163) were determined according to the Kabat numbering.

A gene encoding a chimeric antibody H chain containing the rat antibody H chain variable domain linked to a human antibody IgG1 chain constant domain, and a gene encoding a chimeric antibody L chain containing the rat antibody L chain variable domain linked to a human antibody kappa chain constant domain were integrated to expression vectors for animal cells. The prepared expression vectors were used for the expression and purification of the CE115 chimeric antibody (Reference Example 9).

10.3. Preparation of EGFR_ERY22_CE115

Next, IgG against a cancer antigen (EGFR) was used as a backbone to prepare a molecule in a form with one Fab replaced with CD3 epsilon-binding domains. In this operation, silent Fc having attenuated binding activity against FcgR (Fc gamma receptor) was used, as in the case mentioned above, as Fc of the backbone IgG. Cetuximab-VH (SEQ ID NO: 164) and Cetuximab-VL (SEQ ID NO: 165) constituting the variable region of cetuximab were used as EGFR-binding domains. G1d derived from IgG1 by the deletion of C-terminal Gly and Lys, A5 derived from G1d by the introduction of D356K and H435R mutations, and B3 derived from G1d by the introduction of a K439E mutation were used as antibody H chain constant domains and each combined with Cetuximab-VH to prepare Cetuximab-VH-G1d (SEQ ID NO: 166), Cetuximab-VH-A5 (SEQ ID NO: 167), and Cetuximab-VH-B3 (SEQ ID NO: 168) according to the method of Reference Example 9. When the antibody H chain constant domain was designated as H1, the sequence corresponding to the antibody H chain having Cetuximab-VH as a variable domain was represented by Cetuximab-VH-H1.

In this context, the alteration of an amino acid is represented by, for example, D356K. The first alphabet (which corresponds to D in D356K) means an alphabet that represents the one-letter code of the amino acid residue before the alteration. The number (which corresponds to 356 in D356K) following the alphabet means the EU numbering position of this altered residue. The last alphabet (which corresponds to K in D356K) means an alphabet that represents the one-letter code of an amino acid residue after the alteration.

EGFR_ERY22_CE115 (FIG. 23) was prepared by the exchange between the VH domain and the VL domain of Fab against EGFR. Specifically, a series of expression vectors having an insert of each polynucleotide encoding EGFR_ERY22_Hk (SEQ ID NO: 169), EGFR_ERY22_L (SEQ ID NO: 170), CE115_ERY22_Hh (SEQ ID NO: 171), or CE115_ERY22_L (SEQ ID NO: 172) was prepared by a method generally known to those skilled in the art, such as PCR, using primers with an appropriate sequence added in the same way as the aforementioned method.

The expression vectors were transferred in the following combination to FreeStyle 293-F cells where each molecule of interest was transiently expressed: Molecule of interest: EGFR_ERY22_CE115 Polypeptides encoded by the polynucleotides inserted in the expression vectors: EGFR ERY22_Hk, EGFR_ERY22_L, CE115_ERY22_Hh, and CE115_ERY22_L

10.4. Purification of EGFR_ERY22_CE115

The obtained culture supernatant was added to Anti FLAG M2 column (Sigma-Aldrich Corp.), and the column was washed, followed by elution with 0.1 mg/mL FLAG peptide (Sigma-Aldrich Corp.). The fraction containing the molecule of interest was added to HisTrap HP column (GE Healthcare Japan Corp.), and the column was washed, followed by elution with the concentration gradient of imidazole. The fraction containing the molecule of interest was concentrated by ultrafiltration. Then, this fraction was added to Superdex 200 column (GE Healthcare Japan Corp.). Only a monomer fraction was recovered from the eluate to obtain each purified molecule of interest.

10.5. Measurement of Cytotoxic Activity Using Human Peripheral Blood Mononuclear Cell

10.5.1. Preparation of Human Peripheral Blood Mononuclear Cell (PBMC) Solution

50 mL of peripheral blood was collected from each healthy volunteer (adult) using a syringe pre-filled with 100 micro L of 1,000 units/mL of a heparin solution (Novo-Heparin 5,000 units for Injection, Novo Nordisk A/S). The peripheral blood was diluted 2-fold with PBS(−) and then divided into four equal parts, which were then added to Leucosep lymphocyte separation tubes (Cat. No. 227290, Greiner Bio-One GmbH) pre-filled with 15 mL of Ficoll-Paque PLUS and centrifuged in advance. After centrifugation (2,150 rpm, 10 minutes, room temperature) of the separation tubes, a mononuclear cell fraction layer was separated. The cells in the mononuclear cell fraction were washed once with Dulbecco's Modified Eagle's Medium containing 10% FBS (Sigma-Aldrich Corp.; hereinafter, referred to as 10% FBS/D-MEM). Then, the cells were adjusted to a cell density of 4×10⁶ cells/mL with 10% FBS/D-MEM. The cell solution thus prepared was used as a human PBMC solution in the subsequent test.

10.5.2. Measurement of Cytotoxic Activity

The cytotoxic activity was evaluated on the basis of the rate of cell growth inhibition using xCELLigence real-time cell analyzer (Roche Diagnostics). The target cells used were an SK-pca13a cell line established by forcing an SK-HEP-1 cell line to express human EGFR. SK-pca13a was dissociated from the dish and inoculated at 100 micro L/well (1×10⁴cells/well) to an E-Plate 96 plate (Roche Diagnostics) to start the assay of live cells using the xCELLigence real-time cell analyzer. On the next day, the plate was taken out of the xCELLigence real-time cell analyzer, and 50 micro L of each antibody adjusted to each concentration (0.004, 0.04, 0.4, and 4 nM) was added to the plate. After reaction at room temperature for 15 minutes, 50 micro L (2×10⁵ cells/well) of the human PBMC solution prepared in the preceding paragraph 10.5.1 was added thereto. This plate was reloaded to the xCELLigence real-time cell analyzer to start the assay of live cells. The reaction was carried out under conditions of 5% CO₂ and 37 degrees C. 72 hours after the addition of human PBMC. The rate of cell growth inhibition (%) was determined from the cell index value according to the expression given below. A numeric value after normalization against the cell index value immediately before the addition of the antibody defined as 1 was used as the cell index value in this calculation.

Rate of cell growth inhibition (%)=(A−B)×100/(A−1), wherein

A represents the average cell index value of wells non-supplemented with the antibody (only the target cells and human PBMC), and B represents the average cell index value of the wells supplemented with each antibody. The test was conducted in triplicate.

The cytotoxic activity of EGFR_ERY22_CE115 containing CE115 was measured with PBMC prepared from human blood as effector cells. As a result, very strong activity was confirmed (FIG. 24).

### [Reference Example 11] Antibody Alteration for Preparation of Antibody Binding to CD3 and Second Antigen

11.1. Study on Insertion Site and Length of Peptide Capable of Binding to Second Antigen

A study was conducted to obtain a dual binding Fab molecule capable of binding to a cancer antigen through one variable region (Fab) and binding to the first antigen CD3 and the second antigen through the other variable region, but not capable of binding to CD3 and the second antigen at the same time. A GGS peptide was inserted to the heavy chain loop of the CD3 epsilon-binding antibody CE115 to prepare each heterodimerized antibody having EGFR-binding domains in one Fab and CD3-binding domains in the other Fab according to Reference Example 9.

Specifically, EGFR_ERY22_Hk/EGFR_ERY22_L/CE115_CE31 ERY22_Hh/CE115_ERY22_L ((SEQ ID NO: 169/170/173/172) with GGS inserted between K52B and S52c in CDR2, EGFR_ERY22_Hk/EGFR_ERY22_L/CE115_CE32 ERY22_Hh/CE115_ERY22_L ((SEQ ID NO: 169/170/174/172) with a GGSGGS peptide (SEQ ID NO: 175) inserted at this position, and EGFR_ERY22_Hk/EGFR ERY22_L/CE115_CE33 ERY22_Hh/CE115_ERY22_L ((SEQ ID NO: 169/170/176/172) with a GGSGGSGGS peptide (SEQ ID NO: 177) inserted at this position were prepared. Likewise, EGFR_ERY22_Hk/EGFR_ERY22_L/CE115_CE34 ERY22_Hh/CE115_ERY22_L ((SEQ ID NO: 169/170/178/172) with GGS inserted between D72 and D73 (loop) in FR3, EGFR_ERY22_Hk/EGFR ERY22_L/CE115_CE35 ERY22_Hh/CE115_ERY22_L ((SEQ ID NO: 169/170/179/172) with a GGSGGS peptide (SEQ ID NO: 175) inserted at this position, and EGFR_ERY22_Hk/EGFR_ERY22_L/CE115_CE36 ERY22_Hh/CE115_ERY22_L ((SEQ ID NO: 169/170/180/172) with a GGSGGSGGS peptide (SEQ ID NO: 177) inserted at this position were prepared. In addition, EGFR_ERY22_Hk/EGFR ERY22_L/CE115_CE37 ERY22_Hh/CE115_ERY22_L ((SEQ ID NO: 169/170/181/172) with GGS inserted between A99 and Y100 in CDR3, EGFR ERY22_Hk/EGFR_ERY22_L/CE115_CE38 ERY22_Hh/CE115_ERY22_L ((SEQ ID NO: 169/170/182/172) with a GGSGGS peptide inserted at this position, and EGFR ERY22_Hk/EGFR_ERY22_L/CE115_CE39 ERY22_Hh/CE115_ERY22_L ((SEQ ID NO: 169/170/183/172) with a GGSGGSGGS peptide inserted at this position were prepared.

11.2. Confirmation of Binding of GGS Peptide-Inserted CE115 Antibody to CD3 Epsilon

The binding activity of each prepared antibody against CD3 epsilon was confirmed using Biacore T100. A biotinylated CD3 epsilon epitope peptide was immobilized to a CM5 chip via streptavidin, and the prepared antibody was injected thereto as an analyte and analyzed for its binding affinity.

The results are shown in Table 16. The binding affinity of CE35, CE36, CE37, CE38, and CE39 for CD3 epsilon was equivalent to the parent antibody CE115. This indicated that a peptide binding to the second antigen can be inserted into their loops. The binding affinity was not reduced in GGSGGSGGS-inserted CE36 or CE39. This indicated that the insertion of a peptide up to at least 9 amino acids to these sites does not influence the binding activity against CD3 epsilon.

TABLE 16

| Sample | ka | kd | KD | Insertion | |
|---|---|---|---|---|---|
| CE115_M | 1.5E+05 | 9.8E−03 | 6.7E−08 | position | Linker |
| CE31 | 2.3E+05 | 3.5E−02 | 1.5E−07 | K52b-S52c | GS3 |
| CE32 | 8.5E+04 | 1.8E−02 | 2.1E−07 | K52b-S52c | GS6 |
| CE33 | 4.9E+05 | 1.1E−01 | 2.3E−07 | K52b-S52c | GS9 |
| CE34 | 1.1E+05 | 1.3E−02 | 1.2E−07 | D72-D73 | GS3 |
| CE35 | 1.3E+05 | 1.1E−02 | 8.7E−08 | D72-D73 | GS6 |
| CE36 | 1.2E+05 | 1.2E−02 | 9.9E−08 | D72-D73 | GS9 |
| CE37 | 2.2E+05 | 2.0E−02 | 9.4E−08 | A99-Y100 | GS3 |
| CE38 | 2.0E+05 | 1.7E−02 | 8.7E−08 | A99-Y100 | GS6 |
| CE39 | 1.6E+05 | 1.4E−02 | 9.1E−08 | A99-Y100 | GS9 |

These results indicated that the antibody capable of binding to CD3 and the second antigen, but does not bind to these antigens at the same time can be prepared by obtaining an antibody binding to the second antigen using such peptide-inserted CE115.

In this context, a library can be prepared by altering at random the amino acid sequence of the peptide for use in insertion or substitution according to a method known in the art such as site-directed mutagenesis (Kunkel et al., Proc. Natl. Acad. Sci. U.S.A. (1985) 82, 488-492) or overlap extension PCR, and comparing the binding activity, etc., of each altered form according to the aforementioned method to determine an insertion or substitution site that permits exertion of the activity of interest even after alteration of the amino acid sequence, and the types and length of amino acids of this site.

### [Reference Example 12] Library Design for Obtaining Antibody Binding to CD3 and Second Antigen

12.1. Antibody Library for Obtaining Antibody Binding to CD3 and Second Antigen (Also Referred to as Dual Fab Library)

In the case of selecting CD3 (CD3 epsilon) as the first antigen, examples of a method for obtaining an antibody binding to CD3 (CD3 epsilon) and an arbitrary second antigen include the following 6 methods:

1. a method which involves inserting a peptide or a polypeptide binding to the second antigen to a Fab domain binding to the first antigen (this method includes the peptide insertion shown in Example 3 or 4 in WO2016076345A1 (or, as well as a G-CSF insertion method illustrated in Angew Chem Int Ed Engl. 2013 Aug. 5; 52 (32): 8295-8), wherein the binding peptide or polypeptide may be obtained from a peptideor polypeptide-displaying library, or the whole or a portion of a naturally occurring protein may be used;

2. a method which involves preparing an antibody library such that various amino acids appear positions that permit alteration to a larger length (extension) of Fab loops as shown in Example 5 in WO2016076345A1, and obtaining Fab having binding activity against an arbitrary second antigen from the antibody library by using the binding activity against the antigen as an index;

3. a method which involves identifying amino acids that maintain binding activity against CD3 by use of an antibody prepared by site-directed mutagenesis from a Fab domain previously known to bind to CD3, and obtaining Fab having binding activity against an arbitrary second antigen from an antibody library in which the identified amino acids appear by using the binding activity against the antigen as an index;

4. the method 3 which further involves preparing an antibody library such that various amino acids appear positions that permit alteration to a larger length (extension) of Fab loops, and obtaining Fab having binding activity against an arbitrary second antigen from the antibody library by using the binding activity against the antigen as an index;

5. the method 1, 2, 3, or 4 which further involves altering the antibodies such that glycosylation sequences (e.g., NxS and NxT wherein x is an amino acid other than P) appear to add thereto sugar chains that are recognized by sugar chain receptors (e.g., high-mannose-type sugar chains are added thereto and thereby recognized by high-mannose receptors; it is known that the high-mannose-type sugar chains are obtained by the addition of kifunensine at the time of antibody expression (mAbs. 2012 July-August; 4 (4): 475-87)); and

6. the method 1, 2, 3, or 4 which further involves adding thereto domains (polypeptides, sugar chains, and nucleic acids typified by TLR agonists) each binding to the second antigen through a covalent bond by inserting Cys, Lys, or a non-natural amino acid to loops or sites found to be alterable to various amino acids or substituting these sites with Cys, Lys, or a non-natural amino acid (this method is typified by antibody drug conjugates and is a method for conjugation to Cys, Lys, or a non-natural amino acid through a covalent bond (described in mAbs 6: 1, 34-45; January/February 2014; WO2009/134891 A2; and Bioconjug Chem. 2014 Feb. 19; 25 (2): 351-61)). The dual binding Fab that binds to the first antigen and the second antigen, but does not bind to these antigens at the same time is obtained by use of any of these methods, and can be combined with domains binding to an arbitrary third antigen by a method generally known to those skilled in the art, for example, common L chains, CrossMab, or Fab arm exchange.

12.2. Preparation of One-Amino Acid Alteration Antibody of CD3 (CD3 Epsilon)-Binding Antibody Using Site-Directed Mutagenesis

A VH domain CE115HA000 (SEQ ID NO: 184) and a VL domain GLS3000 (SEQ ID NO: 185) were selected as template sequences for a CD3 (CD3 epsilon)-binding antibody. Each domain was subjected to amino acid alteration at a site presumed to participate in antigen binding according to Reference Example 9. Also, pE22Hh (sequence derived from natural IgG1 CH1 and subsequent sequences by the alteration of L234A, L235A, N297A, D356C, T366S, L368A, and Y407V, the deletion of a C-terminal GK sequence, and the addition of a DYKDDDDK sequence (SEQ ID NO: 200); SEQ ID NO: 186) was used as an H chain constant domain, and a kappa chain (SEQ ID NO: 187) was used as an L chain constant domain. The alteration sites are shown in Table 17. For CD3 (CD3 epsilon)-binding activity evaluation, each one-amino acid alteration antibody was obtained as a one-arm antibody (naturally occurring IgG antibody lacking one of the Fab domains). Specifically, in the case of H chain alteration, the altered H chain linked to the constant domain pE22Hh, and Kn010G3 (naturally occurring IgG1 amino acid sequence from position 216 to the C terminus having C220S, Y349C, T366W, and H435R alterations; SEQ ID NO: 188) were used as H chains, and GLS3000 linked at the 3′ side to the kappa chain was used as an L chain. In the case of L chain alteration, the altered L chain linked at the 3′ side to the kappa chain was used as an L chain, and CE115HA000 linked at the 3′ side to pE22Hh, and Kn010G3 were used as H chains. These sequences were expressed and purified in FreeStyle 293 cells (which employed the method of Reference Example 9).

TABLE 17
H chain alteration site

| | Domain | | | | | | | | | | | | | | | | | | | | | |
|---|---|---|---|---|---|---|---|---|---|---|---|---|---|---|---|---|---|---|---|---|---|---|
| | FR1 | | | | | | CDR1 | | | | FR2 | CDR2 | | | | | | | | | | |
| Kabat | 11 | 16 | 19 | 28 | 29 | 30 | 31 | 32 | 33 | 35 | 43 | 50 | 51 | 52 | 52a | 52b | 52c | 53 | 54 | 55 | 56 | 57 |
| numbering | | | | | | | | | | | | | | | | | | | | | | |
| Amino | V | R | R | T | F | S | N | A | W | H | K | Q | I | K | A | K | S | N | N | Y | A | T |
| acid before | | | | | | | | | | | | | | | | | | | | | | |
| substitution | | | | | | | | | | | | | | | | | | | | | | |
| | Domain | | | | | | | | | | | | | | | | | | | | | |
| | CDR2 | | | | | FR3 | | | | | | | | CDR3 | | | | | | | | |
| Kabat | 58 | 59 | 60 | 61 | 62 | 64 | 65 | 73 | 74 | 75 | 76 | 77 | 78 | 82a | 95 | 96 | 97 | 98 | 99 | | 100 | |
| numbering | | | | | | | | | | | | | | | | | | | | | | |
| Amino | Y | Y | A | E | S | K | G | D | S | K | N | S | L | N | V | H | Y | G | A | | Y | |
| acid before | | | | | | | | | | | | | | | | | | | | | | |
| substitution | | | | | | | | | | | | | | | | | | | | | | |

L chain alteration site

| | Domain | | | | | | | | | | | | | | | | | | | | | |
|---|---|---|---|---|---|---|---|---|---|---|---|---|---|---|---|---|---|---|---|---|---|---|
| | CDR3 | | | | FR4 | CDR1 | | | | | | | | | | | | | | | | |
| Kabat | 100a | 100b | 100c | 101 | 102 | 105 | 24 | 25 | 26 | 27 | 27a | 27b | 27c | 27d | 27e | 28 | 29 | 30 | 31 | 32 | 33 | 34 |
| numbering | | | | | | | | | | | | | | | | | | | | | | |
| Amino | Y | G | V | D | A | Q | R | S | S | Q | S | L | V | H | S | N | R | N | T | Y | L | H |
| acid before | | | | | | | | | | | | | | | | | | | | | | |
| substitution | | | | | | | | | | | | | | | | | | | | | | |

| | Domain | | | | | | | | | | | | | | | | | | | |
|---|---|---|---|---|---|---|---|---|---|---|---|---|---|---|---|---|---|---|---|---|
| | FR2 | CDR2 | | | | | | | FR3 | | CDR3 | | | | | | | | | FR4 |
| Kabat | 45 | 50 | 51 | 52 | 53 | 54 | 55 | 56 | 74 | 77 | 89 | 90 | 91 | 92 | 93 | 94 | 95 | 96 | 97 | 107 |
| numbering | | | | | | | | | | | | | | | | | | | | |
| Amino | Q | K | V | S | N | R | F | S | K | R | G | Q | G | T | Q | V | P | Y | T | K |
| acid before | | | | | | | | | | | | | | | | | | | | |
| substitution | | | | | | | | | | | | | | | | | | | | |

12.3. Evaluation of Binding of One-Amino Acid Alteration Antibody to CD3

Each one-amino acid altered form constructed, expressed, and purified in the paragraph 12.2. was evaluated using Biacore T200 (GE Healthcare Japan Corp.). An appropriate amount of CD3 epsilon homodimer protein was immobilized onto Sensor chip CM4 (GE Healthcare Japan Corp.) by the amine coupling method. Then, the antibody having an appropriate concentration was injected thereto as an analyte and allowed to interact with the CD3 epsilon homodimer protein on the sensor chip. Then, the sensor chip was regenerated by the injection of 10 mmol/L glycine-HCl (pH 1.5). The assay was conducted at 25 degrees C., and HBS-EP+(GE Healthcare Japan Corp.) was used as a running buffer. From the assay results, the dissociation constant K_{D} (M) was calculated using single-cycle kinetics model (1:1 binding RI=0) for the amount bound and the sensorgram obtained in the assay. Each parameter was calculated using Biacore T200 Evaluation Software (GE Healthcare Japan Corp.).

12.3.1. Alteration of H Chain

Table 18 shows the results of the ratio of the amount of each H chain altered form bound to the amount of the corresponding unaltered antibody CE115HA000 bound. Specifically, when the amount of the antibody comprising CE115HA000 bound was defined as X and the amount of the H chain one-amino acid altered form bound was defined as Y, a value of Z (ratio of amounts bound)=Y/X was used. As shown in FIG. 25, a very small amount bound was observed in the sensorgram for Z of less than 0.8, suggesting the possibility that the dissociation constant K_{D} (M) cannot be calculated correctly. Table 19 shows the dissociation constant K_{D} (M) ratio of each H chain altered form to CE115HA000 (=KD value of CE115HA000/KD value of the altered form).

When Z shown in Table 18 is 0.8 or more, the altered form is considered to maintain the binding relative to the corresponding unaltered antibody CE115HA000. Therefore, an antibody library designed such that these amino acids appear can serve as a dual Fab library.

TABLE 18

| | Domain | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|
| | FR1 | | | | | | CDR1 | | | | FR2 | CDR2 | | | | | | | | | | | | | | | | | |
| | Kabat numbering | | | | | | | | | | | | | | | | | | | | | | | | | | | | |
| | 11 | 16 | 19 | 28 | 29 | 30 | 31 | 32 | 33 | 35 | 43 | 50 | 51 | 52 | 52a | 52b | 52c | 53 | 54 | 55 | 56 | 57 | 58 | 59 | 60 | 61 | 62 | 64 | 65 |
| | Amino acid before substitution | | | | | | | | | | | | | | | | | | | | | | | | | | | | |
| (wt) | V | R | R | T | F | S | N | A | W | H | K | Q | I | K | A | K | S | N | N | Y | A | T | Y | Y | A | E | S | K | G |
| A | | | | | | | 0.5 | | 0.1 | 0.17 | | 0.24 | | | | 0.67 | 0.96 | 0.7 | 0.85 | | | 0.98 | 0.22 | 0.85 | | 1.09 | | 0.82 | |
| D | | | | 0.56 | 0.86 | 0.37 | | 0.1 | 0.2 | | 0.27 | 0.29 | 0.25 | 1.34 | 0.27 | 0.6 | 0.39 | 0.62 | 0.45 | 0.51 | | 0.11 | 0.7 | 0.99 | 0.91 | 0.92 | 0.72 | 0.76 | |
| E | | 0.88 | | | | | | | | 0.19 | 0.9 | 0.26 | 0.55 | 0.26 | 0.57 | | | | | | | | | 0.66 | 0.94 | | 0.92 | 0.74 | 0.78 |
| F | | | | | | | 0.62 | | 0.65 | 0.21 | | | | 0.17 | | | 1.13 | | 1.12 | | | | | | | | | | |
| G | | | | | | 1.01 | 0.39 | | | 0.22 | | | | | 0.81 | | 0.97 | 0.5 | 0.98 | 0.55 | 0.61 | | | | | | | | |
| H | | | | | | | 0.68 | | 0.13 | | | | | 0.22 | | | | 0.76 | | | | | | | | | | | |
| I | | | | | | | 0.81 | | | 0.12 | | 0.4 | | 0.33 | | | | 0.68 | | | | 0.61 | | | | | | | |
| K | | | | | | | 1.01 | | | 0.15 | | 0.33 | | | | | 1.19 | 0.78 | 1.2 | | 1.35 | 1.32 | 0.3 | | | | 1.19 | | |
| L | 1 | | | | | | | | 0.1 | 0.11 | | 0.23 | | | | 0.61 | 0.98 | | 0.94 | | 0.8 | | 0.27 | | | | | | |
| M | | | | | | | | | | 0.29 | | | | | | | | | | | | | | | | | | | |
| N | | | | | | | | | | 0.35 | | 0.17 | | 0.34 | | 0.27 | | | | | 0.87 | 0.97 | 0.33 | | | | | | |
| P | | | | | | | | | | | | | | | 0.15 | | | | | | | | | | | 1.07 | 1 | | |
| Q | | 0.9 | | | | | 0.49 | | | 0.13 | 0.99 | | | | | 0.6 | 1.04 | 1.1 | 0.84 | | 0.76 | | 0.19 | | | 1.07 | | 0.89 | |
| R | | | | | | | 1.14 | | | 0.14 | | | | 0.91 | | 1.11 | | | | | | | | | | | | | |
| S | | | | 0.91 | | | 0.81 | | | 0.23 | | 0.24 | | 0.28 | 1.05 | 0.68 | | 0.83 | 0.84 | 0.26 | | | 0.18 | 0.94 | | | | 0.84 | |
| T | | | 0.8 | | | | | | | 0.26 | | | | | | | | | | | | | | | | | | | |
| V | | | | | 0.36 | | | | | 0.22 | | | | | 0.52 | | 0.93 | | | | | | | | | | | | |
| W | | | | | | | 0.63 | | | 0.22 | | | | 0.22 | | | | 0.88 | | | | | | | | | | | |
| Y | | | | | | | 0.64 | 0.33 | 0.66 | 0.16 | | 0.25 | | 0.18 | 0.31 | 0.74 | 1.11 | 0.63 | 1.09 | | 0.66 | | | | | | | | |
| | | | | | | | | | | Domain | | | | | | | | | | | | | | | | | | | |
| | | | | | | | | | | FR3 | | | | | | | | CDR3 | | | | | | | | | | | FR4 |
| | | | | | | | | | | Kabat numbering | | | | | | | | | | | | | | | | | | | |
| | | | | | | | | | | 72 | 73 | 74 | 75 | 76 | 77 | 78 | 82a | 95 | 96 | 97 | 98 | 99 | 100 | 100a | 100b | 100c | 101 | 102 | 105 |
| | | | | | | | | | | Amino acid before substitution | | | | | | | | | | | | | | | | | | | |
| | | | | | | | | | (wt) | D | D | S | K | N | S | L | N | V | H | Y | G | A | Y | Y | G | V | D | A | Q |
| | | | | | | | | | A | 1.41 | 0.83 | 1.05 | | | | | | 0.11 | 0.35 | 0.16 | 1.1 | | 0.9 | 0.62 | | | 1.26 | | |
| | | | | | | | | | D | | | | | | | | 0.73 | 0.24 | 0.09 | 0.24 | 0.26 | 0.28 | 0.52 | 0.31 | 0.27 | 0.44 | | | |
| | | | | | | | | | E | 1.05 | 0.73 | | | | | | | 0.24 | | | | | | | 0.26 | 0.46 | | | 0.94 |
| | | | | | | | | | F | | | | | | | | | | | | | 1.43 | | 0.87 | 0.3 | | 0.75 | | |
| | | | | | | | | | G | | 1.07 | | | | | | | 0.19 | 0.43 | 0.18 | | | 1.07 | 1.23 | | | 1.38 | | |
| | | | | | | | | | H | | | | | | | | | | | | 1.58 | | | | | | 1.21 | | |
| | | | | | | | | | I | | | | | | | 1.34 | | | | | | | | | | | 1.18 | 1.48 | |
| | | | | | | | | | K | | 0.87 | | | | | | | 0.64 | 0.38 | | 2.83 | 1.48 | 1.07 | 0.9 | | | 0.63 | | |
| | | | | | | | | | L | | | | | | | | | | 0.14 | | | 1.13 | 0.7 | 0.48 | 0.27 | | 0.62 | | |
| | | | | | | | | | M | | | | | | | | | | | | | | | | | | 1.2 | | |
| | | | | | | | | | N | | 0.94 | | | | | | | | | | | | | | | | 2.02 | | |
| | | | | | | | | | P | | | 0.91 | | | | | | | 0.12 | 0.11 | 1.02 | | 0.48 | | | 0.2 | 0.2 | 0.14 | |
| | | | | | | | | | Q | | | | | | | | | | 0.42 | | 1.22 | 0.91 | 0.8 | 0.56 | | | 2.35 | | |
| | | | | | | | | | R | | 1.04 | | 1.01 | | | | | | 0.46 | 0.27 | 2.96 | | | | | | 0.24 | | |
| | | | | | | | | | S | | | | | 0.92 | | | | 0.22 | 0.44 | 0.18 | 1.01 | 0.82 | 0.81 | 0.64 | 0.52 | | 1.16 | | |
| | | | | | | | | | T | | 0.63 | | 0.84 | | 0.9 | | | | | | 1.05 | 0.84 | | | | | 0.79 | | |
| | | | | | | | | | V | 1.43 | | | | | | | | | | | | | 0.6 | | | | 1.33 | 1.43 | |
| | | | | | | | | | W | | | | | | | | | | | | | | 1.03 | | | | | | |
| | | | | | | | | | Y | | | | | | | | | | 0.17 | | 2.22 | 1.59 | | | 0.23 | | 0.49 | 0.91 | |

TABLE 19

| | Domain | | | | | | | | | | | | | | | | | | | | | | | | |
|---|---|---|---|---|---|---|---|---|---|---|---|---|---|---|---|---|---|---|---|---|---|---|---|---|---|
| | FR1 | | | | | | CDR1 | | | | FR2 | CDR2 | | | | | | | | | | | | | |
| | Kabat numbering | | | | | | | | | | | | | | | | | | | | | | | | |
| | 11 | 16 | 19 | 28 | 29 | 30 | 31 | 32 | 33 | 35 | 43 | 50 | 51 | 52 | 52a | 52b | 52c | 53 | 54 | 55 | 56 | 57 | 58 | 59 | 60 |
| | Amino acid before substitution | | | | | | | | | | | | | | | | | | | | | | | | |
| (wt) | V | R | R | T | F | S | N | A | W | H | K | Q | I | K | A | K | S | N | N | Y | A | T | Y | Y | A |
| A | | | | | | | 0.96 | | 29.99 | 25.04 | | 22.63 | | | | 0.58 | 0.87 | 0.55 | 0.58 | | | 0.87 | 1.06 | 0.74 | |
| D | | | | 0.93 | | 0.79 | 1.14 | | 1693.03 | 68.99 | | 75.37 | 6.37 | 166.47 | 1.35 | 0.56 | 0.55 | 0.55 | 0.59 | 0.89 | 0.71 | | 4.81 | 0.66 | 0.94 |
| E | | 0.74 | | | | | | | | 70.35 | 0.88 | 16738.09 | 0.54 | 19.38 | 0.89 | | | | | | | | | 0.61 | 0.88 |
| F | | | | | | | 1.24 | | 0.66 | 53.59 | | | | 4.04 | | | 0.93 | | 0.97 | | | | | | |
| G | | | | | | 0.93 | 1.37 | | | 45.77 | | | | | 0.61 | | 0.81 | 0.95 | 0.84 | 0.99 | 0.59 | | | | |
| H | | | | | | | 0.96 | | 4.96 | | | | | 2.65 | | | | 0.55 | | | | | | | |
| I | | | | | | | 0.62 | | | 7.23 | | 1.21 | | 3.54 | | | | 0.57 | | | | 0.81 | | | |
| K | | | | | | | 0.97 | | | 14.45 | | 0.71 | | | | | 0.86 | 0.79 | 0.82 | | 1.32 | 1.22 | 0.66 | | |
| L | 0.83 | | | | | | | | 56573.23 | 4.8 | | 1.41 | | | | 0.61 | 0.94 | | 0.91 | | 0.77 | | 1.21 | | |
| M | | | | | | | | | | 3.98 | | | | | | | | | | | | | | | |
| N | | | | | | | | | | 2.88 | | 1.48 | | 3.29 | | 0.43 | | | | | 0.84 | 0.9 | 1.86 | | |
| P | | | | | | | | | | | | | | | 5 | | | | | | | | | | |
| Q | | 0.87 | | | | | 0.94 | | | 4.8 | 0.89 | | | | | 0.62 | 0.97 | 1.05 | 0.8 | | 0.74 | | 1.24 | | |
| R | | | | | | | 0.98 | | | 15429.77 | | | | 0.8 | | 0.91 | | | | | | | | | |
| S | | | | 0.79 | | | 0.67 | | | 2.93 | | 47.38 | | 92.1 | 0.82 | 0.58 | | 0.59 | 0.57 | 5.65 | | | 1.22 | 0.79 | |
| T | | | 0.81 | | | | | | | 4.4 | | | | | | | | | | | | | | | |
| V | | | | | 2.94 | | | | | 28.08 | | | | | 0.95 | | 0.82 | | | | | | | | |
| W | | | | | | | 1.07 | | | 50.42 | | | | 2.69 | | | | 0.69 | | | | | | | |
| Y | | | | | | | 1.1 | 2.11 | 0.69 | 119458.13 | | 49.09 | | 6.47 | 7.71 | 0.61 | 0.87 | 0.94 | 1.03 | | 0.63 | | | | |
| | | Domain | | | | | | | | | | | | | | | | | | | | | | | |
| | | CDR2 | | | | FR3 | | | | | | | | CDR3 | | | | | | | | | | | FR4 |
| | | Kabat numbering | | | | | | | | | | | | | | | | | | | | | | | |
| | | 61 | 62 | 64 | 65 | 72 | 73 | 74 | 75 | 76 | 77 | 78 | 82a | 95 | 96 | 97 | 98 | 99 | 100 | 100a | 100b | 100c | 101 | 102 | 105 |
| | | Amino acid before substitution | | | | | | | | | | | | | | | | | | | | | | | |
| | (wt) | E | S | K | G | D | D | S | K | N | S | L | N | V | H | Y | G | A | Y | Y | G | V | D | A | Q |
| | A | 0.94 | | 0.81 | | 1.19 | 0.73 | 0.77 | | | | | | 3.15 | 1 | 41309 | 0.98 | 0.92 | 0.66 | | | 0.86 | | | |
| | D | 0.9 | 0.87 | 0.76 | 0.61 | | | | | | | | 0.56 | 108.01 | 7.27 | 65.7 | 2.36 | 1.03 | 0.63 | 1.2 | 6.25 | 1.64 | | | |
| | E | | 0.82 | 0.84 | 0.61 | 0.73 | 0.56 | | | | | | | 50.46 | | | | | | | 7.29 | 1.31 | | | 0.89 |
| | F | | | | | | | | | | | | | | | | | 1.15 | | 0.98 | 4.37 | | 0.73 | | |
| | G | | | | | | 0.78 | | | | | | | 78256.33 | 0.8 | 47213 | | | 0.97 | 1.01 | | | 3.16 | | |
| | H | | | | | | | | | | | | | | | | 1.14 | | | | | | 0.91 | | |
| | I | | | | | | | | | | | 1.08 | | | | | | | | | | | 1.73 | 1.29 | |
| | K | | 0.99 | | | | 0.74 | | | | | | | 1.15 | 1.56 | | 4.85 | 1.4 | 0.93 | 0.79 | | | 4.37 | | |
| | L | | | | | | | | | | | | | | 3.14 | | | 1 | 0.67 | 0.57 | 5.84 | | 0.71 | | |
| | M | | | | | | | | | | | | | | | | | | | | | | 1.94 | | |
| | N | | | | | | 0.7 | | | | | | | | | | | | | | | | 2.28 | | |
| | P | 0.82 | 0.77 | | | | | 0.7 | | | | | | | 87044.4 | 12429 | 0.88 | | 1.3 | | | 0.97 | 43.42 | 3.51 | |
| | Q | 0.65 | | 0.87 | | | | | | | | | | | 1.36 | | 1.04 | 0.85 | 0.77 | 0.51 | | | 3.55 | | |
| | R | | | | | | 0.79 | | 0.88 | | | | | | 1.59 | 23180 | 4.69 | | | | | | 5.56 | | |
| | S | | | 0.85 | | | | | | 0.84 | | | | 4.61 | 1.15 | 1178 | 0.98 | 0.76 | 0.7 | 0.59 | 1.25 | | 0.91 | | |
| | T | | | | | | 0.78 | | 0.75 | | 0.83 | | | | | | 0.93 | 0.93 | | | | | 0.62 | | |
| | V | | | | | 1.17 | | | | | | | | | | | | | 0.92 | | | | 1.18 | 1.27 | |
| | W | | | | | | | | | | | | | | | | | | 0.96 | | | | | | |
| | Y | | | | | | | | | | | | | | 6.67 | | 2.75 | 1.25 | | | 51.41 | | 0.97 | 1 | |

12.3.2. Alteration of L Chain

Table 20 shows the results of the ratio of the amount of each L chain altered form bound to the amount of the corresponding unaltered antibody GLS3000 bound. Specifically, when the amount of the GLS3000-containing antibody bound was defined as X and the amount of the L chain one-amino acid altered form bound was defined as Y, a value of Z (ratio of amounts bound)=Y/X was used. As shown in FIG. 25, a very small amount bound was observed in the sensorgram for Z of less than 0.8, suggesting the possibility that the dissociation constant K_{D} (M) cannot be calculated correctly. Table 21 shows the dissociation constant K_{D} (M) ratio of each L chain altered form to GLS3000.

When Z shown in Table 20 is 0.8 or more, the altered form is considered to maintain the binding relative to the corresponding unaltered antibody GLS3000. Therefore, an antibody library designed such that these amino acids appear can serve as a dual Fab library.

TABLE 20

| | Domain | | | | | | | | | | | | | | | | | | |
|---|---|---|---|---|---|---|---|---|---|---|---|---|---|---|---|---|---|---|---|
| | | CDR1 | | | | | | | | | | | | | | | | FR2 | |
| | Kabat numbering | | | | | | | | | | | | | | | | | | |
| | 24 | 25 | 26 | 27 | 27a | 27b | 27c | 27d | 27e | 28 | 29 | 30 | 31 | 32 | 33 | 34 | | 45 | |
| | Amino acid before substitution | | | | | | | | | | | | | | | | | | |
| | R | S | S | Q | S | L | V | H | S | N | R | N | T | Y | L | H | | Q | |
| A | 0.86 | 0.92 | | | | | | 0.48 | 1.03 | 0.25 | 0.63 | 0.5 | | 0.24 | 0.85 | 1.06 | | | |
| D | 0.75 | 0.18 | 0.86 | 0.85 | 0.79 | 0.17 | 0.32 | 0.22 | 0.69 | 0.19 | 0.41 | 0.34 | 0.23 | 0.23 | 0.17 | 0.22 | | 0.77 | |
| E | 0.83 | 0.21 | 0.74 | 0.88 | 0.81 | 0.17 | 0.61 | 0.23 | 0.76 | 0.4 | | 0.44 | 0.49 | | 0.72 | 0.23 | | 0.75 | |
| F | | | | | | 0.42 | | 0.63 | 1.32 | 0.46 | | 1.1 | | 0.29 | 0.78 | 0.27 | | | |
| G | 0.89 | | | | 1.03 | | | 0.3 | 1.04 | 0.46 | 0.67 | 0.47 | | | | 1.02 | | | |
| H | | | | | | | | | 1.23 | 0.42 | | 0.98 | | | | | | | |
| I | | 0.53 | | | 1 | 1.19 | 0.96 | 0.26 | 1.07 | 0.44 | 0.37 | 0.61 | 0.97 | | 0.83 | 0.65 | | | |
| K | | | | | | | | 0.29 | 1.59 | 0.44 | | 1.65 | 1.04 | | | 2.17 | | | |
| L | | 0.24 | | 0.92 | | | 0.84 | 0.3 | 1.17 | 0.39 | 0.56 | 0.7 | | | | 0.59 | | | |
| M | | 0.31 | | | | 0.71 | | 0.3 | 1.23 | 0.39 | | 0.8 | | | 0.93 | 0.35 | | | |
| N | | | | | 1.1 | | | 0.3 | 1.16 | | | | 0.32 | | | 0.65 | | | |
| P | 0.7 | 1.01 | 0.78 | 0.29 | 0.99 | 0.91 | 0.3 | 0.24 | 1.26 | 0.36 | 0.31 | 0.31 | 0.31 | 0.24 | 0.3 | 0.34 | | | |
| Q | 0.9 | | | | | | | 0.25 | 1.1 | 0.37 | | 0.87 | | | | 0.25 | | 0.86 | |
| R | | | | 1.19 | | | | 0.31 | 1.58 | | | 1.86 | | | | 0.2 | | | |
| S | 0.89 | | | | | 0.71 | 0.51 | 0.32 | | 0.32 | 0.68 | 0.29 | | | | 0.78 | | | |
| T | 0.88 | 0.83 | | | | | | 0.29 | 0.97 | 0.45 | 0.63 | 0.29 | | | | 0.89 | | | |
| V | | 0.73 | | | | 1.12 | | 0.3 | 1.08 | 0.36 | 0.34 | 0.61 | | | 1.05 | 0.85 | | | |
| W | | | | | | 0.26 | | 0.39 | 1.55 | 0.41 | | 0.99 | | | | 0.24 | | | |
| Y | 0.87 | | | | 1.1 | 0.25 | 0.77 | 0.64 | 1.2 | 0.26 | 0.69 | 1.04 | | | | 0.59 | | | |
| | Domain | | | | | | | | | | | | | | | | | | |
| | CDR2 | | | | | | | FR3 | | CDR3 | | | | | | | | | FR4 |
| | Kabat numbering | | | | | | | | | | | | | | | | | | |
| | 50 | 51 | 52 | 53 | 54 | 55 | 56 | 74 | 77 | 89 | 90 | 91 | 92 | 93 | 94 | 95 | 96 | 97 | 107 |
| | Amino acid before substitution | | | | | | | | | | | | | | | | | | |
| | K | V | S | N | R | F | S | K | R | G | Q | G | T | Q | V | P | Y | T | K |
| A | 0.23 | | 0.93 | 0.61 | 0.69 | 1.13 | 1.16 | | | 1.13 | 0.5 | 0.27 | 0.63 | 0.85 | 1.05 | 0.63 | | | |
| D | 0.22 | 0.33 | 0.63 | 0.34 | 0.36 | 0.65 | 0.77 | | | 0.33 | 0.19 | 0.16 | 0.18 | 0.72 | 0.89 | 0.24 | 0.17 | | |
| E | 0.24 | 0.64 | | 0.54 | 0.58 | 0.72 | 0.71 | | | 0.26 | 0.86 | 0.16 | 0.17 | 0.75 | 0.5 | 0.39 | 0.17 | | 0.94 |
| F | | | | 0.69 | | | 1.32 | | | 1.09 | | | | | 0.71 | | 1.17 | | |
| G | 0.16 | 0.84 | | 0.76 | 0.67 | 1.31 | 0.92 | | | | | | | | 0.48 | 0.37 | | | |
| H | | | | 1.18 | | 0.94 | 1.05 | | | 0.7 | | | | | 0.78 | | 0.23 | | |
| I | | 0.81 | | 0.5 | | 0.82 | 0.99 | | | 1.07 | | | 0.34 | | 0.66 | | | | |
| K | | | | 1.08 | | 1.33 | 1.46 | | | 0.4 | | | | | 0.57 | | | | |
| L | 0.24 | | | 0.56 | | 0.76 | 1.02 | | | 0.94 | | | 0.42 | | 0.44 | 0.24 | 0.32 | | |
| M | | | | 0.62 | | 0.8 | 1.05 | | | 0.52 | | | | | 0.44 | | | | |
| N | | | | | | 0.98 | 0.92 | | | 0.8 | | | | | 1.05 | | | | |
| P | 0.3 | 0.32 | 0.33 | 0.81 | 0.84 | 1.16 | 0.95 | | | 0.35 | 0.27 | 0.27 | 0.26 | 0.25 | 1.26 | | | 0.31 | |
| Q | 0.18 | | 1.05 | 0.77 | 0.68 | 0.91 | 1.04 | | | 0.38 | | | | | 0.76 | | | | |
| R | 0.5 | | | 1.58 | | 1.31 | 1.36 | | | 0.19 | | | | 1.13 | 0.66 | | | | |
| S | 0.23 | | | 0.69 | 0.79 | 0.69 | | | 0.92 | | 0.73 | 0.26 | 0.96 | 0.96 | 0.93 | 0.43 | | | |
| T | 0.19 | | | 0.56 | 0.65 | 0.41 | 0.97 | 0.84 | | 1.03 | | 0.26 | | | 0.93 | | | | |
| V | | | | 0.56 | | 0.71 | 0.95 | | | 1.63 | | | | | | | | | |
| W | | | | 0.81 | 0.78 | 0.69 | 1.38 | | | 0.5 | | | | | 0.58 | | | | |
| Y | 0.24 | | 1.12 | 0.67 | | 0.92 | 1.46 | | | 1.19 | 0.17 | 0.17 | 0.33 | 0.87 | 0.63 | | | | |

TABLE 21

| | Domain | | | | | | | | | | | | | | | | | | |
|---|---|---|---|---|---|---|---|---|---|---|---|---|---|---|---|---|---|---|---|
| | CDR1 | | | | | | | | | | | | | | | | | FR2 | |
| | Kabat numbering | | | | | | | | | | | | | | | | | | |
| | 24 | 25 | 26 | 27 | 27a | 27b | 27c | 27d | 27e | 28 | 29 | 30 | 31 | 32 | 33 | 34 | | 45 | |
| | Amino acid before substitution | | | | | | | | | | | | | | | | | | |
| | R | S | S | Q | S | L | V | H | S | N | R | N | T | Y | L | H | | Q | |
| | Affinity up | | | | | | | | | | | | | | | | | | |
| | 24 | 25 | 26 | 27 | 27a | 27b | 27c | 27d | 27e | 28 | 29 | 30 | 31 | 32 | 33 | 34 | | 45 | |
| A | 1 | 0.73 | | | | | | 2.57 | 1.01 | 4.18 | 1.15 | 1.16 | | 66.77 | 0.82 | 1.18 | | | |
| D | 0.83 | 8.86 | 1.06 | 0.89 | 0.94 | 25.07 | 3.21 | 13641 | 1.23 | 4455.11 | 1.58 | 3.82 | 30.86 | 25.92 | 37.53 | 2100 | | 0.86 | |
| E | 0.89 | 8.54 | 0.9 | 0.99 | 0.94 | 28.75 | 1.1 | 42.28 | 1.04 | 5.47 | | 2.83 | 1.59 | | 0.83 | 8.03 | | 1.01 | |
| F | | | | | | 2.67 | | 2.05 | 1.16 | 2.59 | | f | | 4.51 | 0.65 | 3.5 | | | |
| G | 0.92 | | | | 0.8 | | | 3.51 | 1.03 | 2.41 | 0.62 | 2.1 | | | | 1.08 | | | |
| H | | | | | | | | | 1.09 | 3 | | 1.08 | | | | | | | |
| I | | 0.67 | | | 0.87 | 1.17 | 1.03 | 7.77 | 1.05 | 2.81 | 1.6 | 1.24 | 1.1 | | 0.86 | 0.89 | | | |
| K | | | | | | | | 3.8 | 1.32 | 2.34 | | 1.35 | 0.88 | | | 4.1 | | | |
| L | | 4.93 | | 0.86 | | | 0.81 | 3.37 | 1.06 | 3.34 | 0.9 | 1.19 | | | | 1.03 | | | |
| M | | 1.6 | | | | 1.31 | | 3.43 | 1.11 | 3.29 | | 1.2 | | | 0.9 | 3.16 | | | |
| N | | | | | 0.98 | | | 3.43 | 1.01 | | | | 4.46 | | | 2.84 | | | |
| P | 0.34 | 0.79 | 0.67 | 2.16 | 1.01 | 0.96 | 3.71 | 9.21 | 1.06 | 4.18 | 14.01 | 12.14 | 10.82 | 61.98 | 32.86 | 1.22 | | | |
| Q | 0.87 | | | | | | | 7.48 | 1.08 | 3.48 | | 1 | | | | 4.6 | | 0.98 | |
| R | | | | 1.06 | | | | 2.35 | 1.35 | | | 1.73 | | | | 85764 | | | |
| S | 0.97 | | | | | 0.9 | 3.04 | 3.05 | | 4.3 | 1.05 | 10.64 | | | | 1.24 | | | |
| T | 1.03 | 0.75 | | | | | | 12973 | 0.98 | 2.67 | 1.02 | 12.72 | | | | 1.1 | | | |
| V | | 0.74 | | | | 1.11 | | 353.86 | 0.95 | 3.73 | 2.25 | 2.62 | | | 1.26 | 1.04 | | | |
| W | | | | | | 23.6 | | 1.86 | 1.32 | 3.17 | | 0.97 | | | | 8.45 | | | |
| Y | 0.94 | | | | 0.93 | 22.2 | 1.25 | 1.98 | 1.1 | 3.89 | 1.08 | 1.03 | | | | 2.44 | | | |
| | Domain | | | | | | | | | | | | | | | | | | |
| | CDR2 | | | | | | | FR3 | | CDR3 | | | | | | | | | FR4 |
| | Kabat numbering | | | | | | | | | | | | | | | | | | |
| | 50 | 51 | 52 | 53 | 54 | 55 | 56 | 74 | 77 | 89 | 90 | 91 | 92 | 93 | 94 | 95 | 96 | 97 | 107 |
| | Amino acid before substitution | | | | | | | | | | | | | | | | | | |
| | K | V | S | N | R | F | S | K | R | G | Q | G | T | Q | V | P | Y | T | K |
| | Affinity up | | | | | | | | | | | | | | | | | | |
| | 50 | 51 | 52 | 53 | 54 | 55 | 56 | 74 | 77 | 89 | 90 | 91 | 92 | 93 | 94 | 95 | 96 | 97 | 107 |
| A | 59.5 | | 0.9 | 0.82 | 0.85 | 1.16 | 1.18 | | | 1.1 | 0.89 | 28.14 | 1.35 | 0.65 | 1.05 | 0.87 | | | |
| D | 114 | 1.5 | 0.94 | 2.8 | 1.8 | 1.02 | 1.11 | | | 1.96 | 11.13 | 44.76 | 11.19 | 0.72 | 1.05 | 2.37 | 40.88 | | |
| E | 57.2 | 0.88 | | 2.47 | 0.84 | 0.92 | 0.91 | | | 34.63 | 0.91 | 48.54 | 19.56 | 1.05 | 1.18 | 1.01 | 46.81 | | 0.95 |
| F | | | | 0.96 | | | 1.12 | | | 3.34 | | | | | 1.75 | | 0.86 | | |
| G | 42.4 | 0.83 | | 1.33 | 0.88 | 1.15 | 0.99 | | | | | | | | 2.59 | 1.94 | | | |
| H | | | | 1.31 | | 1.02 | 0.96 | | | 1.44 | | | | | 1.16 | | 80.34 | | |
| I | | 0.69 | | 2.69 | | 1.28 | 1.01 | | | 1.11 | | | 1.91 | | 1.46 | | | | |
| K | | | | 1.05 | | 1.55 | 1.21 | | | 1.8 | | | | | 0.91 | | | | |
| L | 36.4 | | | 1.62 | | 1.43 | 1.03 | | | 2.38 | | | 1.61 | | 3.06 | 11.66 | 1.84 | | |
| M | | | | 1.21 | | 1.29 | 0.93 | | | 1.96 | | | | | 2.74 | | | | |
| N | | | | | | 0.91 | 0.9 | | | 1.2 | | | | | 0.96 | | | | |
| P | 27.7 | 6 | 7.38 | 0.98 | 1.05 | 1.15 | 0.98 | | | 1.8 | 15.86 | 23.05 | 26.71 | 39.54 | 1.1 | | | 3.35 | |
| Q | 8.13 | | 1.01 | 1.28 | 1.04 | 1.09 | 0.97 | | | 2.11 | | | | | 1.1 | | | | |
| R | 1.83 | | | 1.56 | | 1.27 | 1.15 | | | 4127.4 | | | | 0.79 | 1.11 | | | | |
| S | 45.3 | | | 0.88 | 0.78 | 1.15 | | | 0.94 | | 0.96 | 72076 | 0.81 | 0.75 | 0.81 | 1.19 | | | |
| T | 25.1 | | | 2.68 | 0.89 | 2.42 | 1.01 | 0.85 | | 1.1 | | 39.87 | | | 1.06 | | | | |
| V | | | | 2.14 | | 1.12 | 0.94 | | | 1.4 | | | | | | | | | |
| W | | | | 1.01 | 0.65 | 1.72 | 1.12 | | | 2.2 | | | | | 1.81 | | | | |
| Y | 195 | | 1.02 | 0.99 | | 1.13 | 1.1 | | | 1.12 | 38.29 | 33.84 | 2.55 | 0.76 | 2.45 | | | | |

12.4. Evaluation of Binding of One-Amino Acid Alteration Antibody to ECM (Extracellular Matrix)

ECM (extracellular matrix) is an extracellular constituent and resides at various sites in vivo. Therefore, an antibody strongly binding to ECM is known to have poorer kinetics in blood (shorter half-life) (WO2012093704 A1). Thus, amino acids that do not enhance ECM binding are preferably selected as the amino acids that appear in the antibody library.

Each antibody was obtained as an H chain or L chain altered form by the method described in the Reference Example 1.2. Next, its ECM binding was evaluated according to the method of Reference Example 14. The ECM binding value (ECL reaction) of each altered form was divided by the ECM binding value of the antibody MRA (H chain: SEQ ID NO: 189, L chain: SEQ ID NO: 190) obtained in the same plate or at the same execution date, and the resulting value is shown in Tables 22 (H chain) and 23 (L chain). As shown in Tables 22 and 23, some alterations were confirmed to have tendency to enhance ECM binding.

Of the values shown in Tables 22 (H chain) and 23 (L chain), an effective value up to 10 times was adopted to the dual Fab library in consideration of the effect of enhancing ECM binding by a plurality of alterations.

TABLE 22

| | Domain | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|
| | FR1 | | | | | | CDR1 | | | | FR2 | CDR2 | | | | | | | | | | | | | | | | | |
| | Kabat numbering | | | | | | | | | | | | | | | | | | | | | | | | | | | | |
| | 11 | 16 | 19 | 28 | 29 | 30 | 31 | 32 | 33 | 35 | 43 | 50 | 51 | 52 | 52a | 52b | 52c | 53 | 54 | 55 | 56 | 57 | 58 | 59 | 60 | 61 | 62 | 64 | 65 |
| | Amino acid before substitution | | | | | | | | | | | | | | | | | | | | | | | | | | | | |
| | V | R | R | T | F | S | N | A | W | H | K | Q | I | K | A | K | S | N | N | Y | A | T | Y | Y | A | E | S | K | G |
| A | | | | | | | | | | | | | | | | | 2.95 | | 4.5 | | | 4.67 | | 5.82 | | 7.23 | | 2.08 | |
| D | | | | 0.91 | | 1.11 | | | | 1.1 | | 1.06 | 4.75 | 1.07 | 1.66 | | | | | | | | | 2.77 | 4.02 | 3.23 | 4.4 | 1.23 | 0.91 |
| E | | 1.14 | | | | | | | | 1.04 | 1.8 | 1.08 | 4.55 | 1.18 | 1.19 | | | | | | | | | 2.33 | 4.36 | | 2.75 | 1.33 | 2.13 |
| F | | | | | | | | | | 2.62 | | | | | | | 10.46 | | 15.16 | | | | | | | | | | |
| G | | | | | | 3.32 | | | | 8.82 | | | | | 4.72 | | 5.41 | | 4.43 | | | | | | | | | | |
| H | | | | | | | | | | | | | | | | | | | | | | | | | | | | | |
| I | | | | | | | 2.51 | | | | | | | | | | | | | | | | | | | | | | |
| K | | | | | | | 41.37 | | | | | | | | | | 58.7 | | 85.86 | | 32.07 | 16.29 | | | | | 4.07 | | |
| L | 3.41 | | | | | | | | | | | | | | | | 4.07 | | 6.02 | | 3.56 | | | | | | | | |
| M | | | | | | | | | | 4.69 | | | | | | | | | | | | | | | | | | | |
| N | | | | | | | | | | 3.06 | | | | | | | | | | | 4.07 | 4.49 | | | | | | | |
| P | | | | | | | | | | 51.18 | | | | | | | | | | | | | | | | 9.99 | 3.83 | | |
| Q | | 1.55 | | | | | | | | | 2 | | | | | | 4.99 | 3.18 | 3.23 | | | | | | | 9.29 | | 1.91 | |
| R | | | | | | | 71.66 | | | | | | | 11.19 | | 7.28 | | | | | | | | | | | | | |
| S | | | | 2.32 | | | 0.95 | | | | | | | | 3.34 | | | 3.71 | 4.33 | | | | | 6.58 | | | | 1.89 | |
| T | | | 1.17 | | | | | | | 3.49 | | | | | | | | | | | | | | | | | | | |
| V | | | | | 17.13 | | | | | 7.32 | | | | | | | 3.23 | | | | | | | | | | | | |
| W | | | | | | | | | | 8.8 | | | | | | | | 23.56 | | | | | | | | | | | |
| Y | | | | | | | | | | | | | | | | | 19.56 | | 17.47 | | | | | | | | | | |
| | | | | | | Domain | | | | | | | | | | | | | | | | | | | | | | | |
| | | | | | | FR3 | | | | | | | | CDR3 | | | | | | | | | | | | | | | FR4 |
| | | | | | | Kabat numbering | | | | | | | | | | | | | | | | | | | | | | | |
| | | | | | | 72 | 73 | 74 | 75 | 76 | 77 | 78 | 82a | 95 | 96 | 97 | 98 | 99 | 100 | | 100a | | 100b | | 100c | | 101 | 102 | 105 |
| | | | | | | Amino acid before substitution | | | | | | | | | | | | | | | | | | | | | | | |
| | | | | | | D | D | S | K | N | S | L | N | V | H | Y | G | A | Y | | Y | | G | | V | | D | A | Q |
| | | | | | A | 22.3 | | | | | | | | | | | 2.7 | | 1.46 | | | | | | | | 66.85 | | |
| | | | | | D | | | | | | | | 1.12 | 0.96 | | 0.65 | | | | | | | 0.98 | | 1.18 | | | | |
| | | | | | E | | | | | | | | | 0.76 | | | | | | | | | 1.2 | | 1.3 | | | | 1.33 |
| | | | | | F | | | | | | | | | | | | | 16.97 | | | 2.81 | | | | | | | | |
| | | | | | G | | | | | | | | | | | | | | 1 | | 2.61 | | | | | | 56.66 | | |
| | | | | | H | | | | | | | | | | | | 2.12 | | | | | | | | | | 16.16 | | |
| | | | | | I | | | | | | | | | | | | | | | | | | | | | | 63.16 | 6.63 | |
| | | | | | K | | | | | | | | | | | | 32.29 | 57.13 | 8.2 | | 10.3 | | | | | | 38.94 | | |
| | | | | | L | | | | | | | | | | | | | 6.94 | | | | | | | | | | | |
| | | | | | M | | | | | | | | | | | | | | | | | | | | | | 123.87 | | |
| | | | | | N | | | | | | | | | | | | | | | | | | | | | | 90.66 | | |
| | | | | | P | | | | | | | | | | | | 3 | | | | | | | | | | | | |
| | | | | | Q | | | | | | | | | | | | 2.99 | 2.12 | 0.94 | | | | | | | | 130.29 | | |
| | | | | | R | | | | 2.92 | | | | | | | | 48.83 | | | | | | | | | | | | |
| | | | | | S | | | | | 1.93 | | | | | | | 2.41 | 3.34 | 1 | | | | | | | | 58.7 | | |
| | | | | | T | | | | 1.2 | | 2.31 | | | | | | 1.6 | 2.54 | | | | | | | | | | | |
| | | | | | V | | | | | | | | | | | | | | | | | | | | | | 48.47 | 6.29 | |
| | | | | | W | | | | | | | | | | | | | | 10.83 | | | | | | | | | | |
| | | | | | Y | | | | | | | | | | | | 27.01 | 30.37 | | | | | | | | | | 2.82 | |

TABLE 23

| | Domain | | | | | | | | | | | | | | | | | | |
|---|---|---|---|---|---|---|---|---|---|---|---|---|---|---|---|---|---|---|---|
| | CDR1 | | | | | | | | | | | | | | | | | FR2 | |
| | Kabat numbering | | | | | | | | | | | | | | | | | | |
| | 24 | 25 | 26 | 27 | 27a | 27b | 27c | 27d | 27e | 28 | 29 | 30 | 31 | 32 | 33 | 34 | | 45 | |
| | Amino acid before substitution | | | | | | | | | | | | | | | | | | |
| | R | S | S | Q | S | L | V | H | S | N | R | N | T | Y | L | H | | Q | |
| A | 2.62 | 2.28 | | | | | | | 3.25 | | 0.87 | | | 2.21 | 5.92 | 2.61 | | | |
| D | 1.86 | 1.01 | 1.31 | 1.3 | 1.03 | 1.18 | 0.76 | | | | 0.64 | | 0.66 | 0.98 | 0.6 | | | 0.99 | |
| E | 2.02 | 1.16 | 1.22 | 1.24 | 1.12 | 1.04 | 0.72 | | 1.19 | | | | 0.79 | | 1.45 | | | 1.15 | |
| F | | | | | | | | | 16.43 | | | 5.79 | | 1.55 | | | | | |
| G | 1.53 | | | | 10.04 | | | | 5.42 | | | | | | | 3.9 | | | |
| H | | | | | | | | | 13.64 | | | 8.6 | | | | | | | |
| I | | | | | 11.11 | 2.68 | 56.75 | | 4.28 | | | | 2.87 | | 4.74 | | | | |
| K | | | | | | | | | 34.74 | | | 31.93 | 59.62 | | | 84.66 | | | |
| L | | | | 11.8 | | | 3.16 | | 5.89 | | | | | | | | | | |
| M | | | | | | | | | 6.53 | | | 3.32 | | | 19.8 | | | | |
| N | | | | | 48.45 | | | | 4.63 | | | | | | | | | | |
| P | | 2.83 | | | 2.3 | 2.7 | | | 7.26 | | | | | | | | | | |
| Q | 1.26 | | | | | | | | 2.58 | | | 3.45 | | | | | | | |
| R | | | | 18.19 | | | | | 74.03 | | | 69.62 | | | | | | | |
| S | 2.65 | | | | | 3.3 | | | | | | | | | | 2.17 | | | |
| T | 1.8 | 2.7 | | | | | | | 2.32 | | 0.63 | | | | | 4.51 | | | |
| V | | | | | | 2.82 | | | 2.31 | | | | | | 2.68 | 6.43 | | | |
| W | | | | | | | | | 46.73 | | | 11.21 | | | | | | | |
| Y | 1.89 | | | | 42.7 | | | | 30.66 | | | 3.08 | | | | | | | |
| | Domain | | | | | | | | | | | | | | | | | | |
| | CDR2 | | | | | | | FR3 | | CDR3 | | | | | | | | | FR4 |
| | Kabat numbering | | | | | | | | | | | | | | | | | | |
| | 50 | 51 | 52 | 53 | 54 | 55 | 56 | 74 | 77 | 89 | 90 | 91 | 92 | 93 | 94 | 95 | 96 | 97 | 107 |
| | Amino acid before substitution | | | | | | | | | | | | | | | | | | |
| | K | V | S | N | R | F | S | K | R | G | Q | G | T | Q | V | P | Y | T | K |
| A | 0.83 | | 3.65 | | 1.78 | 2.41 | 16.89 | | | 8.43 | 3.14 | | 3.11 | 3.34 | 3.22 | | | | |
| D | 0.84 | 1.01 | | | 1.44 | | | | | | 1.37 | 0.8 | 0.84 | | 0.88 | 4.38 | 0.66 | | |
| E | | 0.95 | | | 0.94 | | | | | | 2.55 | 1 | 0.67 | 0.85 | | 3.71 | 0.59 | | 2.96 |
| F | | | | | | | 10.25 | | | 31.93 | | | | | | | 3.44 | | |
| G | 0.67 | 3.5 | | | 1.19 | 6.49 | 3.26 | | | | | | | | | | | | |
| H | | | | 4.88 | | 7.39 | 6.83 | | | | | | | | | | | | |
| I | | 2.11 | | | | 23.25 | 5.1 | | | 14.58 | | | | | | | | | |
| K | | | | 19.31 | | 5.18 | 31.33 | | | | | | | | | | | | |
| L | | | | | | | 5.65 | | | 18.53 | | | | | | | | | |
| M | | | | | | 12.14 | 5.15 | | | | | | | | | | | | |
| N | | | | | | 7.83 | 3.96 | | | 4.96 | | | | | 3.01 | | | | |
| P | | | | 4.72 | 5.49 | 5.16 | 6 | | | | | | | | 10.7 | | | | |
| Q | 0.76 | | 2.37 | | 1.33 | 35.06 | 2.96 | | | | | | | | | | | | |
| R | | | | 34.13 | | 16.5 | 19.76 | | | | | | | 44.29 | | | | | |
| S | 0.84 | | | | | | | | 2.37 | | 4.37 | | 3.12 | 3.82 | 3.78 | | | | |
| T | 1.03 | | | | 1.39 | | 2.48 | 2.05 | | 6.79 | | | | | 2.63 | | | | |
| V | | | | | | | 4 | | | 26.88 | | | | | | | | | |
| W | | | | 2.19 | | | 26.63 | | | | | | | | | | | | |
| Y | 0.88 | | 6.28 | | | 6.18 | 3.87 | | | 28.25 | 3.75 | 3.26 | 2.96 | 14.49 | | | | | |

12.5. Study on Insertion Site and Length of Peptide for Enhancing Diversity of Library

Reference Example 11 showed that a peptide can be inserted to each site using a GGS sequence without canceling binding to CD3 (CD3 epsilon). If loop extension is possible for the dual Fab library, the resulting library might include more types of molecules (or have larger diversity) and permit obtainment of Fab domains binding to diverse second antigens. Thus, in view of presumed reduction in binding activity caused by peptide insertion, V11L/D72A/L78I/D101Q alteration to enhance binding activity against CD3 epsilon was added to the CE115HA000 sequence, which was further linked to pE22Hh. A molecule was prepared by the insertion of the GGS linker to this sequence, as in Reference Example 11, and evaluated for its CD3 binding. The GGS sequence was inserted between Kabat numbering positions 99 and 100. The antibody molecule was expressed as a one-arm antibody. Specifically, the GGS linker-containing H chain mentioned above and Kn010G3 (SEQ ID NO: 188) were used as H chains, and GLS3000 (SEQ ID NO: 185) linked to the kappa sequence (SEQ ID NO: 187) was adopted as an L chain. These sequences were expressed and purified according to Reference Example 9.

12.6. Confirmation of Binding of GGS Peptide-Inserted CE115 Antibody to CD3

The binding of the GGS peptide-inserted altered antibody to CD3 epsilon was confirmed using Biacore by the method described in Reference Example 11. As shown in Table 24, the results demonstrated that the GGS linker can be inserted to loops. Particularly, the GGS linker was able to be inserted to the H chain CDR3 region, which is important for antigen binding, and the binding to CD3 epsilon was maintained as a result of any of the 3-, 6-, and 9-amino acid insertions. Although this study was conducted using the GGS linker, an antibody library in which various amino acids other than GGS appear may be acceptable.

TABLE 24

| Inserted amino acid | |
|---|---|
| sequence (99-100) | CD3_KD [M] |
| GGS | 6.31E−08 |
| GGSGGS (SEQ ID NO: 175) | 3.46E−08 |
| GGSGGS (SEQ ID NO: 175) | 3.105E−08 |
| GGSGGGS (SEQ ID NO: 191) | 4.352E−08 |
| GGSGGGS (SEQ ID NO: 191) | 3.429E−08 |
| GGGSGGGS (SEQ ID NO: 192) | 4.129E−08 |
| GGGSGGGS (SEQ ID NO: 192) | 3.753E−08 |
| GGSGGSGGS (SEQ ID NO: 177) | 4.39E−08 |
| GGSGGSGGS (SEQ ID NO: 177) | 3.537E−08 |
| No insertion | 6.961E−09 |
| CE115HA000 | 1.097E−07 |

12.7. Study on Insertion for Library to H Chain CDR3 Using NNS Nucleotide Sequence

The paragraph (12.6) showed that the 3, 6, or 9 amino acids can be inserted using the GGS linker, and inferred that a library having the 3-, 6-, or 9-amino acid insertion can be prepared to obtain an antibody binding to the second antigen by use of a usual antibody obtainment method typified by the phage display method. Thus, a study was conducted on whether the 6-amino acid insertion to CDR3 could maintain binding to CD3 even if various amino acids appeared at the 6-amino acid insertion site using an NNS nucleotide sequence (which allows every type of amino acid to appear). In view of presumed reduction in binding activity, primers were designed using the NNS nucleotide sequence such that 6 amino acids were inserted between positions 99 and 100 (Kabat numbering) in CDR3 of a CE115HA340 sequence (SEQ ID NO: 193) having higher CD3 epsilon-binding activity than that of CE115HA000. The antibody molecule was expressed as a one-arm antibody.

Specifically, the altered H chain mentioned above and Kn010G3 (SEQ ID NO: 188) were used as H chains, and GLS3000 (SEQ ID NO: 185) linked to the kappa sequence (SEQ ID NO: 187) was adopted as an L chain. These sequences were expressed and purified according to Reference Example 9. The obtained altered antibody was evaluated for its binding by the method described in the Reference Example 12.6. The results are shown in Table 25. The results demonstrated that the binding activity against CD3 (CD3 epsilon) is maintained even if various amino acids appear at the site extended with the amino acids. Table 26 shows results of further evaluating the presence or absence of enhancement in nonspecific binding by the method described in Reference Example 10. As a result, the binding to ECM was enhanced if the extended loop of CDR3 was rich in amino acids having a positively charged side chain. Therefore, it was desired that three or more amino acids having a positively charged side chain should not appear in the loop.

TABLE 25

| | CD3_ | CDR3 | | | | | | | | | | | | | | | | | | |
|---|---|---|---|---|---|---|---|---|---|---|---|---|---|---|---|---|---|---|---|---|
| VH | KD[M] | 9 | | | | | 1 | 0 | | | | | | | | | | | | |
| CE115HA340 | 2.0E−08 | 5 | 6 | 7 | 8 | 9 | 0 | a | b | c | d | e | f | g | h | i | k | 1 | 1 | 2 |
| CE115HA340 | 2.7E−08 | V | H | Y | A | A | X | X | X | X | X | X | Y | Y | G | V | — | — | D | A |
| NNS6f17 | 7.4E−08 | . | . | . | . | . | W | G | E | G | V | V | . | . | . | . | . | . | . | . |
| NNS6f27 | 3.8E−08 | . | . | . | . | . | V | W | G | S | V | W | . | . | . | . | . | . | . | . |
| NNS6f29 | 9.0E−08 | . | . | . | . | . | I | Y | Y | P | T | N | . | . | . | . | . | . | . | . |
| NNS6f47 | 3.1E−08 | . | . | . | . | . | H | F | M | W | W | G | . | . | . | . | . | . | . | . |
| NNS6f50 | 7.1E−08 | . | . | . | . | . | L | T | G | G | L | G | . | . | . | . | . | . | . | . |
| NNS6f51 | 3.1E−08 | . | . | . | . | . | G | F | L | V | L | W | . | . | . | . | . | . | . | . |
| NNS6f52 | 5.2E−08 | . | . | . | . | . | Y | M | L | G | L | G | . | . | . | . | . | . | . | . |
| NNS6f54 | 2.9E−08 | . | . | . | . | . | F | E | W | V | G | W | . | . | . | . | . | . | . | . |
| NNS6f55 | 3.1E−08 | . | . | . | . | . | A | G | R | W | L | A | . | . | . | . | . | . | . | . |
| NNS6f56 | 2.1E−08 | . | . | . | . | . | R | E | A | T | R | W | . | . | . | . | . | . | . | . |
| NNS6f58 | 4.4E−08 | . | . | . | . | . | S | W | Q | V | S | R | . | . | . | . | . | . | . | . |
| NNS6f59 | 2.0E−07 | . | . | . | . | . | L | L | V | Q | E | G | . | . | . | . | . | . | . | . |
| NNS6f62 | 6.1E−08 | . | . | . | . | . | N | G | G | T | R | H | . | . | . | . | . | . | . | . |
| NNS6f63 | 6.9E−08 | . | . | . | . | . | G | G | G | G | W | V | . | . | . | . | . | . | . | . |
| NNS6f64 | 7.8E−08 | . | . | . | . | . | L | V | S | L | T | V | . | . | . | . | . | . | . | . |
| NNS6f67 | 3.6E−08 | . | . | . | . | . | G | L | L | R | A | A | . | . | . | . | . | . | . | . |
| NNS6f68 | 4.5E−08 | . | . | . | . | . | V | E | W | G | R | W | . | . | . | . | . | . | . | . |
| NNS6f71 | 5.1E−08 | . | . | . | . | . | G | W | V | L | G | S | . | . | . | . | . | . | . | . |
| NNS6f72 | 1.5E−07 | . | . | . | . | . | E | G | I | W | W | G | . | . | . | . | . | . | . | . |
| NNS6f73 | 2.6E−08 | . | . | . | . | . | W | V | V | G | V | R | . | . | . | . | . | . | . | . |

TABLE 26

| | ECL reaction | | Ratio | CDR 3 | | | | | | | | | | | | | | | | | | |
|---|---|---|---|---|---|---|---|---|---|---|---|---|---|---|---|---|---|---|---|---|---|---|
| | ECM | | ECM vs | 9 | | | | | 1 | 0 | | | | | | | | | | | | |
| H chain | 3 μg/ml | MRA | MRA | 5 | 6 | 7 | 8 | 9 | 0 | a | b | c | d | e | f | g | h | i | k | 1 | 1 | 2 |
| CE115HA340 | 394 | 448 | 0.9 | V | H | Y | A | A | X | X | X | X | X | X | Y | Y | G | V | — | — | D | A |
| NNS6f17 | 409 | 448 | 0.9 | . | . | . | . | . | W | G | E | G | V | V | . | . | . | . | . | . | . | . |
| NNS6f27 | 3444 | 448 | 7.7 | . | . | . | . | . | V | W | G | S | V | W | . | . | . | . | . | . | . | . |
| NNS6f29 | 481 | 448 | 1.1 | . | . | . | . | . | I | Y | Y | P | T | N | . | . | . | . | . | . | . | . |
| NNS6f47 | 94137 | 448 | 210.3 | . | . | . | . | . | H | F | M | W | W | G | . | . | . | . | . | . | . | . |
| NNS6f50 | 385 | 564 | 0.7 | . | . | . | . | . | L | T | G | G | L | G | . | . | . | . | . | . | . | . |
| NNS6f51 | 20148 | 564 | 35.7 | . | . | . | . | . | G | F | L | V | L | W | . | . | . | . | . | . | . | . |
| NNS6f52 | 790 | 564 | 1.4 | . | . | . | . | . | Y | M | L | G | L | G | . | . | . | . | . | . | . | . |
| NNS6f54 | 1824 | 564 | 3.2 | . | . | . | . | . | F | E | W | V | G | W | . | . | . | . | . | . | . | . |
| NNS6f55 | 14183 | 564 | 25.1 | . | . | . | . | . | A | G | R | W | L | A | . | . | . | . | . | . | . | . |
| NNS6f56 | 6534 | 564 | 11.6 | . | . | . | . | . | R | E | A | T | R | W | . | . | . | . | . | . | . | . |
| NNS6f58 | 2700 | 564 | 4.8 | . | . | . | . | . | S | W | Q | V | S | R | . | . | . | . | . | . | . | . |
| NNS6f59 | 388 | 564 | 0.7 | . | . | . | . | . | L | L | V | Q | E | G | . | . | . | . | . | . | . | . |
| NNS6f62 | 554 | 564 | 1.0 | . | . | . | . | . | N | G | G | T | R | H | . | . | . | . | . | . | . | . |
| NNS6f63 | 624 | 564 | 1.1 | . | . | . | . | . | G | G | G | G | W | V | . | . | . | . | . | . | . | . |
| NNS6f64 | 603 | 564 | 1.1 | . | . | . | . | . | L | V | S | L | T | V | . | . | . | . | . | . | . | . |
| NNS6f67 | 1292 | 564 | 2.3 | . | . | . | . | . | G | L | L | R | A | A | . | . | . | . | . | . | . | . |
| NNS6f68 | 2789 | 564 | 4.9 | . | . | . | . | . | V | E | W | G | R | W | . | . | . | . | . | . | . | . |
| NNS6f71 | 618 | 564 | 1.1 | . | . | . | . | . | G | W | V | L | G | S | . | . | . | . | . | . | . | . |
| NNS6f72 | 536 | 564 | 0.9 | . | . | . | . | . | E | G | I | W | W | G | . | . | . | . | . | . | . | . |
| NNS6f73 | 2193 | 564 | 3.9 | . | . | . | . | . | W | V | V | G | V | R | . | . | . | . | . | . | . | . |

12.8. Design and Construction of Dual Fab Library

On the basis of the study described in Reference Example 12, an antibody library (dual Fab library) for obtaining an antibody binding to CD3 and the second antigen was designed as follows:

step 1: selecting amino acids that maintain the ability to bind to CD3 (CD3 epsilon) (to secure 80% or more of the amount of CE115HA000 bound to CD3);

step 2: selecting amino acids that keep ECM binding within 10 times that of MRA compared with before alteration; and

step 3: inserting 6 amino acids to between positions 99 and 100 (Kabat numbering) in H chain CDR3.

The antigen-binding site of Fab can be diversified by merely performing the step 1. The resulting library can therefore be used for identifying an antigen-binding molecule binding to the second antigen. The antigen-binding site of Fab can be diversified by merely performing the steps 1 and 3. The resulting library can therefore be used for identifying an antigen-binding molecule binding to the second antigen. Even library design without the step 2 allows an obtained molecule to be assayed and evaluated for ECM binding.

Thus, for the dual Fab library, sequences derived from CE115HA000 by adding the V11L/L78I mutation to FR (framework) and further diversifying CDRs as shown in Table 27 were used as H chains, and sequences derived from GLS3000 by diversifying CDRs as shown in Table 28 were used as L chains. These antibody library fragments can be synthesized by a DNA synthesis method generally known to those skilled in the art. The dual Fab library may be prepared as (1) a library in which H chains are diversified as shown in Table 27 while L chains are fixed to the original sequence GLS3000 or the L chain having enhanced CD3 epsilon binding described in Reference Example 12, (2) a library in which H chains are fixed to the original sequence (CE115HA000) or the H chain having enhanced CD3 epsilon binding described in Reference Example 1 while L chains are diversified as shown in Table 28, and (3) a library in which H chains are diversified as shown in Table 27 while L chains are diversified as shown in Table 28. The H chain library sequences derived from CE115HA000 by adding the V11L/L78I mutation to FR (framework) and further diversifying CDRs as shown in Table 27 were entrusted to the DNA synthesizing company DNA2.0, Inc. to obtain antibody library fragments (DNA fragments). The obtained antibody library fragments were inserted to phagemids for phage display amplified by PCR. GLS3000 was selected as L chains. The constructed phagemids for phage display were transferred to E. coli by electroporation to prepare E. coli harboring the antibody library fragments.

Based on Table 28 we designed the new diversified library for GLS3000 as shown in Table 29. The L chain library sequences was derived from GLS3000 and diversified as shown in Table 29 (DNA library). The DNA library was constructed by DNA synthesizing company. Then the L chain library containing various GLS3000 derived sequences and the H chain library containing various CE115HA000 derived sequences were inserted into phagemid to construct phage display library.

TABLE 27

| | CDR1 | | | | | CDR2 | | | | | | | | | | | | | | | | | | |
|---|---|---|---|---|---|---|---|---|---|---|---|---|---|---|---|---|---|---|---|---|---|---|---|---|
| Kabat | 3 | | | | | 5 | | 5 | | | | | | | | | | | 6 | | | | | |
| numbering | 1 | 2 | 3 | 4 | 5 | 0 | 1 | 2 | a | b | c | 3 | 4 | 5 | 6 | 7 | 8 | 9 | 0 | 1 | 2 | 3 | 4 | 5 |
| Before | N | A | W | M | H | Q | I | K | A | K | S | N | N | Y | A | T | Y | Y | A | E | S | V | K | G |
| substitution | | | | | | | | | | | | | | | | | | | | | | | | |
| Library | I | A | W | M | H | Q | I | K | D | R | A | Q | A | Y | L | A | Y | Y | A | P | S | V | K | G |
| | N | | | | | | | | | K | G | S | G | | N | N | | | | E | | | | |
| | S | | | | | | | | | | L | N | L | | A | T | | | | | | | | |
| | | | | | | | | | | | Q | | Q | | | | | | | | | | | |
| | | | | | | | | | | | V | | S | | | | | | | | | | | |
| | | | | | | | | | | | S | | N | | | | | | | | | | | |

| | CDR 3 | | | | | | | | | | | | | | | | |
|---|---|---|---|---|---|---|---|---|---|---|---|---|---|---|---|---|---|
| Kabat | 9 | | | | | 1 | 0 | | | | | | | | | | |
| numbering | 5 | 6 | 7 | 8 | 9 | 0 | a | b | c | d | e | f | g | h | i | 1 | 2 |
| Before | V | H | Y | G | A | x | x | x | x | x | x | Y | Y | G | V | D | A |
| substitution | | | | | | | | | | | | | | | | | |
| Library | V | H | Y | A | A | A | A | G | A | L | P | A | V | G | V | D | A |
| | | | | G | L | V | V | S | V | G | G | S | F | | | | |
| | | | | P | S | G | G | T | L | S | S | Q | G | | | | |
| | | | | S | Q | S | S | Y | G | Y | Y | K | | | | | |
| | | | | T | T | T | T | F | S | F | F | Y | | | | | |
| | | | | Q | | Y | Y | D | Y | | | G | | | | | |
| | | | | H | | F | F | | F | | | | | | | | |
| | | | | | | | D | | | | | | | | | | |

TABLE 28

| | Domain | | | | | | | | | | | | | | | | | | | | | | | |
|---|---|---|---|---|---|---|---|---|---|---|---|---|---|---|---|---|---|---|---|---|---|---|---|---|
| | CDR1 | | | | | | | | | | | | | | | | FR2 | CDR2 | | | | | | |
| Kabat numbering | 2 | | | | | | | | | | | 3 | | | | | 4 | 5 | | | | | | |
| | 4 | 5 | 6 | 7 | a | b | c | d | e | 8 | 9 | 0 | 1 | 2 | 3 | 4 | 5 | 0 | 1 | 2 | 3 | 4 | 5 | 6 |
| Before | R | S | S | Q | S | L | V | H | S | N | R | N | T | Y | L | H | Q | K | V | S | N | R | F | S |
| substitution | | | | | | | | | | | | | | | | | | | | | | | | |
| Library | R | S | S | Q | S | L | V | H | S | N | R | N | T | Y | L | H | Q | K | V | S | N | R | F | S |
| | A | A | D | D | E | I | L | | A | | | F | I | | A | A | | | G | A | P | P | A | G |
| | E | P | | E | P | P | | | G | | | H | | | I | G | | | I | Q | W | | G | H |
| | G | T | | | | V | | | I | | | M | | | V | T | | | | Y | | | H | I |
| | Q | | | | | | | | L | | | Q | | | | V | | | | | | | K | L |
| | S | | | | | | | | M | | | Y | | | | | | | | | | | N | M |
| | T | | | | | | | | N | | | | | | | | | | | | | | P | N |
| | Y | | | | | | | | P | | | | | | | | | | | | | | Y | P |
| | | | | | | | | | Q | | | | | | | | | | | | | | | Q |
| | | | | | | | | | T | | | | | | | | | | | | | | | T |
| | | | | | | | | | V | | | | | | | | | | | | | | | V |
| | | | | | | | | | | | | | | | | | | | | | | | | Y |
| | | | | | | | | | | | | | Domain | | | | | | | | | | | |
| | | | | | | | | | | | | | FR3 | | CDR3 | | | | | | | | | FR4 |
| | | | | | | | | Kabat numbering | | | | | 7 | | | 9 | | | | | | | | 10 |
| | | | | | | | | | | | | | 4 | 7 | 9 | 0 | 1 | 2 | 3 | 4 | 5 | 6 | 7 | 7 |
| | | | | | | | | Before | | | | | K | R | G | Q | G | T | Q | V | P | Y | T | K |
| | | | | | | | | substitution | | | | | | | | | | | | | | | | |
| | | | | | | | | Library | | | | | K | R | G | Q | G | T | Q | V | P | Y | T | K |
| | | | | | | | | | | | | | T | S | A | E | | S | A | A | | F | | E |
| | | | | | | | | | | | | | | | N | | | | S | D | | | | |
| | | | | | | | | | | | | | | | T | | | | | N | | | | |
| | | | | | | | | | | | | | | | | | | | | S | | | | |
| | | | | | | | | | | | | | | | | | | | | T | | | | |

TABLE 29

| | Region | | | | | | | | | | | | | | | |
|---|---|---|---|---|---|---|---|---|---|---|---|---|---|---|---|---|
| | CDR1 | | | | | | | | | | | | | | | |
| Kabat numbering | 2 | | | | | | | | | | | 3 | | | | |
| | 4 | 5 | 6 | 7 | a | b | c | d | e | 8 | 9 | 0 | 1 | 2 | 3 | 4 |
| Original | R | S | S | Q | S | L | V | H | S | N | R | N | T | Y | L | H |
| Library | R | S | S | Q | S | L | V | H | S | N | R | N | T | Y | L | H |
| | A | A | D | | E | | L | | A | | | F | I | | | A |
| | E | T | | E | | | | | G | | | H | | | | G |
| | G | | | | | | | | I | | | M | | | | T |
| | Q | | | | | | | | L | | | Q | | | | V |
| | S | | | | | | | | M | | | Y | | | | |
| | T | | | | | | | | N | | | | | | | |
| | Y | | | | | | | | | | | | | | | |
| | | | | | | | | | Q | | | | | | | |
| | | | | | | | | | T | | | | | | | |
| | | | | | | | | | V | | | | | | | |
| | Region | | | | | | | | | | | | | | | |
| | CDR2 | | | | | | | CDR3 | | | | | | | | |
| Kabat numbering | 5 | | | | | | | | 9 | | | | | | | |
| | 0 | 1 | 2 | 3 | 4 | 5 | 6 | 9 | 0 | 1 | 2 | 3 | 4 | 5 | 6 | 7 |
| Original | K | V | S | N | R | F | S | G | Q | G | T | Q | V | P | Y | T |
| Library | K | V | S | N | R | F | S | G | Q | G | T | Q | V | P | Y | T |
| | | | A | | | | G | A | E | | S | A | A | | F | |
| | | | Q | | | | H | N | | | | S | D | | | |
| | | | Y | | | | I | T | | | | | N | | | |
| | | | | | | | L | | | | | | S | | | |
| | | | | | | | M | | | | | | T | | | |
| | | | | | | | N | | | | | | | | | |
| | | | | | | | Q | | | | | | | | | |
| | | | | | | | T | | | | | | | | | |
| | | | | | | | V | | | | | | | | | |
| | | | | | | | Y | | | | | | | | | |

### [Reference Example 13] Experimental Cell Lines

The human GPC3 gene was integrated into the chromosome of the mouse colorectal cancer cell line CT-26 (ATCC No. CRL-2638) by a method well known to those skilled in the art to obtain the high expression CT26-GPC3 cell line. The expression level of human GPC3 (2.3×10⁵/cell) was determined using the QIFI kit (Dako) by the manufacturer's recommended method. To maintain the human GPC3 gene, these recombinant cell lines were cultured in ATCC-recommended media by adding Geneticin (GIBCO) at 200 micro g/ml for CT26-GPC3. After culturing, these cells were detached using 2.5 g/L trypsin-1 mM EDTA (nacalai tesque), and then used for each of the experiments. The transfectant cell line is herein referred to as SKpca60a. The human CD137 gene was integrated into the chromosome of the Chinese Hamster Ovary cell line CHO-DG44 by a method well known to those skilled in the art to obtain the high expression CHO-hCD137 cell line. The expression level of human CD137 was determined by FACS analysis using the PE anti-human CD137 (4-1BB) Antibody (BioLegend, Cat. No. 309803) by the manufacturer's instructions. NCI-H446 and Huh7 cell lines were maintained in RPMI1640 (Gibco) and DMEM (low glucose) respectively. Both media were supplemented with 10% fetal bovine serum (Bovogen Biologicals), 100 units/mL of penicillin and 100 micro g/mL of streptomycin and cells were cultured at 37° C. with 5% CO₂.

### [Reference Example 14] Evaluation of Binding of Antibody to ECM (Extracellular Matrix)

The binding of each antibody to ECM (extracellular matrix) was evaluated by the following procedures with reference to WO2012093704 A1: ECM Phenol red free (BD Matrigel #356237) was diluted to 2 mg/mL with TBS and added dropwise at 5 micro L/well to the center of each well of a plate for ECL assay (L15XB-3, MSD K.K., high bind) cooled on ice. Then, the plate was capped with a plate seal and left standing overnight at 4 degrees C. The ECM-immobilized plate was brought to room temperature. An ECL blocking buffer (PBS supplemented with 0.5% BSA and 0.05% Tween 20) was added thereto at 150 micro L/well, and the plate was left standing at room temperature for 2 hours or longer or overnight at 4 degrees C. Next, each antibody sample was diluted to 9 micro g/mL with PBS-T (PBS supplemented with 0.05% Tween 20). A secondary antibody was diluted to 2 micro g/mL with ECLDB (PBS supplemented with 0.1% BSA and 0.01% Tween 20). 20 micro L of the antibody solution and 30 micro L of the secondary antibody solution were added to each well of a round-bottomed plate containing ECLDB dispensed at 10 micro L/well and stirred at room temperature for 1 hour while shielded from light. The ECL blocking buffer was removed by inverting the ECM plate containing the ECL blocking buffer. To this plate, a mixed solution of the aforementioned antibody and secondary antibody was added at 50 micro L/well. Then, the plate was left standing at room temperature for 1 hour while shielded from light. The sample was removed by inverting the plate, and READ buffer (MSD K.K.) was then added thereto at 150 micro L/well, followed by the detection of the luminescence signal of the sulfo-tag using Sector Imager 2400 (MSD K.K.).

### [Reference Example 15] Assessment of the Off-Target Cytotoxicity of AntiGPC3/CD3/Human CD137 Trispecific Antibodies and Anti-GPC3/Dual-Fab Antibodies

### (15-1) Preparation of Anti-GPC3/CD3/Human CD137 Trispecific Antibodies

To investigate target independent cytotoxicity and cytokine release, trispecific antibodies were generated by utilizing CrossMab and FAE technology (FIG. 2.1). Tetravalent IgG-like molecule, Antibody A (mAb A) which of each arm has two binding domains resulting in four binding domains in one molecular was generated with CrossMab as mentioned above. Bivalent IgG, Antibody B (mAb B) is the same format as a conventional IgG. Fc region of both mAb A and mAb B was a Fc gamma R silent with attenuated affinity for Fc gamma receptor and deglycosylated and applicable for FAE. Six trispecific antibodies were constructed. The target antigen of each Fv region in six trispecific antibodies was shown in Table 30. The naming rule of each of binding domain of mAb A, mAb B, and mAb AB are shown in FIG. 2.2. The pair of mAb A and mAb B to generate each of six trispecific antibodies, mAb AB, and their SEQ ID NOs were shown in Table 31 and Table 32, respectively. Antibody CD3 D(2)_i121 which was described in WO2005/035584A1 (abbreviated as AN121) was used as anti-CD3 epsilon antibody. All six trispecific antibodies were expressed and purified by the method described above.

TABLE 30
Target of each arm of antibodies

| Name of mAb AB | Fv A1 | Fv A2 | Fv B |
|---|---|---|---|
| GPC3/CD137 × CD3 | Anti-CD137 | Anti-CD3ε | Anti-GPC3 |
| GPC3/CD137 × Ctrl | Anti-CD137 | Ctrl | Anti-GPC3 |
| GPC3/Ctrl × CD3 | Ctrl | Anti-CD3ε | Anti-GPC3 |
| Ctrl/CD137 × CD3 | Anti-CD137 | Anti-CD3ε | Ctrl |
| Ctrl/CD137 × Ctrl | Anti-CD137 | Ctrl | Ctrl |
| Ctrl/Ctrl × CD3 | Ctrl | Anti-CD3ε | Ctrl |

TABLE 3

| | Name of mAb A to | VHA1 | VLA1 | VHA2 | VLA2 | Name of mAb B to | VHB | VLB |
|---|---|---|---|---|---|---|---|---|
| Name of mAb AB | generate mAb AB | (SEQ ID NO) | (SEQ ID NO) | (SEQ ID NO) | (SEQ ID NO) | generate mAb AB | (SEQ ID NO) | (SEQ ID NO) |
| GPC3/CD137 × CD3 | CD137 × CD3 | 85 | 86 | 87 | 88 | GPC3 | 89 | 90 |
| GPC3/CD137 × Ctrl | CD137 × Ctrl | 85 | 86 | Ctrl | Ctrl | GPC3 | 89 | 90 |
| GPC3/Ctrl × CD3 | Ctrl × CD3 | Ctrl | Ctrl | 87 | 88 | GPC3 | 89 | 90 |
| Ctrl/CD137 × CD3 | CD137 × CD3 | 85 | 86 | 87 | 88 | Ctrl | Ctrl | Ctrl |
| Ctrl/CD137 × Ctrl | CD137 × Ctrl | 85 | 86 | Ctrl | Ctrl | Ctrl | Ctrl | Ctrl |
| Ctrl/Ctrl × CD3 | Ctrl × CD3 | Ctrl | Ctrl | 87 | 88 | Ctrl | Ctrl | Ctrl |

TABLE 32

| Name | SEQ | |
|---|---|---|
| of VH | ID | |
| or VL | NO | Amino acid sequence |
| CD137 | 85 | QVQLQQWGAGLLKPSETLSLTCAVYGGSFSGYYWS |
| VHA1 | | WIRQSPEKGLEWIGEINHGGYVTYNPSLESRVTIS |
| | | VDTSKNQFSLKLSSVTAADTAVYYCARDYGPGNYD |
| | | WYFDLWGRGTLVTVSS |
| CD137 | 86 | EIVLTQSPATLSLSPGERATLSCRASQSVSSYLAW |
| VLA1 | | YQQKPGQAPRLLIYDASNRATGIPARFSGSGSGTD |
| | | FTLTISSLEPEDFAVYYCQQRSNWPPALTFGGGTK |
| | | VEIK |
| CD3 | 87 | QVQLVESGGGLVQPGRSLRLSCAASGFTFSNAWMH |
| VHA2 | | WVRQAPGKGLEWVAQIKDRANSYNTYYAESVKGRF |
| | | TISRDDSKNSIYLQMNSLKTEDTAVYYCRYVHYTT |
| | | YAGSSFSYGVDAWGQGTTVTVSS |
| CD3 | 88 | DIVMTQSPLSLPVTPGEPASISCRSSQPLVHSNRN |
| VLA2 | | TYLHWYQQKPGQAPRLLIYKVSNRFSGVPDRFSGS |
| | | GSGTDFTLKISRVEAEDVGVYYCGQGTQVPYTFGQ |
| | | GTKLEIK |
| GPC3 | 89 | QVQLVQSGAEVKKPGASVTVSCKASGYTFTDYEMH |
| VHB | | WIRQPPGEGLEWIGAIDGPTPDTAYSEKFKGRVTL |
| | | TADKSTSTAYMELSSLTSEDTAVYYCTRFYSYTYW |
| | | GQGTLVTVSS |
| GPC3 | 90 | DIVMTQSPLSLPVTPGEPASISCRSSQPLVHSNRN |
| VLB | | TYLHWYQQKPGQAPRLLIYKVSNRFSGVPDRFSGS |
| | | GSGTDFTLKISRVEAEDVGVYYCGQGTQVPYTFGQ |
| | | GTKLEIK |

(15-2) Evaluation of the Binding of GPC3/CD3/Human CD137 Trispecific Antibodies Binding Affinity of Trispecific Antibodies to Human CD3 and CD137 were Assessed at 37 Degrees C. Using
Binding affinity of trispecific antibodies to human CD3 and CD137 were assessed at 37 degrees C. using Biacore T200 instrument (GE Healthcare). Anti-human Fc antibody (GE Healthcare) was immobilized onto all flow cells of a CM4 sensor chip using amine coupling kit (GE Healthcare). Antibodies were captured onto the anti-Fc sensor surfaces, then recombinant human CD3 or CD137 was injected over the flow cell. All antibodies and analytes were prepared in ACES pH 7.4 containing 20 mM ACES, 150 mM NaCl, 0.05% Tween 20, 0.005% NaN3. Sensor surface was regenerated each cycle with 3M MgCl2. Binding affinity were determined by processing and fitting the data to 1:1 binding model using Biacore T200 Evaluation software, version 2.0 (GE Healthcare).
Binding affinity of trispecific antibodies to recombinant human CD3 and CD137 are shown in Table 33.

TABLE 33

| | CD137 | | | CD3 | | |
|---|---|---|---|---|---|---|
| | ka (M⁻¹s⁻¹) | kd (s⁻¹) | KD (M) | ka (M⁻¹s⁻¹) | kd (s⁻¹) | KD (M) |
| GPC3/CD137 × CD3 | 5.47E+05 | 2.06E−02 | 3.77E−08 | 8.18E+04 | 1.61E−03 | 1.97E−08 |
| GPC3/CD137 × Ctrl | 5.72E+05 | 2.04E−02 | 3.57E−08 | no binding | | |
| GPC3/Ctrl × CD3 | no binding | | | 8.50E+04 | 1.51E−03 | 1.78E−08 |
| Ctrl/CD137 × CD3 | 5.48E+05 | 1.82E−02 | 3.31E−08 | 8.24E+04 | 1.52E−03 | 1.85E−08 |

| | | | | |
|---|---|---|---|---|
| Ctrl/CD137 × Ctrl | 5.59E+05 | 1.79E−02 | 3.21E−08 | no binding |

| | | | | |
|---|---|---|---|---|
| Ctrl/Ctrl × CD3 | no binding | 8.37E+04 | 1.47E−03 | 1.75E−08 |

### (15-3) Evaluation of the Simultaneous Binding of GPC3/CD137×CD3 Trispecific Antibodies and Anti-GPC3/Dual-Fab to Human CD137 and CD3

Biacore in-tandem blocking assay was performed to characterize simultaneous binding of Trispecific antibodies or Dual-Fab antibodies for both CD3 and CD137. The assay was performed on Biacore T200 instrument (GE Healthcare) at 25 degrees C. in ACES pH 7.4 buffer containing 20 mM ACES, 150 mM NaCl, 0.05% Tween 20, 0.005% NaN3. Anti-human Fc antibody (GE Healthcare) was immobilized onto all flow cells of a CM4 sensor chip using amine coupling kit (GE Healthcare). Antibodies were captured onto the anti-Fc sensor surfaces, then 8 micro M CD3 was injected over the flow cell followed by an identical injection of 8 micro M CD137 in the presence of 8 micro M CD3. An increased of binding response for second injection was indicative of binding to different paratopes therefore a simultaneous binding interactions; whereas no enhancement or decreased of binding response for the 2nd injection was indicative of binding to the same or overlapping or adjacent paratopes, therefore a non-simultaneous binding interactions.

Results of this assay are shown in FIG. 26 where GPC3/CD137×CD3 Trispecific antibody but not anti-GPC3/Dual-Fab antibody showed simultaneous binding characteristics to CD3 and CD137.

### (15-4) Evaluation of the Binding of GPC3/CD137×CD3 Trispecific Antibodies and Anti-GPC3/Dual-Fab Antibodies to Human CD137 Expressing CHO Cells or Jurkat Cells

FIG. 27 show binding of tri-specific antibodies and Dual-Fab antibodies to hCD137 transfectant, parental CHO cells generated in Reference Example 13 or binding to hCD3 expressed on Jurkat cells (reference Example 6-2) determined by FACS analysis. Briefly, tri-specific antibodies and Dual-Fab antibodies were incubated with each cell line for 2 hours at room temperature and washed with FACS buffer (2% FBS, 2 mM EDTA in PBS). Goat F(ab′)2 anti-Human IgG, Mouse ads-PE (Southern Biotech, Cat. 2043-09) was then added and incubated for 30 minutes at 4 degrees C. and washed with FACS buffer. Data acquisition was performed on an FACS Verse (Becton Dickinson), followed by analysis using the FlowJo software (Tree Star).

FIG. 27 shows that 50 nM of anti-GPC3/H183L072 (black line) antibody binds hCD137 specifically on hCD137 transfectant (FIG. 27a) but no binding is observed for CHO parental cells (FIG. 27b), relative to Ctrl antibody (grey filled). Similarly, 2 nM of anti-GPC3/CD137×CD3 (dark grey filled) and anti-GPC3/CD137×Ctrl (black line) tri-specific antibodies showed specific binding to hCD137 on transfectant cells (FIG. 27c) relative to Ctrl/Ctrl×CD3 tri-specific control antibody (light grey, filled). No non-specific binding was observed in CHO parental cells (FIG. 27d).

50 nM of both anti-GPC3/H183L072 (black line) antibodies in FIG. 27e and GPC3/CD137×CD3 (dark grey filled) or GPC3/CD137×Ctrl (black line) trispecific antibodies in FIG. 27f was shown to bind CD3 expressed on Jurkat cells relative to their respective controls (light grey filled).

### (15-5) Assessment of CD3 Activation on T Cell to Human GPC3 Expression Cells of GPC3/CD137×CD3 Tri-Specific Antibodies and Anti-GPC3/Dual-Fab Tri-Specific Antibodies.

To investigate if both formats of tri-specific antibodies and anti-GPC3/Dual-Fab antibodies can activate effector cells in a target-dependent manner, NFAT-luc2 Jurkat luciferase assay was conducted as described in Reference Example 6-2. 5.00E+03 SKpca60 cells (Reference Example 13) were used as target cells and co-cultured with 2.50E+04 NFAT-luc2 Jurkat cells for 24 hours in the presence of 0.1, 1 and 10 nM of tri-specific antibodies or Dual-Fab antibodies. 24 hours later, luciferase activity was detected with Bio-Glo luciferase assay system (Promega, G7940) according to manufacturer's instructions. Luminescence (units) was detected using GloMax (registered trademark) Explorer System (Promega #GM3500) and captured values were plotted using Graphpad Prism 7. As shown in FIG. 28, only tri-specific antibodies which comprised of both anti-GPC3 and anti-CD3 binding such as GPC3/CD137×CD3, GPC3/Ctrl×CD3 or anti-GPC3/H183L072 resulted in dose-dependent activation of Jurkat cells in the presence of target cells. Of note, anti-GPC3/H183L072 antibodies could elicit similar extent of Jurkat activation as GPC3/CD137×CD3 or GPC3/Ctrl×CD3 antibodies even though binding of anti-GPC3/H183L072 antibodies on Jurkat cells by FACS analysis in Reference Example (15-4) is weaker. Altogether, both tri-specific antibodies and anti-GPC3/Dual-Fab antibodies can result in target dependent activation of effector cells.

(15-6) Assessment of CD3 Activation on T Cell to Human CD137 Expression Cells of GPC3/CD137×CD3 Tri-Specific Antibodies and Anti-GPC3/Dual-Fab Antibodies.

To investigate if both tri-specific antibody formats and anti-GPC3/Dual-Fab antibodies can result in cross-linking of hCD137 expressing cells to hCD3 expressing effector cells, 5.00E+03 hCD137 expressing CHO was co-cultured with 2.50E+04 NFAT-luc2 Jurkat cells for 24 hours in the presence of 0.1, 1 and 10 nM of tri-specific antibodies as described in Reference Example (15-5). FIG. 29 showed no non-specific activation of Jurkat cells by all tri-specific antibodies when co-cultured with parental CHO cells. However, it was observed that both GPC3/CD137×CD3 and Ctrl/CD137×CD3 trispecific antibodies can activate Jurkat cells in the presence of hCD137 expressing CHO cells. Anti-GPC3/H183L072 antibodies did not result in activation of Jurkat cells when co-cultured with hCD137 expressing CHO cells. AntiGPC3/H183L072 antibody with 10 nM showed about 0.96% Luminescense of that of GPC3/CD137×CD3 trispecific antibody with 10 nM and anti-GPC3/H183L072 antibody with 1 nM showed about 1.93% Luminescence of that of GPC3/CD137×CD3 trispecific antibody with 1 nM. When it compared with the CD3 activation against GPC3 positive cells evaluated in Reference Example 15-5, about 1.36% or 1.89% Luminescence were detected against CD137 positive cells when 10 nM or 1 nM of antiGPC3/H183L072 antibodies were used although GPC3/CD137×CD3 trispecific antibody with 10 and 1 nM showed about 127.77% and 107.22% Luminescence against CD137 positive cells compared to that against GPC3 positive cells respectively.

Taken together, this suggests that tri-specific format GPC3/CD137×CD3, which binds to CD3 and CD137 at the same time, can result in Jurkat cell activation against hCD137 expressing cells independent of target or tumor antigen binding, giving rise to off-target cytotoxicity unlike that of anti-GPC3/Dual-Fab format which does not bind to CD3 and CD137 at the same time. Those results shown in Reference Example 8, 15-5 and 15-6 prove that only antibodies which does not bind to CD3 and CD137 at the same time can kill target antigen expressing cells specifically.

(15-7) Assessment of Off Target Cytokine Release of Ctrl/CD137×CD3 Tri-Specific Antibodies and Ctrl/Dual-Fab Antibodies from PBMCs

Comparison of tri-specific antibody formats and Dual-Fab antibodies for off-target toxicity was also assessed using human PBMC solution. Briefly, 2.00E+05 PBMCs prepared as described in Reference Example (7-2-1) were incubated with 80, 16 and 3.2 nM of tri-specific antibodies or Dual-Fab antibodies in the absence of target cells for 48 hours. IL-2, IFN gamma and TNF alpha in the supernatant was measured using cytokine release assay as described in Reference Example (7-2-2). As shown in FIG. 30, Ctrl/CD137×CD3 trispecific antibodies but Ctrl/Dual-Fab antibodies can result in IL-2, IFN gamma and TNF alpha release from PBMCs. 80 nM Ctrl/Dual-Fab antibodies showed about 50% IL-2 concentration of that of 80 nM Ctrl/CD137×CD3 trispecific antibodies and less than 10% IL-2 concentration was observed when 16 nM antibodies were used. As for IFN gamma and TNF alpha, Ctrl/Dual-Fab antibodies showed less than 10% IL-2 concentration of that with Ctrl/CD137×CD3 trispecific antibodies in each antibody concentration.

These results suggest that Ctrl/CD137×CD3 tri-specific format resulted in non-specific activation of PBMCs in the absence of target cells. Finally, the data showed that Dual-Fab format can confer target-specific effector cell activation without off-target toxicity.

### INDUSTRIAL APPLICABILITY

The present invention provides antigen-binding molecules capable of binding to CD3 and CD137 (4-1BB) but not binding to CD3 and CD137 at the same time. The antigen-binding molecules of the present invention exhibit enhanced T-cell dependent cytotoxity activity induced by these antigen-binding molecules through binding to the three different antigens.

1. An antigen-binding molecule comprising:
an antibody variable region that is capable of binding to CD3 and CD137, but does not bind to CD3 and CD137 at the same time;
wherein the antigen-binding molecule binds to CD137 with an equilibrium dissociation constant (KD) of less than 5×10⁻⁶ M, preferably as measured by SPR at the following condition:
37 degrees C., pH 7.4, 20 mM ACES, 150 mM NaCl, 0.05% Tween 20, 0.005% NaN3; the antigen-binding molecule is immobilized on a CM4 sensor chip, the antigen serves as analyte.

2. The antigen-binding molecule of claim 1, wherein the antigen-binding molecule binds to:
(a) at least one, two, three or more amino acid residues of the extracellular domain of CD3 epsilon (CD3 epsilon) comprising the amino acid sequence of SEQ ID NO: 159; and/or
(b) at least one, two, three or more amino acid residues of the N-terminal region of CD137 comprising the amino acid sequence of LQDPCSNCPAGTFCDNNRNQICSPCPPNSFSSAGGQRTCDICRQC KGVFRTRKECSSTSNAEC (SEQ ID NO: 152), preferably LQDPCSN, NNRNQI and/or GQRTCDI of human CD137.

3. The antigen-binding molecule of claim 1 or 2, wherein the antibody variable region comprises any one of the following:
(a1) a heavy chain complementarity determining region 1 (HCDR1) comprising an amino acid sequence that is at least 70%, 80% or 90% identical to SEQ ID NO: 16, a heavy chain complementarity determining region 2 (HCDR2) comprising an amino acid sequence that is at least 70%, 80% or 90% identical to SEQ ID NO: 30, a heavy chain complementarity determining region 3 (HCDR3) comprising an amino acid sequence that is at least 70%, 80% or 90% identical to SEQ ID NO: 44, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;
(a2) a heavy chain complementarity determining region 1 (HCDR1) comprising an amino acid sequence that is at least 70%, 80% or 90% identical to SEQ ID NO: 17, a heavy chain complementarity determining region 2 (HCDR2) comprising an amino acid sequence that is at least 70%, 80% or 90% identical to SEQ ID NO: 31, a heavy chain complementarity determining region 3 (HCDR3) comprising an amino acid sequence that is at least 70%, 80% or 90% identical to SEQ ID NO: 45, a light chain complementarity determining region 1 (LCDR1) comprising an amino acid sequence that is at least 70%, 80% or 90% identical to SEQ ID NO: 64, a light chain complementarity determining region 2 (LCDR2) comprising an amino acid sequence that is at least 70%, 80% or 90% identical to SEQ ID NO: 69, and a light chain complementarity determining region 3 (LCDR3) comprising an amino acid sequence that is at least 70%, 80% or 90% identical to SEQ ID NO: 74;
(a3) a heavy chain complementarity determining region 1 (HCDR1) comprising an amino acid sequence that is at least 70%, 80% or 90% identical to SEQ ID NO: 18, a heavy chain complementarity determining region 2 (HCDR2) comprising an amino acid sequence that is at least 70%, 80% or 90% identical to SEQ ID NO: 32, a heavy chain complementarity determining region 3 (HCDR3) comprising an amino acid sequence that is at least 70%, 80% or 90% identical to SEQ ID NO: 46, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;
(a4) a heavy chain complementarity determining region 1 (HCDR1) comprising an amino acid sequence that is at least 70%, 80% or 90% identical to SEQ ID NO: 19, a heavy chain complementarity determining region 2 (HCDR2) comprising an amino acid sequence that is at least 70%, 80% or 90% identical to SEQ ID NO: 33, a heavy chain complementarity determining region 3 (HCDR3) comprising an amino acid sequence that is at least 70%, 80% or 90% identical to SEQ ID NO: 47, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;
(a5) a heavy chain complementarity determining region 1 (HCDR1) comprising an amino acid sequence that is at least 70%, 80% or 90% identical to SEQ ID NO: 19, a heavy chain complementarity determining region 2 (HCDR2) comprising an amino acid sequence that is at least 70%, 80% or 90% identical to SEQ ID NO:33, a heavy chain complementarity determining region 3 (HCDR3) comprising an amino acid sequence that is at least 70%, 80% or 90% identical to SEQ ID NO: 47, a light chain complementarity determining region 1 (LCDR1) comprising an amino acid sequence that is at least 70%, 80% or 90% identical to SEQ ID NO: 65, a light chain complementarity determining region 2 (LCDR2) comprising an amino acid sequence that is at least 70%, 80% or 90% identical to SEQ ID NO: 70, and a light chain complementarity determining region 3 (LCDR3) comprising an amino acid sequence that is at least 70%, 80% or 90% identical to SEQ ID NO: 75;
(a6) a heavy chain complementarity determining region 1 (HCDR1) comprising an amino acid sequence that is at least 70%, 80% or 90% identical to SEQ ID NO: 20, a heavy chain complementarity determining region 2 (HCDR2) comprising an amino acid sequence that is at least 70%, 80% or 90% identical to SEQ ID NO: 34, a heavy chain complementarity determining region 3 (HCDR3) comprising an amino acid sequence that is at least 70%, 80% or 90% identical to SEQ ID NO: 48, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;
(a7) a heavy chain complementarity determining region 1 (HCDR1) comprising an amino acid sequence that is at least 70%, 80% or 90% identical to SEQ ID NO: 22, a heavy chain complementarity determining region 2 (HCDR2) comprising an amino acid sequence that is at least 70%, 80% or 90% identical to SEQ ID NO: 36, a heavy chain complementarity determining region 3 (HCDR3) comprising an amino acid sequence that is at least 70%, 80% or 90% identical to SEQ ID NO: 50, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;
(a8) a heavy chain complementarity determining region 1 (HCDR1) comprising an amino acid sequence that is at least 70%, 80% or 90% identical to SEQ ID NO: 23, a heavy chain complementarity determining region 2 (HCDR2) comprising an amino acid sequence that is at least 70%, 80% or 90% identical to SEQ ID NO: 37, a heavy chain complementarity determining region 3 (HCDR3) comprising an amino acid sequence that is at least 70%, 80% or 90% identical to SEQ ID NO: 51, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;
(a9) a heavy chain complementarity determining region 1 (HCDR1) comprising an amino acid sequence that is at least 70%, 80% or 90% identical to SEQ ID NO: 23, a heavy chain complementarity determining region 2 (HCDR2) comprising an amino acid sequence that is at least 70%, 80% or 90% identical to SEQ ID NO: 37, a heavy chain complementarity determining region 3 (HCDR3) comprising an amino acid sequence that is at least 70%, 80% or 90% identical to SEQ ID NO: 51, a light chain complementarity determining region 1 (LCDR1) comprising an amino acid sequence that is at least 70%, 80% or 90% identical to SEQ ID NO: 66, a light chain complementarity determining region 2 (LCDR2) comprising an amino acid sequence that is at least 70%, 80% or 90% identical to SEQ ID NO: 71, and a light chain complementarity determining region 3 (LCDR3) comprising an amino acid sequence that is at least 70%, 80% or 90% identical to SEQ ID NO: 76;
(a10) a heavy chain complementarity determining region 1 (HCDR1) comprising an amino acid sequence that is at least 70%, 80% or 90% identical to SEQ ID NO: 24, a heavy chain complementarity determining region 2 (HCDR2) comprising an amino acid sequence that is at least 70%, 80% or 90% identical to SEQ ID NO: 38, a heavy chain complementarity determining region 3 (HCDR3) comprising an amino acid sequence that is at least 70%, 80% or 90% identical to SEQ ID NO: 52, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;
(a11) a heavy chain complementarity determining region 1 (HCDR1) comprising an amino acid sequence that is at least 70%, 80% or 90% identical to SEQ ID NO: 25, a heavy chain complementarity determining region 2 (HCDR2) comprising an amino acid sequence that is at least 70%, 80% or 90% identical to SEQ ID NO: 39, a heavy chain complementarity determining region 3 (HCDR3) comprising an amino acid sequence that is at least 70%, 80% or 90% identical to SEQ ID NO: 53, a light chain complementarity determining region 1 (LCDR1) comprising an amino acid sequence that is at least 70%, 80% or 90% identical to SEQ ID NO: 66, a light chain complementarity determining region 2 (LCDR2) comprising an amino acid sequence that is at least 70%, 80% or 90% identical to SEQ ID NO: 71, and a light chain complementarity determining region 3 (LCDR3) comprising an amino acid sequence that is at least 70%, 80% or 90% identical to SEQ ID NO: 76;
(a12) a heavy chain complementarity determining region 1 (HCDR1) comprising an amino acid sequence that is at least 70%, 80% or 90% identical to SEQ ID NO: 26, a heavy chain complementarity determining region 2 (HCDR2) comprising an amino acid sequence that is at least 70%, 80% or 90% identical to SEQ ID NO: 40, a heavy chain complementarity determining region 3 (HCDR3) comprising an amino acid sequence that is at least 70%, 80% or 90% identical to SEQ ID NO: 54, a light chain complementarity determining region 1 (LCDR1) comprising an amino acid sequence that is at least 70%, 80% or 90% identical to SEQ ID NO: 66, a light chain complementarity determining region 2 (LCDR2) comprising an amino acid sequence that is at least 70%, 80% or 90% identical to SEQ ID NO: 71, and a light chain complementarity determining region 3 (LCDR3) comprising an amino acid sequence that is at least 70%, 80% or 90% identical to SEQ ID NO: 76;
(a13) a heavy chain complementarity determining region 1 (HCDR1) comprising an amino acid sequence that is at least 70%, 80% or 90% identical to SEQ ID NO: 26, a heavy chain complementarity determining region 2 (HCDR2) comprising an amino acid sequence that is at least 70%, 80% or 90% identical to SEQ ID NO: 40, a heavy chain complementarity determining region 3 (HCDR3) comprising an amino acid sequence that is at least 70%, 80% or 90% identical to SEQ ID NO: 54, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;
(a14) a heavy chain complementarity determining region 1 (HCDR1) comprising an amino acid sequence that is at least 70%, 80% or 90% identical to SEQ ID NO:27, a heavy chain complementarity determining region 2 (HCDR2) comprising an amino acid sequence that is at least 70%, 80% or 90% identical to SEQ ID NO: 41, a heavy chain complementarity determining region 3 (HCDR3) comprising an amino acid sequence that is at least 70%, 80% or 90% identical to SEQ ID NO: 55, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;
(a15) a heavy chain complementarity determining region 1 (HCDR1) comprising an amino acid sequence that is at least 70%, 80% or 90% identical to SEQ ID NO: 28, a heavy chain complementarity determining region 2 (HCDR2) comprising an amino acid sequence that is at least 70%, 80% or 90% identical to SEQ ID NO: 42, a heavy chain complementarity determining region 3 (HCDR3) comprising an amino acid sequence that is at least 70%, 80% or 90% identical to SEQ ID NO: 56, a light chain complementarity determining region 1 (LCDR1) comprising an amino acid sequence that is at least 70%, 80% or 90% identical to SEQ ID NO: 63, a light chain complementarity determining region 2 (LCDR2) comprising an amino acid sequence that is at least 70%, 80% or 90% identical to SEQ ID NO: 68, and a light chain complementarity determining region 3 (LCDR3) comprising an amino acid sequence that is at least 70%, 80% or 90% identical to SEQ ID NO: 73;
(b1) a HCDR1 comprising an amino acid sequence of SEQ ID NO: 16, a HCDR2 comprising an amino acid sequence of SEQ ID NO: 30, a HCDR3 comprising an amino acid sequence of SEQ ID NO: 44, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b2) a HCDR1 comprising an amino acid sequence of SEQ ID NO: 17, a HCDR2 comprising an amino acid sequence of SEQ ID NO: 31, a HCDR3 comprising an amino acid sequence of SEQ ID NO: 45, a LCDR1 comprising an amino acid sequence of SEQ ID NO: 64, a LCDR2 comprising an amino acid sequence of SEQ ID NO: 69, and a LCDR3 comprising an amino acid sequence of SEQ ID NO: 74;
(b3) a HCDR1 comprising an amino acid sequence of SEQ ID NO: 18, a HCDR2 comprising an amino acid sequence of SEQ ID NO: 32, a HCDR3 comprising an amino acid sequence of SEQ ID NO: 46, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b4) a HCDR1 comprising an amino acid sequence of SEQ ID NO: 19, a HCDR2 comprising an amino acid sequence of SEQ ID NO: 33, a HCDR3 comprising an amino acid sequence of SEQ ID NO: 47, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b5) a HCDR1 comprising an amino acid sequence of SEQ ID NO: 19, a HCDR2 comprising an amino acid sequence of SEQ ID NO: 33, a HCDR3 comprising an amino acid sequence of SEQ ID NO: 47, a LCDR1 comprising an amino acid sequence of SEQ ID NO: 65, a LCDR2 comprising an amino acid sequence of SEQ ID NO: 70, and a LCDR3 comprising an amino acid sequence of SEQ ID NO: 75;
(b6) a HCDR1 comprising an amino acid sequence of SEQ ID NO: 20, a HCDR2 comprising an amino acid sequence of SEQ ID NO: 34, a HCDR3 comprising an amino acid sequence of SEQ ID NO: 48, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b7) a HCDR1 comprising an amino acid sequence of SEQ ID NO: 22, a HCDR2 comprising an amino acid sequence of SEQ ID NO: 36, a HCDR3 comprising an amino acid sequence of SEQ ID NO: 50, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b8) a HCDR1 comprising an amino acid sequence of SEQ ID NO: 23, a HCDR2 comprising an amino acid sequence of SEQ ID NO: 37, a HCDR3 comprising an amino acid sequence of SEQ ID NO: 51, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b9) a HCDR1 comprising an amino acid sequence of SEQ ID NO: 23, a HCDR2 comprising an amino acid sequence of SEQ ID NO: 37, a HCDR3 comprising an amino acid sequence of SEQ ID NO: 51, a LCDR1 comprising an amino acid sequence of SEQ ID NO: 66, a LCDR2 comprising an amino acid sequence of SEQ ID NO: 71, and a LCDR3 comprising an amino acid sequence of SEQ ID NO: 76;
(b10) a HCDR1 comprising an amino acid sequence of SEQ ID NO:
24, a HCDR2 comprising an amino acid sequence of SEQ ID NO: 38, a HCDR3 comprising an amino acid sequence of SEQ ID NO: 52, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b11) a HCDR1 comprising an amino acid sequence of SEQ ID NO: 25, a HCDR2 comprising an amino acid sequence of SEQ ID NO: 39, a HCDR3 comprising an amino acid sequence of SEQ ID NO: 53, a LCDR1 comprising an amino acid sequence of SEQ ID NO: 66, a LCDR2 comprising an amino acid sequence of SEQ ID NO: 71, and a LCDR3 comprising an amino acid sequence of SEQ ID NO: 76;
(b12) a HCDR1 comprising an amino acid sequence of SEQ ID NO: 26, a HCDR2 comprising an amino acid sequence of SEQ ID NO: 40, a HCDR3 comprising an amino acid sequence of SEQ ID NO: 54, a LCDR1 comprising an amino acid sequence of SEQ ID NO: 66, a LCDR2 comprising an amino acid sequence of SEQ ID NO: 71, and a LCDR3 comprising an amino acid sequence of SEQ ID NO: 76;
(b13) a HCDR1 comprising an amino acid sequence of SEQ ID NO: 26, a HCDR2 comprising an amino acid sequence of SEQ ID NO: 40, a HCDR3 comprising an amino acid sequence of SEQ ID NO: 54, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b14) a HCDR1 comprising an amino acid sequence of SEQ ID NO: 27, a HCDR2 comprising an amino acid sequence of SEQ ID NO: 41, a HCDR3 comprising an amino acid sequence of SEQ ID NO: 55, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(b15) a HCDR1 comprising an amino acid sequence of SEQ ID NO: 28, a HCDR2 comprising an amino acid sequence of SEQ ID NO: 42, a HCDR3 comprising an amino acid sequence of SEQ ID NO: 56, a LCDR1 comprising an amino acid sequence of SEQ ID NO: 63, a LCDR2 comprising an amino acid sequence of SEQ ID NO: 68, and a LCDR3 comprising an amino acid sequence of SEQ ID NO: 73;
(c1) a heavy chain variable domain (VH) comprising an amino acid sequence that is at least 70%, 80% or 90% identical to SEQ ID NO: 2, and a light chain variable domain (VL) comprising an amino acid sequence that is at least 70%, 80% or 90% identical to SEQ ID NO: 58;
(c2) a heavy chain variable domain (VH) comprising an amino acid sequence that is at least 70%, 80% or 90% identical to SEQ ID NO: 3, and a light chain variable domain (VL) comprising an amino acid sequence that is at least 70%, 80% or 90% identical to SEQ ID NO: 59;
(c3) a heavy chain variable domain (VH) comprising an amino acid sequence that is at least 70%, 80% or 90% identical to SEQ ID NO: 4, and a light chain variable domain (VL) comprising an amino acid sequence that is at least 70%, 80% or 90% identical to SEQ ID NO: 58;
(c4) a heavy chain variable domain (VH) comprising an amino acid sequence that is at least 70%, 80% or 90% identical to SEQ ID NO: 5, and a light chain variable domain (VL) comprising an amino acid sequence that is at least 70%, 80% or 90% identical to SEQ ID NO: 58;
(c5) a heavy chain variable domain (VH) comprising an amino acid sequence that is at least 70%, 80% or 90% identical to SEQ ID NO: 5, and a light chain variable domain (VL) comprising an amino acid sequence that is at least 70%, 80% or 90% identical to SEQ ID NO: 60;
(c6) a heavy chain variable domain (VH) comprising an amino acid sequence that is at least 70%, 80% or 90% identical to SEQ ID NO: 6, and a light chain variable domain (VL) comprising an amino acid sequence that is at least 70%, 80% or 90% identical to SEQ ID NO: 58;
(c7) a heavy chain variable domain (VH) comprising an amino acid sequence that is at least 70%, 80% or 90% identical to SEQ ID NO: 8, and a light chain variable domain (VL) comprising an amino acid sequence that is at least 70%, 80% or 90% identical to SEQ ID NO: 58;
(c8) a heavy chain variable domain (VH) comprising an amino acid sequence that is at least 70%, 80% or 90% identical to SEQ ID NO: 9, and a light chain variable domain (VL) comprising an amino acid sequence that is at least 70%, 80% or 90% identical to SEQ ID NO: 58;
(c9) a heavy chain variable domain (VH) comprising an amino acid sequence that is at least 70%, 80% or 90% identical to SEQ ID NO: 9, and a light chain variable domain (VL) comprising an amino acid sequence that is at least 70%, 80% or 90% identical to SEQ ID NO: 61;
(c10) a heavy chain variable domain (VH) comprising an amino acid sequence that is at least 70%, 80% or 90% identical to SEQ ID NO: 10, and a light chain variable domain (VL) comprising an amino acid sequence that is at least 70%, 80% or 90% identical to SEQ ID NO: 58;
(c11) a heavy chain variable domain (VH) comprising an amino acid sequence that is at least 70%, 80% or 90% identical to SEQ ID NO: 11, and a light chain variable domain (VL) comprising an amino acid sequence that is at least 70%, 80% or 90% identical to SEQ ID NO: 61;
(c12) a heavy chain variable domain (VH) comprising an amino acid sequence that is at least 70%, 80% or 90% identical to SEQ ID NO: 12, and a light chain variable domain (VL) comprising an amino acid sequence that is at least 70%, 80% or 90% identical to SEQ ID NO: 61;
(c13) a heavy chain variable domain (VH) comprising an amino acid sequence that is at least 70%, 80% or 90% identical to SEQ ID NO: 12, and a light chain variable domain (VL) comprising an amino acid sequence that is at least 70%, 80% or 90% identical to SEQ ID NO: 58;
(c14) a heavy chain variable domain (VH) comprising an amino acid sequence that is at least 70%, 80% or 90% identical to SEQ ID NO: 13, and a light chain variable domain (VL) comprising an amino acid sequence that is at least 70%, 80% or 90% identical to SEQ ID NO: 58;
(c15) a heavy chain variable domain (VH) comprising an amino acid sequence that is at least 70%, 80% or 90% identical to SEQ ID NO: 14, and a light chain variable domain (VL) comprising an amino acid sequence that is at least 70%, 80% or 90% identical to SEQ ID NO: 58;
(d1) a heavy chain variable domain (VH) of SEQ ID NO: 2, and a light chain variable domain (VL) of SEQ ID NO: 58;
(d2) a heavy chain variable domain (VH) of SEQ ID NO: 3, and a light chain variable domain (VL) of SEQ ID NO: 59;
(d3) a heavy chain variable domain (VH) of SEQ ID NO: 4, and a light chain variable domain (VL) of SEQ ID NO: 58;
(d4) a heavy chain variable domain (VH) of SEQ ID NO: 5, and a light chain variable domain (VL) of SEQ ID NO: 58;
(d5) a heavy chain variable domain (VH) of SEQ ID NO: 5, and a light chain variable domain (VL) of SEQ ID NO: 60;
(d6) a heavy chain variable domain (VH) of SEQ ID NO: 6, and a light chain variable domain (VL) of SEQ ID NO: 58;
(d7) a heavy chain variable domain (VH) of SEQ ID NO: 8, and a light chain variable domain (VL) of SEQ ID NO: 58;
(d8) a heavy chain variable domain (VH) of SEQ ID NO: 9, and a light chain variable domain (VL) of SEQ ID NO: 58;
(d9) a heavy chain variable domain (VH) of SEQ ID NO: 9, and a light chain variable domain (VL) of SEQ ID NO: 61;
(d10) a heavy chain variable domain (VH) of SEQ ID NO: 10, and a light chain variable domain (VL) of SEQ ID NO: 58;
(d11) a heavy chain variable domain (VH) of SEQ ID NO: 11, and a light chain variable domain (VL) of SEQ ID NO: 61;
(d12) a heavy chain variable domain (VH) of SEQ ID NO: 12, and a light chain variable domain (VL) of SEQ ID NO: 61;
(d13) a heavy chain variable domain (VH) of SEQ ID NO: 12, and a light chain variable domain (VL) of SEQ ID NO: 58;
(d14) a heavy chain variable domain (VH) of SEQ ID NO: 13, and a light chain variable domain (VL) of SEQ ID NO: 58;
(d15) a heavy chain variable domain (VH) of SEQ ID NO: 14, and a light chain variable domain (VL) of SEQ ID NO: 58;
(e) an antibody variable region that competes for binding to CD3 with any one of the antibody variable regions of (a1) to (d15);
(f) an antibody variable region that competes for binding to CD137 with any one of the antibody variable regions of (a1) to (d15);
(g) an antibody variable region that binds to the same epitope on CD3 with any one of the antibody variable regions of (a1) to (d15);
(h) an antibody variable region that binds to the same epitope on CD137 with any one of the antibody variable regions of (a1) to (d15).

4. The antigen-binding molecule of any one of claim 3 (a1)-(a15) or (c1)-(c15), which comprises:
(a) a heavy chain variable domain amino acid sequence comprising, at each of the following positions (all by Kabat numbering), one or more of the following amino acid residues indicated for that position:
A, D, E, I, G, K, L, M, N, R, T, W or Y at the amino acid position 26;
D, F, G, I, M or L, at the amino acid position 27;
D, E, F, G, H, I, K, L, M, N, P, Q, R, S, T, V, W or Y at the amino acid position 28;
F or W at the amino acid position 29;
A, D, E, F, G, H, I, K, L, M, N, P, Q, R, S, T, V, W or Y at the amino acid position 30;
F, I, N, R, S, T or V at the amino acid position 31;
A, H, I, K, L, N, Q, R, S, T or V at the amino acid position 32;
W at the amino acid position 33;
F, I, L, M or V at the amino acid position 34;
F, H, S, T, V or Y at the amino acid position 35;
E, F, H, I, K, L, M, N, Q, S, T, W or Y at the amino acid position 50;
I, K or V at the amino acid position 51;
K, M, R, or T at the amino acid position 52;
A, E, F, G, H, I, K, L, M, N, P, Q, R, S, V, W or Y at the amino acid position 52b;
A, D, E, F, G, H, I, K, L, M, N, P, Q, R, S, T, V, W or Y at the amino acid position 52c;
A, E, F, H, K, L, M, N, Q, R, S, T, V, W or Y at the amino acid position 53;
A, D, E, F, G, H, I, K, L, M, N, Q, R, S, T, V, W or Y at the amino acid position 54;
E, F, G, H, L, M, N, Q, W or Y at the amino acid position 55;
A, D, E, F, G, H, I, K, L, M, N, Q, R, S, T, V, W or Y at the amino acid position 56;
A, D, E, G, H, I, K, L, M, N, P, Q, R, S, T or V at the amino acid position 57;
A, F, H, K, N, P, R or Y at the amino acid position 58;
A, D, E, F, G, H, I, K, L, M, N, P, Q, R, S, T, V, W or Y at the amino acid position 59;
A, D, E, F, G, H, I, K, L, M, N, P, Q, R, S, T, V, W or Y at the amino acid position 60;
A, D, E, F, G, H, I, K, L, M, N, P, Q, R, S, T, V, W or Y at the amino acid position 61;
A, D, E, F, G, H, I, K, L, M, N, P, Q, R, S, T, V, W or Y at the amino acid position 62;
A, D, E, F, G, H, I, K, L, M, N, P, Q, R, S, T, V, W or Y at the amino acid position 63;
A, D, E, F, G, H, I, K, L, M, N, P, Q, R, S, T, V, W or Y at the amino acid position 64;
A, D, E, F, G, H, I, K, L, M, N, P, Q, R, S, T, V, W or Y at the amino acid position 65;
H or R at the amino acid position 93;
F, G, H, L, M, S, T, V or Y at the amino acid position 94;
I or V at the amino acid position 95;
F, H, I, K, L, M, T, V, W or Y at the amino acid position 96;
F, Y or W at the amino acid position 97;
A, F, G, H, I, K, L, M, N, Q, R, S, T, V, W or Y at the amino acid position 98;
A, F, G, H, I, K, L, M, N, P, Q, R, S, T, V, W or Y at the amino acid position 99;
A, D, E, F, G, H, I, K, L, M, N, P, Q, R, S, T, V, W or Y at the amino acid position 100;
A, D, E, F, G, H, I, K, L, M, N, P, Q, R, S, T, V, W or Y at the amino acid position 100a;
A, D, E, F, G, H, I, K, L, M, N, P, Q, R, S, T, V, W or Y at the amino acid position 100b;
A, D, E, F, G, H, I, K, L, M, N, P, Q, R, S, T, V, W or Y at the amino acid position 100c;
A, D, E, F, G, H, I, K, L, M, N, P, Q, R, S, T, V, W or Y at the amino acid position 100d;
A, D, E, F, G, H, I, K, L, M, P, Q, R, S, T, V, W or Y at the amino acid position 100e;
A, E, F, G, H, I, K, L, M, N, P, Q, R, S, T, V, W or Y at the amino acid position 100f;
A, E, F, G, H, I, K, L, M, N, P, Q, R, S, T, V, W or Y at the amino acid position 100g;
A, D, E, G, H, I, L, M, N, P, S, T or V at the amino acid position 100h;
A, D, E, F, G, H, I, K, L, M, N, P, Q, R, S, T, V, W or Y at the amino acid position 100i;
A, D, F, I, L, M, N, Q, S, T or V at the amino acid position 101;
A, D, E, F, G, H, I K, L, M, N, Q, R, S, T, V, W or Y at the amino acid position 102;
and/or
(b) a light chain variable domain amino acid sequence comprising, at each of the following positions (all by Kabat numbering), one or more of the following amino acid residues indicated for that position:
A, D, F, G, H, I, K, L, M, N, Q, R, S, T, V, W or Y at the amino acid position 24;
A, G, N, P, S, T or V at the amino acid position 25;
A, D, E, F, G, I, K, L, M, N, Q, R, S, T or V at the amino acid position 26;
A, D, E, F, G, H, I, K, L, M, N, Q, R, S, T, V, W or Y at the amino acid position 27;
A, D, E, F, G, H, I, K, L, M, N, P, Q, R, S, T, V, W or Y at the amino acid position 27a;
A, I, L, M, P, T or V at the amino acid position 27b;
A, E, F, H, I, K, L, M, N, P, Q, R, T, W or Y at the amino acid position 27c;
A, E, G, H, I, K, L, M, N, P, Q, R, S, T, V, W or Y at the amino acid position 27d;
A, D, E, F, G, H, I, K, L, M, N, P, Q, R, S, T, V, W or Y at the amino acid position 27e;
G, N, S or T at the amino acid position 28;
A, F, G, H, K, L, M, N, Q, R, S, T, W or Y at the amino acid position 29;
A, F, G, H, I, K, L, M, N, Q, R, V, W or Y at the amino acid position 30;
I, L, Q, S, T or V at the amino acid position 31;
F, W or Y at the amino acid position 32;
A, F, H, L, M, Q or V at the amino acid position 33;
A, H or S at the amino acid position 34;
I, K, L, M or R at the amino acid position 50;
A, E, I, K, L, M, Q, R, S, T or V at the amino acid position 51;
A, D, E, F, G, H, I, K, L, M, N, Q, R, S, T, V, W or Y at the amino acid position 52;
A, E, F, G, H, K, L, M, N, P, Q, R, S, V, W or Y at the amino acid position 53;
A, D, E, F, G, H, I, K, L, M, N, P, Q, R, S, T, V, W or Y at the amino acid position 54;
A, D, E, F, G, H, I, K, L, M, N, P, Q, R, S, T, V or Y at the amino acid position 55;
A, D, E, F, G, H, I, K, L, M, N, P, Q, R, S, T, V, W or Y at the amino acid position 56;
A, G, K, S or Y at the amino acid position 89;
Q at the amino acid position 90;
G at the amino acid position 91;
A, D, H, K, N, Q, R, S or T at the amino acid position 92;
A, D, E, F, G, H, I, K, L, M, N, Q, R, S, T, V, W or Y at the amino acid position 93;
A, D, H, I, M, N, P, Q, R, S, T or V at the amino acid position 94;
P at the amino acid position 95;
F or Y at the amino acid position 96; and
A, D, E, G, H, I, K, L, M, N, Q, R, S, T or V at the amino acid position 97.

5. The antigen-binding molecule of any one of claims 1 to 4, wherein the antigen-binding molecule has at least one characteristic selected from the group consisting of (1) to (3) below:
(1) the antigen-binding molecule does not bind to CD3 and CD137 each expressed on a different cell, at the same time.
(2) the antigen-binding molecule has an agonistic activity against CD137; and
(3) the antigen-binding molecule has equivalent or 10-fold, 20-fold, 50-fold, 100-fold lower KD value for binding to human CD137, as compared to a reference antibody comprising a VH sequence of SEQ ID NO: 1 and a VL sequence of SEQ ID NO: 57, wherein the KD value is preferably measured by SPR at the following condition: 37 degrees C., pH 7.4, 20 mM ACES, 150 mM NaCl, 0.05% Tween 20, 0.005% NaN3; the antigen-binding molecule is immobilized on a CM4 sensor chip, the antigen serves as analyte.

6. The antigen-binding molecule of any one of claims 1 to 5, which further comprises an antibody variable region that is capable of binding to a third antigen different from CD3 and CD137.

7. The antigen-binding molecule of claim 6, wherein the third antigen is a molecule specifically expressed in a cancer tissue.

8. The antigen-binding molecule of any one of claims 1 to 7, further comprising an antibody Fc region.

9. The antigen-binding molecule of claim 8, wherein the Fc region is an Fc region having reduced binding activity against Fc gamma R as compared with the Fc region of a naturally occurring human IgG1 antibody.

10. A pharmaceutical composition comprising the antigen-binding molecule according to any one of claims 1 to 9, and a pharmaceutically acceptable carrier.

11. An isolated polynucleotide comprising a nucleotide sequence that encodes the antigen-binding molecule of any one of claims 1 to 9.

12. An expression vector comprising the polynucleotide according to claim 11.

13. A host cell transformed or transfected with the polynucleotide according to claim 11 or the expression vector according to claim 12.

14. A method of producing a multispecific antigen-binding molecule or a multispecific antibody, comprising culturing the host cell of claim 13.

15. A method of obtaining or screening for an antibody variable region that is capable of binding to CD3 and CD137, but does not bind to CD3 and CD137 at the same time, comprising:
(a) providing a library comprising a plurality of antibody variable region,
(b) contacting the library provided in step (a) with either CD3 or CD137 as a first antigen and collecting antibody variable regions bound to the first antigen,
(c) contacting the antibody variable regions collected in step (b) with a second antigen out of CD3 and CD137 and collecting antibody variable regions bound to the second antigen, and
(d) selecting an antibody variable region which:
(1) binds to CD137 with an equilibrium dissociation constant (KD) of less than about 5×10⁻⁶ M or between 5×10⁻⁶ M and 3×10⁻⁸M, preferably as measured by SPR at the following condition: 37 degrees C., pH 7.4, 20 mM ACES, 150 mM NaCl, 0.05% Tween 20, 0.005% NaN3; the antigen-binding molecule is immobilized on a CM4 sensor chip, the antigen serves as analyte; and/or
(2) binds to CD3 with an equilibrium dissociation constant (KD) of between 2×10⁻⁶ M and 1×10⁻⁸ M, preferably as measured by SPR at the following condition: 25 degrees C., pH 7.4, 20 mM ACES, 150 mM NaCl, 0.05% Tween 20, 0.005% NaN3; the antigen-binding molecule is immobilized on a CM4 sensor chip, the antigen serves as analyte.


SEQUENCE LISTING (229 sequences)

SEQ ID NO: 1 - protein, 128 aa, Artificial Sequence
  An artificially synthesized sequence
QVQLVESGGGLVQPGRSLRLSCAASGFTFSNAWMHWVRQAPGKGLEWVAQIKDKGNAYAAYYAPSVKGRFTISRDDSKNSIYLQMNSLKTEDTAVYYCHYVHYASASTVLPAFGVDAWGQGTTVTVSS

SEQ ID NO: 2 - protein, 128 aa, Artificial Sequence
  An artificially synthesized sequence
QVQLVESGGGLVQPGRSLRLSCAASGFKFSNVWMHWVRQAPGKGLEWVAQIKDKYNAYAAYYAPSVKGRFTISRDDSKNSIYLQMNSLKTEDTAVYYCHYVHYASASTLLPAFGVDAWGQGTTVTVSS

SEQ ID NO: 3 - protein, 128 aa, Artificial Sequence
  An artificially synthesized sequence
QVQLVESGGGLVQPGRSLRLSCAASGFKFSNVWMHWVRQAPGKGLEWVAQIKDKYNAYAAYYAPSVKGRFTISRDDSKNSIYLQMNSLKTEDTAVYYCHYIHYASASTLLPAFGVDAWGQGTTVTVSS

SEQ ID NO: 4 - protein, 128 aa, Artificial Sequence
  An artificially synthesized sequence
QVQLVESGGGLVQPGRSLRLSCAASGFKFSNVWFHWVRQAPGKGLEWVAQIKDKYNAYATYYAPSVKGRFTISRDDSKNSIYLQMNSLKTEDTAVYYCHYVHYASASTLLPAFGVDAWGQGTTVTVSS

SEQ ID NO: 5 - protein, 128 aa, Artificial Sequence
  An artificially synthesized sequence
QVQLVESGGGLVQPGRSLRLSCAASGFVFSNVWMHWVRQAPGKGLEWVAQIKDKWNAYAAYYAPSVKGRFTISRDDSKNSIYLQMNSLKTEDTAVYYCHYIHYASASTLLPAEGIDAWGQGTTVTVSS

SEQ ID NO: 6 - protein, 128 aa, Artificial Sequence
  An artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSS

SEQ ID NO: 7 - skipped

SEQ ID NO: 8 - protein, 128 aa, Artificial Sequence
  An artificially synthesized sequence
QVQLVESGGGLVQPGRSLRLSCAASGFKFSNTWFHWVRQAPGKGLEWVAQIKDYYNDYAAYYAPSVKGRFTISRDDSKNSIYLQMNSLKTEDTAVYYCHYVHYASASTLLPAEGVDAWGQGTTVTVSS

SEQ ID NO: 9 - protein, 128 aa, Artificial Sequence
  An artificially synthesized sequence
QVQLVESGGGLVQPGRSLRLSCAASGFVFSNVWFHWVRQAPGKGLEWVAQIKDKYNAYADYYAPSVKERFTISRDDSKNSIYLQMNSLKTEDTAVYYCHYVHYASASTLLPAEGVDAWGQGTTVTVSS

SEQ ID NO: 10 - protein, 128 aa, Artificial Sequence
  An artificially synthesized sequence
QVQLVESGGGLVQPGRSLRLSCAASGFVFSNVWFHWVRQAPGKGLEWVAQIKDKYNAYADYYAPSVEGRFTISRDDSKNSIYLQMNSLKTEDTAVYYCHYVHYASASTLLPAEGVDAWGQGTTVTVSS

SEQ ID NO: 11 - protein, 128 aa, Artificial Sequence
  An artificially synthesized sequence
QVQLVESGGGLVQPGRSLRLSCAASGFVFSNVWFHWVRQAPGKGLEWVAQIKDYYNAYADYYAPSVEGRFTISRDDSKNSIYLQMNSLKTEDTAVYYCHYVHYASASTLLPAEGVDAWGQGTTVTVSS

SEQ ID NO: 12 - protein, 128 aa, Artificial Sequence
  An artificially synthesized sequence
QVQLVESGGGLVQPGRSLRLSCAASGFVFSNVWFHWVRQAPGKGLEWVAQIKDKWNAYADYYAPSVKERFTISRDDSKNSIYLQMNSLKTEDTAVYYCHYIHYASASTLLPAEGIDAWGQGTTVTVSS

SEQ ID NO: 13 - protein, 128 aa, Artificial Sequence
  An artificially synthesized sequence
QVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASTLLPAEGVDAWGQGTTVTVSS

SEQ ID NO: 14 - protein, 128 aa, Artificial Sequence
  An artificially synthesized sequence
QVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSS

SEQ ID NO: 15 - protein, 5 aa, Artificial Sequence
  An artificially synthesized sequence
NAWMH

SEQ ID NO: 16 - protein, 5 aa, Artificial Sequence
  An artificially synthesized sequence
NVWMH

SEQ ID NO: 17 - protein, 5 aa, Artificial Sequence
  An artificially synthesized sequence
NVWMH

SEQ ID NO: 18 - protein, 5 aa, Artificial Sequence
  An artificially synthesized sequence
NVWFH

SEQ ID NO: 19 - protein, 5 aa, Artificial Sequence
  An artificially synthesized sequence
NVWMH

SEQ ID NO: 20 - protein, 5 aa, Artificial Sequence
  An artificially synthesized sequence
NVWFH

SEQ ID NO: 21 - skipped

SEQ ID NO: 22 - protein, 5 aa, Artificial Sequence
  An artificially synthesized sequence
NTWFH

SEQ ID NO: 23 - protein, 5 aa, Artificial Sequence
  An artificially synthesized sequence
NVWFH

SEQ ID NO: 24 - protein, 5 aa, Artificial Sequence
  An artificially synthesized sequence
NVWFH

SEQ ID NO: 25 - protein, 5 aa, Artificial Sequence
  An artificially synthesized sequence
NVWFH

SEQ ID NO: 26 - protein, 5 aa, Artificial Sequence
  An artificially synthesized sequence
NVWFH

SEQ ID NO: 27 - protein, 5 aa, Artificial Sequence
  An artificially synthesized sequence
NVWFH

SEQ ID NO: 28 - protein, 5 aa, Artificial Sequence
  An artificially synthesized sequence
NVWFH

SEQ ID NO: 29 - protein, 19 aa, Artificial Sequence
  An artificially synthesized sequence
QIKDKGNAYAAYYAPSVKG

SEQ ID NO: 30 - protein, 19 aa, Artificial Sequence
  An artificially synthesized sequence
QIKDKYNAYAAYYAPSVKG

SEQ ID NO: 31 - protein, 19 aa, Artificial Sequence
  An artificially synthesized sequence
QIKDKYNAYAAYYAPSVKG

SEQ ID NO: 32 - protein, 19 aa, Artificial Sequence
  An artificially synthesized sequence
QIKDKYNAYATYYAPSVKG

SEQ ID NO: 33 - protein, 19 aa, Artificial Sequence
  An artificially synthesized sequence
QIKDKWNAYAAYYAPSVKG

SEQ ID NO: 34 - protein, 19 aa, Artificial Sequence
  An artificially synthesized sequence
QIKDYYNAYAAYYAPSVKG

SEQ ID NO: 35 - skipped

SEQ ID NO: 36 - protein, 19 aa, Artificial Sequence
  An artificially synthesized sequence
QIKDYYNDYAAYYAPSVKG

SEQ ID NO: 37 - protein, 19 aa, Artificial Sequence
  An artificially synthesized sequence
QIKDKYNAYADYYAPSVKE

SEQ ID NO: 38 - protein, 19 aa, Artificial Sequence
  An artificially synthesized sequence
QIKDKYNAYADYYAPSVEG

SEQ ID NO: 39 - protein, 19 aa, Artificial Sequence
  An artificially synthesized sequence
QIKDYYNAYADYYAPSVEG

SEQ ID NO: 40 - protein, 19 aa, Artificial Sequence
  An artificially synthesized sequence
QIKDKWNAYADYYAPSVKE

SEQ ID NO: 41 - protein, 19 aa, Artificial Sequence
  An artificially synthesized sequence
QIKDYYNAYAGYYHPSVKG

SEQ ID NO: 42 - protein, 19 aa, Artificial Sequence
  An artificially synthesized sequence
QIKDYYNAYAGYYHPSVKG

SEQ ID NO: 43 - protein, 17 aa, Artificial Sequence
  An artificially synthesized sequence
VHYASASTVLPAFGVDA

SEQ ID NO: 44 - protein, 17 aa, Artificial Sequence
  An artificially synthesized sequence
VHYASASTLLPAFGVDA

SEQ ID NO: 45 - protein, 17 aa, Artificial Sequence
  An artificially synthesized sequence
IHYASASTLLPAFGVDA

SEQ ID NO: 46 - protein, 17 aa, Artificial Sequence
  An artificially synthesized sequence
VHYASASTLLPAFGVDA

SEQ ID NO: 47 - protein, 17 aa, Artificial Sequence
  An artificially synthesized sequence
IHYASASTLLPAEGIDA

SEQ ID NO: 48 - protein, 17 aa, Artificial Sequence
  An artificially synthesized sequence
VHYASASTLLPAEGVDA

SEQ ID NO: 49 - skipped

SEQ ID NO: 50 - protein, 17 aa, Artificial Sequence
  An artificially synthesized sequence
VHYASASTLLPAEGVDA

SEQ ID NO: 51 - protein, 17 aa, Artificial Sequence
  An artificially synthesized sequence
VHYASASTLLPAEGVDA

SEQ ID NO: 52 - protein, 17 aa, Artificial Sequence
  An artificially synthesized sequence
VHYASASTLLPAEGVDA

SEQ ID NO: 53 - protein, 17 aa, Artificial Sequence
  An artificially synthesized sequence
VHYASASTLLPAEGVDA

SEQ ID NO: 54 - protein, 17 aa, Artificial Sequence
  An artificially synthesized sequence
IHYASASTLLPAEGIDA

SEQ ID NO: 55 - protein, 17 aa, Artificial Sequence
  An artificially synthesized sequence
VHYAAASTLLPAEGVDA

SEQ ID NO: 56 - protein, 17 aa, Artificial Sequence
  An artificially synthesized sequence
VHYAAASQLLPAEGVDA

SEQ ID NO: 57 - protein, 112 aa, Artificial Sequence
  An artificially synthesized sequence
DIVMTQSPLSLPVTPGEPASISCQASQELVHMNRNTYLHWYQQKPGQAPRLLIYKVSNRFPGVPDRFSGSGSGTDFTLKISRVEAEDVGVYYCAQGTSVPFTFGQGTKLEIK

SEQ ID NO: 58 - protein, 112 aa, Artificial Sequence
  An artificially synthesized sequence
DIVMTQSPLSLPVTPGEPASISCQPSQEVVHMNRNTYLHWYQQKPGQAPRLLIYKVSNRFPGVPDRFSGSGSGTDFTLKISRVEAEDVGVYYCAQGTSHPFTFGQGTKLEIK

SEQ ID NO: 59 - protein, 112 aa, Artificial Sequence
  An artificially synthesized sequence
DIVMTQSPLSLPVTPGEPASISCQPSQEVVHMNNVVYLHWYQQKPGQAPRLLIYKVSNRFPGVPDRFSGSGSGTDFTLKISRVEAEDVGVYYCAQGTSHPFTFGQGTKLEIK

SEQ ID NO: 60 - protein, 112 aa, Artificial Sequence
  An artificially synthesized sequence
DIVMTQSPLSLPVTPGEPASISCQPSQEVVHMNRNTYLHWYQQKPGQAPRLLIYKVSNVFPGVPDRFSGSGSGTDFTLKISRVEAEDVGVYYCAQGTHHPFTFGQGTKLEIK

SEQ ID NO: 61 - protein, 112 aa, Artificial Sequence
  An artificially synthesized sequence
DIVMTQSPLSLPVTPGEPASISCQPSEEVVHMNRNTYLHWYQQKPGQAPRLLIYKVSNLFPGVPDRFSGSGSGTDFTLKISRVEAEDVGVYYCAQGTHHPFTFGQGTKLEIK

SEQ ID NO: 62 - protein, 16 aa, Artificial Sequence
  An artificially synthesized sequence
QASQELVHMNRNTYLH

SEQ ID NO: 63 - protein, 16 aa, Artificial Sequence
  An artificially synthesized sequence
QPSQEVVHMNRNTYLH

SEQ ID NO: 64 - protein, 16 aa, Artificial Sequence
  An artificially synthesized sequence
QPSQEVVHMNNVVYLH

SEQ ID NO: 65 - protein, 16 aa, Artificial Sequence
  An artificially synthesized sequence
QPSQEVVHMNRNTYLH

SEQ ID NO: 66 - protein, 16 aa, Artificial Sequence
  An artificially synthesized sequence
QPSEEVVHMNRNTYLH

SEQ ID NO: 67 - protein, 7 aa, Artificial Sequence
  An artificially synthesized sequence
KVSNRFP

SEQ ID NO: 68 - protein, 7 aa, Artificial Sequence
  An artificially synthesized sequence
KVSNRFP

SEQ ID NO: 69 - protein, 7 aa, Artificial Sequence
  An artificially synthesized sequence
KVSNRFP

SEQ ID NO: 70 - protein, 7 aa, Artificial Sequence
  An artificially synthesized sequence
KVSNVFP

SEQ ID NO: 71 - protein, 7 aa, Artificial Sequence
  An artificially synthesized sequence
KVSNLFP

SEQ ID NO: 72 - protein, 9 aa, Artificial Sequence
  An artificially synthesized sequence
AQGTSVPFT

SEQ ID NO: 73 - protein, 9 aa, Artificial Sequence
  An artificially synthesized sequence
AQGTSHPFT

SEQ ID NO: 74 - protein, 9 aa, Artificial Sequence
  An artificially synthesized sequence
AQGTSHPFT

SEQ ID NO: 75 - protein, 9 aa, Artificial Sequence
  An artificially synthesized sequence
AQGTHHPFT

SEQ ID NO: 76 - protein, 9 aa, Artificial Sequence
  An artificially synthesized sequence
AQGTHHPFT

SEQ ID NO: 77 - protein, 122 aa, Artificial Sequence
  An artificially synthesized sequence
QVQLVESGGGVVQPGGSLRLSCAASGFTFSNAWMHWVRQAPGKGLEWVAQIKDKSQNYATYVAESVKGRFTISRADSKNSIYLQMNSLKTEDTAVYYCRYVHYAAGYGVDIWGQGTTVTVSS

SEQ ID NO: 78 - protein, 112 aa, Artificial Sequence
  An artificially synthesized sequence
DIVMTQSPLSLPVTPGEPASISCRSSQPLVHSNRNTYLHWYQQKPGQAPRLLIYKVSNRFSGVPDRFSGSGSGTDFTLKISRVEAEDVGVYYCGQGTQVPYTFGQGTKLEIK

SEQ ID NO: 79 - protein, 121 aa, Artificial Sequence [sequence omitted]

SEQ ID NO: 80 - protein, 109 aa, Artificial Sequence [sequence omitted]

SEQ ID NO: 81 - protein, 399 aa, Artificial Sequence
  An artificially synthesized sequence
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQDIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 82 - protein, 444 aa, Artificial Sequence
  An artificially synthesized sequence
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSFEDPEVKFNWYVDGVEVHNAKTKPREEPYNSTYRVVSVLTVLHQDWLNGKEYKCKVSNKAAPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 83 - protein, 214 aa, Artificial Sequence
  An artificially synthesized sequence
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 84 - protein, 212 aa, Artificial Sequence
  An artificially synthesized sequence
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVGSADDAKKDAAKKDDAKKDDAKKDGSQSIKGNHLVKVYDYQEDGSVLLTCDAEAKNITWFKDGKMIGFLTEDKKKWNLGSNAKDPRGMYQCKGSQNKSKPLQVYYRMDYKDDDDK

SEQ ID NO: 85 - protein, 112 aa, Artificial Sequence
  An artificially synthesized sequence
DIVMTQSPLSLPVTPGEPASISCRSSQSLVHSNRNTYLHWYQQKPGQAPRLLIYKVSNRFSGVPDRFSGSGSGTDFTLKISRVEAEDVGVYYCGQGTQVPYTFGQGTKLEIK

SEQ ID NO: 86 - protein, 15 aa, Artificial Sequence
  An artificially synthesized sequence
QDGNEEMGGITQTPY

SEQ ID NO: 87 - protein, 112 aa, Artificial Sequence
  An artificially synthesized sequence
DIVMTQSPMSLPVTPGEPASISCQSSESLLHANRNTYLHWYQQKPGQAPRLLIYKVSNRFNGVPDRFSGSGSGTDFTLKISRVEAEDVGVYYCAQGTANPYTFGQGTKLEIK

SEQ ID NO: 88 - protein, 112 aa, Artificial Sequence
  An artificially synthesized sequence
DIVMTQSPLSLPVTPGEPASISCAASESLLHTNRNTYLHWYQQKPGQAPRLLIYKVSNRFSGVPDRFSGSGSGTDFTLKISRVEAEDVGVYYCAQGTQSPFTFGQGTKLEIK

SEQ ID NO: 89 - protein, 112 aa, Artificial Sequence
  An artificially synthesized sequence
DIVMTQSPLSLPVTPGEPASISCTSSQSLLHANRNTYLHWYQQKPGQAPRLLIYKVSNRFSGVPDRFSGSGSGTDFTLKISRVEAEDVGVYYCAQGTSAPFTFGQGTKLEIK

SEQ ID NO: 90 - protein, 399 aa, Artificial Sequence
  An artificially synthesized sequence
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQDIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 91 - protein, 270 aa, Artificial Sequence
  An artificially synthesized sequence
MKSLFKVTLLATTMAVALHAPITFAAEAAKPATAADSKAAFKNDDQKSAYALGASLGRYMENSLKEQEKLGIKLDKDQLIAGVQDAFADKSKLSDQEIEQTLQAFEARVKSSAQAKMEKDAADNEAKGKEYREKFAKEKGVKTSSTGLVYQVVEAGKGEAPKDSDTVVVNYKGTLIDGKEFDNSYTRGEPLSFRLDGVIPGWTEGLKNIKKGGKIKLVIPPELAYGKAGVPGIPPNSTLVFDVELLDVKPAPKADAKPEADAKAADSAKK

SEQ ID NO: 92 - protein, 163 aa, Artificial Sequence
  An artificially synthesized sequence
LQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQ

SEQ ID NO: 93 - protein, 121 aa, Artificial Sequence [sequence omitted]

SEQ ID NO: 94 - protein, 109 aa, Artificial Sequence [sequence omitted]

SEQ ID NO: 95 - protein, 373 aa, Artificial Sequence
  An artificially synthesized sequence
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDDIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGSGENLYFQGSGGGLNDIFEAQKIEWHE

SEQ ID NO: 96 - protein, 323 aa, Artificial Sequence
  An artificially synthesized sequence
FKIPIEELEDRVFVNCNTSITWVEGTVGTLLSDITRLDLGKRILDPRGIYRCNGTDIYKDKESTVQVHYRMCQSCVELDDIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPDYKDDDDK

SEQ ID NO: 97 - protein, 128 aa, Artificial Sequence
  An artificially synthesized sequence
QVQLVESGGGLVQPGRSLRLSCAASGFTFSNAWMHWVRQAPGKGLEWVAQIKDKGNAYAAYYAPSVKGRFTISRDDSKNSIYLQMNSLKTEDTAVYYCHYVHYASAAGGLPGFGVDAWGQGTTVTVSS

SEQ ID NO: 98 - protein, 128 aa, Artificial Sequence
  An artificially synthesized sequence
QVQLVESGGGLVQPGRSLRLSCAASGFTFSNAWMHWVRQAPGKGLEWVAQIKDRSQLYAAYYAPSVKGRFTISRDDSKNSIYLQMNSLKTEDTAVYYCHYVHYASASTVLGAYGVDAWGQGTTVTVSS

SEQ ID NO: 99 - protein, 128 aa, Artificial Sequence
  An artificially synthesized sequence
QVQLVESGGGLVQPGRSLRLSCAASGFTFSNAWMHWVRQAPGKGLEWVAQIKDRANSYNAYYAPSVKGRFTISRDDSKNSIYLQMNSLKTEDTAVYYCHYVHYSAASTLLPSFGVDAWGQGTTVTVSS

SEQ ID NO: 100 - protein, 128 aa, Artificial Sequence
  An artificially synthesized sequence
QVQLVESGGGLVQPGRSLRLSCAASGFTFSNAWMHWVRQAPGKGLEWVAQIKDKQNLYNAYYAESVKGRFTISRDDSKNSIYLQMNSLKTEDTAVYYCHYVHYASASTVLPAFGVDAWGQGTTVTVSS

SEQ ID NO: 101 - protein, 128 aa, Artificial Sequence
  An artificially synthesized sequence
QVQLVESGGGLVQPGRSLRLSCAASGFTFSIAWMHWVRQAPGKGLEWVAQIKDKVNGYTAYYAESVKGRFTISRDDSKNSIYLQMNSLKTEDTAVYYCRYVHYAAYYDVLGYGGVDAWGQGTTVTVSS

SEQ ID NO: 102 - protein, 128 aa, Artificial Sequence
  An artificially synthesized sequence
QVQLVESGGGLVQPGRSLRLSCAASGFTFSIAWMHWVRQAPGKGLEWVAQIKDRVNAYEAYYAPSVKGRFTISRDDSKNSIYLQMNSLKTEDTAVYYCRYVHYASYYDLLGYGGVDAWGQGTTVTVSS

SEQ ID NO: 103 - protein, 128 aa, Artificial Sequence
  An artificially synthesized sequence
QVQLVESGGGLVQPGRSLRLSCAASGFTFSNAWMHWVRQAPGKGLEWVAQIKDRGQQYNAYYAPSVKGRFTISRDDSKNSIYLQMNSLKTEDTAVYYCHYVHYASASSVLPGYGVDAWGQGTTVTVSS

SEQ ID NO: 104 - protein, 128 aa, Artificial Sequence; identical to SEQ ID NO: 1
  An artificially synthesized sequence

SEQ ID NO: 105 - protein, 128 aa, Artificial Sequence
  An artificially synthesized sequence
QVQLVESGGGLVQPGRSLRLSCAASGFTFSNAWMHWVRQAPGKGLEWVAQIKDRSNQYATYYAPSVKGRFTISRDDSKNSIYLQMNSLKTEDTAVYYCHYVHYASASTVLPAFGVDAWGQGTTVTVSS

SEQ ID NO: 106 - protein, 128 aa, Artificial Sequence
  An artificially synthesized sequence
QVQLVESGGGLVQPGRSLRLSCAASGFTFSNAWMHWVRQAPGKGLEWVAQIKDRGNAYNAYYAESVKGRFTISRDDSKNSIYLQMNSLKTEDTAVYYCHYVHYASASTVLPAFGVDAWGQGTTVTVSS

SEQ ID NO: 107 - protein, 128 aa, Artificial Sequence
  An artificially synthesized sequence
QVQLVESGGGLVQPGRSLRLSCAASGFTFSNAWMHWVRQAPGKGLEWVAQIKDRSNAYAAYYAPSVKGRFTISRDDSKNSIYLQMNSLKTEDTAVYYCHYVHYASASTVLPAFGVDAWGQGTTVTVSS

SEQ ID NO: 108 - protein, 128 aa, Artificial Sequence
  An artificially synthesized sequence
QVQLVESGGGLVQPGRSLRLSCAASGFTFSNAWMHWVRQAPGKGLEWVAQIKDKQSGYNTYYAPSVKGRFTISRDDSKNSIYLQMNSLKTEDTAVYYCHYVHYASASTVLPAFGVDAWGQGTTVTVSS

SEQ ID NO: 109 - protein, 128 aa, Artificial Sequence
  An artificially synthesized sequence
QVQLVESGGGLVQPGRSLRLSCAASGFTFSNAWMHWVRQAPGKGLEWVAQIKDRSNNYATYYAESVKGRFTISRDDSKNSIYLQMNSLKTEDTAVYYCHYVHYASASTVLPAFGVDAWGQGTTVTVSS

SEQ ID NO: 110 - protein, 128 aa, Artificial Sequence
  An artificially synthesized sequence
QVQLVESGGGLVQPGRSLRLSCAASGFTFSNAWMHWVRQAPGKGLEWVAQIKDRSQLYAAYYAPSVKGRFTISRDDSKNSIYLQMNSLKTEDTAVYYCHYVHYASASTVLPAFGVDAWGQGTTVTVSS

SEQ ID NO: 111 - protein, 128 aa, Artificial Sequence
  An artificially synthesized sequence
QVQLVESGGGLVQPGRSLRLSCAASGFTFSNAWMHWVRQAPGKGLEWVAQIKDRSNQYATYYAPSVKGRFTISRDDSKNSIYLQMNSLKTEDTAVYYCHYVHYSAASTLLPSFGVDAWGQGTTVTVSS

SEQ ID NO: 112 - protein, 126 aa, Artificial Sequence
  An artificially synthesized sequence
QVQLVESGGGLVQPGRSLRLSCAASGFTFSNAWMHWVRQAPGKGLEWVAQIKDRQQGYATYYAPSVKGRFTISRDDSKNSIYLQMNSLKTEDTAVYYCRFVHYHLAGYGYYGVDAWGQGTTVTVSS

SEQ ID NO: 113 - protein, 127 aa, Artificial Sequence
  An artificially synthesized sequence
QVQLVESGGGLVQPGRSLRLSCAASGFTFSNAWMHWVRQAPGKGLEWVAQIKDRGNAYNAYYAESVKGRFTISRDDSKNSIYLQMNSLKTEDTAVYYCRFVHYRHPLLHIQYGVDAWGQGTTVTVSS

SEQ ID NO: 114 - protein, 128 aa, Artificial Sequence
  An artificially synthesized sequence
QVQLVESGGGLVQPGRSLRLSCAASGFTFSNAWMHWVRQAPGKGLEWIAQIKDRSNQYATYYAPSVKGRFTISRDDSKNSIYLQMNSLKTEDTAVYYCHYVHYAQASSLLPSFGVDAWGQGTTVTVSS

SEQ ID NO: 115 - protein, 128 aa, Artificial Sequence
  An artificially synthesized sequence
QVQLVESGGGLVQPGRSLRLSCAASGFTFSNAWMHWVRQAPGKGLEWVAQIKDKGNLYAAYYAPSVKGRFTISRDDSKNSIYLQMNSLKTEDTAVYYCHYVHYASAGGGLPGFGVDAWGQGTTVTVSS

SEQ ID NO: 116 - protein, 128 aa, Artificial Sequence
  An artificially synthesized sequence
QVQLVESGGGLVQPGRSLRLSCAASGFTFSNAWMHWVRQAPGKGLEWVAQIKDKGNLYAAYYAPSVKGRFTISRDDSKNSIYLQMNSLKTEDTAVYYCHYVHYASAGGGLSAYGVDAWGQGTTVTVSS

SEQ ID NO: 117 - protein, 128 aa, Artificial Sequence
  An artificially synthesized sequence
QVQLVESGGGLVQPGRSLRLSCAASGFTFSSAWMHWVRQAPGKGLEWVAQIKDRASAYNAYYAESVKGRFTISRDDSKNSIYLQMNSLKTEDTAVYYCRYVHYPSYVSFLPYYGVDAWGQGTTVTVSS

SEQ ID NO: 118 - protein, 128 aa, Artificial Sequence
  An artificially synthesized sequence
QVQLVESGGGLVQPGRSLRLSCAASGFTFSNAWMHWVRQAPGKGLEWIAQIKDRGNNYNNYYAPSVKGRFTISRDDSKNSIYLQMNSLKTEDTAVYYCHYVHYATASTLLGAYGVDAWGQGTTVTVSS

SEQ ID NO: 119 - protein, 128 aa, Artificial Sequence
  An artificially synthesized sequence
QVQLVESGGGLVQPGRSLRLSCAASGFTFSNAWMHWVRQAPGKGLEWVAQIKDRSNQYATYYAPSVKGRFTISRDDSKNSIYLQMNSLKTEDTAVYYCHYVHYAQASTVLPAFGVDAWGQGTTVTVSS

SEQ ID NO: 120 - protein, 128 aa, Artificial Sequence
  An artificially synthesized sequence
QVQLVESGGGLVQPGRSLRLSCAASGFTFSNAWMHWVRQAPGKGLEWVAQIKDRSSSYLTYYAESVKGRFTISRDDSKNSIYLQMNSLKTEDTAVYYCHYVHYASSSTLLPSFGVDAWGQGTTVTVSS

SEQ ID NO: 121 - protein, 128 aa, Artificial Sequence
  An artificially synthesized sequence
QVQLVESGGGLVQPGRSLRLSCAASGFTFSNAWMHWVRQAPGKGLEWVAQIKDKGNAYAAYYAPSVKGRFTISRDDSKNSIYLQMNSLKTEDTAVYYCHYVHYSAASTVLPAFGVDAWGQGTTVTVSS

SEQ ID NO: 122 - protein, 128 aa, Artificial Sequence
  An artificially synthesized sequence
QVQLVESGGGLVQPGRSLRLSCAASGFTISSAWMHWVRQAPGKGLEWVAQIKDRANGYAAYYAPSVKGRFTISRDDSKNSIYLQMNSLKTEDTAVYYCRYVHYATAYDGFYGFGVDAWGQGTTVTVSS

SEQ ID NO: 123 - protein, 112 aa, Artificial Sequence
  An artificially synthesized sequence
DIVMTQSPLSLPVTPGEPASISCRSSQELVHTNRHTYLHWYQQKPGQAPRLLIYKVSNRFPGVPDRFSGSGSGTDFTLKISRVEAEDVGVYYCAQGTQVPFTFGQGTKLEIK

SEQ ID NO: 124 - protein, 112 aa, Artificial Sequence; identical to SEQ ID NO: 57
  An artificially synthesized sequence

SEQ ID NO: 125 - protein, 112 aa, Artificial Sequence
  An artificially synthesized sequence
DIVMTQSPLSLPVTPGEPASISCSASESLLHNNRHTYLHWYQQKPGQAPRLLIYRVSNRFNGVPDRFSGSGSGTDFTLKISRVEAEDVGVYYCGQGTQVPFTFGQGTKLEIK

SEQ ID NO: 126 - protein, 112 aa, Artificial Sequence
  An artificially synthesized sequence
DIVMTQSPLSLPVTPGEPASISCRTSQELVHINRYIFLHWYQQKPGQAPRLLIYKVYNRFQGVPDRFSGSGSGTDFTLKISRVEAEDVGVYYCAQGTSVPYTFGQGTKLEIK

SEQ ID NO: 127 - protein, 112 aa, Artificial Sequence
  An artificially synthesized sequence
DIVMTQSPLSLPVTPGEPASISCAASEELVHINRYIFLHWYQQKPGQAPRLLIYKVSNRFIGVPDRFSGSGSGTDFTLKISRVEAEDVGVYYCAQGSQVPFTFGQGTKLEIK

SEQ ID NO: 128 - protein, 112 aa, Artificial Sequence
  An artificially synthesized sequence
DIVMTQSPLSLPVTPGEPASISCSTSEELLHINRFIFLHWYQQKPGQAPRLLIYKVANRFMGVPDRFSGSGSGTDFTLKISRVEAEDVGVYYCAQGSQVPFTFGQGTKLEIK

SEQ ID NO: 129 - protein, 112 aa, Artificial Sequence
  An artificially synthesized sequence
DIVMTQSPLSLPVTPGEPASISCSSSQELVHANRQIYLHWYQQKPGQAPRLLIYKVANRFPGVPDRFSGSGSGTDFTLKISRVEAEDVGVYYCAQGTQVPFTFGQGTKLEIK

SEQ ID NO: 130 - protein, 112 aa, Artificial Sequence
  An artificially synthesized sequence
DIVMTQSPLSLPVTPGEPASISCTASQELVHINRFIFLHWYQQKPGQAPRLLIYKVSNRFIGVPDRFSGSGSGTDFTLKISRVEAEDVGVYYCAQGSQVPFTFGQGTKLEIK

SEQ ID NO: 131 - protein, 112 aa, Artificial Sequence
  An artificially synthesized sequence
DIVMTQSPLSLPVTPGEPASISCQSSESLVHVNRHTYLHWYQQKPGQAPRLLIYKVYNRFPGVPDRFSGSGSGTDFTLKISRVEAEDVGVYYCAQGTSVPFTFGQGTKLEIK

SEQ ID NO: 132 - protein, 112 aa, Artificial Sequence
  An artificially synthesized sequence
DIVMTQSPLSLPVTPGEPASISCSASEELVHINRYIFLHWYQQKPGQAPRLLIYKVYNRFMGVPDRFSGSGSGTDFTLKISRVEAEDVGVYYCGQGSSVPFTFGQGTKLEIK

SEQ ID NO: 133 - protein, 112 aa, Artificial Sequence
  An artificially synthesized sequence
DIVMTQSPLSLPVTPGEPASISCRTSEELLHINRFTFLHWYQQKPGQAPRLLIYKVSNRFMGVPDRFSGSGSGTDFTLKISRVEAEDVGVYYCAQGSAVPFTFGQGTKLEIK

SEQ ID NO: 134 - protein, 112 aa, Artificial Sequence
  An artificially synthesized sequence
DIVMTQSPLSLPVTPGEPASISCTSDQELVHMNRHTYLHWYQQKPGQAPRLLIYKVYNRFPGVPDRFSGSGSGTDFTLKISRVEAEDVGVYYCAQGTSVPFTFGQGTKLEIK

SEQ ID NO: 135 - protein, 112 aa, Artificial Sequence
  An artificially synthesized sequence
DIVMTQSPLSLPVTPGEPASISCRTSEELLHINRYIFLHWYQQKPGQAPRLLIYKVQNRFSGVPDRFSGSGSGTDFTLKISRVEAEDVGVYYCAQGSAVPFTFGQGTKLEIK

SEQ ID NO: 136 - protein, 112 aa, Artificial Sequence
  An artificially synthesized sequence
DIVMTQSPLSLPVTPGEPASISCYASEELLHINRHIYLHWYQQKPGQAPRLLIYKVYNRFNGVPDRFSGSGSGTDFTLKISRVEAEDVGVYYCAQGSSVPFTFGQGTKLEIK

SEQ ID NO: 137 - protein, 112 aa, Artificial Sequence
  An artificially synthesized sequence
DIVMTQSPLSLPVTPGEPASISCRASEELLHINRHIFLHWYQQKPGQAPRLLIYKVYNRFTGVPDRFSGSGSGTDFTLKISRVEAEDVGVYYCAQGTSVPFTFGQGTKLEIK

SEQ ID NO: 138 - protein, 112 aa, Artificial Sequence
  An artificially synthesized sequence
DIVMTQSPLSLPVTPGEPASISCQTSEELVHINRFIFLHWYQQKPGQAPRLLIYKVSNRFSGVPDRFSGSGSGTDFTLKISRVEAEDVGVYYCAQGTSVPFTFGQGTKLEIK

SEQ ID NO: 139 - protein, 115 aa, Artificial Sequence
  An artificially synthesized sequence
QVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSS

SEQ ID NO: 140 - protein, 112 aa, Artificial Sequence
  An artificially synthesized sequence
DIVMTQSPLSLPVTPGEPASISCRSSQSLVHSNRNTYLHWYQQKPGQAPRLLIYKVSNRFSGVPDRFSGSGSGTDFTLKISRVEAEDVGVYYCSQNTHVPPTFGQGTKLEIK

SEQ ID NO: 141 - protein, 450 aa, Artificial Sequence
  An artificially synthesized sequence
DIVMTQSPLSLPVTPGEPASISCRSSQSLVHSNRNTYLHWYQQKPGQAPRLLIYKVSNRFSGVPDRFSGSGSGTDFTLKISRVEAEDVGVYYCSQNTHVPPTFGQGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPHHHHHHHH

SEQ ID NO: 142 - protein, 449 aa, Artificial Sequence
  An artificially synthesized sequence
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 143 - protein, 222 aa, Artificial Sequence
  An artificially synthesized sequence
QVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 216 aa, Artificial Sequence
  An artificially synthesized sequence
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 122 aa, Artificial Sequence
  An artificially synthesized sequence
EVQLVETGGSLVQPGKSLKLTCATSGFTFSNAWMHWVRQSPEKQLEWVAQIKAKSNNYATYYAESVKGRFTISRDDSKSNIYLQMNSLKEEDTAVYYCRYVHYGAYYGVDAWGQGTSVTVSS

SEQ ID NO: 146 - protein, 112 aa, Artificial Sequence; identical to SEQ ID NO: 85
  An artificially synthesized sequence

SEQ ID NO: 147 - protein, 22 aa, Artificial Sequence
  An artificially synthesized sequence
LQDPCSNCPAGTFCDNNRNQIC

SEQ ID NO: 148 - protein, 41 aa, Artificial Sequence
  An artificially synthesized sequence
SPCPPNSFSSAGGQRTCDICRQCKGVFRTRKECSSTSNAEC

SEQ ID NO: 149 - protein, 31 aa, Artificial Sequence
  An artificially synthesized sequence
DCTPGFHCLGAGCSMCEQDCKQGQELTKKGC

SEQ ID NO: 150 - protein, 69 aa, Artificial Sequence
  An artificially synthesized sequence
KDCCFGTFNDQKRGICRPWTNCSLDGKSVLVNGTKERDVVCGPSPADLSPGASSVTPPAPAREPGHSPQ

SEQ ID NO: 151 - protein, 94 aa, Artificial Sequence
  An artificially synthesized sequence
LQDPCSNCPAGTFCDNNRNQICSPCPPNSFSSAGGQRTCDICRQCKGVFRTRKECSSTSNAECDCTPGFHCLGAGCSMCEQDCKQGQELTKKGC

SEQ ID NO: 152 - protein, 63 aa, Artificial Sequence
  An artificially synthesized sequence
LQDPCSNCPAGTFCDNNRNQICSPCPPNSFSSAGGQRTCDICRQCKGVFRTRKECSSTSNAEC

SEQ ID NO: 153 - protein, 141 aa, Artificial Sequence
  An artificially synthesized sequence
SPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQ

SEQ ID NO: 154 - protein, 72 aa, Artificial Sequence
  An artificially synthesized sequence
SPCPPNSFSSAGGQRTCDICRQCKGVFRTRKECSSTSNAECDCTPGFHCLGAGCSMCEQDCKQGQELTKKGC

SEQ ID NO: 155 - protein, 100 aa, Artificial Sequence
  An artificially synthesized sequence
DCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQ

SEQ ID NO: 156 - protein, 336 aa, Artificial Sequence
  An artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPDYKDDDDK

SEQ ID NO: 157 - protein, 107 aa, Artificial Sequence [sequence omitted]

SEQ ID NO: 158 - protein, 112 aa, Artificial Sequence; identical to SEQ ID NO: 78
  An artificially synthesized sequence

SEQ ID NO: 159 - protein, 15 aa, Artificial Sequence
  An artificially synthesized sequence
QDGNEEMGGITQTPY

SEQ ID NO: 160 - protein, 128 aa, Artificial Sequence
  An artificially synthesized sequence
QVQLVESGGGLVQPGRSLRLSCAASGFTFSNAWMHWVRQAPGKGLEWVAQIKDKSNNYATYYAESVKGRFTISRDDSKNSIYLQMNSLKTEDTAVYYCRYVHYGAGGSGGSYYGVDAWGQGTTVTVSS

SEQ ID NO: 161 - protein, 112 aa, Artificial Sequence; identical to SEQ ID NO: 85
  An artificially synthesized sequence

SEQ ID NO: 162 - protein, 122 aa, Artificial Sequence; identical to SEQ ID NO: 145
  An artificially synthesized sequence

SEQ ID NO: 163 - protein, 112 aa, Artificial Sequence
  An artificially synthesized sequence
DVVMTQTPVSMSVSLGGQVSISCRSSQSLVHNNGNTYVSWYIQKPSQSPQLLIYKVSNRFSGISDRFSGSGSGTDFTLKISRVEPDDLGVYYCGQGTQDPYTFGAGTKLELK

SEQ ID NO: 164 - protein, 119 aa, Artificial Sequence
  An artificially synthesized sequence
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 165 - protein, 107 aa, Artificial Sequence
  An artificially synthesized sequence
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 166 - protein, 447 aa, Artificial Sequence
  An artificially synthesized sequence
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 167 - protein, 447 aa, Artificial Sequence
  An artificially synthesized sequence
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLDSDGSFFLYSKLTVDKSRWQQGNVFSCSVMHEALHNRYTQKSLSLSP

SEQ ID NO: 168 - protein, 447 aa, Artificial Sequence
  An artificially synthesized sequence
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 169 - protein, 445 aa, Artificial Sequence
  An artificially synthesized sequence
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNRYTQKSLSLSPHHHHHHHH

SEQ ID NO: 170 - protein, 226 aa, Artificial Sequence
  An artificially synthesized sequence
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSARTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - protein, 458 aa, Artificial Sequence
  An artificially synthesized sequence
EVQLVETGGSLVQPGKSLKLTCATSGFTFSNAWMHWVRQSPEKQLEWVAQIKAKSNNYATYYAESVKGRFTISRDDSKSNIYLQMNSLKEEDTAVYYCRYVHYGAYYGVDAWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPDYKDDDDK

SEQ ID NO: 172 - protein, 219 aa, Artificial Sequence
  An artificially synthesized sequence
DVVMTQTPVSMSVSLGGQVSISCRSSQSLVHNNGNTYVSWYIQKPSQSPQLLIYKVSNRFSGISDRFSGSGSGTDFTLKISRVEPDDLGVYYCGQGTQDPYTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - protein, 461 aa, Artificial Sequence
  An artificially synthesized sequence
EVQLVETGGSLVQPGKSLKLTCATSGFTFSNAWMHWVRQSPEKQLEWVAQIKAKGGSSNNYATYYAESVKGRFTISRDDSKSNIYLQMNSLKEEDTAVYYCRYVHYGAYYGVDAWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPDYKDDDDK

SEQ ID NO: 174 - protein, 464 aa, Artificial Sequence
  An artificially synthesized sequence
EVQLVETGGSLVQPGKSLKLTCATSGFTFSNAWMHWVRQSPEKQLEWVAQIKAKGGSGGSSNNYATYYAESVKGRFTISRDDSKSNIYLQMNSLKEEDTAVYYCRYVHYGAYYGVDAWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPDYKDDDDK

SEQ ID NO: 175 - protein, 6 aa, Artificial Sequence
  An artificially synthesized sequence
GGSGGS

SEQ ID NO: 176 - protein, 467 aa, Artificial Sequence
  An artificially synthesized sequence
EVQLVETGGSLVQPGKSLKLTCATSGFTFSNAWMHWVRQSPEKQLEWVAQIKAKGGSGGSGGSSNNYATYYAESVKGRFTISRDDSKSNIYLQMNSLKEEDTAVYYCRYVHYGAYYGVDAWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPDYKDDDDK

SEQ ID NO: 177 - protein, 9 aa, Artificial Sequence
  An artificially synthesized sequence
GGSGGSGGS

SEQ ID NO: 178 - protein, 461 aa, Artificial Sequence
  An artificially synthesized sequence
EVQLVETGGSLVQPGKSLKLTCATSGFTFSNAWMHWVRQSPEKQLEWVAQIKAKSNNYATYYAESVKGRFTISRDGGSDSKSNIYLQMNSLKEEDTAVYYCRYVHYGAYYGVDAWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPDYKDDDDK

SEQ ID NO: 179 - protein, 464 aa, Artificial Sequence
  An artificially synthesized sequence
EVQLVETGGSLVQPGKSLKLTCATSGFTFSNAWMHWVRQSPEKQLEWVAQIKAKSNNYATYYAESVKGRFTISRDGGSGGSDSKSNIYLQMNSLKEEDTAVYYCRYVHYGAYYGVDAWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPDYKDDDDK

SEQ ID NO: 180 - protein, 467 aa, Artificial Sequence
  An artificially synthesized sequence
EVQLVETGGSLVQPGKSLKLTCATSGFTFSNAWMHWVRQSPEKQLEWVAQIKAKSNNYATYYAESVKGRFTISRDGGSGGSGGSDSKSNIYLQMNSLKEEDTAVYYCRYVHYGAYYGVDAWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPDYKDDDDK

SEQ ID NO: 181 - protein, 461 aa, Artificial Sequence
  An artificially synthesized sequence
EVQLVETGGSLVQPGKSLKLTCATSGFTFSNAWMHWVRQSPEKQLEWVAQIKAKSNNYATYYAESVKGRFTISRDDSKSNIYLQMNSLKEEDTAVYYCRYVHYGAGGSYYGVDAWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPDYKDDDDK

SEQ ID NO: 182 - protein, 464 aa, Artificial Sequence
  An artificially synthesized sequence
EVQLVETGGSLVQPGKSLKLTCATSGFTFSNAWMHWVRQSPEKQLEWVAQIKAKSNNYATYYAESVKGRFTISRDDSKSNIYLQMNSLKEEDTAVYYCRYVHYGAGGSGGSYYGVDAWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPDYKDDDDK

SEQ ID NO: 183 - protein, 467 aa, Artificial Sequence
  An artificially synthesized sequence
EVQLVETGGSLVQPGKSLKLTCATSGFTFSNAWMHWVRQSPEKQLEWVAQIKAKSNNYATYYAESVKGRFTISRDDSKSNIYLQMNSLKEEDTAVYYCRYVHYGAGGSGGSGGSYYGVDAWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPDYKDDDDK

SEQ ID NO: 184 - protein, 122 aa, Artificial Sequence
  An artificially synthesized sequence
QVQLVESGGGVVQPGRSLRLSCAASGFTFSNAWMHWVRQAPGKGLEWVAQIKAKSNNYATYYAESVKGRFTISRDDSKNSLYLQMNSLKTEDTAVYYCRYVHYGAYYGVDAWGQGTTVTVSS

SEQ ID NO: 185 - protein, 112 aa, Artificial Sequence; identical to SEQ ID NO: 85
  An artificially synthesized sequence

SEQ ID NO: 186 - protein, 336 aa, Artificial Sequence; identical to SEQ ID NO: 156
  An artificially synthesized sequence

SEQ ID NO: 187 - protein, 107 aa, Artificial Sequence [sequence omitted]

SEQ ID NO: 188 - protein, 230 aa, Artificial Sequence
  An artificially synthesized sequence
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNRYTQKSLSLSP

SEQ ID NO: 189 - protein, 449 aa, Artificial Sequence
  An artificially synthesized sequence
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 214 aa, Artificial Sequence [sequence omitted]

SEQ ID NO: 191 - protein, 7 aa, Artificial Sequence
  An artificially synthesized sequence
GGSGGGS

SEQ ID NO: 192 - protein, 8 aa, Artificial Sequence
  An artificially synthesized sequence
GGGSGGGS

SEQ ID NO: 193 - protein, 458 aa, Artificial Sequence
  An artificially synthesized sequence
QVQLVESGGGLVQPGRSLRLSCAASGFTFSNAWMHWVRQAPGKGLEWVAQIKDKSNNYATYYAESVKGRFTISRDDSKNSIYLQMNSLKTEDTAVYYCRYVHYAAYYGVDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPDYKDDDDK

SEQ ID NO: 194 - protein, 28 aa, Artificial Sequence
  An artificially synthesized sequence
  misc_feature (1)..(1): Xaa can be any naturally occurring amino acid
XDGNEEMGGITQTPYKVSISGTTVILTC

SEQ ID NO: 195 - protein, 163 aa, Artificial Sequence
  An artificially synthesized sequence
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQ

SEQ ID NO: 196 - DNA, 18 bp, Artificial Sequence
  An artificially synthesized sequence
gcgtcacactttgctatg

SEQ ID NO: 197 - DNA, 21 bp, Artificial Sequence
  An artificially synthesized sequence
tgagttccacgacaccgtcac

SEQ ID NO: 198 - DNA, 55 bp, Artificial Sequence
  An artificially synthesized sequence
gttattactcgcggcccagccggccatggcggatatcgtgatgacccagtctccg

SEQ ID NO: 199 - DNA, 27 bp, Artificial Sequence
  An artificially synthesized sequence
tttaatttccagtttggtaccttgacc

SEQ ID NO: 200 - protein, 8 aa, Artificial Sequence
  An artificially synthesized sequence
DYKDDDDK

SEQ ID NO: 201 - protein, 189 aa, Artificial Sequence
  An artificially synthesized sequence
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQHHHHHHGGGGSGLNDIFEAQKIEWHE

SEQ ID NO: 202 - protein, 121 aa, Artificial Sequence [sequence omitted]

SEQ ID NO: 203 - protein, 109 aa, Artificial Sequence [sequence omitted]

SEQ ID NO: 204 - protein, 128 aa, Artificial Sequence
  An artificially synthesized sequence
QVQLVESGGGLVQPGRSLRLSCAASGFTFSNAWMHWVRQAPGKGLEWVAQIKDRANSYNTYYAESVKGRFTISRDDSKNSIYLQMNSLKTEDTAVYYCRYVHYTTYAGSSFSYGVDAWGQGTTVTVSS

SEQ ID NO: 205 - protein, 112 aa, Artificial Sequence; identical to SEQ ID NO: 78
  An artificially synthesized sequence

SEQ ID NO: 206 - protein, 115 aa, Artificial Sequence
  An artificially synthesized sequence
QVQLVQSGAEVKKPGASVTVSCKASGYTFTDYEMHWIRQPPGEGLEWIGAIDGPTPDTAYSEKFKGRVTLTADKSTSTAYMELSSLTSEDTAVYYCTRFYSYTYWGQGTLVTVSS

SEQ ID NO: 207 - protein, 112 aa, Artificial Sequence; identical to SEQ ID NO: 78
  An artificially synthesized sequence

SEQ ID NO: 208 - protein, 330 aa, Artificial Sequence [sequence omitted]

SEQ ID NO: 209 - protein, 326 aa, Artificial Sequence
  An artificially synthesized sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 210 - protein, 377 aa, Artificial Sequence
  An artificially synthesized sequence
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 211 - protein, 327 aa, Artificial Sequence
  An artificially synthesized sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 212 - protein, 330 aa, Artificial Sequence [sequence omitted]

SEQ ID NO: 213 - protein, 326 aa, Artificial Sequence; identical to SEQ ID NO: 209
  An artificially synthesized sequence

SEQ ID NO: 214 - protein, 377 aa, Artificial Sequence; identical to SEQ ID NO: 210
  An artificially synthesized sequence

SEQ ID NO: 215 - protein, 327 aa, Artificial Sequence; identical to SEQ ID NO: 211
  An artificially synthesized sequence

SEQ ID NO: 216 - protein, 4 aa, Artificial Sequence
  An artificially synthesized sequence
GGGS

SEQ ID NO: 217 - protein, 4 aa, Artificial Sequence
  An artificially synthesized sequence
SGGG

SEQ ID NO: 218 - protein, 5 aa, Artificial Sequence
  An artificially synthesized sequence
GGGGS

SEQ ID NO: 219 - protein, 5 aa, Artificial Sequence
  An artificially synthesized sequence
SGGGG

SEQ ID NO: 220 - protein, 6 aa, Artificial Sequence
  An artificially synthesized sequence
GGGGGS

SEQ ID NO: 221 - protein, 6 aa, Artificial Sequence
  An artificially synthesized sequence
SGGGGG

SEQ ID NO: 222 - protein, 7 aa, Artificial Sequence
  An artificially synthesized sequence
GGGGGGS

SEQ ID NO: 223 - protein, 7 aa, Artificial Sequence
  An artificially synthesized sequence
SGGGGGG

SEQ ID NO: 224 - DNA, 549 bp, Artificial Sequence
  An artificially synthesized sequence
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaattga

SEQ ID NO: 225 - protein, 182 aa, Artificial Sequence
  An artificially synthesized sequence
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 226 - DNA, 516 bp, Artificial Sequence
  An artificially synthesized sequence
atggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaagtga

SEQ ID NO: 227 - protein, 171 aa, Artificial Sequence
  An artificially synthesized sequence
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 228 - DNA, 624 bp, Artificial Sequence
  An artificially synthesized sequence
atgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctga

SEQ ID NO: 229 - protein, 207 aa, Artificial Sequence
  An artificially synthesized sequence
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI